(19) United States
(12) Patent Application Publication
(10) Pub. No.: US 2025/0195722 A1
(43) Pub. Date: Jun. 19, 2025

(54) ELONGATED COMPOSITE FOR AN IMPLANT

(71) Applicant: 206 Ortho, Inc., Deerfield, NH (US)

(72) Inventors: Jeffrey A. D'AGOSTINO, Deerfield, NH (US); Stephen S. KEANEY, Groton, MA (US); Elizabeth NELSON, Wellesley, MA (US)

(73) Assignee: 206 Ortho, Inc., Deerfield, NH (US)

(21) Appl. No.: 18/252,849

(22) PCT Filed: Nov. 12, 2021
(86) PCT No.: PCT/US2021/059209

§ 371 Date: May 12, 2023

Related U.S. Application Data
(63) Continuation of application No. PCT/US2020/060612, filed on Nov. 13, 2020.
(60) Provisional application No. 63/190,748, filed on May 19, 2021.
(60) Provisional application No. 63/187,899, filed on May 12, 2021.

(51) Int. Cl. A61L 27/44

(52) U.S. Cl. CPC A61L 27/446; A61L 2430/02

(57) ABSTRACT

An elongated composite for an implant comprising: one or more composite elements includes a core region including a plurality of fibrous bundles, which are preferably braided, woven, bound, interlaced and/or interlocked by bias fiber elements, and a polymerizable material or polymeric material which fills gaps between the fibrous bundles; and a covering over the one or more composite elements. The elongated composite and composite elements have a longitudinal axis and the fibrous bundles include a plurality of filaments that are twisted along the longitudinal axis and a polymeric/polymerizable matrix.

### CROSS-REFERENCE TO RELATED APPLICATIONS

This patent application is related to U.S. Provisional Patent Application No. 63/187,899, filed on May 12, 2021, and U.S. Provisional Patent Application No. 63/190,748, filed on May 19, 2021, all of which are incorporated by reference herein in their entirety. Additionally, this patent application is related to PCT Patent Application No. PCT/US2020/060612, filed on Nov. 13, 2020, which claims priority to U.S. Provisional Patent Application No. 62/934,922, filed on Nov. 13, 2019; U.S. Provisional Patent Application No. 62/935,001, filed on Nov. 13, 2019; and U.S. Provisional Patent Application No. 63/093,659, filed on Oct. 19, 2020, all of which are incorporated by reference herein in their entirety.

### FIELD

This invention relates to designs, materials, and methods of manufacturing composite materials comprising hierarchical structures that integrate architectural elements across multiple and within scale lengths.

In addition, this invention relates to methods and apparatus for treating bones, and more particularly to methods and apparatus for treating bone fractures and/or for fortifying and/or augmenting bone in mammals.

In addition, this invention relates to methods and apparatus for treating bones, and more particularly to methods and apparatus for treating bone fractures and/or for fortifying and/or augmenting bone in mammals and relates to novel composite structures which may be used for medical and non-medical applications.

### BACKGROUND

Conventional materials are subject to a number of trade-offs with respect to their properties. Rigid materials (e.g., higher Young's modulus) tend to exhibit brittle failure while elastic materials (e.g., lower Young's modulus) tend to exhibit ductile failure. In end-use applications where load-bearing capacity is required, rigid materials may be chosen but at the expense of inviting brittle failure. In some end-use applications, brittle failure is not desired. Furthermore, in some end-use applications, biodegradable and/or bioabsorbable materials are desired. Biodegradable and/or bioabsorbable materials typically are not sought for end-use applications where load-bearing capacity is required because they tend to lack the requisite rigidity. With respect to biodegradability and/or bioabsorbability, some end-use applications seek materials with an adequate rate of material degradation that does not result in compromising load-bearing properties before the useful life of the material has ended. Therefore, in end-use applications where rigidity is required for load-bearing purposes, ductile failure is a preferable failure mode, biodegradability and/or bioabsorbability is required, and a particular rate of material degradation is sought, no single material can bridge the gap between all of the aforementioned end-use requirements. This problem is communicated hereunder through the exemplary lens of composite implants for medical use.

In the event of bone fractures and certain medical conditions (e.g., osteoporosis), there are several conventional tools to support the bone during healing and/or fortify/augment a bone so it can withstand forces exerted during normal physical activity. External stabilizers (e.g., plaster casts, braces, etc.) tend to interfere with a patient's normal daily activities. In the field of veterinary medicine, some modes of fractures are difficult to apply external stabilizers to. Furthermore, shortly after application of the external stabilizer, the patient's intervening soft tissue begins to atrophy through disuse, thereby requiring further rehabilitation for the patient. Internal stabilizers (e.g., screws, bone plates, intramedullary nails, etc.) provide a more effective stabilization of the fracture than external stabilizers since they are able to directly interface with the bone. However, installing these internal stabilizers requires an invasive surgical procedure (i.e., a relatively large opening (e.g., incision) and displacement of tissue/organs/bone) and sometimes requires an additional procedure to remove the stabilizer.

Internal stabilizers (“orthopedic implants”) are typically fabricated from metals, polymers, and degradable biocomposites. With metal implants, removal surgery is often required or else they become permanent foreign objects in the body. Furthermore, pain is caused to the patient—with the intensity being dependent on where the implant is located. With polymeric implants, load bearing strength is lesser than metal implants and removal surgery is still required. With biocomposites, materials are substantially limited. As a result, it is difficult to achieve mechanical properties of the composite that are many times stronger than the loads expected during use. If a biocomposite were to fail, brittle failure is very traumatic to the patient and could injure the patient. Thus, it is important for the implant to exhibit ductile failure, which may still cause some trauma but would be far more preferable than brittle failure.

Biocomposite hardware (e.g., pins, plates, screws, etc.), while absorbed into the body over time and not needing to be removed, tend to be brittle and do not handle load bearing as well as metal implants. Brittleness may be attributed to the properties of biodegradable polymer and low aspect ratio fillers that make up biocomposite pins. This often results in breakage during the insertion procedure, adding cost and complexity to the procedure as well as trauma to the patient. In some circumstances, bone cements (e.g., polymer-based cements, calcium salt-based cements) are injected into the interior of the bone in an attempt to stabilize the bone. However, while these bone cements are typically capable of withstanding significant compressive loading, they are also extremely brittle and typically cannot withstand significant tensile loading. This limits their application in instances where the loading on the bone may include a tensile component, which is particularly seen in long bones (e.g., tibia).

The removable implants discussed above do not adequately meet the mechanical property needs to enable patients to perform normal physical activity without restriction. To do so, the materials require low density, stiffness, strength, and fracture resistance. However, conventional materials that achieve these mechanical properties are brittle. Thus, it can be appreciated that different materials and physical construction of implants pose trade-offs with respect to suitability for prolonged presence in the body and mechanical properties. Thus, it will be seen that a new approach is needed for designing and making implants with enhanced properties to meet the mechanical properties required for orthopedic implants.

In addition to mechanical properties, control of degradation rate is critical to the adequate performance of a degradable implant. If the implant degrades too rapidly, the implant may not have the strength required to provide structural support. If the implant degrades too slowly, a lag in degradation may result in negative changes to the environment in which the implant resides. There are three main stages of degradation which should be accounted for when designing a degradable implant. The initial degradation upon implantation drives the initial release of the implant's material into the body. Once the initial portion of the degradation profile has occurred, creating a steady state at which the degradation occurs over time is necessary. During steady state degradation, the transfer of the properties of the implant back to the healing bone occurs. This degradation rate needs to be tuned for the specific application. The last step of degradation is the final transfer from implant to the healed bone. Here it is important to design the implant such that the material is completely absorbed by the body. Ultimately the implant must be strong enough for the length of healthy process and then disappear. Conventional degradable implants struggle to cover all three stages of degradation, especially creating an implant that maintains the strength and/or transfers the strength back to the bone over the duration of the healing time.

It would be desirable to provide an implant that is bioabsorbable. It would be desirable to provide an implant that can withstand mechanical loads typically exerted through normal daily activity. It would be desirable to provide an implant that exhibits a ductile failure mode. It would be desirable to provide an implant with a tailored degradation. It would be desirable to provide an implant that can be formed into various types of hardware (e.g., pins, screws, plates, etc.). It would be desirable to provide an implant that is constructed with materials and processes that are commercially scalable.

### SUMMARY

The teachings herein relate to composite materials, to unique materials which may be employed in a composite material, and to methods for producing the composite materials or a component of the composite material. Due to unique properties of the materials, unique combinations of materials, and unique constructions of the composite materials, it is now possible to solve a variety of problems, particularly where biodegradability and/or bioabsorbability of the composite or one or more components of the composite is desired. These teachings find applicability in both medical applications and non-medical applications.

The present teachings provide for a composite that may address at least some of the needs identified herein. The composite may comprise a degradable and bioabsorbable polymeric matrix material, and a plurality of fiber bundles dispersed in the polymeric matrix material. The plurality of fiber bundles may include a plurality of degradable fibers (which can include a plurality of filaments). The polymeric matrix material, the degradable fibers, or both may be configured to be degradable according to a predetermined degradation profile, so the composite maintains a sufficient compressive load and, in the event of failure, fails in a ductile failure mode from a time of implantation until at least about 24 weeks after initiation of degradation. The composite may occupy an envelope defined by a perimetric surface geometry and/or a volume of the composite. The remaining discussion in this Summary section includes general teachings applicable to all embodiments. The various features may be combined with each other to define a composite. Additionally, properties and characteristics (e.g., degradation rates, mechanical properties or otherwise) that are described in the Detailed Description herein should be regarded as applicable to the various teachings and combinations in this Summary section as well as the various teachings and combinations of the Detailed Description. Likewise, unless clearly set forth as otherwise, the various structures depicted throughout the drawings can be modified to include in their combinations any of the features of this Summary and in the Detailed Description.

For all embodiments, the polymeric matrix material may include a degradable and/or resorbable polymer. The polymer may be selected from poly(lactic acid) (e.g., PLA, PDLA, PLLA; most preferably PDLA 70/30), poly(lactic-co-glycolic acid) (e.g., PLGA 94/6), polyurethane, poly(glycolic acid), polyhydroxyalkanoates, citric acid based polymers, or any combination thereof.

The polymeric matrix material may optionally include a particulate filler, in addition to the plurality of degradable fibers dispersed within the polymeric matrix material.

The plurality of degradable fibers and the optional particulate filler may comprise glass, magnesium hydroxide, or both. The plurality of degradable fibers and the optional particulate filler may comprise some other compound that is degradable, such as an inorganic compound (e.g., an oxide, a silicate, a phosphate, or any combination).

The plurality of degradable fibers and/or the optional particulate filler, during degradation, may release ionic species into an aqueous environment within the envelope.

An identity of the ionic species and/or a concentration of the ionic species in the aqueous environment may alter or otherwise modulate a pH of the aqueous environment. This may be done for assuring biocompatibility, for controlling a degradation rate of an implant, for promoting bone or tissue growth or any combination thereof.

The polymeric matrix material, the plurality of fiber bundles, the plurality of degradable fibers, the optional particulate filler, or any combination thereof may be coated and/or filled with one or more compatibilizers that function to promote osseointegration. Such compatibilizer may be selected and employed in suitable amounts to assure a local environment (in the region of the implant) that fosters biocompatibility, to provide a nutrient for promoting bone or tissue growth or both.

The polymeric matrix material, the plurality of fiber bundles, the plurality of degradable fibers, the optional particulate filler, or any combination thereof may comprise two or more distinct regions of material composition, each of the two or more distinct regions being configured to degrade at different rates.

Two or more distinct regions may degrade (e.g., relative to each other) in a generally sequential manner (i.e., one after another), in a generally staggered manner (i.e., overlapping), or both.

Two or more distinct regions may vary relative to each other in chemical composition over time during degradation.

At least one stage of a degradation profile may include a first stage during which the composite includes a surface texture and/or a surface porosity allowing biological materials between about 0.1 nm and 1,000 nm in their largest dimension (e.g., proteins) to enter into the envelope.

At least one stage of a degradation profile may include a first stage or second stage during which the composite includes a surface texture and/or a surface porosity allowing biological materials between about 1 μm and 30 μm in their largest dimension (e.g., macrophages) to enter into the envelope.

At least one stage of a degradation profile may include an additional stage (e.g., third stage) during which the composite includes a surface texture and/or a surface porosity allowing biological materials between about 30 μm and 600 μm (e.g., between about 30 μm and 300 μm) in their largest dimension (e.g., tissue and/or bone) to enter into the envelope.

A degradation profile may include at least one stage in which there is a surface texture and/or a surface porosity in the envelope. The surface texture and/or the surface porosity may permit washout of ionic species and/or other degradation byproducts (e.g., hydrolyzed polymeric matrix material) from within the envelope and into a surrounding environment of the envelope. The surrounding environment may be no more than about 1 mm from the envelope, more preferably no more than 2 mm from the envelope, more preferably no more than 3 mm from the envelope, or even more preferably no more than about 4 mm from the envelope.

Surface texture and/or the surface porosity may provide for washout of the ionic species and/or other degradation byproducts from within the envelope to the surrounding environment in a sufficient amount so that the pH within the envelope remains within a range of about 5.5 to 10 (e.g., about 5.5 to 7.5), for a period of at least about 24 weeks after initiation of degradation.

A degradation profile may include at least one stage in which a plurality of passages are formed in the composite, the plurality of passages providing washout from the composite and into a surrounding environment, for eliminating ionic species and/or other degradation byproducts from within the envelope.

One or a plurality of passages (e.g., throughholes, channels, divots, other apertures or the like) may provide for washout of ionic species and/or the other degradation byproducts from materials of the composite within the envelope to the surrounding environment. The washout enabled by the structure of a composite (e.g., implant) may be in a sufficient amount so that the pH within the envelope remains within a range of about 5.5 to 10 (e.g., about 5.5 to 7.5), for a period of at least about 24 weeks after initiation of degradation.

A plurality of passages may include a plurality of axial passages (co-axial with the longitudinal axis of the composite), a plurality of transverse passages (co-axial with the transverse axis of the composite), a plurality of radial passages (radially oriented at an angle to the axial/transverse passages), or any combination thereof.

A predetermined degradation profile may include one or more stages characterized by a degradation onset, one or more degradation rates, or both.

A modulus of elasticity of the composite at least about 4 weeks after initiation of degradation may be between about 8,000 MPa and 40,000 MPa, more preferably between about 9,000 MPa and 30,000 MPa, and more preferably between about 9,500 MPa and 25,000 MPa.

A poly dispersion index of the polymeric matrix material at least about 4 weeks after initiation of degradation may be about 50% of the poly dispersion index of the polymeric matrix material prior to initiation of degradation.

At least about 4 weeks from initiation of degradation, a volume of the composite may be more than about 40%, more preferably more than about 25%, or even more preferably about more than about 10% less than the volume of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to measuring volume.

At about 4 weeks from initiation of degradation, a weight of the composite may be no more than about 10%, more preferably 5%, or even more preferably about 2% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

At about 12 weeks from initiation of degradation, a weight of the composite may be no more than about 20% less, more preferably about 15% less, or even more preferably about 12% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

At about 24 weeks from initiation of degradation, a weight of the composite may be about 30% less, more preferably about 25% less, or even more preferably about 22% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

At about 52 weeks from initiation of degradation, a weight of the composite may be no less than about 50% less, more preferably about 35% less, or even more preferably about 20% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

At about 102 weeks from initiation of degradation a weight of the composite may be no less than about 80% less, more preferably about 70% less, or even more preferably about 50% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

At about 4 weeks from initiation of degradation, a compressive modulus of the composite may be about 10 GPa or more, more preferably 15 GPa or more, or more preferably 20 GPa or more, (e.g., between about 10 MPa and 800 MPa) as measured by storing the composite in a fixed volume of buffered saline solution maintained at 37° C. exposed to circulation/agitation (e.g., stir bar or pumped fluid) and an initial pH of about 7.5, and drying the composite prior to compressive testing.

At about 4 weeks from initiation of degradation, a strain at failure of the composite may be about 10% or more, more preferably 15% or more, or more preferably 20% or more in bending, torsion, and/or compression, as measured by storing the composite orthopedic implant in a fixed volume of buffered saline solution maintained at 37° C. exposed to circulation/agitation (e.g., stir bar or pumped fluid) and an initial pH of about 7.5, as measured by storing the composite in a fixed volume of buffered saline solution maintained at 37° C. exposed to circulation/agitation (e.g., stir bar or pumped fluid) and an initial pH of about 7.5, and drying the composite prior to compressive testing.

At about 4 weeks from initiation of degradation, a strain at failure of the composite may be about 5% or more, more preferably 10% or more, or more preferably 25% or more in bending, torsion, and/or compression, as measured by storing the composite orthopedic implant in a fixed volume of buffered saline solution maintained at 37° C. exposed to circulation/agitation (e.g., stir bar or pumped fluid) and an initial pH of about 7.5, as measured by storing the composite in a fixed volume of buffered saline solution maintained at 37° C. exposed to circulation/agitation (e.g., stir bar or pumped fluid) and an initial pH of about 7.5, and drying the composite prior to compressive testing.

As discussed herein, a composite may include a composite (e.g., orthopedic) implant for implantation into a living being. If not specifically stated, mention of “composite” herein contemplates a composite implant, which in turn contemplates an orthopedic implant. The polymeric matrix material, the degradable fibers, or any combination thereof may be configured to be degradable in vivo after implantation into the living being according to a predetermined degradation profile that corresponds with a bone and tissue ingrowth profile so that from the time of implantation until the wound site is healed, the composite implant maintains a sufficient compressive load and, in the event of failure, fails in a ductile failure mode.

The composite orthopedic implant may be characterized by a volume of between about 50 mm³ and 4 cm³ (i.e., “small volume implant”) or a volume of between about 4 cm³ and 25 cm³ (i.e., “medium volume implant”), or a volume of between about 25 cm³ and 300 cm³ (i.e., “large volume implant”).

The composite orthopedic implant may be configured to resist torque, bending, or both.

The composite orthopedic implant may be configured to affix bone, tissue, or both.

The composite orthopedic implant may be in the form of a pin, a screw, an anchor, a nail, an assembly introduced into a containment bag in vivo (e.g., splint), a plate, or any combination thereof.

The composite implant may be a screw, pin, anchor, nail, or plate having or not having one or more apertures. Any such aperture may be throughhole. Any such aperture may be a divot. Any such aperture may extend along a longitudinal axis of the implant, along a transverse axis of the implant or both. The composite implant may define a structure having a higher surface area to volume ratio than the same shaped implant without any apertures. The composite implant may define a structure having a higher surface area to volume ratio than the same shaped implant without any aperture that is higher by a factor of at least 2 times, more preferably 3 times, and still more preferably 5 times.

The apertures may be defined by openings between bias fiber bundles. Bias fiber bundles may be interlocked.

The apertures may be defined by openings between axial fiber bundles. The axial fiber bundles may be interlocked by bias fiber bundles. The apertures may be defined by openings between bias fiber bundles and axial fiber bundles.

The axial fiber bundles may comprise more than one fiber or filament. An axial bundle comprises between 2 and 60 or 120 fibers. An axial bundle may comprise up to 120 fibers in a bundle.

The fibers in the axial fiber bundle may be bound together by bias fiber bundles.

The present teachings provide for a method of forming a reinforcement element rod. The method may comprise feeding one or more bundles of axial fibers through a braider having multiple carriers. The method may comprise forming a braid around the bundles using bias fibers. One or more carriers of the braider may not employed in the braiding so that a partially open braid structure is formed. About 5% or more (optionally about 10% or more, or about 15% or more) of the carriers may not employed in the braiding.

### BRIEF DESCRIPTION OF THE DRAWINGS

FIG. 1 is a plan view of a bone.

FIG. 2 is a perspective view of a composite orthopedic implant in the form of a pin.

FIG. 3 is a sectional view, along line A-A of FIG. 2, of a composite orthopedic implant in the form of a pin.

FIG. 4 is a perspective view of a composite orthopedic implant in the form of a pin.

FIG. 5 is a sectional view, along line B-B of FIG. 4, of a composite orthopedic implant in the form of a pin.

FIG. 6 is a perspective view of a composite orthopedic implant in the form of a screw.

FIG. 7 is a perspective view of a composite orthopedic implant in the form of a screw.

FIG. 8 is a perspective view of a composite orthopedic implant in the form of a screw.

FIG. 9 is a sectional view of a composite orthopedic implant along its transverse axis.

FIG. 10 is a sectional view of a composite orthopedic implant along its transverse axis.

FIG. 11 is a sectional view of a composite orthopedic implant along its transverse axis.

FIG. 12 is a sectional view of a composite orthopedic implant in the segment W of FIG. 10.

FIG. 13 is a sectional view of a composite orthopedic implant along its transverse axis.

FIG. 14 is a sectional view of a composite orthopedic implant in the segment X of FIG. 13.

FIG. 15 is a sectional view of a composite orthopedic implant in the segment X of FIG. 13.

FIG. 16 is a sectional view of a reinforcement element in the form of tape.

FIG. 17 is a sectional view of a reinforcement element in the form of tape.

FIG. 18 is a plan view of a reinforcement element in the form of a rod.

FIG. 19 is a sectional view of a reinforcement element in the form of a rod along line C-C of FIG. 18.

FIG. 20 is a plan view of a reinforcement element in the form of a textile.

FIG. 21 is a plan view of a reinforcement element in the form of a textile.

FIG. 22 is a plan view of a reinforcement element in the form of a sheet.

FIG. 23 is a sectional view of a reinforcement element in the form of a roll.

FIG. 24 illustrates sectional views of composites.

FIG. 25 illustrates various arrangements of fiber bundles in reinforcement elements.

FIG. 26 illustrates various arrangements of axial fiber bundles.

FIG. 27 is a sectional view of a fiber composite.

FIG. 28 is a sectional view of a screw.

FIG. 29 is a sectional view of a screw.

FIG. 30 illustrates a chart.

FIG. 31 illustrates a chart.

FIG. 32 illustrates a chart.

FIG. 33 illustrates a chart.

FIG. 34 illustrates a chart.

FIG. 35 illustrates a chart.

FIG. 36 illustrates a testing apparatus.

FIG. 37 illustrates a chart.

FIG. 38 illustrates a chart.

FIG. 39 illustrates a chart.

FIG. 40 illustrates a chart.

FIG. 41 illustrates a chart.

FIG. 42 illustrates a chart.

FIG. 43 illustrates a chart.

FIG. 44 illustrates a chart.

FIG. 45 illustrates a chart.

FIG. 46 illustrates a chart.

FIG. 47 illustrates a braided structure.

FIG. 48 illustrates an open braided structure.

FIG. 49 illustrates a braided structure.

FIG. 50A illustrates a uniaxial braided structure.

FIG. 50B illustrates a twisted filament braided structure.

FIG. 50C illustrates a uniaxial, twisted filament braided structure.

FIG. 51 illustrates a sectional view of a braided structure.

FIG. 52 illustrates a sectional view of a braided structure.

### DETAILED DESCRIPTION

### Introduction

As will be appreciated, and as mentioned previously, the following includes details about various features of the teachings herein. Unless the context or description clearly and unambiguously indicates to the contrary, features depicted in one combination of features may be employed in other combinations. Properties or material characteristics are general to all combinations. Structural features depicted for illustration in one combination are applicable to other combinations. By way of example, illustration of a combination with a particular polymer or glass is not intended as limiting. Other polymers or glasses taught herein for the same purpose may be used in place of the illustrated material. An open architecture structure, though it may be illustrated in the context of a screw, can be employed for other parts (e.g., pins, rods, nails, plates, etc.). Further, to the extent any illustration or example herein depicts a non-degradable material, it is contemplated that any of the disclosed degradable materials can be substituted in place of the non-degradable material. If not so stated, or if the particular material is specified to the contrary, references to “degradable” materials should also be deemed to include resorbable materials.

Conventional materials are subject to trade-offs with respect to their mechanical properties. Relatively rigid materials (e.g., those having a relatively high modulus, whether compressive, flexural, tensile, and/or torsional) tend to exhibit brittle failure. In other words, they tend not to elongate measurably in plastic deformation before rupture. Relatively elastic materials (e.g., those having a relatively low modulus, whether compressive, flexural, tensile, and/or torsional lower modulus) tend to exhibit ductile failure. In other words, they tend to elongate in plastic deformation before rupture. In end-use applications where load-bearing properties are required, rigid materials may be chosen but at the expense of inviting a possible brittle failure. In some end-use applications, brittle failure is not desired. Therefore, in end-use applications where rigidity is required for load-bearing purposes, ductile failure is a preferable failure mode.

As will be discussed, among various unique features attainable with the present teachings is a controlled failure mode in the event that a composite (e.g., an implant such as a composite orthopedic implant) is subjected to a load that exceeds the ultimate strength exhibited by one or more of the constituent materials of the composite. For example, it is possible that upon being subject to a load that exceeds the maximum compressive strength of a matrix material within a composite of the present teachings, the matrix material will form a crack that propagates until it reaches a fiber, fiber bundle, or reinforcement element, at which point energy from the load will be dissipated by the fiber, fiber bundle, or reinforcement element. This may be due to, at least in part, the travel of adjoining fibers relative to each other, fiber bundles relative to each other, adjoining reinforcement elements relative to each other, or any combination thereof. As a result of such relative travel, it is possible that the composite will exhibit a ductility that exceeds the ductility of individual components.

Among various benefits of the present teachings is the possibility for composites that exhibit attractive load-bearing characteristics. This makes the materials, material combinations, and material systems of the present teachings attractive for any of a variety of different applications, such as a medical device (e.g., a composite orthopedic implant) or a device used for non-medical applications (e.g., a fastener used in commercial or residential construction). For example, among the possible technical benefits is the ability to realize a composite material, structure made therefrom, or both that exhibits (i) a flexural modulus of at least 10 GPa, 15 GPa, or even 20 GPa; (ii) a strain at yield in compression, torsion, and/or tension of at least about 0.02, 0.03, or even 0.05; (iii) a strain at failure in compression, torsion, and/or tension of at least about 0.03, 0.05, or even 0.10; (iv) or any combination of (i), (ii) and (iii).

In general, the composites of the present teachings may find beneficial use for non-load-bearing applications. However, among various benefits of the present teachings, in general, is that composites in accordance with the present teachings can be readily designed, fabricated, and/or used for load-bearing applications.

Furthermore, in some end-use applications, biodegradable and/or bioabsorbable materials are desired. Biodegradable and/or bioabsorbable materials typically are not sought for end-use applications where load-bearing properties are required because these materials tend to lack the requisite rigidity. Typically, highly cross-linked polymers are used for load-bearing applications, but a high degree of cross-linking may cause the material to not, if at all, readily degrade in ambient conditions. Biodegradable and/or bioabsorbable materials may also be susceptible to localized degradation that may result in loss of load-bearing properties, fragmentation, or both. Degradation may also result in a localized accumulation of acidic or basic chemicals that are byproducts of the degradation (e.g., via hydrolysis). Some end-use applications seek materials with an adequate rate of material degradation that does not result in compromising load-bearing properties before the useful life of the material has ended. In some end-use applications, biodegradable and/or bioabsorbable materials with a particular rate of material degradation may be sought.

Until the present teachings, composite materials have been generally unable to bridge the gap between all of the aforementioned end-use requirements, and to simultaneously address each of the various competing concerns. Some composite materials, while able to bridge the gap by providing some of the aforementioned end-use requirements fall short in providing all of them simultaneously. For example, some composites may offer rigidity and biodegradability but also exhibit brittle failure, which may be undesirable. Thus, the present teachings provide for a composite comprising materials constructed in novel configurations that provide rigidity, ductile failure, biodegradability and/or bioabsorbability, and a controlled rate of degradation that have not been achieved by conventional composites.

The present teachings pertain generally to new materials, new structures, new combinations of materials and structures, new systems of materials and structures, or any combination thereof. The teachings in general may find particular suitability in the design, manufacture, or use of composites, or any combination thereof. For example, the teachings generally may find particular suitability in the design, manufacture, or use of composites, or any combination thereof, the composites comprising a plurality of reinforcement elements. The reinforcement elements may comprise fibers, fiber bundles, matrix materials, or any combination thereof, in contact with one another by over at least a portion of their length.

The present teachings provide for a composite that may take the form of a composite implant for medical applications, such as orthopedics. When employed as an implant, the composites of the present disclosure are also able to provide an attractive structure to foster implant compatibility with adjoining tissue and/or bone. For instance, as will be addressed, materials of the present teachings can be employed to achieve an interconnected network of bone and/or tissue with the structure of the implant. Such structure may change over time as the implant degrades.

By way of illustration, the composites of the present teachings may find use as orthopedic devices. The composites of the present teachings may find use as orthopedic devices that are implantable and/or implanted within a living being to repair a fracture of a bone (e.g., a partial and/or complete fracture) soft tissue repair (e.g., ligaments and tendons), or both. The fracture of a bone may include a transverse fracture, spiral fracture, comminuted fracture, impacted fracture, segmented fracture, greenstick fracture, oblique fracture, stress fracture, compression fracture, avulsion fracture, or any combination thereof.

The teachings herein generally contemplate a repaired bone fracture site that includes a first bone portion, second bone portion, and composite (e.g., a composite implant device of the present teachings) connecting the first and second bone portions. It is possible, for example, to locate a composite implant to span a gap between a first and second bone portion that has arisen from a fracture.

In accordance with a general aspect of the present teachings, applicable to all embodiments (unless otherwise expressly stated), some or all of the fibers and/or matrix material may be biodegradable and/or bioabsorbable. For example, some or all of the fibers and/or matrix material may be bioabsorbable within a living being (e.g., a human or animal), so that they may be implanted into such living being and following implantation they will be generally non-toxic to the living being, will not be rejected by the living being (e.g., cause a negative immune response by the living being), and need not thereafter be removed from the living being.

It is anticipated that following a period of time after implantation into a living being, the composite implant of the present disclosure may degrade and become absorbed by the body of the living being. The composite implant of the present disclosure may degrade at a rate until the structure as implanted ceases to exist. Such period of time may be at least 1 week, 4 weeks, 8 weeks, 12 weeks, 18 weeks, 24 weeks, 36 weeks, 48 weeks, 102 weeks, 128 weeks, 156 weeks, 224 weeks, or even longer. The rate may be generally linear, non-linear, or both. The rate may change over time.

Within and/or adjoining the volume originally occupied by the composite implant (e.g., the region of a treatment site), there will be an interpenetrating network of bone, tissue, other biological matter, or any combination thereof. The interpenetrating network of bone, tissue, other biological matter, or any combination thereof may develop generally proportionally to the degradation of the composite implant. The interpenetrating network of bone, tissue, biological matter, or any combination thereof may include materials from which fibers and/or matrix materials are fabricated according to the present teachings. By way of example, minerals originating from the composite implant may be incorporated into an interpenetrating network of bone.

Following a period of 1 week from the time of implantation, an edge of bone in contact or proximal (e.g., within about 7 cm, 5 cm, 3 cm, or 1 cm) to the implant may show an enrichment of certain chemicals (e.g., sodium, calcium, phosphorus, or other element) as compared with bone located remotely (e.g., greater than about 10 cm, 15 cm or more).

Within the medical device field, use as an implant is contemplated. Such implant may include a pin, plate, screw, anchor, spinal implant, brace, splint, stent, valve, dental implant, or orthopedic implant. The dental implant may include an endosteal, subperiosteal, or zygomatic implant. For example, without limitation, the implant may be implanted in a mandible, maxilla, or otherwise. As an orthopedic implant, composites of the present teachings find use for the repair of bones (e.g., within a human or animal, any cranial bones, jaw bones, spinal bones, leg bones, feet bones, arm bones, hand bones, rib bones, shoulder bones, or the like). For example, without limitation, the implant may be implanted in a clavicle, humerus, radius, tibia, ankle, hand, foot, cranium, rib, or otherwise.

Use in the repair of load-bearing bones is a particularly attractive aspect of the present teachings. For example, leg bones (e.g., a femur and/or fibula) may be repaired to include a composite implant of the present teachings connecting a first bone portion with a second bone portion, as described previously. The composite implant may enable use of the repaired leg bone, by the patient, for at least limited activity at least 1 week, 2 weeks, 3 weeks, or even 1 month after implantation. The enabled use of repaired bone is particularly advantageous in the healing process because it is known that limited movement of broken bones promotes proper healing.

The composite of the present teachings address difficulties in the art in terms of use of individual elements of composites. That is, by way of example, it is often necessary for a surgeon to locate an implant or individual elements of an implant within small confines of a body, which requires the individual elements to be readily flexed and manipulated within said confines. Furthermore, the individual elements need to have suitable dimensions (e.g., length and width) to be employed with medical devices (e.g., catheters) that locate the individual elements within the body of a living being. The present teachings illustrate examples of individual elements that achieve this result. Moreover, it will be seen that the teachings address a rapid approach to repairing a bone fracture. It will also be seen that the teachings are suitable for end-use applications outside of the field of orthopedic implants.

As the teachings herein will discuss, fibers, fiber bundles, matrix materials, reinforcement elements, assemblies thereof, or any combination thereof, may be provided to a user in a size and/or shape that affords ease of insertion within relatively small confines within a fracture site of a live being. By way of illustration, one or more fibers, matrix materials, reinforcement elements, and/or assemblies thereof may be defined in size and/or shape to enable the same to be introduced through a relatively small opening (e.g., less than about 20 cm, 10 cm, 6 cm, 4 cm, or even 1 cm). By way of illustration, fibers, matrix materials, reinforcement elements, and/or assemblies thereof may be defined in size and shape to enable the same to be manipulated by a user (e.g., a surgeon) in vivo around a surface having a relatively small radius of curvature of less than (e.g., less than about 10 cm, 6 cm, 4 cm, or even 1 cm). By way of illustration, fibers, matrix materials, reinforcement elements, and/or assemblies thereof may be defined in size and/or shape to enable the same to be manipulated by a user in vivo within a fracture site having a gap (between a first and second bone portion) of less than about 10 cm, 6 cm, 4 cm, or even 1 cm). Accordingly, from the above (and as will be discussed in further detail herein), the present teachings lend themselves well to minimally invasive surgical techniques and attendant benefits of such techniques.

Among many benefits of the present teachings is the reduction in the quantity of stock keeping units (SKUs) in surgical departments. Conventionally, 100 or more, 150 or more, or even 200 or more stock keeping units are employed in surgical departments, the stock keeping units being associated with medical implants or constituent parts thereof having different dimensions and/or configurations. These quantities of stock keeping units are employed so the medical implants may be tailored to patients with differently dimensioned bones and/or tissue, as well as tailored to the particular bones in which the medical implants are to be located during surgery. In contrast, the composite implant of the present teachings (e.g., a splint) may be modular so only 2 or more, 4 or more, or even 6 or more individual SKUs may be provided to surgical departments and physicians may construct a composite implant, in vivo and/or ex vivo, that is tailored to patients with differently dimensioned bones and/or tissue, as well as tailored to the particular bones in which the composite implant is to be located during surgery.

Among many benefits possible from the present teachings are the attractive capabilities of materials, material combinations, and material systems to the in vivo fabrication of implants.

Among many benefits possible from the present teachings are the attractive capabilities of materials, material combinations, and material systems to the ex vivo fabrication of implants. Implants fabricated ex vivo can thereafter be introduced within a living being.

Among many benefits possible from the present teachings are the attractive capabilities of materials, material combinations, and material systems to the partial ex vivo fabrication of implants and thereafter finished fabrication of implants in vivo. Implants partially fabricated ex vivo can thereafter be introduced within a live being.

In one aspect of the present teachings, it is contemplated that kits may be provided to a user (e.g., physician). A kit may include a plurality of implant precursor elements for introduction and assembly, at least partially or entirely, within a living being. The implant precursor elements may include one or more fibers, fiber bundles, matrix materials (e.g., in the form of a reactant, pre-polymer, or otherwise), reinforcement elements, assemblies of any of the foregoing, or any combination thereof.

Among the attractive capabilities of materials, material combinations, and/or material systems in accordance with the present teachings are the ability to implant them within a living being so that they become a composite orthopedic implant in vivo, only after introduction into a live being. In view of the materials, material combinations and material systems in accordance with the present teachings, comparatively fast orthopedic implants can be realized from a point of time from when an opening (e.g., incision) is made. It is possible, for example, from a time when any reactants, precursor elements, or other polymeric-containing or polymeric forming ingredients are activated following introduction into the living being until the material solidifies, hardens, or otherwise rigidifies, a time elapses that is less than about 120 minutes, 100 minutes, 80 minutes, 60 minutes, 40 minutes, 30 minutes, 20 minutes, or even 10 minutes. In this manner, procedure times may be shortened, and a user (e.g., a surgeon) may be able to direct attention much more quickly to demands of other patients.

One or more matrix materials may be introduced into the live being to contact the assembly. The one or more matrix materials, after introduction, may be controlled within the living being so a temperature does not exceed 80° C., 70° C., 60° C., 50° C., or even 40° C. at the interface of the implant and tissue. In other words, the curing of matrix material may be controlled so as to avoid burning or otherwise injuring or causing discomfort to a patient. Conditions within the living being may be controlled so a reaction or other transformation occurs for realizing a stabilized structure (e.g., by solidifying or hardening, either with or without an accompanying chemical reaction) that includes a polymeric-containing matrix material at least partially contacting or at least partially surrounding at least a portion, if not all, of the fibers, fiber bundles, matrix material, reinforcement elements, or any combination thereof that had been introduced into the living being.

Within one illustrative application of the present teachings (namely, an orthopedic implant and method of making the implant for repairing a fracture), a unique mechanical property profile can be achieved that affords attractive mechanical properties for facilitating healing of a bone fracture (e.g., a stiffness characterized by a flexural modulus of about 10 GPa or more, preferably about 15 GPa or more, or even more preferably about 20 GPa or more; a ductility characterized by a strain at failure in torsion and/or tension of about 0.02 or more, preferably about 0.05 or more, or even more preferably about 0.10 or more; or both) for a period of at least 1 week, 2 weeks, 3 weeks, or even 4 weeks from time of implantation in vivo. These mechanical properties must also be exhibited at the time of implantation, where the implant must withstand torsion and tension as the implant is being placed in vivo.

The mechanical properties discussed herein (e.g., elastic modulus, strain at yield, strain at failure, tensile modulus, the like, or any combination thereof) may be attributed to a composite article as a bulk structure comprising fibers, fiber bundles, matrix material, reinforcement elements, or any combination thereof.

The teachings illustrate various approaches toward achieving a composite (e.g., a composite orthopedic implant), which may be degradable, and methods of making and using the same, that is capable of achieving excellent mechanical properties for a sustained period before degradation occurs that would impair structural properties of the composite. For example, a composite orthopedic implant (and methods to make and use the same) that is capable of achieving excellent mechanical properties for a sustained period before degradation occurs so that interpenetrating bone and/or tissue growth may occur. The teachings illustrate various approaches toward achieving a composite (e.g., a composite orthopedic implant) and methods of making and using the same, that is capable of achieving excellent mechanical properties for a sustained period in vivo that interpenetrating bone and/or tissue growth may occur without compromising structural integrity needed for the duration of a healing process.

As is discussed, among advantageous features of the present teachings is the ability to realize in a degradable composite, following its deployment into its intended environment (e.g., in vivo implantation), the compressive, tensile, torsional and/or flexural properties described. Among advantageous features of the present teachings is the ability to realize in a degradable composite, following its deployment into its intended environment (e.g., in vivo implantation), a ductile failure mode.

Among factors contributing to the structural properties of the composites (e.g., implants) are the selection of materials used, the amounts of materials used and/or the arrangement of materials used. For example, it is believed that some or all of the compressive, tensile, torsional and/or flexural properties described herein can be achieved as a result of product designs that use of filaments, fibers and/or fiber bundles formed and arranged for a controlled response to a load in service. It is believed that designs herein may achieve properties that exceed theoretical properties that otherwise would be achieved according to the predictive rule of mixtures principle of composite technologies. Without intending to be bound by theory, it is believed that teachings herein result in structures that may exhibit a mismatch of material properties (e.g., elastic material properties) between adjoining bodies (e.g., adjoining filaments, fibers, fiber bundles, polymeric matrix, filler, etc.) within the composite, such that a free-edge effect is realized. Additionally, it is believed that teachings herein result in structures that may exhibit a mismatch of material properties (e.g., elastic material properties) between adjoining bodies (e.g., adjoining filaments, fibers, fiber bundles, polymeric matrix, filler, etc.) with a high degree of homogeneity within the composite, such that a free-edge effect is minimized. The free edge effect, being characterized by the concentrated occurrence of three-dimensional and singular stress fields at the free edges in the interfaces of the adjoining bodies, may be used to control force and/or energy distribution occasioned by a load. As a result, it is possible to employ the teachings to enable a sequence of local material translation and/or deformation that effects a generally predetermined deformation mode. The predetermined deformation mode may include delamination, crack deflection, and crack bridging. Employment of this methodology advantageously can help reduce the likelihood of a brittle failure of the composite.

The free edge effect may be minimized at least by modulating the thickness of matrix rich regions between adjacent fibers, the dimension of holes in braids (e.g., holes defined by gaps between intersecting bias fibers that may be occupied by matrix), the diameter of filaments, fibers, and/or fiber bundles, or any combination thereof. A thickness of matrix rich regions between adjacent filaments may be less than or equal to the diameter of the filaments (e.g., the diameter of filaments being between about 10 μm and 20 μm). In this manner, the number of total interfaces between the matrix material and fibers may be increased. As fiber diameter is reduced, flexibility of the fiber may be increased, the number of interfaces between fibers may be increased, crimp (out of plane loading) may be increased, and the size of matrix rich regions may be decreased, thus resulting in a more homogenous structure. Nesting of smaller fibers may increase cell size, decrease crimp (out of plane loading), and decrease the size of matrix rich regions, thus resulting in a more homogenous structure. The dimension of holes (e.g., holes defined by gaps between intersecting bias fibers that may be occupied by matrix) in braids may be about 0.5 mm or less, 0.1 mm or less, or even 0.01 mm or less. Decreasing the dimension of holes may decrease the size of matrix rich regions therein. Modulating the cell size may increase or decrease interfaces (e.g., picks per inch) between fibers and/or fiber bundles. Increasing the number of interfaces may decreases mechanical properties of elements in one plane but increase mechanical properties of elements in other planes. For example, decreasing the fiber bundle diameter, may reduce the thickness of a layer comprising the fiber bundles, which may improve through-thickness mechanical properties. The smaller bundle diameter may result in an increased picks per inch (intersections) and crimp (out of plane loading) but these effects may be mitigated by maintaining the cell size of the architecture (e.g., by nesting multiple fibers adjacent to one another to maintain the width of the reinforcement element). the number of total interfaces between the fibers and/or fiber bundles in the braid may be increased or decreased or the same. The diameter of filaments and/or filaments bundles may be reduced (e.g., for filaments about 20 μm or less, 15 μm or less, 13 μm or less, or even 11 μm or less; for fiber bundles about 500 μm or less, 250 μm or less, or even 100 μm or less). In this manner, the total number of interfaces between matrix material and fibers and/or fiber bundles may be increased. For example, the thickness of a structure, fiber, fiber bundle, layer and or region, or combinations of structures can be reduced to improve properties in one plane while the overall architecture is maintained in another plane, e.g., cell size.

By increasing the number of interfaces within a composite article, the number of stress concentrators may be increased. In other words, stresses may translate through the transverse cross-sectional dimension of the composite article and the stress may be distributed across a larger number of stress concentrators as compared to a composite with a comparatively lesser quantity of stress concentrators (i.e., minimizing the free edge effect). Examples within the present teachings will be described herein in which it will be seen that resulting implants can be characterized by an attractive relatively high level of surface area per unit volume occupied by the “footprint” of the implant. This may be achieved in any of a number of ways, including the formation of channels, divots, holes, surface roughening, the like, or any combination thereof. It will also be seen herein that after a period of time following formation of an implant in vivo, an interpenetrating network will be realized as between at least a portion of the implant and bone or tissue that has grown within channels, divots, holes, surface voids defining its roughness, or any combination thereof. The material of the implant and that of the bone and/or tissue will include plural protuberances resulting from the newly formed bone or tissue.

The composite article may be constructed for use in medical applications. The composite article may include physician/dentist examination room or surgical supplies (e.g., tongue depressors, surgical scalpels, ointment jars, cotton-tipped applicators, plastic cups, nasopharyngeal applicators, drape sheets, tissue wipes (e.g., Kimwipes® or facial tissue), gloves, protective instrument covers, bed linens, towels, sponge bowls, emery boards, examination table paper, scopettes, catheters, syringes, needles, needle block foam, syringe tips, scissors, sutures, forceps, retractors, fenestrated drapes, gauze sponges, prep sponges, scrub brushes, surgical clippers, uterine aspirator tubing, adaptors, swabs, mosquito needle holders, bags, twist ties, burr kits, endodontic files, gingivectomy knives, chisels, hatchets, dental scalers, and nasal cannula). The composite article may include wound care supplies (e.g., pressure bandages, surgical tape, sterile gauze, adhesive bandages, post-surgical bras, elastic skin closures, leukostrips, sutures, non-adherent dressings, flexible bandages, hypo allergenic paper tape, non-adhesive membrane pads, non-sterile gauze sponges, porous tape, bandage roll, chest seal, packing strips, sponges, waterproof tape, blister pads, debridement pads, and wound cleansing sponges).

The composite article may be constructed for use in outdoor sporting applications. The outdoor sporting applications may include fishing, hunting, paintballing, and the like. The composite article may include fishing line, nets, bait, lures, flies, hooks, bobbers, filet knives, fishing line weights, crabbing harness rings, crabbing trap lines, crabbing bait holders, paintballs, arrows, tips, nocks, fletching, shotgun shells, shotgun shell wads, clay pigeons, shotgun shot, shotgun slugs, targets, and the like.

The composite article may be constructed for use as household consumer products. The household consumer products may include diapers, sanitary napkins, milk lactation pads, culinary utensils, culinary serving ware, cups, bottles, straws, trays, garbage bags, and the like.

The composite article may be constructed for use as agricultural tools and/or agricultural textiles. The agricultural tools and/or agricultural textiles may include soil wall reinforcement, embankment basal reinforcement, filtration layers (e.g., textile or granules), twine, stakes, mulch, horticultural film, row covers, nursery containers, seed trays, fertilizer bags, pesticide containers, livestock food containers/bags and the like.

The composite article may be constructed for use as fabrics. The fabrics may be employed in the construction of towels, clothes, blinds, couches, seats, bed sheets, pillows, blankets, and the like.

The composite article may be constructed for use as abrasive blasting media.

The composite article may be constructed for use as items to reduce marine waste such as food containers, packaging, plastic bags, rope, fishing traps and nets, buoys, and the like.

### Composite Article

The present teachings provide for a composite article. The composite article may function as a structural reinforcement. The composite article may function to degrade and/or be absorbed during or after its useful life. The composite article may be employed for load-bearing applications, non-load-bearing applications, or both. The composite article may be employed for medical end-use applications, non-medical end-use applications, or both.

The composite article may comprise one or more fibers, fiber bundles, fiber composites, matrix materials, reinforcement elements, fillers, sizings, or any combination thereof. One or any combination of the foregoing may be referred to herein as precursor elements. The composite article may be fabricated from a hierarchal construction of precursor elements.

As referred to herein, hierarchal construction may mean arranging precursor elements together to form a structure, arranging those structures together to form other structures, and so on. By way of example, a plurality of fibers may be arranged together to form a fiber bundle, the fiber bundle may be fixated with matrix material to form a fiber composite, a plurality of fiber composites may be arranged together and fixated with matrix material to form reinforcement element, and a plurality of reinforcement elements may be arranged together and fixated with matrix material to form a core, and a plurality of cores may be arranged together to form a composite article.

The hierarchal construction may provide for a commercially scalable composite article. Precursor elements may be arranged together in a variety of different arrangements to suit a variety of commercial needs. By way of example, precursor elements may be arranged together to form an orthopedic implant in the form of a pin, screw, plates, or anchor by generally employing the same fabrication methodologies, as discussed herein. The composite article may be fabricated to various lengths, widths, thicknesses, volumes, densities, cross-sectional shapes, surface roughness, porosity, passages, or any combination thereof.

The precursor elements may be arranged deliberately to fabricate a composite article with particular properties. The material from which precursor elements are fabricated may be selected deliberately to fabricate a composite article with particular properties. The precursor elements may be arranged to fabricate a composite article which simultaneously provides two or more, four or more, six or more, or even eight or more different properties. By way of example, a composite article can be fabricated to exhibit strength, rigidity, responsiveness, ductile failure, elasticity, biodegradability and/or bioabsorbability, a controlled degradation rate, or any combination thereof.

The mechanical properties of the composite article may include tensile strength, tensile stiffness, compressive strength, compressive stiffness, flexural strength, flexural stiffness, torsional strength, torsional stiffness, sheer strength, sheer stiffness, strain at yield (in compression, stretching, torsion, and bending), strain at failure (in compression, stretching, torsion, and bending), elastic modulus, flexural modulus, bending-stretching coupling, fracture toughness, responsiveness, mechanical strength, Poisson's ratio, tensile strength, ductility, brittleness, damping, pull-out force, driving torque, or any combination thereof. The mechanical properties of the composite article may be modulated by selecting particular precursor elements and arranging the precursor elements in particular ways.

A composite article of the present teachings may have a strain at yield of 2% or more, 3% or more, 5% or more, 7% or more, 10% or more, 15% or more, or even 20% or more in bending, torsion, compression, or any combination thereof.

A composite article of the present teachings may have a strain at failure of about 2% or more, 5% or more, 10% or more, 12.5% or more, 15% or more, or even 20% or more in bending, torsion, compression, or any combination thereof.

A composite article of the present teachings may exhibit at least 10°, 20°, 30°, 45°, 60°, 90°, or even 1200 of angular deflection in bending, torsion, compression, or any combination thereof before yielding.

A composite article of the present teachings may have a failure mode that is not catastrophic at about 2% or more, 5% or more, 10% or more, 12.5% or more, 15% or more, or even 20% or more strain in bending, torsion, compression, or any combination thereof. A composite article of the present teachings may exhibit a failure mode that is ductile.

A composite article of the present teachings may have an elastic modulus that is at least about 8 GPa, 10 GPa, 12 GPa, 15 GPa, 20 GPa, or even 25 GPa in bending, torsion, compression, or any combination thereof.

A composite article of the present teachings may have a tensile modulus of less than about 60 GPa.

Bone typically breaks at 1.5% to 2% strain. Secondary bone healing typically occurs between 2% to 10% strain. It may be particularly advantageous to provide a composite implant with a strain at yield that approaches or even surpasses the performance of bone and that will allow secondary healing to occur.

A composite article of the present teachings may be characterized as exhibiting a stress strain curve, in response to tensile loading, compression loading, or torsional loading, that includes a first portion from which the modulus can be determined. Following the first portion, and from a portion of the curve when yield starts to occur, the stress-strain curve may include a portion that exhibits a sawtooth pattern. The sawtooth pattern may arise from the sequential yielding and/or fracturing of individual structures (e.g., filaments, fibers, fiber bundles, reinforcement elements) that occur as the composite article is stressed. While individual structures may yield and/or fracture at relatively low strains (e.g., between about 1% and about 10%), the yield and/or fracture of the overall composite article may occur at a relatively high strain (e.g., between about 15% and 50%). That is, there may be a plurality (e.g., at least 2, 3, 4, 5, 6, or even 7 or more) fluctuations of stress over a range of strain values, prior to failure of the overall composite article. The onset, frequency, and/or duration sawtooth pattern may be dependent on the tensile strength and flexibility of the individual structures (e.g., filaments, fibers, fiber bundles, reinforcement elements). By arranging fibers in axial bundles, the composite may be toughened due to bridging of stresses, crack deflection, and slip between axial filaments therein. While axial bundles may contribute to a large portion of the overall strength of the composite, an even stronger composite may be realized by combining bias fibers with axial bundles. By combining bias fibers with axial bundles (e.g., binding and/or interlocking), buckling of the axial bundles may be at least partially mitigated and therefore the overall composite may be toughened.

The composite article of the present teachings may be characterized by Poisson's ratio. Poisson's ratio may be the ratio of transverse strain at axial (in compression or extension) or radial (in torsion) strain. Poisson's ratio may describe the property of materials to neck when placed under stress. Necking may refer to the reduction of at least one dimension of the material (e.g., width) as comparted to the material under no stress. Necking may indicate a ductile failure mode of the composite article.

The performance of a composite article may be improved by one or more of the following: modular construction of precursor elements, polymeric material filling interstitial spaces between precursor elements, interlocking and/or bound precursor elements, material selection of the polymeric material, incorporation of energy absorbing fillers, or any combination thereof.

The composite article may be fabricated from materials that are biodegradable and/or bioabsorbable. The composite article may be fabricated to have a predetermined degradation profile. The degradation profile may include one or more, two or more, three or more, or even four or more stages. In one or more stages, the composite article may have a surface texture and/or surface porosity. In general, the surface texture and/or surface porosity may increase in size (e.g., pore diameter) as time progresses. In one or more stages, the composite article may have a plurality of internal passages. In general, the passages may increase in size and numerosity as time progresses. In one or more stages, the composite article may be characterized by a degradation rate. The degradation rate of the composite article in one or more stages may be generally equal to, less than, or greater than the degradation rate in one or more other stages. In general, the degradation rate may increase or decrease as the surface area of the composite article increases due to an increase in the surface texture, surface porosity, passages, or any combination thereof. That is, in some respects (e.g., for certain polymeric material, glass material) an increased surface area may increase the degradation rate by exposing a larger surface area of the composite to degradation conditions. In other respects (e.g., for certain polymeric material, glass material, composite constructions, and the like), an increased surface area may decrease the degradation rate by washing out degradation byproducts that would have otherwise contributed to a pH that promotes degradation. In other words, some polymers are not susceptible to degradation in approximately neutral (e.g., 6.5 to 8.5) pH conditions.

Degradation of the composite article may be modulated by controlling for the volume of matrix rich regions therein. Generally, matrix material degrades slower than glass. Increasing matrix rich regions may increase the component of the composite that degrades slower and reduces the overall degradation rate of the composite article.

The teachings herein reference a composite implant for medical applications. However, the teachings herein are generally applicable to other composite articles for non-medical applications. Accordingly, the following teachings should be regarded as generalized teachings for composite articles and not limited to a composite implant. Moreover, the various teachings are readily adaptable for use with material systems that may not necessarily be completely biodegradable and/or bioabsorbable.

Composites (e.g., composite implants) in accordance with the present teachings may have an elongated structure having a longitudinal axis in the direction of elongation. Such composites may include filaments, fibers, and/or bundles that are arranged to be oriented generally in a direction along the longitudinal axis. Such composites may include filaments, fibers, and/or fiber bundles that are arranged to be twisted about the longitudinal axis or an axis generally parallel to the longitudinal axis. Such composites having twisted filaments, fibers, and/or fiber bundles may have a helical orientation in relation to the longitudinal axis. Therefore, it can be seen, in this aspect of the present teachings, that the filaments, fibers, and/or fiber bundles may not necessarily be uniaxially aligned.

Within the above teachings, there may be a number of examples of construction. For instance, the composite may include a number of fiber bundles, fibers, and/or filaments that may have a first twist rate resulting in an overall fiber bundle, fiber, and/or filament length (i.e., when not twisted is uniaxially extended as compared to when not twisted) or may not include a twist. The twist may be clockwise or counterclockwise. Multiple fiber bundles, fibers, and/or filaments may go in different directions.

Composites may include a number of fiber bundles, fibers, and/or filaments that may be braided, wrapped, into a unitary textile element. The textile element may include one or more axial and/or bias elements intersecting at one or more different angles (e.g. where each of the elements may be bundles, fibers, and/or filaments), the bias elements oriented at an angle of between more than about 10 degrees, 20 degrees, or even 30 degrees or 45 degrees, and less than about 90 degrees, 80 degrees, 70 degrees, or even 60 degrees, relative to the axial. An overall fiber bundle, fiber, and/or filament length may be comparatively smaller when not braided and uniaxially extended as compared to when braided. That is, in a given construction of a composite article having a fixed length, the uniaxially extended element (e.g., fiber) may have a length that is generally equal to the length of the article while a bias element (e.g., fiber) may extend a length that is greater than the length of the article.

Conventional composites may employ a high fiber volume to increase mechanical properties but as a result, are generally brittle. The present teachings overcome the limitations of conventional composites by layering sub-structures hierarchically. For example, a composite according to the present teachings may comprise one or more bundles of fibers impregnated with matrix material, nest the one or more bundles of fibers together, combine the bundles of fibers (axial) with bias fibers that bind and/or interlock, and impregnate the resulting structure with matrix material. As a result, the mechanical properties of all of the individual sub-components (e.g., axial, bias, and matrix material) provides for a composite that exhibits excellent mechanical properties as well as a ductile failure mode. In other words, individual sub-components according to the present disclosure may be chosen and combined to provide a composite having mechanical properties and failure mode that surpasses the mechanical properties and failure mode of the individual sub-components alone. Some types of sub-components may compensate for the mechanical properties and failure mode of other sub-components. Axial bundles may provide improved tensile strength, improved strain rate, and a brittle failure mode. Nesting of multiple axial bundles may provide increased bend strength, increased strain at yield, increased compressive strength, increased stiffness, increased strain at failure, and at least partially contribute to ductile failure mode. Matrix material may provide improved strain rate and at least partially contribute to a ductile failure mode. Applying twist to bundles (axial or bias) may improve load distribution and at least partially contribute to a ductile failure mode.

### Composite Implant

The composite article may include a composite implant. The composite implant may be used for medical applications. The medical applications may include human medicine, veterinary medicine, or both. The composite implant may be employed for treating bone fractures, fortifying bone that is weakened due to malnutrition or disease, attaching tissue (e.g., ligaments or tendons) to bone, or any combination thereof. The composite implant may do so while imposing minimum inconvenience to a patient. As referred to herein, patient may mean a human or animal within which a composite implant is located. The composite implant may be placed in a patient via a minimally invasive procedure (e.g., laparoscopic procedure).

The composite implant may include any suitable form typically utilized for medical implants. The forms may include, but not limited to, pins, screws, nails (e.g., Enders nail), washers, anchors, plates, braces, splints, spinal fixation rods (e.g., distraction rod or compression rod), the like, or any combination thereof. The composite implant may be located on an exterior surface, interior surface, or within an intramedullary canal of a bone, or any combination thereof. The composite implant may extend into and/or through a bone, intramedullary canal of bone, tendon, ligament, or any combination thereof. The composite implant may extend through cortical bone, cancellous bone, or both. The composite implant may be located within a pre-formed opening in a bone. The pre-formed opening may be formed by a physician, such as via drilling.

The composite implant may be characterized as a small volume implant or a large volume implant. A small volume implant may have a volume of between about 50 mm³ and 4 cm³. A medium volume implant may have a volume of between about 4 cm³ and 25 cm³. A large volume implant may have a volume of between about 25 cm³ and 300 cm³. The composite implant may degrade within the body of a living being. Degradation may produce ions and/or molecules that are absorbed by the body of a living being. The ions and/or molecules may accumulate to a concentration within the circulatory system of a living being. Living beings, depending on age, sex, weight, or otherwise, may have a particular physiological threshold tolerance for ions and/or molecules, in excess of which clinically detrimental conditions may arise. At least some implants may have a volume (e.g., small volume implants) whereby even if the implant were to degrade rapidly and contribute to an immediate acute elevation in circulatory system concentration of ions and/or molecules, said concentration would not impose clinically detrimental conditions. At least some implants may have a volume (e.g., large volume implants) whereby if the implant were to degrade rapidly and contribute to an immediate acute elevation in circulatory system concentration of ions and/or molecules, said concentration would impose clinically detrimental conditions. The degradation profile of large volume implants may be modulated according to the present teachings in order to prevent acute elevation of ions and/or molecules and consequently avoid clinically detrimental conditions.

The composite implant of the present disclosure may be in the form of a pin. The pin may include an elongate structure with two opposing ends. The pin may be generally straight along its longitudinal axis.

The pin may include any suitable cross-sectional shape. The cross-sectional shape may be circular, ovoid, triangular, quadrilateral, pentagonal, hexagonal, octagonal, or any combination thereof.

The pin may extend through cortical bone, cancellous bone, an intramedullary canal, or any combination thereof. The pin may be employed to span a gap between a first bone portion and a second bone portion, the gap resulting from a fracture. The pin may extend through two or more bones, three or more bones, or even four or more bones. The pin may be employed to impose an alignment between two or more bones. For example, the pin may be employed to align hammer toe.

The pin may include one or more facets. The facets may be defined by one or more flat edges as viewed in a transverse cross-section of the pin. The facets may correspond to a geometric shape, truncated geometric shape, or both. The shape may include any 3-sided, 4-sided, 5-sided, 6-sided, 7-sided, or even 8-sided polygon. By way of example, a composite with a hexagonal cross-sectional shape may include six facets. The facets may meet at corners.

The pin may include one or more corners. The corners may be defined by the intersection of two edges or facets as views in a transverse cross-section of the pin. The corners may be pointed, tapered, or both.

The pin may include one or more tapered ends. The tapered ends may function to provide for easy insertion of a composite implant into bone or other suitable substrate. The tapered ends may be formed at opposing ends of a composite implant.

The pin may include one or more lobes. The lobes may function to minimize stress when a composite article is inserted into bone or other suitable substrate. The lobes may be defined by a curved or rounded projection including a radius. The lobes may be formed by fiber bundles. For example, fiber bundles located around the perimeter of a composite implant may define the radius of the lobes. The lobes may be fabricated by molding. For example, a core may be overmolded to fabricate lobes. The lobes may be fabricated by machining. For example, an outer region may be lathed to form lobes.

The pin may comprise one or more cannulations. The cannulation may function to provide for ingress of fluid, facilitate an inside-out degradation, or both. The cannulation may be a hollow shaft within a pin. The cannulation may extend at least partially between and/or through distal ends of a pin. The cannulation may extend longitudinally through the pin, through the center of the pin, between two distal ends of the pin, or any combination thereof. The cannulation may have a cross-sectional shape. The shape may be circular, ovoid, triangular, quadrilateral, pentagonal, hexagonal, octagonal, the like, or any combination thereof. The cannulation may be formed by locating a plurality of precursor elements around a removable mandrel.

The pin may be defined by a length, width, diameter, major diameter, minor diameter, cross-sectional length, cross-sectional width, aspect ratio (i.e., ratio of length to width), cross-sectional aspect ratio (i.e., ratio of cross-sectional length to cross-sectional width), or any combination thereof. As referred to herein, the cross-sectional length may mean the largest dimension of a cross-section, or the length along a major axis. As referred to herein, the cross-sectional width may mean the smallest dimension of a cross-section, or a length along a minor axis.

The length of the pin may be about 10 mm or more, 20 mm or more, or even 30 mm or more. The length of the pin may be about 60 mm or less, 50 mm or less, or even 40 mm or less.

The diameter of the pin may be about 1 mm or more, 2 mm or more, or even 3 mm or more. The diameter of the pin may be about 7 mm or less, 6 mm or less, or even 5 mm or less. The major diameter of the pin may be about 1 mm or more, 2 mm or more, or even 3 mm or more. The major diameter of the pin may be about 7 mm or less, 6 mm or less, or even 5 mm or less. The minor diameter of the pin may be about 1 mm or more, 2 mm or more, or even 3 mm or more. The minor diameter of the pin may be about 7 mm or less, 6 mm or less, or even 5 mm or less.

The cross-sectional length of the pin may be about 0.5 mm or more, 1 mm or more, or even 2 mm or more. The cross-sectional length of the pin may be about 5 mm or less, 4 mm or less, or even 3 mm or less. The cross-sectional width of the pin may be about 0.5 mm or more, 1 mm or more, or even 2 mm or more. The cross-sectional width of the pin may be about 5 mm or less, 4 mm or less, or even 3 mm or less.

The aspect ratio of the pin may be about 1:50 or more, 1:40 or more, or even 1:30 or more. The aspect ratio of the pin may be about 1:5 or less, 1:10 or less, or even 1:20 or less. The cross-sectional aspect ratio of the pin may be about 1:30 or more, 1:20 or more, or even 1:10 or more. The cross-sectional aspect ratio of the pin may be about 1:1 or less, 1:3 or less, or even 1:5 or less.

In one general aspect, the present teachings provide for a bone pin comprising 6 axial fiber bundles bound and interlocked with bias fiber bundles. The cross-sectional shape of the bone pin may be circular. The ratio of axial fiber bundles to bias fiber bundles may be about 1:1. The volume of axial fibers in the bone pin may be about 47% of all fibers. The volume of bias fibers in the bone pin may be about 53% of all fibers. The bias fiber bundles may be oriented at about ±45°. The bias fiber bundles may be disposed as two separate layers of braiding, one layer over braided onto the other. The bone pin may have a weight per area of about 1.3 g/ft2. The bone pin may have a fiber volume of about 50%. The axial fiber bundles may comprise a plurality of fibers. The fibers may have a diameter of about 10 μm. Each of the axial fiber bundles may comprise about 2,000 fibers. The axial fiber bundles may have a twist of about 0.7 twists per inch. The axial fiber bundles may have a cross-sectional shape that is circular. The axial fiber bundles may have a diameter of about 0.25 mm. The fiber volume of the axial fiber bundles may be about 60%. The distance between fibers may be about 5 μm or less.

The composite implant of the present disclosure may be in the form of a screw. The screw may include an elongate structure with two opposing ends. The screw may be generally straight along its longitudinal axis. One end may include a head. The head may be configured to cooperate with a driver (e.g., screwdriver). The screw may include a drive socket. The drive socket may be located in the head. The other end may include a tip. The tip may be pointed, flat, truncated, or rounded. Threading may extend at least partially around the tip. The major diameter of threading may gradually increase as it extends from the tip to the head.

The screw may be a lag screw, compression screw, or interference screw.

The screw may extend through cortical bone, cancellous bone, an intramedullary canal, or any combination thereof. The screw may be employed to span a gap between a first bone portion and a second bone portion, the gap resulting from a fracture. The screw may extend through two or more bones, three or more bones, or even four or more bones. The screw may be employed to impose an alignment between two or more bones. For example, the screw may be employed to align hammer toe.

The screw may include a shank. The shank may be a non-threaded portion disposed between two portions of threading, between threading and a tip, between threading and a head, or any combination thereof).

The screw may be defined by a length, diameter, aspect ratio (i.e., ratio of length to width), or any combination thereof.

The length of the screw may be about 10 mm or more, 20 mm or more, or even 30 mm or more. The length of the screw may be about 60 mm or less, 50 mm or less, or even 40 mm or less.

The diameter of the screw may be about 1 mm or more, 2 mm or more, or even 3 mm or more. The diameter of the screw may be about 7 mm or less, 6 mm or less, or even 5 mm or less.

The aspect ratio of the screw may be about 1:50 or more, 1:40 or more, or even 1:30 or more. The aspect ratio of the screw may be about 1:5 or less, 1:10 or less, or even 1:20 or less.

The screw may include threading. The threading may have a thread angle, pitch, crest, root, major diameter, minor diameter, or any combination thereof. The thread angle may be measured between opposing surfaces of adjacent threads, as viewed along a transverse axis of the screw. The pitch may be the distance between crests of adjacent threads, as viewed along a transverse axis of the screw. The crest may be the most radially distanced point of the threads, as viewed along a transverse axis of the screw. The root may oppose the crest. The major diameter may be the cross-sectional diameter of the root. The minor diameter may be the cross-sectional diameter of the crest. One or more apertures may be present on the root.

The thread angle of the threading may be about 10° or more, 20° or more, 30° or more, or even 40° or more. The thread angle of the threading may be about 80° or less, 70° or less, 60° or less, or even 50° or less.

The major diameter of the threading may be about 1 mm or more, 2 mm or more, or even 3 mm or more. The major diameter of the threading may be about 7 mm or less, 6 mm or less, or even 5 mm or less. The minor diameter of the threading may be about 1 mm or more, 2 mm or more, or even 3 mm or more. The minor diameter of the threading may be about 7 mm or less, 6 mm or less, or even 5 mm or less.

The pitch of the threading may be about 1 mm or more, 1.3 mm or more, 1.6 mm or more, or even 1.9 mm or more. The pitch of the threading may be about 3 mm or less, 2.7 mm or less, 2.4 mm or less, or even 2.1 mm or less.

The crest of the screw may have a curvature. The curvature may prevent fracturing that may otherwise occur if the crest terminated at a sharp point. The curvature may be defined by a radius. The radius of curvature may be about 0.01 mm or more, 0.05 mm or more, or even 0.1 mm or more. The radius of curvature may be about 1.5 mm or less, 1 mm or less, or even 0.5 mm or less.

The threading may include a first side and a second side, the first side oriented toward the tip of the screw and the second side oriented toward the head of the screw. The first side and second side may extend at an angle between the crest and the root. The angle of the first side may be the same as or different from the second side. The angle may be measured from a transverse axis of the screw that is orthogonal to the longitudinal axis of the screw to an axis parallel to the surface of the first side or second side. The angle may be about 0° or more, 5° or more, 10° or more, or even 30° or more. The angle may be about 60° or less, 50° or less, or even 40° or less.

The threading may be fabricated by material addition, material removal, or both. The material addition may include molding, overmolding, 3D printing deposition, dip coating, braiding, the like, or any combination thereof. 3D printing deposition may apply an outer region to a core and pronounce portions of the outer region where the threading is to be formed. Cores may be located within a mold and an outer region including threading may be overmolded onto the core, the mold defining the threading.

The threading may be fabricated by helically winding fiber bundles around a core. The helical windings may be overlaid with one or more layers of tape. An outer region may be applied to the core and helical windings.

The material removal may include milling, laser etching, laser engraving, weaving, or any combination thereof. Material may be removed from one or more cores and/or outer regions to form threading. Threading may be formed on a preform of one or more cores and then an outer region may be applied to the preform by material addition as discussed herein.

The screw may comprise one or more cannulations. The cannulation may function to provide for ingress of fluid, to facilitate an inside-out degradation, as a driver socket, or any combination thereof. The cannulation may be a hollow shaft within a screw. The cannulation may extend at least partially between and/or through distal ends of a screw. The cannulation may extend longitudinally through the screw, through the center of the screw, between two distal ends of the screw, or any combination thereof. The cannulation may have a cross-sectional shape. The shape may be circular, ovoid, triangular, quadrilateral, pentagonal, hexagonal, octagonal, the like, or any combination thereof. The cannulation may be formed by locating a plurality of precursor elements around a removable mandrel. The cannulation may function as or define a driver socket. The driver socket may aid introduction of the screw into bone or other suitable substrate using a driver tool. The cannulation may extend at least partially through a screw head. The cannulation may taper from the head to the tip. The taper may provide a positive seating between the cannulation and driver tool.

The screw may include a cortical screw. The cortical screw may be employed to extend at least partially through cortical bone. Cortical bone typically comprises about 10% or less of soft tissue and is denser as compared to cancellous bone. The cortical screw may have a smaller major diameter and pitch as compared to a cancellous screw. This may be attributed, at least in part, to the density of the bone into which the cortical screw is configured to be located. For example, threading on a self-tapping cortical screw may be met with greater resistance in cortical bone due to the density thereof. The major diameter and pitch of a cortical screw generally need not be as large as that of a cancellous screw in order to achieve a clinically acceptable pull-out strength.

The screw may include a cancellous screw. The cancellous screw may be employed to extend at least partially through cancellous bone. Cancellous bone typically comprises about 35% or less of soft tissue and is less dense as compared to cortical bone. The cancellous screw may have a larger major diameter and pitch as compared to a cortical screw. This may be attributed, at least in part, to the density of the bone into which the cancellous screw is configured to be located. For example, threading on a self-tapping cancellous screw may be met with lesser resistance in cancellous bone due to the density thereof. The major diameter and pitch of a cancellous screw may be generally greater than that of a cortical screw in order to achieve a clinically acceptable pull-out strength due to the lower density of the bone matrix in which the cancellous screw is configured to reside.

The screw may or may not be self-tapping. Self-tapping screws may comprise a pointed tip and threading located at least partially on the tip, which gradually increases in its major diameter. Non-self-tapping screws may comprise a flat, truncated, or rounded tip.

The screw may or may not include a cannulation. The cannulation may be employed as a drive socket. The cannulation may be employed to accept a Kirschner wire (K-wire) that extends therethrough. The K-wire may function to locate screws into contact with the pre-formed hole in which they are configured to occupy. By way of example, K-wire may be located in a pre-formed hole and it may be threaded through a cannulation so the screw may translate along the K-wire.

The bone screw may include a lag screw. The lag screw may include a length of shaft that is free of threading. The lag screw may be free of threading from the head to a length from the head. The lag screw may include threading from the tip to a length from the tip. Conventionally, physicians avoid the location of threads in contact with or proximal (e.g., 2 mm or less or even 1 mm or less) to a fracture line.

The screw may include a soft tissue fixation screw. The soft tissue fixation screw may be employed for soft tissue fixation to bone. A hole may be pre-formed in bone. A portion of soft tissue may be located in the pre-formed hole. The soft tissue fixation screw may be located within the pre-formed hole and fixate the soft tissue between the soft tissue fixation screw and the bone.

The soft tissue fixation screw may be fabricated from one or more axial fiber bundles. The axial fiber bundles may be bound (e.g., over braided by a textile). The axial fiber bundles may have an ovoid cross-section. The ovoid cross-section may transfer torque more suitably.

In one aspect of the present teachings, the soft tissue fixation screw may include one or more apertures. The apertures may be formed, at least in part, by interstitial spaces between axial fiber bundles. The apertures may be formed, at least in part, by spacing between axial fiber bundles. The apertures may be formed, at least in part, by spaces between binding. The apertures may be formed, at least in part, by gaps in between intersecting and adjacent fibers in a braid. The apertures may be formed, at least in part, by removal of a sacrificial material from a mandrel.

By way of example, a screw that otherwise may have included 6 axial fiber bundles may have 3 axial fiber bundles removed and the interstitial space between the remaining fibers may form the transverse extent of the apertures at least in part. Interlocking and/or binding bias fibers may form the longitudinal extent of the apertures. Interlocking and/or binding bias fibers may compensate for the loss of mechanical properties by the interstitial spaces between axial bundles. The configuration of bias fibers (e.g., layers of bias fibers) may be modulated based upon the desired size of the apertures.

The screw may comprise threading. In one aspect of the present teachings, the threading may be fabricating by helically twisting a fiber bundle around the screw. The threading may be located so the apertures are located in the root of the threading.

In one aspect of the present teachings, an outer region may circumscribe the axial fiber bundles. The outer region may comprise polymeric material. The outer region may be machined to define threading. After machining the threads, a coating may be applied.

The apertures may result in a screw with a lower failure torque as compared to a screw with the same construction but free of apertures. However, by constructing the screws in accordance with the present teachings, the mechanical properties of the screw may meet or even surpass clinical requirements. The profile of the apertures may be modulated to improve the mechanical properties of the screw. The ratio of open surface area (i.e., surface area occupied by apertures) to closed surface area (i.e., surface area not occupied by apertures) may be modulated to improve the mechanical properties of the screw. The ratio of open surface area to closed surface area may be about 1:2 or more, 1:3 or more, or even 1:4 or more. The ratio of open surface area to closed surface area may be about 1:12 or less, 1:10 or less, or even 1:8 or less. Generally, the mechanical properties may increase as the ratio of open surface area to closed surface area decreases. For a given set of required mechanical property thresholds, the open:closed ratio may not decrease below a certain threshold. For articles constructed solely from degradable polymers, as disclosed herein, but not constructed according to composite structures of the present disclosure, the mechanical properties of such degradable polymer articles is less as compared to non-degradable polymers (e.g., PEEK) at a given open:closed ratio. However, it has been found that by employing the composite structures of the present disclosure, the mechanical properties of degradable polymers may meet or even surpass the mechanical properties of non-degradable polymers (e.g., PEEK) at a given open:closed ratio.

In constructing a screw (or other elongated article (e.g., pin)), one or more first fiber bundles (axial fiber bundles) may be nested around a removable mandrel, defining one or more cores; one or more second fiber bundles (bias fiber bundles) may then be wound around the first fiber bundles, defining threads. The second fiber bundles may be wound helically around the first fiber bundles; then the mandrel may be removed, leaving behind a cannulation. Before or after removal of the mandrel, the first and second fiber bundles may be coated, defining an outer region.

In constructing a screw with apertures, one or more first fiber bundles (axial fiber bundles) may be nested around a degradable mandrel. The degradable mandrels may include surface projections defining apertures. One or more second fiber bundles (bias fiber bundles) may bind, interlock, and/or wrap around the first fiber bundles. Threading may be fabricated according to fabrication techniques discussed herein. After implantation in a living being, the degradable mandrel may degrade rapidly (e.g., 1 week or less, 2 weeks or less, or even 3 weeks or less) revealing apertures that were previously formed and occupied by the surface projections of the mandrel.

The screw may include a composite core. The composite core may be fabricated from one or more, two or more, three or more, or even four or more fiber bundles. The fiber bundles may comprise axially aligned fibers fixated with matrix material. The fiber bundles may be bound with one or more layers of bias fiber bundles. The bias fiber bundles may be oriented at an angle of 45 degrees to the longitudinal axis of the composite core. The axial fibers and bias fibers may extend from the screw tip to the screw head. Other combinations of nested, bound, and interlocked fibers and/or reinforcement elements may be combined to produce cores for screws made in this manner.

Due to the torsional shear applied to the head of a screw applied by a driver (e.g., screwdriver), it may be particularly advantageous to provide a screw with additional reinforcement to increase torsional stiffness of the screw head.

The composite implant of the present disclosure may be in the form of a washer. The washer may be an annular, generally flat member. The washer may include a through-hole. The through-hole may be located in the center of the washer. The screw may be employed in cooperation with a washer. The washer may function to distribute stress imposed by a screw head as it is in contact with and rotated against bone. The washer may prevent splitting of cortical bone.

The composite implant of the present disclosure may be in the form of an anchor. The anchor may include an elongate structure with two opposing ends. The anchor may be generally straight along its longitudinal axis. The anchor may include threading, as disclosed hereinbefore. The anchor may include a drive socket. The drive socket may be defined by a cannulation, as disclosed hereinbefore. The drive socket may be formed in one end of the anchor.

The anchor may be employed to couple tissue to bone. The anchor may be employed to couple ligaments, tendons, or both to bone. Sutures may be coupled to the anchor. The anchor may be located within a bone. The sutures may extend from the bone and couple to tissue (e.g., ligament or tendon). The anchor and sutures may cooperate to couple tissue to bone.

The anchor may include any suitable cross-sectional shape. The cross-sectional shape may be circular, ovoid, triangular, quadrilateral, pentagonal, hexagonal, octagonal, or any combination thereof. The anchor may be defined by a length, diameter, aspect ratio (i.e., ratio of length to width), or any combination thereof.

The length of the anchor may be about 10 mm or more, 20 mm or more, or even 30 mm or more. The length of the anchor may be about 60 mm or less, 50 mm or less, or even 40 mm or less.

The diameter of the anchor may be about 1 mm or more, 2 mm or more, or even 3 mm or more. The diameter may be about 7 mm or less, 6 mm or less, or even 5 mm or less.

The aspect ratio of the anchor may be about 1:50 or more, 1:40 or more, or even 1:30 or more. The aspect ratio of the anchor may be about 1:5 or less, 1:10 or less, or even 1:20 or less.

The composite implant of the present disclosure may be in the form of a plate. The plate may be generally planar or at least include one or more planar segments. The plate may be bent in one or more locations. The plate may include a curvature. The plate may be pre-contoured. The plate may include an I-shape, L-shape, T-shape, H-shape, triangular shape, or square shape. The plate may be contoured to a perimetric surface of one or more bones. The plate may span a gap between two or more portions of bone, the gap resulting from a fracture. The plate may span two or more different bones. The plate may comprise one or more eyelets. The plate may cooperate with one or more screws, sutures, or both. The screws, sutures, or both may extend through the eyelets and into bone, tissue, or both.

The plate may include a compression plate (i.e., employed for fractures stable in compression), neutralization plate (i.e., employed to protect a fracture from normal bending, rotation, and/or axial forces), buttress plate (i.e., employed to support bone unstable in compression or axial loading). The plate may be used to treat clavicle fracture.

The composite implant of the present disclosure may be in the form of a splint. The splint may be located within an intramedullary canal. The splint may be constructed in vivo, ex vivo, or both. The splint may include an elongate structure with two opposing ends. The perimeter of the splint may contour the intramedullary canal.

One or more reinforcement elements may be arranged in vivo, ex vivo, or both to construct the splint. One or more reinforcement elements, or assemblies thereof arranged ex vivo, may be sequentially added into a patient and a composite implant may be constructed in situ. To this end, the reinforcement elements may have a size and flexibility to be delivered through a catheter. The catheter may have an inner diameter of at least at about 1 mm, 2 mm, or even 3 mm. The catheter may have an inner diameter of at least about 6 mm, 5 mm or even 4 mm. Each of the reinforcement elements may be hierarchically structured so the splint may be custom tailored for the intramedullary canal in which it is to be located. In this manner, the splint may be fabricated to suit different lengths, widths, cross-sectional dimensions, or any combination thereof.

A plurality of reinforcement elements may be provided to a physician. The plurality of reinforcement elements may have different dimensions, configurations, mechanical properties, or any combination thereof. The physician may select the reinforcement elements to construction a splint therefrom. The reinforcement elements may be sequentially introduced into a bone of the patient. The physician may select reinforcement elements based upon the identity of the bone and/or tissue to which the composite implant is to be located, size of the catheter, size of the bone cavity in which the reinforcement elements are to be introduced, the severity of the bone fracture, age of the patient, gender of the patient, underlying medical conditions of the patient (e.g., osteoporosis), or any combination thereof. The reinforcement elements may be chosen to provide desired mechanical properties and/or degradation profile according to the needs of individual patients.

The splint may be employed with a containment bag. The containment bag may be located within an intramedullary canal and the constituent elements of the splint may be located within the bag. The containment bag may function to protect the splint from ingress of blood and/or other bodily fluids that might interfere with the deployment of precursor elements, solidification of polymeric material, accelerate degradation, or any combination thereof. The containment bag may function to constrain the flow of polymeric material while the polymeric material is in an injectable (flowable) state. The containment bag may have sufficient strength to allow the polymeric material to be injected into the containment bag under substantial pressure so as to ensure sufficient interfacial contact between the polymeric material and other precursor elements. Pressure may be employed to minimize voids within the containment bag. The containment bag may be flexible or rigid. The containment bag may be sufficiently flexible so that the containment bag can be inserted into an opening and then expand so that a pressure is applied to a wall of the opening.

The one or more reinforcement elements may be introduced into a containment bag by means of a delivery catheter or sheath. The reinforcement elements may have sufficient strength to allow longitudinal delivery into a containment bag by pushing. The reinforcement elements may be flexible. The reinforcement elements may be pre-cured prior to introduction into a patient. The reinforcement elements may have smooth outer surfaces and/or tapered ends to facilitate movement past one another and/or intervening structures while being delivered into the containment bag. Sheets, in the form of rolls, may be radially compressed by other reinforcement elements and/or intervening structures while being delivered into the containment bag.

The containment bag may be fabricated from a bioabsorbable polymer, fibers, or both. The bioabsorbable polymer may include polyurethane, polylactic acid, glycolic acid, copolymers thereof, or any combination thereof. The fibers may be woven, non-woven, or both. The fibers may be braided and/or knitted. The fibers may form a mesh. The fibers may be fabricated from polylactic acid, polyglycolic acid, polydioxanone, copolymers thereof, bioabsorbable glass, soluble glass, or any combination thereof. The containment bag may be hydrophobic so as to minimize the ingress of bodily fluids into the containment bag.

The containment bag may have a limited porosity to allow some egress of polymeric material out of the containment bag (e.g., to osseointegrate with the surrounding bone). The porosity may be varied across the extent of the containment bag so as to provide regions of greater or lesser porosity to the polymeric material, thus providing control of the ability of the polymeric material to infiltrate the surrounding bone. The containment bag may have an average pore size of about 0.0001 μm or more, 0.001 μm or more, 0.01 μm or more, or even 0.1 μm or more. The containment bag may have an average pore size of about 1000 μm or less, 100 μm or less, 10 μm or less, or even 1 μm or less. The containment bag may have a thickness (wall thickness) of about 0.01 mm or more 0.1 mm or more, or even 1 mm or more. The containment bag may have a thickness (wall thickness) of about 5 mm or less, 3 mm or less, or even 2 mm or less.

The splint may comprise one or more cannulations. The cannulation may function to provide for ingress of fluid, facilitate an inside-out degradation, or both. The cannulation may be a hollow shaft within a splint. The cannulation may extend at least partially between and/or through distal ends of a splint. The cannulation may extend longitudinally through the splint, through the center of the pin, between two distal ends of the splint, or any combination thereof. The cannulation may have a cross-sectional shape. The shape may be circular, ovoid, triangular, quadrilateral, pentagonal, hexagonal, octagonal, the like, or any combination thereof. The cannulation may be formed by locating a plurality of precursor elements around a removable structure (e.g., balloon).

One or both opposing ends of the splint may include end-caps that are configured to degrade at a faster rate than the perimeter of the splint in contact with the bone at the walls of the intramedullary canal. The end-caps may function to expose interior portions of the splint to degradation conditions. In this manner, the splint may degrade from the inside-out. It may be particularly advantageous to provide a splint that degrades from the inside-out while maintaining an interface between the splint and the bone at the walls of the intramedullary canal for a period of time to maintain the mechanical properties of the splint during healing. The interface between the splint and the bone at the walls of the intramedullary canal may be maintained for about 1 week or more, 4 weeks or more, 6 weeks or more, 12 weeks or more, 24 weeks or more, 48 weeks or more, or even 60 weeks or more.

The composite implant may include one or more apertures. The apertures may be included in pins, screws, anchors, or any combination thereof. The apertures may protrude inwardly toward a center axis of the composite implant. The apertures may be through-holes. That is, the apertures may extend the complete diameter of the composite implant. The apertures may extend only partially along a radius of the composite article. The apertures may extend 5% or more, 10% or more, 20% or more, or even 30% or more the radius of the composite implant. The apertures may extend 100% or less, 90% or less, 80% or less, or even 70% or less the radius of the composite implant.

The apertures may extend at least partially between opposing distal ends of a composite implant. The apertures may extend radially around a composite. The apertures may helically twist around a composite. The twist may be defined by a pitch of about 0 to 1 revolution per cm of axial length. The pitch may be unitary or may vary along a length of the composite.

The apertures may be located around a perimeter of the composite implant. The apertures may be located along a length of the composite implant. The apertures may be in longitudinal alignment. The apertures may be off-axis longitudinally with respect to each other. The apertures may have a circular profile. The apertures may have elongated profile (e.g., ovoid).

The apertures may be fabricated by material addition, material removal, or both. The material addition may include molding, overmolding, 3D printing deposition, dip coating, braiding, the like, or any combination thereof. 3D printing deposition may apply an outer region to a core and skip portions of the outer region where the apertures are to be formed. Cores may be located within a mold and an outer region may be overmolded onto the cores, the mold defining the apertures. Aperture-shaped molds may be coupled to a core preform, the preform may be subjected to one or more series of dip coating to form an outer region where the molds are not located, and the molds may thereafter be removed to reveal apertures.

The material removal may include milling, laser etching, laser engraving, weaving, or any combination thereof. Material may be removed from one or more cores and/or outer regions to form one or more apertures. One or more apertures may be formed into a preform of one or more cores and then an outer region may be applied to the preform by material addition as discussed herein.

The apertures may be fabricated by braiding. The braiding may be performed by a machine (e.g., rope braiding machine). One or more axial fibers and/or or axial fiber bundles may travel axially in one direction. Spindles containing fibers or fiber bundles may rotate generally radially around the core. The generally radial path may include a series of undulations with one set of spindles undulating in opposing relationship to a second set of spindles. Thus, the fibers or fiber bundles wrap helically around the axials while the opposing undulations cause an alternating overlapping between fibers and fiber bundles of different carriers. In order to form an aperture, one or more carriers may be free of fibers or fiber bundles. Thus, a repeating pattern of apertures may be formed where the carrier free of fibers or fiber bundles would have otherwise located the same. The depth of the apertures may be increased by performing two or more series of braiding in additional layers (e.g., over braiding).

The apertures may occupy a volume of about 5% or more, 10% or more, or even 15% or more, with respect to the total volume of the composite implant. The apertures may occupy a volume of about 40% or less, 30% or less, or even 20% or less, with respect to the total volume of the composite implant. The employment of apertures may increase the surface area to the composite implant.

The composite implants may be employed in highly vascularized regions, low vascularized regions, or both. Generally, highly vascularized regions are subject to a higher degradation rate as compared to low vascularized regions. The highly vascularized regions may include exterior surfaces of bone and intramedullary canal. The low vascularized regions may include cortical bone and cancellous bone. Plates, splints, and washers may be employed in highly vascularized regions. At least portions of screws (e.g., heads) may be employed in highly vascularized regions. In the case that composite implants are located in highly vascularized regions, additional barriers, barriers with lower aqueous permeability, or thicker barriers may be employed. Pins, screws, and nails, or at least portions thereof (e.g., threaded portion of a screw) may be employed in low vascularized regions.

A composite implant may be defined by dimensions including a length, outer diameter, inner diameter (e.g., cannulation diameter), or any combination thereof. Generally, the dimensions of the composite implant direct the configuration of the precursor elements of the composite implant. While the dimensions of composite implants vary widely depending upon the particular application in which they are employed, generally the holes pre-formed in bone to accept the composite implants (e.g., screws, nails, and pins) are between about 0.5 mm and 5 mm in their largest cross-sectional dimension. Generally, holes drilled in bone should not exceed ⅓ the diameter of the bone or portion of the bone in order to avoid compromising the mechanical support of the bone. Cannulations formed in composite implants to accept drivers or guidewires (e.g., K-wires) are typically between about 0.5 mm and 2 mm.

The present disclosure contemplates balancing one or more properties of the composite implant within the dimensional constraints discussed in the preceding paragraph. By balancing these properties, favorable mechanical properties according to the present disclosure may be realized. In general, increasing the fiber volume and decreasing the matrix material volume provides for a load-bearing composite implant. In general, balancing the ratio of axial fibers to bias fibers provides for a composite implant with preferred mechanical properties in bending, compression, and torsion.

### Fibers, Fiber Bundles, Fiber Composites

The composite article (e.g., composite implant) may be fabricated from one or more fibers, fiber bundles, fiber composites, matrix materials, reinforcement elements, fillers, sizings, or any combination thereof. These may be referred to alone or in combination as precursor elements.

The composite article may be fabricated from one or more fibers. In general, the fibers may be continuous fibers, long fibers, short fibers, or any combination thereof. During fabrication continuous fibers may be extruded by a spinneret and loaded onto a spool. Thus, the length of a continuous fiber is extreme and generally indefinite. Long fibers may have a length of between about 5 mm and 200 mm. Short fibers may have a length of about 5 mm or less.

The fibers may have a diameter of about 100 μm or more, 150 μm or more, 200 μm or more, or even 250 μm or more. The fibers may have a diameter of about 500 μm or less, 450 μm or less, 400 μm or less, or even 350 μm or less.

The fibers may comprise two or more filaments. Filaments may be the most basic structure of the composite. The fibers may be fabricated by grouping together filaments from different spools and respooling the fiber on a single spool.

The filaments may or may not have a surface modification applied thereto. Where a surface modification is employed, the surface modification is applied to the filaments prior to being assembled into fibers. It may be particularly advantageous to apply surface modification to the filaments to ensure adequate interfacial contact between filaments and matrix material.

A surface modification may or may not be applied to fiber bundles (an outer perimeter thereof) after assembly of filaments into fiber bundles. It may be particularly advantageous to apply surface modification to the fiber bundles to ensure adequate interfacial contact between fiber bundles and matrix material.

The filaments may have a diameter of about 3 μm or more, 6 μm or more, 9 μm or more, or even 12 μm or more. The filaments may have a diameter of about 24 μm or less, 21 μm or less, 18 μm or less, or even 15 μm or less.

As discussed herein, processing of the fibers into other structures (e.g., fiber bundles) may utilize fibers of the continuous, long, or short variety, or any combination thereof. In one aspect of the present teachings, continuous fibers may be fed through a rope braiding apparatus to form a braided structure, the length of which is limited to the length of continuous fibers on a spool. In another aspect of the present teachings, two or more spools of continuous fibers may be unspooled, combined together to form a fiber bundle, and re-spooled as a fiber bundle. The length of the fiber bundle is limited to the length of continuous fibers used to form the fiber bundle. In yet another aspect of the present teachings, fibers may be cut into long fibers and/or short fibers prior to forming a fiber bundle. In yet another aspect of the present teachings, fiber bundles may be cut into long fibers and/or short fibers after forming a fiber bundle.

In general, a fiber bundle may comprise fibers of different lengths. Bias fibers may be longer than axial fibers due to the helical winding and/or weaving of bias fibers. Bias fibers may be about 5% or more, 10% or more, 15% or more, 20% or more, 30% or more, or even 40% or more longer than axial fibers.

It will be appreciated by the present teachings that composite articles (e.g., composite implants) may comprise fibers that have a length of about 5 mm or more, 10 mm or more, 20 mm or more, 50 mm or more, or even 70 mm or more. The composite articles (e.g., composite implants) may comprise fibers that have a length of about 200 mm or less, 250 mm or less, 200 mm or less, 150 mm or less, or even 100 mm or less. That is, during fabrication of the composite articles, smaller structures may be cut from larger structures to realize fiber lengths between about 5 mm and 200 mm.

The fibers may have a diameter of about 9 μm or more, 15 μm or more, or even 21 μm or more. The fibers may have a diameter of about 35 μm or less, 29 μm or less, or even 23 μm or less.

The fiber, whether coated or uncoated, may have a high modulus, a low elongation, or both, as measured according to ASTM D638.

The fiber, whether coated or uncoated, may have a tensile modulus of about 0.08 GPa or more 0.1 GPa or more, 1 GPa or more, 10 GPa or more, 20 GPa or more, 30 GPa or more, 40 GPa or more, or even 50 GPa or more.

The fiber, whether coated or uncoated, may have a tensile modulus of about 100 GPa or less, 90 GPa or less, 80 GPa or less, 70 GPa or less, or even 60 GPa or less.

The fiber, whether coated or uncoated, may have an elongation of about 2% or more, 10% or more, 30% or more, 70% or more or even 100% or more. The fiber, whether coated or uncoated, may have an elongation of about 500% or less, 400% or less, 300% or less, or even 200% or less.

The fiber, whether coated or uncoated, may have a tensile strength of about 10 MPa or more, 20 MPa or more, 40 MPa or more, or even 60 MPa or more. The fiber, whether coated or uncoated, may have a tensile strength of about 150 MPa or less, 130 MPa or less, 110 MPa or less, 90 MPa or less, or even 70 MPa or less.

The fibers may have an aspect ratio (i.e., ratio of length to width) of from about 1:1 to about 1:10,000, about 1:10 to about 1:1,000, or even about 1:20 to about 1:100.

The fibers may be biodegradable and/or bioabsorbable. The fibers may be fabricated from glass, polymer, or both.

The glass may include borate-based glass, phosphate-based glass, silicon-based glass, or any combination thereof. The glass may include P2O5, P2O3, SiO2, B2O3, Na2O, CaO, ZnO, MgO, Fe2O3, K2O, MnO, NaF, Ce2O3, or any combination thereof. The glass may include additional elements including Cu, Sr, Zn, Fe, Mn, Cr, V, Nb, Mo, W, Ba, Co, S, Al, Ti, Y, Mg, Si, F, Zn, Ni, or any combination thereof.

The polymer may include, but is not limited to, polyglycolic acid (PGA), polylactic acid (PLA), polyglycolide-polylactide copolymers (PGA/PLA), polyhydroxybutyric acid, polycaprolactone, polymalic acid, polydioxanes, polysebacic acid, polyadipic acid, polyglycolide-trimethylene carbonate copolymers (PGA/TMC), poly-L-lactide (PLLA), poly-D-lactide (PDLA), poly-DL-lactide (PDLLA), lactide tetramethylene glycolide copolymers, poly(D,L-lactide-co-trimethylene carbonate), poly(L-lactide-co-trimethylene carbonate), lactide δ-valerolactone copolymers, lactide ε-caprolactone copolymers, polydepsipeptide(glycine-DL-lactide copolymer), polylactide ethylene oxide copolymers, asymmetrically 3,6-substituted poly-1,4-dioxane-2,4-diones, poly-p-hydroxybutyrate (PHBA), PHBA β-hydroxyvalerate copolymers (PHBA/PHVA), poly-p-hydroxypropionate (PHPA), poly-β-dioxanone (PDS), poly-δ-valerolactone, poly-δ-caprolactone, methyl methacrylate-N-vinyl pyrrolidone copolymers, polyester amides, oxalic acid polyesters, polydihydropyrans, polypeptides from α-amino acids, poly-β-maleic acid (PMLA), poly-β-alkanoic acids, polyethylene oxide (PEO), polybutylene succinate, bio-polyethylene, P4HB, P3HB, silk, collagen, hyaluronic acid, chitin, chitosan, starch, spider silk, pullulan, cellulose, gelatin, alginate, the like, copolymers thereof, or any combination thereof. As referred to herein, polylactide may refer to one or any combination of stereoisomers of polylactide including poly-L-lactide (PLLA), poly-D-lactide (PDLA), poly-DL-lactide (PDLLA). Other suitable exemplary and non-limiting polymeric materials may include polyurethanes, acrylics, polyesters, polyamides, polyamines, polyaramides, polyaryletherketones, polysulfones, polyolefins, epoxy, polyurea, polyurea urethane, acrylate, acrylate urethane, propylene glycol fumarate, polycarbonate, polystyrene, polycitrate esters, polyamides, polyphosphates, polyphosphonates, polyphosphazenes, polycyanoacrylates, polyorthoesters, polyacetals, polydihydropyransbiopolymers, copolymers thereof, or any combination thereof.

A plurality of fibers may be arranged into one or more fiber bundles. The fiber bundles may be fabricated from continuous fiber, long fiber, short fiber, or any combination thereof.

The fiber bundles may comprise about 5 or more, 10 or more, 100 or more, or even 1,000 or more fibers. The fiber bundles may comprise about 1,000,000 or less, 500,000 or less, 100,000 or less, or even 10,000 or less fibers.

The fiber bundles may have a cross-sectional length in its largest dimension of about 50 μm or more, 100 μm or more, 200 μm or more, 300 μm or more, or even 400 μm or more. The fiber bundles may have a cross-sectional length in its largest dimension of about 800 μm or less, 700 μm or less, 600 μm or less, or even 500 μm or less.

The fiber bundles may have a fiber volume of about 40% or more, 50% or more, or even 60% or more. The fiber bundles may have a fiber volume of about 95% or less, 90% or less, 80% or less, or even 70% or less. The remaining volume may be occupied by empty space (e.g., air). The empty space may arise from interfacial spaces between adjacent and contacting fibers. The fiber volume may depend from the cross-sectional shape and/or cross-sectional length/width of the fibers and the packing of the fibers together. For example, circular cross-section fibers may give rise to more interstitial spaces as compared to square cross-section fibers.

A twist may or may not be imparted to the fiber bundles. The twist may be clockwise (S), counterclockwise (Z), not twisted (0), or any combination thereof. The twist rate may be about 0 or more, 1 or more, 2 or more, or even 3 or more twists per inch. The twist rate may be about 7 or less, 6 or less, or even 5 or less twists per inch. The best properties are seen with a twist of about 0.3 to 1.5 turns per inch, preferably 0.5 to 1.3 turns per inch. Without a twist, the flexural strength may be about 280-290 MPa. By adding a twist, it is possible to increase the flexural strength to about 400 to 700 MPa, or even more. The twist may impart an ovoid cross-sectional shape to the fiber bundles.

The fiber bundle may comprise continuous fiber, long, fiber, short fiber, or any combination thereof. Continuous fibers may extend an entire length of the fiber bundle. The fibers may be arranged in axial alignment with respect to one another. Axially aligned may refer to alignment along the longitudinal axis of the filaments (parallel).

The fibers may or may not have a surface modification applied thereto before being formed into a fiber bundle. The fiber bundles may or may not have a surface modification applied thereto after formation of the fiber bundles.

The fiber bundles may be constructed of fibers fabricated from a variety of different materials. For example, a thermoplastic fiber may be interspersed within portions of a higher modulus fiber in order to facilitate handling during cutting operations with a hot knife or, through the use of a heat gun, to reduce fiber damage during storage.

A barrier may be applied to a fiber bundle. The barrier may be applied by coating the fiber bundle. The barrier may be applied by cross-head extrusion. Before coating with polymer, the fibers having less than 2000 filaments are flexible and have a bending radius of less than 2 cm and can be wound around a spool having a 2 cm outer diameter. The fibrous bundles may be separated and/or coated by a polymeric material. To increase the adhesion of the barrier to the fiber bundles, the fiber bundles may be slightly roughened/structured.

The fiber bundles may be combined with matrix material to form one or more fiber composites. The fiber bundles may be impregnated and/or coated with matrix material to form fiber composites. The matrix material may be in a flowable state. The matrix material may be applied via extrusion coating (e.g., cross-head die extrusion), dip coating, spraying, rolling, swabbing, brushing, or any combination thereof. In the course of any of these processes, capillary action may cause the matrix material to permeate to inner fibers of the fiber bundle. It may be particularly advantageous to ensure that all fibers of a fiber bundle are evenly coated to avoid mechanically weak points in the structure and/or avoid regions that are subject to rapid degradation due to the absence of matrix material protecting the fibers.

Where extrusion coating is employed, the size of the die may modulate the fiber volume of a fiber composite. The die may compress fibers together and reduce the volume of interstitial space for matrix material to occupy. The die may allow fibers to separate and increase the volume of interstitial space for matrix material to occupy. The fiber volume may decrease where the size of the die increases.

Fibers fabricated from polymeric material according to the present teachings may be intermingled with glass fibers during the arrangement of the fibers into fiber bundles. The fiber bundles may be heated causing the polymeric fibers to melt and coat the glass fibers of the fiber bundle.

Two or more fiber composites may be arranged together to form reinforcement elements. Fiber bundles may be arranged together to form reinforcement elements prior to being combined with matrix material. Reinforcement elements may be fabricated from a combination of fiber bundles and fiber composites.

The fiber composites may have a fiber volume of about 40% or more, 50% or more, or even 60% or more. The fiber composites may have a fiber volume of about 95% or less, 90% or less, 80% or less, or even 70% or less. The remaining volume may be occupied by polymeric material (“polymeric material”) empty space (e.g., air), or both.

In one aspect of the present teachings, fiber bundles and/or fiber composites may be wound around itself while translating a length (back-and-forth) of a rotating mandrel. The winding may proceed until a desired dimension (e.g., major diameter) is achieved. The resulting wrap of fiber bundles and/or fiber composites may be a reinforcement element. After removal of the mandrel, a cannulation may be revealed.

In another aspect of the present teachings, fiber bundles and/or fiber composites may be wound around itself while translating a length (back-and-forth) of a rotating reinforcement element. The winding may proceed until a desired dimension (e.g., major diameter) is achieved.

The fiber bundles and/or fiber composites may include surface projections for improved integration with the polymeric material. The surface projections may be formed by interspersing chopped fibers (e.g., long fibers and/or short fibers) in random orientations throughout the length of the fiber bundles and/or fiber composites such that at least a portion of the randomly oriented fibers extend beyond the outer surface of the reinforcement elements.

### Reinforcement Elements

One or more fibers, fiber bundles, fiber composites, matrix materials, fillers, or any combination thereof may be arranged together to form one or more reinforcement elements. The one or more reinforcement elements may function to construct a composite article (e.g., composite implant), provide desirable mechanical properties to a composite article, or both.

The reinforcement elements may be scaled by modulating the quantity of fibrous bundles included in the reinforcement elements. For example, two or more fibrous bundles may be nested together to form a larger structure of nested fibrous bundles, and those nested fibrous bundles may be assembled (e.g., by nesting) to form a larger structure, and so forth.

The reinforcement elements may be characterized by one or more mechanical properties. The mechanical properties may include tensile strength, compressive strength, flexural strength, torsional strength, ductile failure mode, or any combination thereof. Any of the foregoing mechanical properties may be modulated by the selection of fiber material, the selection of matrix material, fiber count, fiber volume, fiber orientation, braid, braid, layup, twist, cross-sectional shape, cross-sectional thickness, cross-sectional aspect ratio, cell size, or any combination thereof.

The reinforcement elements may include a combination of different materials with varying mechanical properties, degradation profiles, or both. The material makeup of the reinforcement elements may be unitary along a length of the reinforcement element producing mechanical properties and/or a degradation profile that is unitary along the length. The material makeup of the reinforcement elements may be variable along a length of the reinforcement element so that mechanical properties and/or degradation profile may be variable along the length.

The reinforcement elements may comprise a plurality of fibers. The quantity of fibers, alternatively referred to herein as a fiber count, in a reinforcement element may be about 5 or more, 10 or more, 100 or more, or even 1,000 or more. The quantity of fibers in a reinforcement element may be about 10,000,000 or less, 1,000,000 or less, 100,000 or less, or even 10,000 or less. For example, a reinforcement element may comprise 5 fiber bundles, each of the fiber bundles including 100 fibers, and so the resulting reinforcement element may have a fiber count of 500 fibers.

The reinforcement elements may be characterized by a fiber volume. Fiber volume may be defined by the ratio of the volume of fibers to the volume of composite and expressed as a percentage by multiplying the ratio by 100. The volume of composite may be occupied by elements other than fibers including but not limited to matrix material, air, filler, and the like. The fiber volume may be about 5% or more, 10% or more, 20% or more, 40% or more, or even 60% or more. The fiber volume may be about 95% or less, 85% or less, 75% or less, or even 65% or less.

The reinforcement elements may comprise fibers in one or more orientations. The fibers may be oriented axially (i.e., axially aligned with a longitudinal axis of a reinforcement element), at a bias (i.e., oriented at an angle to a longitudinal axis of a reinforcement element), or both. Bias fibers may be oriented at an acute angle to a longitudinal axis of the reinforcement element. The angle may be about ±0° or more 5° or more 15° or more 25° or more, 35° or more or even 45° or more. The angle may be ±90° or less 75° or less, 65° or less, or even 55° or less. Positive and negative angles, as referred to herein, mean offset clockwise or counterclockwise, respectively, with respect to a longitudinal axis of a reinforcement element.

Axial fibers may function to provide column stiffness, compression strength, compression stiffness, or any combination thereof.

Bias fibers may function to provide flexural stiffness and torsional strength. Bias fibers may function to distribute stress in different directions from axial reinforcement elements. Bias fibers may function to provide additional hydrostatic pressure as compared to a composite article including only axial reinforcement elements. Hydrostatic pressure may be beneficial to draw matrix material into composite articles or portions thereof. Bias fibers may be employed to form a composite article with a particular cross-sectional shape without altering the overall cross-sectional footprint of the composite article.

Increasing the angle of bias fiber bundles results in a thicker bias fiber bundle layer.

The thickness (cross-sectional) of bias fiber layers may be about 50 μm or more, 100 μm or more, 150 μm or more, 200 μm or more, 250 μm or more, or even 300 μm or more. The thickness of bias fiber layers may be about 800 μm or less, 700 μm or less, 600 μm or less, 500 μm or less, or even 400 μm or less.

The reinforcement elements may have a ratio of a quantity of axial fibers to a quantity of bias fibers of about 10:90 or more, 10:75 or more, 10:55 or more, 10:35 or more, 10:15 or more, or even 10:10 or more. The reinforcement elements may have a ratio of a quantity of axial fibers to a quantity of bias fibers of about 90:10 or less, 75:10 or less, 55:10 or less, 35:10 or less, or even 15:10 or less. The quantity ratio of axial fibers to bias fibers may modulate the mechanical properties of the reinforcement element.

The twist of fibers from which a reinforcement element is fabricated may modulate the mechanical properties of the reinforcement element. The twist of fibers is discussed in greater detail hereinbefore. The quantity of fibers having different twists (e.g., S and Z twist) may be balanced to provide for better processability.

The reinforcement elements may include a cross-sectional shape. The cross-sectional shape may include the shape of a transverse cross-section. The cross-sectional shape may be defined by the shape formed by an outer perimeter of the reinforcement elements. The cross-sectional shape may be circular, ovoid, elliptical, triangular, quadrangular, rhomboid, pentangular, hexangular, octangular, cruciform, lobed, the like, or any combination thereof.

The cross-sectional shape may be modulated by the arrangement of fiber bundles in reinforcement elements. For example, two fiber bundles may be arranged side-by-side and a third fiber bundle may be arranged on top of and in between the two fiber bundles, forming a triangle. It has been found by the present inventors that triangular cross-section reinforcement elements provide favorable nesting properties for creating other scalable structures. It has been found by the present inventors that triangular cross-section reinforcement elements provide comparatively better torsional properties as compared to circular cross-section reinforcement elements. This may be attributed at least in part by the sliding of adjacent circular cross-sections and interference of abutting sides of triangular cross-sections. As another example, a row of three fiber bundles may be stacked with another row of three fiber bundles, forming a rectangle.

A triangular cross-section may maintain its shape when passed over a mandrel. A circular cross-section may flatten to a rectangle when passed over a mandrel.

The cross-sectional shapes my provide particular functions to the reinforcement elements. By way of example, triangular shapes may be more rigid in bending (compared to triangular shapes) and therefore, for medical applications, will be suitable for insertion into long straight bones such as the humerus, tibia or femur. By way of another example, rectangular or circular shapes may bend around curves and therefore, for medical applications, will be suitable for insertion into curved bones such as the clavicle. It has also been found by the present inventors that triangular shapes strain less in torsion as compared to rectangular or circular shapes.

The cross-sectional shape may influence the interstitial spaces between reinforcement elements and therefore, the quantity, volume, and distribution of matrix rich regions. For example, packing of reinforcement elements with ovoid cross-sections may provide a greater volume of matrix rich regions as compared to packing of reinforcement elements with circular cross-sections. As another example, packing of reinforcement elements with circular cross-sections may provide a greater volume of matrix rich regions as compared to packing of reinforcement elements with triangular cross-sections.

The reinforcement elements may include a cross-sectional thickness. The cross-sectional thickness may be defined by the largest dimension of the cross-section. The cross-sectional thickness may be about 10 μm or more, 50 μm or more, 100 μm or more, or even 500 μm or more. The cross-sectional thickness may be about 10 mm or less, 5 mm or less, 2 mm or less, or even 1 mm or less.

The cross-sectional thickness may influence the interstitial spaces between reinforcement elements and therefore, the quantity, volume, and distribution of matrix rich regions. For example, the larger the cross-sectional thickness the larger the volume of matrix rich regions. As another example, reinforcement elements with smaller cross-sectional thicknesses may be intermingled with reinforcement elements with larger cross-sectional thicknesses, the reinforcement elements with smaller cross-sectional thicknesses filling the interstitial spaces between the reinforcement elements with larger cross-sectional thicknesses, resulting in a lower overall volume of matrix rich regions.

The reinforcement elements may be defined by a cross-sectional aspect ratio. The cross-sectional aspect ratio may be defined by a ratio of the cross-sectional length of the reinforcement element along its major axis (i.e., axis corresponding to its longest cross-sectional length) to the cross-sectional length of the reinforcement element along its minor axis (i.e., axis corresponding to its shortest cross-sectional length. The cross-sectional aspect ratio may be between about 1:1 and 1:100, more preferably between about 1:1 and 1:50, more preferably between about 1:1 and 1:30, or more preferably between about 1:1 and 1:10. The cross-sectional aspect ratio may be about 1:1 or more, 1:2 or more, 1:3 or more, 1:5 or more, 1:10 or more, or even 1:20 or more. The cross-sectional aspect ratio may be about 1:100 or less, 1:90 or less, 1:80 or less, 1:70 or less, 1:60 or less, or even 1:50 or less.

The reinforcement elements may be defined by a cell size. A cell, as referred to herein, means a common repeating unit in a pattern. The pattern may be viewed in a transverse cross-section of a reinforcement element. The cell size may be defined by a length and width of a cell. It has been observed by the present inventors that cell size can be correlated to, and thus altered to modulate properties of a composite article. The cell may be characterized by a volume of reinforcement element, a volume of matrix material, a volume of free space, or any combination thereof. The cell size may be influenced by the packing of elements.

The reinforcement elements may be defined by a braid. Two or more fiber bundles may be braided together to form a reinforcement element. Fiber bundles may form a textile. The braid may be biaxial, triaxial, or quadaxial.

The reinforcement elements may be defined by a layup. Two or more textiles, whether braided or unbraided, may be layed-up one atop another, in any orientation. Layup is a concept typically utilized in laminate composites. The orientation of fibers in distinct layers may be aligned or offset from each other. The number of axes in which fibers are aligned may be the same or different between different distinct layers.

The construction of the reinforcement elements may direct the mechanical properties and/or degradation profiles of the reinforcement elements. The reinforcement elements may comprise a plurality of axial fiber bundles, axial fiber composites, bias fiber bundles, axial fiber composites, or any combination thereof.

The reinforcement elements may be formed into one or more rods, textiles, sheets, tape, or any combination thereof.

The rod may be a generally linear, elongate member. The rod may have a shaped cross-section. The cross-sectional shape may be circular, ovoid, elliptical, triangular, quadrangular, rhomboid, pentangular, hexangular, octangular, cruciform, lobed, the like, or any combination thereof.

The rod may be fabricated by arranging a plurality of axial fiber bundles together and affixing their arrangement. Their arrangement may be fixed by impregnation with matrix material, wrapping with bias fiber bundles. Where both axial fiber bundles and bias fiber bundles are employed, a ratio of the fiber volume of bias fiber bundles to axial fiber bundles may be about 0.5:1 to about 1:0.5, or more preferably about 1:1.

The fiber volume of a rod may modulate mechanical properties of the rod. By example, a rod made with fiber bundles having 400 fibers per bundle may have a flexural modulus of about 27 GPa, and fiber bundles having about 200 fibers per bundle may have a flexural modulus of about 13 GPa. However, it has been found by the present inventors that rods having larger quantities of fibers per bundle may be brittle and exhibit a strain at failure of 2% or less. By employing a smaller quantity of fibers per bundle, the failure mode may be changed from a brittle failure to a ductile failure and the strain at failure may be increased to greater than 2%.

The quantity of fibers per bundle may modulate degradation rate. A smaller quantity of fiber bundles may exhibit a slower degradation rate.

The fiber volume of a rod may be about 55% for the thermoplastic based samples and about 60% for the thermoset based samples.

The rods based on the smaller fiber bundles resulted in a more homogeneous structure, with about 2% lower fiber volume, yet still having about the same stiffness/modulus compared to the large bundles. The most homogeneous structure is seen with small fiber bundles having a twist.

In the construction of screws, it may be particularly advantageous to provide rods with an elliptical cross-section to distribute torsional loads.

The reinforcement elements may be in the form of textiles. The textile may be braided, non-braided, or both. The non-braided textile may be fabricated from randomly oriented fibers. Non-braided textiles may be fabricated from continuous fibers, long fibers, short fibers, or any combination thereof. A braided textile may be biaxially braided, triaxially braided, quadaxially braided, or any combination thereof. In fabricating the textile, bias fiber bundles may interlock axial fiber bundles. Employing interlocking bias fiber bundles may increase the flexural modulus of the resulting structure. The mechanical properties of the textile may be modified by modulating the material fiber material, fiber orientation, quantity of axes, fiber bundle twist, or any combination thereof of the fibers within the textile.

The textile may comprise axial fiber bundles and/or axial fiber composites, bias fiber bundles and/or bias fiber composites, or any combination thereof. Bias fibers and/or bias fiber bundles may be oriented at an angle to axial fibers and/or axial fiber bundles. The angle may be about ±0° or more 5° or more 15° or more 25° or more, 35° or more or even 45° or more. The angle may be ±90° or less 75° or less, 65° or less, or even 55° or less. 75°.

A higher ratio of axial to bias fibers may strengthen the resistance to bending and axial forces (e.g., compression or tension). A lower ratio of axial fibers to bias fibers may increase hoop strength and torsional resistance.

The textile may comprise fiber bundles that are twisted and/or fiber bundles that are untwisted (i.e, otherwise referred to as tow). Twisted fiber bundles and/or fiber composites may maintain their cross-sectional shape during and after weaving. Tow may spread out and decrease in cross-sectional aspect ratio during and after weaving. Twisting a fiber bundle and/or fiber composite that originally has a circular cross-section may result in an ovoid cross-section.

The braided textile may be biaxially braided. The biaxial braid may comprise one or more axial fiber bundles and one or more bias fiber bundles. The biaxial braid may be characterized by the number of axial fiber elements (e.g., bundles) per bias fiber elements (e.g., bundles) in the braid. The biaxial braid may include a 1×1, 2×1, or even 3×1 braid.

The braided textile may include a tight braid or loose braid. A tight braid may refer to a substantial absence of gaps between fiber bundles. A loose braid may refer to the presence of gaps between fiber bundles.

The braid may be defined by a unit cell. The unit cell may be defined by a length and width of the most basic repeating unit pattern within the braid. The unit cell may be about 1×1, 2×2, or 3×3. The unit cell may determine how load is shared between fibers. A unit cell of 1×1 provides better crack inhibition as compared to 2×2 or 3×3 due to the greater quantity of contacts between fiber bundles. A unit cell of 2×2 provides better crack inhibition as compared to 3×3 due to the greater quantity of contacts between fiber bundles. Smaller unit cell sizes may result in less load distribution and more intersections between the fibers. Larger unit cell sizes may result in more load distribution and less intersections between the fibers. While load distribution provides a favorable result to the overall mechanical properties of a composite article, more intersections between fibers is also favorable to redirect stresses throughout the composite article. Thus, it may be advantageous to balance the load distribution with the number of intersections between fibers.

As referred to herein with respect to braiding, any reference to filaments, fibers, bundles, or reinforcement elements may be used interchangeably. That is, bias and/or axial elements may be in the form of filaments, fibers, bundles, or reinforcement elements.

The smallest open area in a braid between axial and/or bias fibers may be smaller than a span between bone caused by a fracture. There may be 1 or more, 1.5 or more, or even 2 or more unit cells in contact with the span between bone caused by a fracture. For example, for a fracture span of about 1 mm, the smallest open area in a braid may be about 0.5 to about 1 mm. In this manner full load sharing between axial and/or bias fibers may be ensured across the span of the fracture.

The braided textile may include a planar braid or tubular braid. The tubular braid (i.e., 3D braid) may be formed by running one or more continuous axial fiber bundles may be run through the center of a weaving table and other continuous fiber bundles (bias bundles) may be braided over the axial fiber bundle at an angle to the longitudinal axis of the axial fibrous bundles.

The fibrous bundles may or may not be impregnated with polymeric material. The fibrous bundles may have a fiber volume of about 10% to about 90%, about 40% to about 70%, or even about 50% to about 60%. The remaining volume may be occupied by polymeric material (“polymeric material”).

During the process of arranging bias fibers around axial fibers, tension may be applied to the axial fibers and the tension may be locked into place by the disposition of bias fibers around the axial fibers. For example, pultrusion may impart a tension onto axial fibers and the disposition of bias fibers around the axial fibers during pultrusion may lock in the tension. As another example, a braiding apparatus may put tension on axial fibers and bias fibers disposed around axial fibers during braiding may lock in the tension.

Bias fibers may be flexible so that they can be processed, wound, and braided into a braided structure or other bias structure. Bias fibers have a structure that permits flat contact between elements braided in a positive and negative angle.

Bias fibers may provide channels for the flow of liquid into the fiber bundle structure. Flow may also be accelerated by engineering in of hydrostatic force inducing elements that pull matrix through the full construct, such as by wicking.

The reinforcement elements may be in the form of tapes and/or sheets. Tapes and sheets may differ by their aspect ratios. Tape may have an aspect ratio of about 1:2 or more, 1:10 or more, or even 1:50 or more. Tape may have an aspect ratio of about 1:200 or less, 1:150 or less, or even 1:100 or less. Typically, the aspect ratio of tape is between 1:1 and 1:10. Sheets may have an aspect ratio of about 1:200 or more, 1:300 or more, or even 1:400 or more. Sheets may have an aspect ratio of about 1:1,000 or less, 1:900 or less, or even 1:800 or less. Sheets may be cut to form tapes.

Tapes and/or sheets may be fabricated from textiles. The textiles may be coated with matrix material to form tapes and/or sheets. Two or more textiles may be laid-up together and coated with matrix material to form tapes and/or sheets. Textiles laid-up together may be coated prior to or after layup, or both.

Tapes and/or sheets may be fabricated from a plurality of uni-axial fiber bundles and/or fiber composites. A plurality of uni-axial fiber bundles may be coated with matrix material to form tapes and/or sheets. The uni-axial fiber bundles may be spaced apart from one another. The uni-axial fiber bundles may be spaced by a length of about 0.5 mm or more, 1 mm or more, or even 2 mm or more. The uni-axial fiber bundles may be spaced by a length of about 5 mm or less, 4 mm or less, or even 3 mm or less. The matrix material may fixate the position of the uni-axial fiber.

The sheets may have a generally rectangular cross-section. The sheets may be generally planar.

The sheets may be formed into rolls. The rolls may include two or more layers of a unitary sheet rolled onto itself. The rolls may be compressible by rolling into a tighter arrangement. The rolls may expand. The sheets may be formed into tubes. The tubes may be fabricated by joining opposing edges of a sheet. Tubes may be concentrically arranged one within another. The sheets may wrap around reinforcement elements.

The sheets may comprise 10 or more fibers, 50 or more fibers, 100 or more fibers, or even 500 or more fibers. The sheets may comprise 1,000,000 or less fibers, 100,000 or less fibers, 10,000 or less fibers, or even 1,000 or less fibers.

The sheets may have a minor diameter of about 0.10 mm or more, 0.3 mm or more, or even 0.5 mm or more. The sheets may have a minor diameter of about 1 mm or less, 0.8 mm or less, or even 0.6 mm or less.

The tape may have a generally rectangular or elliptical cross-section. The sheets may be generally planar.

The tape may wrap around fiber bundles, reinforcement elements, or both. The tape may be wound at an angle of about ±0° or more 5° or more 15° or more 25° or more, 35° or more or even 45° or more. The tape may be wound at an angle of about ±90° or less 75° or less, 65° or less, or even 55° or less. The tape may be wound by affixing an edge of the tape to a reinforcement element and rotating the reinforcement element while manipulating the tape along the length of the reinforcement element.

The tape may be wound around itself on a rotating mandrel.

Tape may be wound in one or more layers. Different layers may be wrapped at different angles (e.g., +45°, −45°, and 0°). Combining different angles of bias tape, in addition to axial tape can provide a composite that yields sufficient flexural stiffness and strength, compressive stiffness and strength, and torsional stiffness and strength.

The tape may comprise 10 or more fibers, 50 or more fibers, 100 or more fibers, or even 500 or more fibers. The tape may comprise 1,000,000 or less fibers, 100,000 or less fibers, 10,000 or less fibers, or even 1,000 or less fibers.

The tape may comprise 10 or more fibers per bundle, 50 or more fibers per bundle, 100 or more fibers per bundle, or even 200 or more fibers per bundle. The tape may comprise 1,000 or less fibers per bundle, 800 or less fibers per bundle, 600 or less fibers per bundle, or even 400 or less fibers per bundle.

The tape may form a laminate. The laminate may be fabricated by laying-up layers of tape at any orientation. The layers may be bound together at an elevated temperature and/or pressure. The following are typical examples of stacking sequences for laminate composites. Orienting layers 1, 2, 3, 4, 5, 6, 7, and 8 at angles of 0°, +45°, −45°, 90°, 90°, −45°, +45°, and 0°, respectively. Orienting layers 1, 2, 3, 4, 5, and 6 at angles of +45°, 0°, −45°, +45°, 0°, and −45°, respectively.

The orientation of the layers in the laminate may provide the appropriate properties in multiple directions, such as strength and stiffness in bending, compression, or torsion. Such that the material may have isotropic (i.e., an object having a physical property which has the same value when measured in different directions) or anisotropic (i.e., an object having a physical property that has a different value when measured in different directions) properties.

The tape may have a minor diameter of about 0.10 mm or more, 0.3 mm or more, or even 0.5 mm or more. The tape may have a minor diameter of about 1 mm or less, 0.8 mm or less, or even 0.6 mm or less.

The tape may have between about 50% and 80% of fibers by weight. The tape may have between about 30% and 60% fibers by volume.

Two or more tapes may be laid-up to form a laminate. Two or more tapes may be fed from stock coils of tape and laid-up together. One or more tapes may be fed from stock coils of tape and laid up with one or more layers of previously laid, consolidated, and/or solidified tapes. The lamination may include a heating step, consolidation step, and solidification step. Prior to the heating step, tapes may be placed in contact with one another. Interfacial contact between an entirety of the surfaces of laid-up tapes or at least a substantial portion thereof is important to ensure complete bonding. Interfacial contact may be influenced by one or more rollers. In the heating step, the tape may be subjected to temperatures below the melting temperature of the polymeric material. In the consolidation step, the tape may have pressure applied thereto and the temperature the tapes are subjected to may increase above the melting temperature of the polymeric material. The heating may allow polymer chains to move across the interface between layers and form polymer chain entanglements and/or bonds with an opposing layer. In the solidification step, pressure may continue to be applied and the tapes and the temperature the tapes are subjected to may gradually decrease to below the melting temperature of the polymeric material or even to room temperatures (i.e., between about 20° C. and 25° C.). After the solidification step, a matrix rich layer between fibers in the tape may be present.

Axial fiber bundles may function to provide column stiffness, compression strength, compression stiffness, or any combination thereof.

Bias fiber bundles may function to provide flexural stiffness and torsional strength. Bias fiber bundles may function to distribute stress in different directions from axial fiber bundles. Bias fiber bundles may transmit torque from the inside of the composite article to the outside of the composite article. For example, a driver engaged with a cannulation and causing a composite article to turn may generate torque that may be translated by fiber bundles. Bias fiber bundles may function to provide additional hydrostatic pressure as compared to a composite article including only axial fiber bundles. Hydrostatic pressure may be beneficial to draw matrix material into composite articles or portions thereof. Bias fiber bundles may be employed to form a composite article with a particular cross-sectional shape without altering the overall cross-sectional footprint of the composite article.

Two or more fiber bundles and/or fiber composites may be assembled together by nesting, binding, interlocking, or any combination thereof. Two or more reinforcement elements may be assembled together by nesting, binding, interlocking, or any combination thereof.

In one aspect of the present teachings, the reinforcement elements may be formed by a molding process. Continuous axial fibers may be located inside a mold cavity and a polymeric material may be introduced to flow around and/or impregnate the fibers, and caused to cure, solidify, or both. In another aspect of the present teachings, chopped fiber may be mixed with polymeric material and located inside a mold cavity and caused to cure, solidify, or both.

As referred to herein, nesting may mean axially aligning and bunching together fiber bundles, fiber composites, and/or reinforcement elements. Nesting of axial fiber bundles, axial fiber composites, and/or axial reinforcement elements may provide for the same to have strength in tension and compression. Nesting may provide for additional polymeric material between discrete fiber bundles, fiber composites, and/or reinforcement elements, which allows for slippage between the same, reducing brittle failure. Nested fiber bundles, fiber composites, and/or reinforcement elements may be affixed by coating with matrix material, binding, or interlocking.

The fiber bundles, fiber composites, and/or reinforcement elements may be ordered or not ordered in nesting. Ordering or not ordering may modulate mechanical properties of the composite article. As referred to herein, ordered mean generally uniformly aligned (e.g, an average alignment that deviates from an axis by less than 10°). For example, axial fiber bundles may be arranged side-by-side in uniform layers. Two or more layers may be axially aligned with one another or axially offset from one another. As referred to herein, not ordered may refer to packing in a random spatial distribution. The use of ordered and not ordered fibrous bundles may be selected and used in combination to achieve a desire may cause different wetting out of material throughout the fibrous bundles, during a later step of infiltrating the fiber bundles, fiber composites, and/or reinforcement elements with a polymeric material.

Nesting may be performed to fabricate reinforcement elements with deliberate cross-sectional shapes, as discussed hereinbefore.

As referred to herein, binding may mean wrapping perimetrically with one or more bias fiber bundles, bias fiber composites, or bias reinforcement elements (e.g., tape). Binding of fiber bundles, fiber composites, and/or reinforcement elements may constrain buckling of the same and increase column strength and stiffness. The binding layer may provide for transmission of transverse loads through the composite article and may be helpful in applications subjected to torque off-axis to the axial load.

As referred to herein, interlocking may mean interlocking by weaving. Axial fiber bundles, axial fiber composites, and/or axial reinforcement elements may be interlocked by weaving one or more of the same with one or more bias fiber bundles, bias fiber composites, or bias reinforcement elements (e.g., tape). Interlocking fiber bundles, fiber composites, and/or reinforcement elements may provide for slippage between axials and may interrupt fracture planes in the polymeric material between axials. This reduces crack propagation by absorbing and redirecting energy in the composite article and provides toughness, thus reducing brittle failure. Interlocking may help transmit loads between fiber bundles, fiber composites, and/or reinforcement elements. Interlocking may allow fiber bundles, fiber composites, and/or reinforcement elements absorb transverse loads. Interlocking may interrupt fracture planes in the composite article by placing fiber bundles, fiber composites, and/or reinforcement elements in areas that would normally be comprised of only polymeric material. Interlocking between layers of fiber bundles, fiber composites, and/or reinforcement elements may transfer loads between layers. When placed under torsional loads, the interlocking between layers may transmit torque through the thickness of the composite article more efficiently than a stacked laminate. Interlocking may avoid delamination between layers seen with laminate composites.

Working fiber bundles, fiber composites, and/or reinforcement elements by nesting, binding, interlocking, interlocking, or any combination thereof may contribute to the enhanced mechanical properties of the composite article. Working of reinforcement elements, in cooperation with fiber bundles and/or fiber composites may fabricate new larger reinforcement elements.

The combination of fiber bundles, fiber composites, and/or reinforcement elements in different orientations within a composite article may provide a tougher composite article. Individual fiber bundles, fiber composites, and/or reinforcement elements may behave as a unit to resist mechanical loads. Fiber bundles, fiber composites, and/or reinforcement elements provided in different orientations may redirect fracture lines, dissipate energy, absorb energy, or any combination thereof, resulting in a ductile failure mode.

The flexural modulus of composite articles fabricated from fiber bundles, fiber composites, and/or reinforcement elements can be decreased with a lower angle of bias structures and increased with a higher angle of bias structures.

A volume ratio of axial structures (e.g., fiber bundles, fiber composites, and/or reinforcement elements) to bias structures may be about 0.25:1 or more, about 0.5:1 or more, about 0.75:1 or more, about 0.90 or more, about of 1:1 or more, about 1.2:1 or more, about 1.5:1 or more, or about 2.0:1 or more. The volume ratio may be about 4:1 or less, about 3:1 or less, about 2:1 or more, about 1.5:1 or less, about 1:1 or less, about 0.80 or less, about 0.60:1 or less, or about 0.5:1 or less.

### Matrix Material

The composite article may comprise one or more matrix materials. The matrix material may function to provide a medium to bind and/or affix reinforcement elements, fiber composites, fiber bundles, fibers, filler, or any combination thereof. The matrix material may flow between and/or impregnate reinforcement elements, fiber composites, fiber bundles, fibers, filler, or any combination thereof. The matrix material may coat reinforcement elements, fiber composites, fiber bundles, fibers, filler, or any combination thereof. The matrix material may function to form scaffolding for bone and tissue ingrowth. The matrix material may function to transfer load to reinforcement elements, fiber composites, fiber bundles, fibers, filler, or any combination thereof. The matrix material may function to protect reinforcement elements fiber composites, fiber bundles, fibers, filler, or any combination thereof from environmental exposure, at least for a predetermined amount of time. The matrix material may define surface texture, surface porosity, passages, or any combination thereof.

Matrix material may be applied to the composite article and/or constituent elements thereof during one or more steps of fabrication. Matrix material may be applied to reinforcement elements, fiber composites, fiber bundles, fibers, filler, or any combination thereof. For example, fibers may be assembled into fiber bundles, the fiber bundles may be coated in matrix material, a twist may be applied to fiber bundles, and a matrix material may be applied to the twisted fiber bundles. Applying matrix material to the composite article and/or constituent elements thereof during one or more steps of fabrication may ensure adequate saturation of matrix material and avoid voids of matrix material within the composite article.

The matrix material may biodegrade and/or bioabsorb in response to regional (bodily or environmental) stimuli. The stimuli may include aqueous solution, salinity, pH, soluble ions, naturally or artificially introduced enzymes, or any combination thereof.

The matrix material may include one or more polymeric materials. The polymeric material may include thermoplastics, thermosets, or both. The matrix material may include one or more low modulus materials. The polymeric material may include one or more synthetic polymers, organic polymers, or both.

The polymeric material may be liquid (e.g., flowable or injectable) in a first state and generally solid in a second state. The polymeric material may be activated to transition from the first state to the second state. The polymeric material may be activated by a stimulus. The stimulus may include heat, pressure, chemical exposure, moisture exposure, UV light, the like, or any combination thereof. The polymeric material may include un-crosslinked polymer, crosslinked polymer, or both in the first. The polymeric material may or may not be crosslinked after activation. In medical applications according to the present teachings, the polymeric material may be activated in vivo or ex vivo. The polymeric material may be partially activated ex vivo and completely activated in vivo.

Activation rate may be controlled to maintain the matrix material within a desired temperature range. Activation of matrix material (e.g., a reaction between polyol-isocyanate to form polyurethane) may involve an exothermic reaction. Activation rate may be controlled by modulating an amount of catalyst. In medical applications according to the present teachings, excessive temperatures may harm the patient. Preferably, the matrix material does not cause a temperature to exceed 44° C., more preferably 40° C., or even more preferably 36° C.

The polymeric material may include a one-part or multi-part (e.g., two-part) polymer system. In medical applications according to the present teachings, the multi-part polymer system may be mixed immediately prior to being located within a living being (e.g., static mixing). The multi-part polymer system may be mixed about 1 minute or more, 2 minutes or more, 4 minutes or more, 10 minutes or more, or even 15 minutes or more prior to being located within a living being. The multi-part polymer system may be mixed about 1 hour or less, 50 minutes or less, 40 minutes or less, 30 minutes or less, or even 20 minutes or less prior to being located within a living being.

In medical applications according to the present teachings, the pot life (i.e., useable working and/or application time) of the matrix material may be suitable for typical surgical procedure times. If pot life is too short, then viscosity may increase to an unworkable degree, wettability may be negatively impacted, and cohesion between precursor elements may not occur. If pot life is too long, then procedure times may be unnecessarily prolonged. Pot life of matrix material may be controlled by varying amount of catalyst (e.g., zirconium). Generally, decreasing an amount of catalyst may increase pot life. The pot life may be about 3 minutes or more, 6 minutes or more, 9 minutes or more, or even 12 minutes or more. The pot life may be about 21 minutes or less, 18 minutes or less, or even 15 minutes or less. During the pot life, the viscosity may be about 500 cps or more, 1,000 cps or more, 1,500 cps or more, or even 2,000 cps or more. During the pot life, the viscosity may be about 5,000 cps or less, 4,500 cps or less, 4,000 cps or less, 3,500 cps or less, or even 3,000 cps or less.

The matrix material may be chosen for its ability to wet out, viscosity, moisture content, glass transition temperature, or any combination thereof.

In medical applications according to the present teachings, viscosity may determine how readily the matrix material can be introduced via a syringe and/or catheter, and/or mixed via static mixing. Viscosity may determine the degree of wetting out of precursor elements. If the matrix material includes a multi-part system, it may be particularly advantageous to provide the different parts with approximately similar viscosities to provide for proper static mixing.

Composite strength is determined, in part, by how well the reinforcements in the composite are wet out by the matrix. Poor wetting of the fibers can result in gaps between the giver and the final solidified matrix. These gaps become flaws in the final composite which will reduce its ultimate strength. In general, as a composite gets larger, it becomes more difficult to penetrate the plurality of fibers with matrix. By building the structure in a hierarchal fashion from discrete elements, each smaller element may or may not be pre-wet with matrix prior to assembly. The modularity of the construction also allows for matrix to flow freely between reinforcement elements providing for improved wetting.

The polymeric materials may include, but are not limited to, polyglycolic acid (PGA), polylactic acid (PLA), polyglycolide-polylactide copolymers (PGA/PLA), polyhydroxybutyric acid, polycaprolactone, polymalic acid, polydioxanes, polysebacic acid, polyadipic acid, polyglycolide-trimethylene carbonate copolymers (PGA/TMC), poly-L-lactide (PLLA), poly-D-lactide (PDLA), poly-DL-lactide (PDLLA), lactide tetramethylene glycolide copolymers, poly(D,L-lactide-co-trimethylene carbonate), poly(L-lactide-co-trimethylene carbonate), lactide δ-valerolactone copolymers, lactide ε-caprolactone copolymers, polydepsipeptide(glycine-DL-lactide copolymer), polylactide ethylene oxide copolymers, asymmetrically 3,6-substituted poly-1,4-dioxane-2,4-diones, poly-β-hydroxybutyrate (PHBA), PHBA p-hydroxyvalerate copolymers (PHBA/PHVA), poly-β-hydroxypropionate (PHPA), poly-β-dioxanone (PDS), poly-δ-valerolactone, poly-δ-caprolactone, methyl methacrylate-N-vinyl pyrrolidone copolymers, polyester amides, oxalic acid polyesters, polydihydropyrans, polypeptides from α-amino acids, poly-β-maleic acid (PMLA), poly-β-alkanoic acids, polyethylene oxide (PEO), polybutylene succinate, bio-polyethylene, P4HB, P3HB, silk, collagen, hyaluronic acid, chitin, chitosan, starch, spider silk, pullulan, cellulose, gelatin, alginate, the like, copolymers thereof, or any combination thereof. As referred to herein, polylactide may refer to one or any combination of stereoisomers of polylactide including poly-L-lactide (PLLA), poly-D-lactide (PDLA), poly-DL-lactide (PDLLA). Other suitable exemplary and non-limiting polymeric materials may include polyurethanes, acrylics, polyesters, polyamides, polyamines, polyaramides, polyaryletherketones, polysulfones, polyolefins, epoxy, polyurea, polyurea urethane, acrylate, acrylate urethane, propylene glycol fumarate, polycarbonate, polystyrene, polycitrate esters, polyamides, polyphosphates, polyphosphonates, polyphosphazenes, polycyanoacrylates, polyorthoesters, polyacetals, polydihydropyransbiopolymers, polyhydroxyalkanoates, citric acid based polymers, copolymers thereof, or any combination thereof.

The polymeric material may biodegrade by a series of hydrolysis reactions. Hydrolysis may reduce the molecular weight of the polymeric material. The molecular weight may reduce by a factor of about 4 or more, 6 or more, 8 or more or even about 10 or more. The molecular weight may reduce by a factor of about 30 or less, 25 or less, 20 or less, or even 15 or less. The molecular weight may reduce to a magnitude that avails the remaining polymeric fragments to microbial metabolization or, for medical applications according to the present disclosure, human metabolization. It may be particularly advantageous for the molecular weight to decrease at a controlled rate to avoid fragmentation of the matrix material. The filler of the polymeric material, fibers, or both may comprise soluble glass. The soluble glass may dissolve in aqueous solution over time. The dissolution of soluble glass (e.g., bioglass) may influence the local pH of aqueous solution. The dissolution of soluble glass may influence a change in the local pH of about 0.5 or more, more preferably 1.5 or more, or even more preferably 3 or more. Acidic or basic conditions may accelerate the rate of hydrolysis. By way of example, phosphate anions may impart acidic conditions. By way of another example, sodium cations may impart basic conditions. The rate of hydrolysis may increase in proportion to temperature. Conditions that accelerate the rate of hydrolysis may be referred to herein as catalysts. In general, sodium and potassium ions are relatively more soluble than calcium and magnesium ions. In general, calcium and magnesium ions are more soluble than aluminum and iron ions.

The polymeric material may have a molecular weight ranging from about 10 kDa or more, 250 kDa or more 500 kDa or more, or even 1,000 kDa or more. The polymeric material may have a molecular weight ranging from about 100,000 kDa or less, 10,000 kDa or less, or even 5,000 kDa or less. The polymeric material may have a viscosity of about 1 cps or more, 10 cps or more, 50 cps or more, 100 cps or more, 250 cps or more, or even 500 cps or more. The polymeric material may have a viscosity of about 100,000 cps or less, 50,000 cps or less, 25,000 cps or less, or even 10,000 cps or less. It may be particularly advantageous for the polymeric material to have viscosities less than 3,000 cps. By way of example but not limitation, the matrix can have a polydispersion index (pdi) of less than about 1.1, 1.5, 2, or even 2.5.

In one aspect of the present teachings, the polymeric material may comprise polyurethane. The polyurethane may be produced by reaction of isocyanates having at least two reactive functional groups per molecule (difunctional or polyfunctional) with a molecule having two or more active hydrogen groups (difunctional or polyfunctional) capable of reacting with the isocyanate (e.g., polyol).

The polyurethane may be synthesized from bio-based materials. The polyurethane may be synthesized by reacting a bio-based polyol (e.g., from corn, vegetable oil, or castor oil) and a bio-based isocyanate (e.g., from soy protein).

The hydroxyl number of the polyol can range from 40 to 1000, more preferably from 100 to 800, or even more preferably 200 to 600. The polyol may have a molecular weight ranging from about 50 kDa to about 50,000 kDa, more preferably from about 100 kDa to about 3,000 kDa, or even more preferably from about 200 kDa to about 1,000 kDa. The polyol may have a hydrogen functionality ranging from 2 to 6, more preferably from 2 to 4.

The molecule having two or more hydrogen groups (e.g., polyol) may include primary and secondary aliphatic hydroxyls and amines; primary, secondary, and aromatic amines; aliphatic and aromatic thiols; urethane and urea groups; polyols; or any combination thereof. Examples of suitable polyols may include, but are not limited to, polycaprolactone diol, polycaprolactone triol, ethylene glycol, propylene glycol, butylene glycol, hexylene glycol, polyalkylene oxides, polyvinyl alcohols, polyalkylene oxides (e.g., polyethylene oxide), glycerin, 1,2,4-brutanetriol, trimethylol propane, pentaerythritol, dipentaerythritol, 1,1,4,4-tetrakis(hydroxymethyl)cyclo-hexane, sugars, starches, N,N,N′,N′-tetrakis(2-hydroxyethyl)ethylenediamine, phosphate ester polyol, or any combination thereof.

The polyol may include at least one bioabsorbable group to alter the degradation profile of the resulting branched, functionalized compound. The bioabsorbable group may include, but is not limited to, glycolide, glycolic acid, lactide, lactic acid, caprolactone, dioxanone, trimethylene carbonate, and combinations thereof. The bioabsorbable groups may be present in an amount of from about 7% to 95%, more preferably about 20% to 90%, or even more preferably about 50% to about 85%, by weight of the polyol.

The polyol may be present in an amount of about 30% or more, 35% or more, 40% or more, or even 45% or more, by weight. The polyol may be present in an amount of about 60% or less, 55% or less, 50% or less, or even 45% or less, by weight.

The isocyanate may include aliphatic, cyclic, or aromatic isocyanates. When a biodegradable composite is desired, aliphatic isocyanates are generally favored. When a non-biodegradable composite is desired, aromatic isocyanates are generally favored. The isocyanate index of the isocyanate can range from 5% to 60%, more preferably from 15% to 45%, or even more preferably 25% to 35%.

Examples of suitable isocyanates may include, but are not limited to, 1,2 and 1,4 toluene diisocyanate, 2,4-toluene diisocyanate, 2,6-toluene diisocyanate, 2,2′-diphenylmethane diisocyanate, 2,4′-diphenylmethane diisocyanate, 4,4′-diphenylmethane diisocyanate, diphenyldimethylmethane diisocyanate, dibenzyl diisocyanate, naphthylene diisocyanate, phenylene diisocyanate, xylylene diisocyanate, methylene diphenyl diisocyanate, 4,4′-oxybis(phenylisocyanate), tetramethylxylylene diisocyanate, tetramethylene diisocyanate, hexamethylene diisocyanate, dimethyl diisocyanate, lysine diisocyanate, methyl lysine diisocyanate, lysine triisocyanate, 2-methylpentane-1,5-diisocyanate, 3-methylpentane-1,5-diisocyanate, 2,2,4-trimethylhexamethylene diisocyanate, isophorone diisocyanate, cyclohexane diisocyanate, hydrogenated xylylene diisocyanate, hydrogenated diphenylmethane diisocyanate, hydrogenated trimethylxylylene diisocyanate, 2,4,6-trimethyl 1,3-phenylene diisocyanate, or any combination thereof.

The isocyanate may be present in an amount of about 50% or more, 55% or more, 60% or more, or even 65% or more, by weight. The isocyanate may be present in an amount of about 85% or less, 80% or less, 75% or less, or even 70% or less, by weight.

As a non-limiting example, the polyurethane may be synthesized from a reaction of 10.6 g polycaprolactone diol, 6.0 g polycaprolactone triol, and 23.31 mL isophorone diisocyanate. As another non-limiting example, the polyurethane may be synthesized from a reaction of 15.90 g polycaprolactone diol, 6.00 g polycaprolactone triol, and 27.97 mL isophorone diisocyanate.

The polyurethane may be reacted in the presence of a catalyst. The catalyst may include amine compounds organometallic complexes (e.g., based on mercury, lead, tin, bismuth, titanium, zirconium, or zinc), or both. Examples, of amine catalysts may include, but are not limited to, triethylenediamine (TEDA), dimethylcyclohexylamine (DMCHA), dimethylethanolamine (DMEA), dibutyl tin dilaurate, stannous octoate, 1,8-diazabicyclo[5.4.0]undec-7-ene (DBU), or any combination thereof. The catalyst may be present in an amount of about 0.010% or more, 0.10% or more, or even 0.2% or more, by weight. The catalyst may be present in an amount of about 1% or less, 0.8% or less, 0.6% or less, or even 0.4% or less.

The polyurethane may be synthesized without the use of an isocyanate catalyst. Sorbitan may be treansesterfied with either dimethyl carbonate or propylene carbonate. The resulting decarbonate may be reacted with an amine to form a polyurethane. The reaction may proceed at room temperature.

In another aspect of the present teachings, sorbitan may be esterified to form a polyester. The resulting polyester may be esterified with acetic anhydride to reduce the hydroxyl group content. Reducing the hydroxyl group content may reduce the water sensitivity of the reaction product.

The polymeric material may comprise a solvent. The solvent may be biocompatible. The solvent may function to reduce viscosity of the polymeric material. The solvent may function as a porogen. An exemplary, non-limiting solvent may include dimethyl sulfoxide (DMSO).

The matrix material may comprise one or more fillers. The filler may function to provide porosity, bone and/or tissue ingrowth surfaces, enhanced permeability, enhanced pore connectivity, enhance mechanical properties, resistivity to water permeation, or any combination thereof. The filler may be in the form of fibers, nanofibers, rods, nanotubes, nanorods, flakes, particulates, extractable liquids, or any combination thereof. The filler may be organic, inorganic, or both. The filler may be biocompatible, biodegradable, bioabsorbable, soluble, insoluble, osteoconductive, or any combination thereof. The filler may include porogens,

The filler (e.g., glass particulates) may dissipate energy input into the composite article by stresses (e.g., bending, compression, or torsion).

The filler may be present in a polymeric material, reinforcement elements, or both in an amount of about 1% or more, 5% or more, 10% or more, 20% of more, 25% or more, or even 30% or more, by volume. The filler may be present in a polymeric material, reinforcement elements, or both, in an amount of about 90% or less, 85% or less, 80% or less, 75% or less, 70% or less, or even 65% or less, by volume.

The filler may be about 1 μm or more, 10 μm or more, 30 μm or more, 50 μm or more or even 100 μm or more in their largest dimension (e.g., length or width). The filler may be about 500 μm or less, 400 μm or less, 300 μm or less, or even 200 μm or less in their largest dimension. The largest dimension of filler may refer to the mean size of filler. The filler may include two or more fillers having different mean sizes.

The filler may include one or more porogens. The porogen may function to dissolve and form surface roughness, porosity, passages, or any combination thereof. Dissolution of the porogen may increase the surface area exposed to an aqueous environment, increase degradation of the composite, expose other fillers to an aqueous environment, or any combination thereof. The porogen may include sugars, polysaccharides (e.g., dextran, chitosan, chitosan/PLA, or chitin), soluble salts, degradable polymers apart from the matrix material, or any combination thereof. The porogen may dissolve in the presence of water and leave behind pores in the polymeric material. The pores may provide for tissue ingrowth, faster matrix degradation, or both, by increasing the surface area of polymeric material exposed to the environment. The porogen may be dispersed throughout the matrix material. The porogen (e.g., polysaccharide) may be applied as a coating on a containment bag, precursor elements, or both. The porogen may be in the form of a chopped fiber, flake, particulate, or any combination thereof. After dissolution of the fiber one or more passages defined in the polymeric material may be formed. The degradable polymer may include polylactic acid, polyglycolic acid, polycaprolactone, hydroxybutyrate, hydroxypropionic acid, hydroxyhexanoate, co-polymers thereof, or any combination thereof. The porogen may be present in a polymeric material in an amount of about 15% or more, 20% or more, or even 25% or more, by weight. The porogen may be present in a polymeric material in an amount of about 50% or less, 45% or less, 40% or less, or even 35% or less, by weight.

In the medical applications of the present teachings, the filler may act include one or more therapeutic agents. The therapeutic agent may function to promote bone formation, relieve pain, reduce inflammation, inhibit infection, or any combination thereof. The therapeutic agent may be dispersed throughout the matrix material. The therapeutic agent may be located in a portion of the composite implant that degrades a time after implantation. For example, the therapeutic agent may be located in the matrix material of a reinforcement element that is located toward the central axis of the composite implant.

The therapeutic agent may be delivered locally via a carrier vehicle to provide a protective environment, provide target delivery to cells or within cells, provide locally delivery, timed delivery, staged delivery, or any combination thereof. The therapeutic agent may be tuned for consistent release. The therapeutic agent may be tuned by incorporating into a solubilizing component having a predictable solubility. The therapeutic agent may be released in a clinically effective dose.

The therapeutic agent may include vitamins (e.g., vitamin D), minerals (e.g., Fe, Ca, P, Zn, B, Mg, K, Mn, Ce, Sr), non-steroidal anti-inflammatory drugs (NSAIDS) (e.g., acetaminophen), steroids (e.g., corticosteroids), immune selective anti-inflammatory derivatives (ImSAIDS) (e.g., phenylalanine-glutamine-glycine), narcotics (e.g., opioid-based narcotics such as buprenorphine), local anesthetics (e.g., benzocaine), bone growth activating factors, or any combination thereof. The bone growth activating factors may include bone morphogenetic proteins (e.g., BMP1, BMP2, BMP3, BMP4, BMP5, BMP6, BMP7, BMP8a, BMP8b, BMP10, or BMP15), fibroblast growth factor, vascular endothelial growth factor, platelet derived growth factor, or prostaglandin E2).

The filler may include one or more osteoconductive agents. The osteoconductive agent may function to provide porosity, bone ingrowth surfaces and enhanced permeability or pore connectivity or resistivity to water permeation. The osteoconductive agent may be biocompatible, insoluble, osteoconductive particles, or any combination thereof. The osteoconductive agent may be in the form of particles, short fibers, or both. Examples of suitable osteoconductive agents may include, but are not limited to, monocalcium phosphates, dicalcium phosphates, tricalcium phosphates, tetracalcium phosphates, orthophosphates, amorphous calcium phosphates, biodegradable/bioabsorbable glasses, or any combination thereof.

The filler may include one or more toughening agents. The toughening agent may function to toughen the matrix material's resistance to fracture by absorbing energy. The toughening agent may be in the form of chopped fiber, flake, particulate, or any combination thereof. The toughening agent may be of a lower modulus or higher modulus material than the polymeric material. Higher modulus materials may be included to disperse stresses throughout the matrix by interrupting crack propagation within the structure.

The filler may include plasticizer. The plasticizer may function to impart flexibility to the matrix material. The plasticizer may include a non-reactive aliphatic polyester. The plasticizer may also lower the modulus of the matrix material. The plasticizer may be present in an amount of about 5% or more, 10% or more, or even 15% or more, by weight of the matrix material. The plasticizer may be present in an amount of about 30% or less, 25% or less, or even 20% or less.

The filler may include one or more visualization agents. The visualization agent may function to assist locating a composite implant in a desired position in the body, determine the extent of degradation of a composite implant a time after implantation, or both. The visualization agent may be in the form of particles or liquid. The visualization agent may include any suitable agent typically employed in fluoroscopy. The visualization agent may be radio-opaque. The visualization agent may be visible by employing X-ray imaging. The visualization agent may include, but is not limited to, bismuth oxychloride, bismuth subcarbonate, barium, barium sulfate, ethiodol, tantalum, titanium dioxide, tantalum pentoxide, strontium carbonate, strontium halides, the like, or any combination thereof.

The polymeric material may include other typical ingredients used in composites such as pigments, dyes, adhesives, surfactants, defoamers, thickening agents, or any combination thereof.

Fibers, fiber bundles, fiber composites, and/or reinforcement elements may be coated, impregnated, or suspended in matrix material. Matrix material may fill interstitial spaces between fibers, fiber bundles, fiber composites, and/or reinforcement elements. Matrix material may function to redirect fracture lines, dissipate energy, absorb energy, or any combination thereof, resulting in a ductile failure mode. Matrix material may function to fixate fibers, fiber bundles, fiber composites, and/or reinforcement elements.

Matrix material may contribute to the mechanical properties of a composite article. The matrix material in a cured state may be low modulus and/or high ductility to achieve flexibility. Fibers, fiber bundles, fiber composites, and/or reinforcement elements may have a tensile and/or compressive strength that is greater than a matrix material in which it is located.

Matrix material may be formed into fibers (polymeric fibers), bundled with the other fibers (e.g., glass fiber), and drawn through a heated die to cause melting of the polymeric fibers and flow of the matrix material around the other fibers, thus fixating the other fibers in the matrix material. Fibers (e.g., glass fibers) may be pultruded by drawing them through a bath of matrix material and thereafter through a heated die to accelerate curing of the matrix material. The shape of the die, in either of the aforementioned methods, may determine the cross-sectional shape of the resulting fiber composite.

In preparing the larger reinforcement elements, the smaller reinforcement elements may be combined with matrix material, additional bias fiber elements (such as by binding and/or interlocking), or both.

In each step of scaling, additional bias fiber elements may be provided and/or an additional coating of a polymeric material may be provided. For example, the additional fiber elements may be an additional braid or other braid that goes around the multiples of the smaller component or between the smaller components. The additional coating may join together the smaller components and/or may coat or infiltrate the additional braid.

At least some of the properties of fibers within reinforcement elements may contribute to the wettability of the matrix material as it is applied to reinforcement elements. These properties may include fiber volume, fiber orientation, braid, braid, twist, or any combination thereof.

In one aspect of the present teachings, a polymeric material may be melted in a polymer extruder. Continuous glass reinforcement fibers may be run through the center of a cross-head die attached to the end of the extruder. When the polymer and the fiber meet inside the cross-head die, the fibers may become embedded in the polymer matrix. The polymer and fiber may exit the die in a constant two-dimensional cross-sectional shape determined by the orifice of the die. After exiting the die, the reinforcement element may be cooled and spooled on a take up reel to be used as a component in other composite cores. The extrudate may be in the form of a fiber composite. The extrudate may be cut to any desired length after cooling.

The one or more reinforcement elements may be arranged together in a variety of different configurations to fabricate a composite article. As a non-limiting example, a number of first reinforcement elements may be arranged together to form a core, a number of second reinforcement elements may be arranged around the core to form a layer, and the core and layer assembly may be impregnated with matrix material to fixate the reinforcement elements.

The matrix material may coat filaments, fibers, fiber bundles, fiber composites, reinforcement elements, or any combination thereof. The distance (viewed along a cross-section of the composite) between adjacent fibers occupied by matrix material may be greater than, generally equal to, or less than the cross-sectional length of the fibers in their largest dimension. It may be particularly advantageous for the distance (viewed along a cross-section of the composite) between adjacent fibers occupied by matrix material to be no greater than the cross-sectional length of the fibers in their largest dimension to avoid compromising the mechanical properties of the composite article.

### Composite Article—Regions

The composite article (e.g., composite implant) may comprise one or more regions. The regions may function to provide unique mechanical and/or degradation properties to the composite article. For example, a core may provide tensile strength and an outer region may delay degradation of the core. Arrangements of different regions may be employed for realizing, in a resulting composite (e.g., composite implant), a load bearing structure that exhibits attractive rigidity and ductility characteristics (as described elsewhere herein), degradation (as described elsewhere herein), or both. The regions may be discrete from one another. The regions may be distinguishable from each other on the basis of a defined border, such as a physical interface therebetween. Divisions between outer regions and/or cores may be observed when viewing a transverse cross-section of a composite.

The regions may include one or more cores, one or more outer regions, or both. The regions may be constructed in a hierarchal fashion. The regions may be constructed in a modular fashion. The modularity of the regions may provide for a scalable manufacturing process for the composite. Each of the regions may provide to the composite the same or different mechanical properties (e.g., tensile strength, compressive strength, flexural strength, torsional strength) and/or degradation properties (e.g., degradation rate) as other regions.

In general, the composite article (e.g., composite implant) may be free of any layers that are discernable from one another. Layers, as referred to herein, include discrete strata having a generally constant thickness (e.g., the thickness deviating by no more than about 5% or less, 1% or less, or even 0.5% or less), throughout the strata. Although some stacked structures may define one or more layers, due to different types of packing arrangements the stacked structure does not necessarily result in the structure being a layer. For example, elements may be arranged in an unordered arrangement where no single axis delineates between all elements. The composite article (e.g., composite implant) may be free of any layers containing both matrix and fiber that are discernable from one another. In one aspect of the present teachings, fibers, fiber bundles, fiber composites, and/or reinforcement elements may be assembled in an unordered arrangement to form a core. The unordered arrangement may be free from any discernable layers. In one aspect of the present teachings, fibers, fiber bundles, fiber composites, and/or reinforcement elements may be assembled in an ordered arrangement to form a core. The ordered arrangement may give rise to cross-sectional shapes of fibers, fiber bundles, fiber composites, and/or reinforcement elements that are distributed across the cross-section of the composite article and matrix material may be disposed in the interstitial spaces between these structures. Thus, the composite article may comprise fiber bundles, fiber composites, and/or reinforcement elements of various dimensions that generally may not be delineated by a single layer's thickness. The fiber bundles, fiber composites, and/or reinforcement elements may have a cross-sectional length in their largest cross-sectional dimension that is about 0.3 mm or more, 0.5 mm or more 1 mm or more, or even 1.5 mm or more. The fiber bundles, fiber composites, and/or reinforcement elements may have a cross-sectional length in their largest cross-sectional dimension that is about 4 mm or less, 3.5 mm or less, 3 mm or less, 2.5 mm or less, or even 2 mm or less.

The one or more regions may include one or more cores. The cores may function to provide tensile strength, compressive strength, flexural strength, torsional strength, or any combination thereof to the composite. Two or more cores in a composite may have the same or different mechanical properties and/or degradation properties. The composite article may comprise 1 or more, 2 or more, 3 or more, or even 4 or more cores. The composite article may comprise 10 or less, 9 or less, 8 or less, 7 or less, or even 6 or less cores. Two or more cores may be concentrically arranged one within another.

Divisions between cores and/or between cores and outer regions may be observed when viewing a transverse cross-section of a composite article. The cores may have a thickness of about 0.1 mm or more, 1 mm or more, 5 mm or more, 10 mm or more, or even 20 mm or more. The cores may have a thickness of about 1,000 mm or less, 800 mm or less, 300 mm or less, 100 mm or less, or even 50 mm or less. The thickness may be measured along a transverse axis of the composite. The thickness may be observed when viewing a transverse cross-section of a composite.

The cores may be fabricated from a plurality of reinforcement elements. The cores may comprise 10 or more, 20 or more, 40 or more, or even 60 or more reinforcement elements. The cores may comprise 500 or less, 200 or less, 100 or less, or even 80 or less reinforcement elements. One or more cores may comprise the same or different number of reinforcement elements as compared to one or more other cores.

The reinforcement elements may be oriented axially, at a bias, or both. One or more bias reinforcement elements may be oriented at the same or different angle as compared to one or more other bias reinforcement elements. The reinforcement elements may be in the form of rods, sheets, tape, or any combination thereof.

The reinforcement elements may comprise one or more fibers, fiber bundles, fiber composites, matrix material, fillers, or any combination thereof. Each of the reinforcement elements may comprise the same or different selection of fiber material, the selection of matrix material, fiber count, fiber volume, fiber orientation, braid, layup, twist, cross-sectional shape, cross-sectional thickness, cross-sectional aspect ratio, cell size, or any combination thereof, as compared to other reinforcement elements in the same core. By way of example, one core may include a first type of matrix material and another core may include a second type of matrix material.

The cores may comprise one or more fibers, fiber bundles, fiber composites, matrix materials, fillers, or any combination thereof. The core may comprise 1 or more, 10 or more, 50 or more, or even 100 or more fiber bundles, fiber composites, or both. The core may comprise 100,000 or less, 10,000 or less, 1,000 or less, or even 500 or less fiber bundles, fiber composites, or both.

One or more fiber bundles may bind or interlock a plurality of reinforcement elements, or both. Forces translating across a thickness of a core may be translated by fiber bundles that bind, interlock, or both. Fiber bundles that interlock may substantially prevent delamination of reinforcement elements.

One or more fiber bundles may interlock two or more cores, or both. One or more fiber bundles may interlock one or more cores with one or more outer regions. Fiber bundles that interlock may substantially prevent delamination between two or more cores, between one or more cores and one or more outer regions, or both.

After assembling a plurality of reinforcement elements, fibers, fiber bundles, matrix material, fillers, or any combination thereof, a core may be made to have a first shape. The core may be made to define a preform.

Cores, outer regions, and/or precursor elements (e.g., fiber bundles) may be selected and combined to provide unique properties to the composite article that may not be realized by employing each structure in isolation. For example, axial fiber bundles may be stiff and responsive, braids of fiber bundles may provide resistance to crack propagation, and matrix (and optionally filler) may dissipate energy translated through the composite article. A composite article may be fabricated from a combination of axial fiber bundles, braids of fiber bundles, and matrix (and optionally filler) to provide a composite article with the combined properties of its subparts.

The core may be coated, subjected to a surface treatment, or both. The core may be coated and/or surface treated to add material, remove material, or both. Non-limiting examples of surface treatment may include abrasive blasting, mass finishing, polishing, exposing the surface to radiation to cause a change in structure of the surface (e.g., laser etching or laser engraving), depositing one or more thin films (i.e., angstroms to nanometers in a thickness taken across a section perpendicular to the surface) and/or microparticles on the surface (i.e., microns in thickness taken across a section perpendicular to the surface), chemical etching, machining (e.g., milling, lathing, or grinding). The one or more thin films and/or microparticles may have a peak height relative to the surface of about 1 nm or more, 500 nm or more, or even 1 μm or more. The one or more thin films and/or microparticles may have a peak height relative to the surface of about 1 mm or less, or even 500 μm or less. Surface roughness may be imparted by machining. The surface roughness may be a predetermined and deliberate artifact of tooling employed in the process of machining.

A core may be subjected to material addition. The material addition may form an outer region. The outer region may include a surface roughness, surface porosity, or both. The outer region may include surface features, discussed elsewhere herein. The material addition may form the surface roughness, surface porosity, or both. The material addition may include molding, overmolding, thin film deposition, 3D printing deposition, inkjet deposition, dip coating, spraying, rolling, swabbing, brushing, extrusion coating, the like, or any combination thereof.

After coating, a core may be subjected to material removal. The material removal may be performed by milling, lathing, laser etching, laser engraving, chemical etching, the like, or any combination thereof. The material removal may reveal structures on the surface of a core. The structures may include surface projections, threading, barbs, the like, or any combination thereof.

One or any combination of processes discussed in the preceding paragraphs may be employed on an exposed surface of a core, on an exposed surface of an outer region, or both.

The one or more regions may include one or more outer regions. The one or more outer regions may function as a permeable barrier to control the ingress of fluids into the core, control the degradation rate of the composite article, compatibilize the composite (e.g., implant) with a surrounding environment, deliver one or more functional agents (e.g., a therapeutic medicament, in the case of a composite implant) into a surrounding environment, or any combination thereof. The outer region may circumscribe the one or more cores.

The outer regions may be fabricated from polymeric matrix material. The matrix material may include filler dispersed therein. The filler may be nano-scale filler (e.g., <1 μm in its largest dimension). The nano-scale filler may be fabricated from soluble silicate glass, hydroxyapatite, magnesium hydroxide, magnesium oxide, the like, or any combination thereof.

The size of the filler may be sufficiently small to avoid appreciably increasing the viscosity of the polymeric matrix material. The filler may be a high aspect ratio filler (e.g., 10:1 or more, 20:1 or more, or even 30:1 or more). The filler may include surface modification in order to improve the interface between the filler and polymeric matrix material. The filler may be present in the polymeric matrix material in an amount of about 5% or more, 7% or more, 9% or more, or even 11% or more, by weight of the polymeric matrix material. The filler may be present in the polymeric matrix material in an amount of about 19% or less, 17% or less, 15% or less, or even 13% or less, by weight of the polymeric matrix material.

The composite article may comprise 1 or more, 2 or more, or even 3 or more outer regions. The composite article may comprise 6 or less, 5 or less, or even 4 or less outer regions. Where more than one outer region is employed each of the outer regions may provide the same or different degradation rate as compared to other outer regions.

The outer region may have a thickness (cross-sectional) of about 0.5 μm or more, 1 μm or more, 10 μm or more, 20 μm or more, 40 μm or more, or even 60 μm or more. The outer region may have a thickness of about 150 μm or less, 120 μm or less, 100 μm or less, or even 80 μm or less.

One or more outer regions may comprise the same or different material as one or more other outer regions.

The outer regions may be applied to one or more cores via dip coating, extrusion coating, over molding, or any combination thereof. Overmolding may form structures on the surface of a core. The structures may include surface projections, threading, barbs, the like, or any combination thereof.

After application of one or more outer regions, they may be subjected to material removal. The material removal may be performed by milling, lathing, laser etching, laser engraving, chemical etching, the like, or any combination thereof. The material removal may reveal structures on the surface of an outer regions. The structures may include surface projections, threading, barbs, the like, or any combination thereof.

The outer regions may have a thickness of about 0.01 mm or more, 0.1 mm or more, or even 1 mm or more. The outer regions may have a thickness of about 1 cm or less, 8 mm or less, 5 mm or less, or even 2 mm or less. The thickness may be measured along a transverse axis of a composite.

A ratio of reinforcement elements included in the cores and reinforcement elements included in the outer regions may be about 1:0.01 or more, 1:0.1 or more, or even 1:0.5 or more. A ratio of reinforcement elements included in the cores and reinforcement elements included in the outer regions may be about 1:2 or less, 1:1.5 or less, or even 1:1 or less.

The final composite article (e.g., composite implant), reinforcement elements, or both may have a fiber volume (FV) of about 20% or more, 40% or more, or even 50% or more. The final composite article (e.g., composite implant), reinforcement elements, or both may have a fiber volume (FV) of about 90% or less, 80% or less, 70% or less, or even 60% or less.

### Surface Modifications

Surface modifications may be applied to and/or incorporated into one or more containment bags, fibers, fiber bundles, fiber composites, matrix material, fillers, cores, outer regions, or any combination thereof. For example, a first surface modification may be applied to fibers to enhance the fiber's interaction with polymeric material and a second surface modification may be used on an outer region to enhance the outer region's interaction with bone.

Surface modifications may be applied to one or more constituent parts of the composite article to modulate degradation rates. For example, surface modifications may be applied to individual fibers, individual reinforcing elements, one or more cores, one or more outer regions, or any combination thereof. In this manner, degradation of each constituent element of the composite article may be delayed, prolonging the useful life of the composite article.

The composite article may include one or more surface modifications. The surface modification may provide chemical functionality to the outer surface and/or one or more inner layers of the composite article or constituent elements thereof. For example, a surface modification may be applied to fibers for improved adhesion to matrix material. The surface modification may introduce one or more chemical moieties or functional groups to a surface and/or one or more inner layer of the composite article or constituent elements thereof. The surface modification may functionalize the composite article or constituent elements thereof to adapt the same to a microenvironment in which its use is intended. For example, the surface modification may functionalize the composite article or constituent elements thereof to be more bioactive (e.g., for stimulating osteogenesis or angiogenesis), impart bacterial and/or microbial control (e.g., antibacterial, antimicrobial, bacterial static, or biocidal properties, or any combination thereof), create a gradient of refractive index, create discrete layers of different refractive indexes, or any combination thereof. The surface modification may alter the chemical durability of the composite article or constituent elements thereof. The surface modification may alter mechanical properties of the composite article or constituent elements thereof.

The surface modification may catalyze or otherwise accelerate a polymerization reaction of monomer or prepolymer in contact with the surface modified element of the composite article. For example, a sizing agent applied to fibers may provide improved adhesion between a polymeric material and the fibers, as well as act as a secondary catalyst for the polymerization of monomers in a polymeric material.

The surface modification may include one or more, two or more, three or more, or even four or more similar or different surface modifications on the same composite article or constituent elements thereof.

In one aspect, the surface modification may be applied to fibers. Some, or all of the fibers may include a surface modification. The surface modification may be applied to fibers before or after assembly into a fiber bundle.

The surface modification may penetrate to various depths of the composite article or constituent elements thereof (i.e., depth as measured from the perimetric surface to the center). A composite article or constituent element thereof, having a cross-sectional length (c), may be surface modified to a depth of c or less, 0.5c or less, or even 0.1c or less. A composite article or constituent element thereof, having a cross-sectional length (c), may be surface modified to a depth of 0.0001c or more, 0.001c or more, or even 0.01c or more. Different surface modifications on the same composite article or constituent element thereof may penetrate to the same or different depths. Different surface modifications on the same composite article or constituent element thereof may penetrate to a combined total depth of c. The depth of the surface modification may be about 1 μm or more, 3 μm or more, 10 μm or more, 30 μm or more, or even 60 μm or more. The depth of the surface modification may be about 400 μm, or less, 300 μm or less, 200 μm or less, 150 μm or less, 100 μm or less, or even 80 μm or less.

The composite article or constituent elements thereof may have one or more layers of variable solubility. The variation of the solubility may be gradual, with no defined core or layer boundaries. The variation of the solubility may be step-wise.

The composite article or constituent element thereof, with a surface modification applied thereto, may have a solubility of about 1*10⁻⁹ mg/cm²-h or more, 1*10⁻⁸ mg/cm²-h or more, or even 1*10⁻⁷ mg/cm²-h or more. The composite article or constituent element thereof, with a surface modification applied thereto, may have a solubility of about 1*10⁻¹ mg/cm²-h or less, 1*10⁻² mg/cm²-h or less, or even 1*10⁻³ mg/cm²-h or less.

The composite article or constituent element thereof, with a surface modification applied thereto, may have a solubility that is 10% or more, 30% or more, 50% or more, 70% or more, or even 100% or more, as compared to the original solubility absent the surface modification. The composite article or constituent element thereof, with a surface modification applied thereto, may have a solubility that is 1,000% or less, 700% or less, 500% or less, or even 300% or less, as compared to the original solubility absent the surface modification.

The surface modification may include, without limitation, coating with a sizing agent, coating with a bulking agent, ion exchange, coating with primer, coating with an amino functional material, treatment to provide an oxidized surface (e.g., plasma treatment, corona treatment, ozone treatment, and acidic/basic treatment), treatment to provide hydroxyl groups on the surface (which can react or provide improved adhesion with the matrix material), surface abrasion, or any combination thereof.

Coating may be performed by dip coating, spin coating, spray-on coating, plasma deposition, chemical vapor deposition, the like, or any combination thereof.

The surface modification may be modulated using ions, temperature, incubation time, or any combination thereof.

The surface modification may include coating with a sizing agent. The sizing agent may function as a coupling agent, compatibilizer, barrier, or any combination thereof. The sizing agent may function to enhance bonding with polymeric material, increase reinforcement dimensions, modulate hydro-diffusion access/rates, or any combination thereof. The sizing agent may be less soluble than the material of the constituent element of the composite article. The sizing agent may be insoluble.

The sizing agent may be applied to fibers. The sizing agent may be applied to glass fibers. Fibers may be fabricated with a sizing agent by coating (e.g., dip coating, spray coating, rolling, brushing, swabbing, the like, or any combination thereof). The coating may be performed after formation of fibers, during the cool-down process immediately after formation of fibers, after fibers have been formed into fibrous bundles, or any combination thereof.

The sizing agent may have a thickness (cross-sectional) of about 0.5 μm or more, 1 μm or more, 10 μm or more, 20 μm or more, 40 μm or more, or even 60 μm or more. The sizing agent may have a thickness of about 150 μm or less, 120 μm or less, 100 μm or less, or even 80 μm or less.

The sizing agent may be biodegradable and/or bioabsorbable. The sizing agent may degrade from the outside in.

The sizing agent may be applied to fibers in an in-line process. The sizing agent may be applied to fibers in the same in-line process as matrix material is applied to fibers. The sizing agent may be applied to fibers before or after the application of matrix materials to fibers. The sizing agent may react with the matrix material during the in-line process to provide for improved interfacial bonding between the sizing agent and matrix material.

In general, the thickness of the sizing agent layer around fibers may increase the surface area of the sizing agent and thus increase the degradation rate of the same.

The sizing agent may include aminos, proteins, carbohydrates, or any combination thereof. The aminos may include ureas, y-Aminopropyltriethoxy silane, aminopropylsilane, 3-aminopropylsilane, (3-aminopropyl)trimethoxysilane(APTMS), or any combination thereof. The proteins may include soy proteins, corn proteins, peanut proteins, lysine, the like, or any combination thereof. The proteins may include functional groups such as amine, sulfhydryl, carboxylic acid, or any combination thereof. The carbohydrates may include functional groups such as hydroxyl, amine, or both.

The sizing agent may include organic molecules with multiple hydrogen bonding sites for coupling to fibers. Examples of suitable molecules may include calixarenes, polyhydric alcohols, polyamines, polyamino acids, ployacrylic acids, polyacrylamides, the like, or any combination thereof.

The sizing agent may include metal coating, ceramic coatings, polymeric coatings, inorganic salt coatings, metal phosphates coating, (e.g., phosphates of Fe, Ca, Mg, Zn, Ni, the like, or any combination thereof), or any combination thereof. The sizing agent may be biodegradable, bioabsorbable, or both.

Examples of suitable metal coating may include, but are not limited to, Mg, Ag, Ni, Ti, Ca, alloys thereof, or any combination thereof. The metal coating may react with water to provide basic/alkaline products that can act as buffering and degradation control agents for the matrix material and/or fibers.

Examples of suitable ceramic coating may include ethyoxysilanes such as tetraethoxysilane, methyltriethoxysilane, dimethyldiethoxysilane, or trimethylethoxysilane; polycarbosilane; polysilazanes such as perhydropolysilazane or polysilizane; modified polyamines; or any combination thereof.

Examples of suitable polymeric coatings may include silanes, amino silanes, lysine, polyamines, amino acids, polyamino acids, or any combination thereof.

The sizing agent may comprise one or more compatibilizers (“coupling agent”). The compatibilizer may function to promote chemical adhesion between fibers and matrix material, between fibers and a surrounding environment (e.g., body of a living being), between matrix material and a surrounding environment (e.g., body of a living being), or any combination thereof. The compatibilizers may also improve mechanical properties, physical properties, osseointegration, or any combination thereof.

Examples of suitable compatibilizers and methods of applying the same may include, but are not limited to, calcium phosphate, hydroxyapatite, calcium apatite, fused-silica, aluminum oxide, apatite, wollastonite, glass, bioglass, compounds of calcium salt, phosphorus, sodium salt and/or silicates, maleic anhydride, diisocyanates, epoxides, silanes, cellulose esters, or any combination thereof.

The sizing agent may comprise bulking agents. The bulking agent may function to maintain interfacial contact at least partially between fibers and matrix material. In a composite that is in contact with water or other aqueous solution, fibers may lose contact with the polymer material due to dissolution. This phenomenon may result in an increased degradation rate and loss of mechanical properties. The bulking agent may swell or expand as it absorbs water and maintaining contact between the fibers and the polymeric material.

An example of a suitable bulking agent may include polysaccharide derivatives (e.g., sodium alginate). Layers of sodium alginate may be combined with layers of a calcium slat that is insoluble at neutral pH, but soluble at low pH. As the fibers dissolve, they may release phosphoric acid which can dissolve the calcium salt. The calcium ions generated can diffuse in the alginate coating, forming a swellable alginate gel, with improved mechanical properties compared to sodium alginate. Thus, the multi-layered coatings can act as bulking agent, as a buffering agent, and as a dynamic method of maintain interfacial contact as fibers degrade.

The bulking agent may be included as an additive in the polymeric material.

The matrix material may include an additive that is a water scavenger to either react with or immobilize water upon entry to a matrix material in order to retard water uptake by the matrix material after or during the matrix material is cured and/or hardened.

The surface treatment may include one or more ion exchanges. The ion exchange may function to achieve a desired degradation rate, ion release profile, glass strengthening, or any combination thereof. The ion exchange may be particularly advantageous as applied to glass fibers. The ion exchange may create a gradient of degradation rates and/or packing density of ions in the fibers.

The ion exchange may replace ions in the fiber with ions that are a different size than the ions already present in the fiber. The replacing ion may be larger than, smaller than, or the same size as the ion it is replacing. It may be particularly advantageous, for increased glass strength, to replace ions with larger ions, which puts stress on surrounding atomic bonds. For soluble glasses, ions such as sodium cation or phosphate anion may be replaced with less soluble ions, such as calcium, magnesium, iron cations, silicate anions, or any combination thereof.

The surface modification may include surface abrasion. The surface modification may accelerate the degradation rate of the composite article or constituent elements thereof. Surface abrasion may increase the surface area subject to degradation. Surface abrasion may be performed by acid/base etching, mechanical abrasion, or plasma oxidation/reduction. Surface abrasion may modify the mechanical properties of the composite article or constituent elements thereof. Surface abrasion may modify the biocompatibility and/or osseointegration.

### Degradation

Degradation of the composite article or constituent elements thereof may be deliberately modulated. As referred to herein, degradation may mean the reduction of molecular weight, mass, strength, or any combination thereof. Degradation may be modulated to increase the useful life of the composite article. Degradation may be modulated to provide a composite article with mechanical properties, as taught herein, that persist for a predetermined period of time. The composite article may maintain its mechanical properties for about 1 week or more, more preferably about 6 weeks or more, more preferably 12 weeks or more, more preferably 24 weeks or more, more preferably 48 weeks or more, or even more preferably 60 weeks or more.

Degradation may involve the reduction of weight of the composite article as soluble chemical species are dissolved in aqueous solution, the reduction of the molecular weight of polymeric materials, or both. The molecular weight of polymeric materials may reduce to a sufficient amount to be dispersed in the local environment. The molecular weight of the polymeric material may reduce to a sufficient amount to be metabolized by enzymes. The molecular weight of the polymeric material may reduce to a sufficient amount to be metabolized by the human body.

In medical applications according to the present teachings, degradation may be deliberately modulated to prevent ionic or molecular species to degrade and build up into the local environment in which the composite implant is located to a concentration that would instigate negative physiological responses. For example, a composite implant may include magnesium ions which, if degraded rapidly, may cause blood magnesium concentrations to increase to a level clinically referred to as hypermagnesemia. At least one physiological consequence of hypermagnesemia is cardiac arrest. As another example, polymeric material shed from the composite implant which, if degraded rapidly, may cause a local concentration of polymeric material to increase to a level that may exacerbate an immune response. Thus, the present teachings contemplate deliberately modulating the rate of degradation to release degradation byproducts gradually at a rate that is generally proportional to the metabolization rate of the degradation byproducts. In this manner, the concentration of degradation byproducts may be generally constant or at least not increase above clinically significant blood concentrations.

In medical applications according to the present teachings, degradation may cause large masses of the composite implant to fracture from the composite implant. The large masses may be characterized by a size, in their largest dimension, of about 50 nm or more, 100 nm or more, 500 nm or more, or even 1,000 nm or more. The large masses may be characterized by a size, in their largest dimension, of about 3 mm or less, 1 mm or less, 500 μm or less, 100 μm or less, or even 50 μm or less. Fracturing of large masses may compromise the mechanical properties of the composite implant. Fracturing of large masses may expose a greater surface area of the composite implant to degradation conditions, resulting in an increase in the rate of degradation.

In medical applications according to the present teachings, degradation may be deliberately modulated to maintain mechanical properties above a pre-determined threshold for a period of time during the healing of bone and/or tissue. Maintaining mechanical properties above a pre-determined threshold may allow patients to subject, at least in a limited capacity, parts of their body to loads, activity, movement, or otherwise. Use of body parts that have been injured, at least to a limited extent, may improve healing by promoting proper bone and/or tissue growth, accelerating bone and/or tissue growth, or both.

In medical applications according to the present teachings, degradation may be deliberately modulated to maintain an interface between the composite implant and bone and/or tissue. For a period of about 1 week, more preferably about 4 weeks, more preferably about 6 weeks, more preferably about 12 weeks, more preferably about 24 weeks, more preferably about 48 weeks, or even more preferably about 60 weeks, the composite implant may maintain an interface with bone and/or tissue that is about 70% or more, more preferably 80% or more, more preferably 90% or more, or even more preferably 99% or more of the envelope in which the composite implant resides.

In medical applications according to the present disclosure, soon (e.g., 1 to 4 hours) after implantation into a living being, the body may initiate a foreign-body response to the composite orthopedic implant. The foreign-body response may include an acute phase and chronic phase.

During the acute phase, blood proteins (e.g., albumin), tissue proteins (e.g., fibrinogen), neutrophils, and other biological materials (e.g., complement fragments (fragments of proteins cleaved during immune response to complement the functions of antibodies) and non-specific antibodies) may adsorb to the perimetric surface of the orthopedic implant.

During the chronic phase, the biological materials of the acute phase may initiate the conglomeration of macrophages, monocytes, or both. These may fuse to form multinucleated giant cells. All of the aforementioned biological materials of the acute phase and chronic phase may cooperate to form a fibrous capsule during the chronic phase.

Degradation may be deliberately modulated to maintain an interface between matrix and fillers, matrix and fibers, cores and other cores, cores and outer regions, or any combination thereof. For a period of about 1 week, more preferably about 4 weeks, more preferably about 6 weeks, more preferably about 12 weeks, more preferably about 24 weeks, more preferably about 48 weeks, or even more preferably about 60 weeks, the interface may be about 70% or more, more preferably 80% or more, more preferably 90% or more, or even more preferably 99% or more of the surface area between structures. Loss of interface may result in loss of mechanical properties. This may be due, at least in part, to the loss of load distribution across interfaces. Thus, it is important to ensure that a sufficient interface is maintained during the useful life of the composite article. Loss of interface may result in a higher rate of degradation. This may be due, at least in part, to the increased surface area exposed to aqueous solution.

It may be particularly advantageous to modulate the interface between matrix and fillers because fillers may account for a large amount of surface area in the composite article. The interface may be modulated by applying one or more surface modifications to fillers.

Degradation may be modulated by deliberately selecting materials from which fibers and/or matrix material are fabricated. Different materials may provide different inherent degradation properties. For example, some ions present in glass fibers may be more or less soluble in aqueous solution as compared to other ions. As another example, some polymeric materials may hydrolyze at a higher or lower rate as compared to other polymeric materials.

Degradation may be modulated by influencing the local environment of the composite article. As referred to herein, the local environment may mean a volume of space within the composite (e.g., within internal passages in the composite) and/or a region surrounding the composite. The local environment may be influenced by increasing or decreasing temperature, moisture, pH, or any combination thereof. Increasing temperature may generally increase the solubility of soluble particles in aqueous solution, increase the reaction rate of hydrolysis, or both. Increasing moisture may increase the rate of hydrolysis. Increasing or decreasing pH above or below a range of 7.3-7.5 may increase the rate of hydrolysis.

Dissolution of glass into aqueous solution may increase or decrease pH. For example, degradation of bioglass and silicate glass produces alkaline species. As another example, degradation of phosphate glasses produces acidic species. The choice of cations paired with soluble ions (e.g., phosphate, silicate, or borate) in glass may modulate the solubility of the phosphate. For example, sodium and potassium are more soluble that calcium and magnesium, and aluminum and iron the least soluble. Hydrolysis of polymeric material may produce acidic or basic species (e.g., carboxylic acid or hydroxyl groups).

The duration, intensity, and sequence of the release of degradation byproducts may be deliberately designed to produce pH shifts in a local environment or to release other compounds into the local environment. For example, the degradation profile may include at least one stage in which there is a rapid release of degradation byproducts for a burst of either acidic or basic species to shift pH and/or at least one stage in which degradation byproducts buffer an aqueous solution.

Degradation may be modulated by deliberately selecting materials that degrade enzymatically, nonenzymatically, or both. Some polymeric materials may rely solely or at least primarily on enzymatic degradation. For example, this may be the case for polyhydroxyalkanoates (PHA) (e.g., poly(4-hydroxybutyric acid) (P4HB). A combined nonenzymatic and enzymatic degradation may accelerate the combined rate of degradation.

Additives may be applied to polyhydroxyalkanoates (PHA) (e.g., poly(4-hydroxybutyric acid) (P4HB) to encourage non-enzymatic degradation. The additives may include PLA, PCL, PGA, poly-β hydroxybutyrate (PHBA), PHBA/p-hydroxyvalerate, poly-3-hydroxybutyrate-co-3-hydroxy valerate (PHBV) copolymers (e.g., PHBA/PHVA), poly-β-hydroxypropionate (PHPA), poly-3-hydroxyproprionate (PHP), poly-4-hydroxybutyrate (P4HB) homopolymers and copolymers, or any combination thereof. The additives may shift the local pH, which may initiate non-enzymatic degradation via hydrolysis.

Degradation may be modulated by increasing or decreasing the surface area-to-mass ratio of the composite implant or constituent elements thereof. Generally, increasing the surface area increases exposure to an environment that promotes degradation. This environment may include aqueous solution, buffered solution, acidic solution, basic solution, enzymes, or any combination thereof. The surface area-to-mass ratio may be modulated by the formation of surface roughness, surface porosity, internal passages, or any combination thereof.

The surface area-to-mass ratio of the composite article may increase over time as the composite article biodegrades. During one or more stages of a degradation profile, the composite article may include surface roughness, surface porosity, internal passages, or any combination thereof.

The surface roughness, surface porosity, internal passages, or any combination thereof may be interconnected during at least one stage of degradation. Interconnectivity may increase as degradation progresses. The passages may arise from the degradation of fibers, filler, or both. The shape of the passages during at least one stage during degradation may correspond to the shape of the fibers, filler, or both that previously occupied the passages. Fibers, filler, or both may be uniformly distributed throughout the composite article. As a result, the formation of passages may be generally uniformly distributed throughout the composite article and provide for a generally uniform degradation of the composite article. The composite article may contain one or more regions of higher concentration of fibers, filler, or both as compared to other regions of the composite article. Regions of higher fiber and/or filler concentration may provide those regions with a higher concentration of passages upon degradation thus resulting in a higher degradation rate in those regions. For example, distal ends of the composite article (e.g., composite implant) may include higher concentrations of fibers and/or fillers to promote faster degradation of the ends and exposure of the internal structure of the composite article to degradation conditions.

During at least one stage of the degradation profile, the composite article may include surface roughness, surface porosity, internal passages, or any combination thereof that have a size in their largest dimension of about 1 nm or more, 10 nm or more, 100 nm or more, or even 200 nm or more (nano scale). During at least one stage of the degradation profile, the composite article may include surface roughness, surface porosity, internal passages, or any combination thereof that have a size in their largest dimension of about 990 nm or less, 800 nm or less, 600 nm or less, or even 400 nm or less (nano scale). The composite article may include nano scale surface roughness, surface porosity, internal passages, or any combination thereof prior to being exposed to degradation conditions.

During at least one stage of the degradation profile, the composite article may include surface roughness, surface porosity, internal passages, or any combination thereof that have a size in their largest dimension of about 1 μm or more, 10 μm or more, 100 μm or more, or even 200 μm or more (micro scale). During at least one stage of the degradation profile, the composite article may include surface roughness, surface porosity, internal passages, or any combination thereof that have a size in their largest dimension of about 990 μm or less, 800 μm or less, 600 μm or less, or even 400 μm or less (micro scale). The composite article may include micro scale surface roughness, surface porosity, internal passages, or any combination thereof after being exposed to degradation conditions for a period of time.

During at least one stage of the degradation profile, the composite article may include surface roughness, surface porosity, internal passages, or any combination thereof that have a size in their largest dimension of about 1 mm or more, 1.5 mm or more, 2 mm or more, or even 2.5 mm or more (macro scale). During at least one stage of the degradation profile, the composite article may include surface roughness, surface porosity, internal passages, or any combination thereof that have a size in their largest dimension of about 4 mm or less, 4.5 mm or less, 4 mm or less, or even 3.5 mm or less (macro scale). The composite article may include macro scale surface roughness, surface porosity, internal passages, or any combination thereof after being exposed to degradation conditions for a period of time.

In medical applications according to the present teachings, surface roughness, surface porosity, internal passages, or any combination thereof may function as a scaffold for bone and/or tissue ingrowth. Bone and/or tissue may infiltrate the composite implant from the outside-in and/or inside-out (where the composite implant includes a cannulation). Bone and/or tissue may occupy volume that was previously occupied by fibers, filler, and/or matrix material prior to their degradation. The degradation may be deliberately tailored to balance the volume loss of the composite implant and the volume occupied by newly developed bone and/or tissue.

Surface roughness, surface porosity, internal passages, or any combination thereof may modulate the ingress rate of aqueous solution into the composite article, egress rate of aqueous solution out of the composite article, or both. It may be particularly advantageous to balance the ingress rate and/or egress rate to the rate of hydrolysis and/or soluble species (e.g., ion) dissolution. It may be particularly advantageous for the ingress rate to be greater than the hydrolysis rate in order to provide an adequate concentration of aqueous solution to influence hydrolysis. It may be particularly advantageous for the egress rate to be greater than the rate of hydrolysis in order for hydrolyzed oligomers and/or monomers to be carried to an environment surrounding the composite article and to avoid localized buildup of hydrolyzed oligomers and/or monomers. It may be particularly advantageous for the egress rate to be greater than the rate of dissolution of soluble (e.g., ion) species in order for soluble species in solution to be carried to an environment surrounding the composite article and to avoid localized buildup of soluble species. If the rate of hydrolysis of polymeric material and/or dissolution of soluble species is greater than the egress rate, localized buildup of the same may catalyze hydrolysis to a great extent.

Degradation may be modulated by the addition of glass filler. The glass filler may be in the form of granules, segments, nanotubes, whiskers, nanorods, the like, or any combination thereof. The glass filler may dissolve in aqueous solution leaving behind pores and or passages. Dissolution of glass filler may release ionic species in aqueous solution, influencing acidic or basic conditions. The glass filler may have a relatively high aspect ratio. The glass filler may have an aspect ratio of about 1:1 or more, 5:1 or more, 10:1 or more, or even 20:1 or more. The glass filler may have an aspect ratio of about 100:1 or less, 80:1 or less, 60:1 or less, or even 40:1 or less.

The filler may include nano filler, micro filler, or both. The nano filler may have a length in its largest dimension of about 1 nm or more, 10 nm or more, 50 nm or more, 100 nm or more, or even 200 nm or more. The nano filler may have a length in its largest dimension of about 990 nm or less, 800 nm or less, 600 nm or less, or even 400 nm or less. The micro filler may have a length in its largest dimension of about 1 μm or more, 10 μm or more, 50 μm or more, 100 μm or more, or even 150 μm or more. The micro filler may have a length in its largest dimension of about 500 μm or less, 450 μm or less, 400 μm or less, 350 μm or less, or even 300 μm or less.

Degradation may be modulated by the addition of buffering agents to fibers, polymeric material, or both. The buffering agents may release into an aqueous environment upon dissolution and/or hydrolysis. The buffering agents may be inorganic and/or organic. The buffering agents may comprise a weak acid and it's conjugate base. The buffering agents in aqueous solution may prevent pH of the aqueous solution from increasing or decreasing more than 2 units, more preferably more than 1.5 units, more preferably more than 1 unit, or even more preferably more than 0.5 units. Suitable inorganic bases may include salts, oxides, and/or hydroxides of alkaline metals (e.g., basic mono-phosphates, di-phosphates, and tri-phosphates, calcium oxide, calcium hydroxide, magnesium oxide, magnesium hydroxide, bioglass flakes, calcium phosphate, beta tricalcium phosphate, hydroxyapatite, potassium stearate, and sodium stearate). Suitable organic bases, such as polyamines, bispidines, and proton sponges, are examples of self-buffering agents. The self-buffering or degradation controlling agents can be encapsulated in a micro- or nano-capsule and are released under certain physiological conditions.

Hydrolytic, dissolutive, and/or enzymatic degradation may be initiated at a surface and progress generally inwards.

Additives may be included that promote enzymatic degradation. Some of the enzyme additives can be produced in a fiber form and used, with appropriate compatibilizers, to increase the mechanical properties (e.g., strength and stiffness) of the material, thereby increasing the utility of the material to higher load-bearing applications while still maintaining a biodegradable classification. A suitable enzyme may include lipases, which may be employed to hydrolyze esters and polyesters. A suitable enzyme may include proteases, which may be employed to cleave amide bonds.

Pores may be formed in the matrix material as degradation progresses. Pores may function to provide paths of ingress and/or egress of aqueous solution, provide paths of ingress for bone and/or tissue ingrowth, or both. Generally, the quantity, tortuosity, and/or size of pores increases as degradation progresses. Porosity may increase at a generally linear rate, progressive rate, or exponential rate. Porosity may be formed in the matrix material of a composite article, reinforcement elements, fiber composites, or any combination thereof.

A plurality of pores within the composite article may have a variety of pore sizes. Thus, pore size, as referred to herein, means an average pore size. The average pore size may be about 0.0005 μm or more, 0.0010 μm or more, 0.010 μm or more, or even 0.10 μm or more. The average pore size may be about 1000 μm or less, 500 μm or less, 100 μm or less, 50 μm or less, 10 μm or less, 5 μm or less, or even 1.0 μm or less.

The composite article and/or constituent elements thereof may have a porosity that is generally uniform or non-uniform. The composite article and/or constituent elements thereof may have a porosity that is deliberately formed on one or more regions thereof while one or more other regions are free of porosity. The composite article and/or constituent elements thereof may have a volume of about 10% or more, 20% or more, 30% or more, or even 40% or more occupied by porosity. The composite article and/or constituent elements thereof may have a volume of about 100% or less, 90% or less, 80% or less, or even 70% or less occupied by porosity. The composite article and/or constituent elements thereof may have a surface area of about 10% or more, 20% or more, 30% or more, or even 40% or more occupied by porosity. The composite article and/or constituent elements thereof may have a surface area of about 100% or less, 90% or less, 80% or less, or even 70% or less occupied by porosity.

Porosity may be generated by dissolution and/or absorption of Porosity may be generated by the dissolution and/or absorption of fibers Porosity may develop from solubilizing filler. For example, bone may absorb calcium sulfate, α-tricalcium phosphate, bioglass, or the like. Porosity may develop from the migration of solvents from matrix material.

The matrix material may become porous over time. The matrix material may become porous after implantation into a living being. The pores present in the matrix material may have a diameter of about 50 μm or more, 100 μm or more, 150 μm or more, or even 200 μm or more. The pores present in the matrix material may have a diameter of about 500 μm or less, 45° or less, 400 μm or less, 350 μm or less, or even 300 μm or less. During at least one stage of degradation, the pores may have a diameter of about 100 μm or more to allow bone and/or tissue ingrowth.

The pore size may be modulated by bundle size. During degradation, bundles may degrade prior to the degradation of matrix material. As a result, the space occupied by bundles within the matrix material may give rise to pores having generally the same dimensions (e.g., transverse cross-sectional length) as the bundles.

The pores may be interconnected in their as-manufactured state. The pores may not be interconnected in their as-manufactured state but may become interconnected for defining one or more flow channels (passages) within the composite (e.g., composite implant). A plurality of different flow channels may be formed at a different time period from the initiation of degradation.

The pore size ranges from 0.001 Angstroms to 500 Angstroms or more. The pore size can help to determine the rate of degradation. The sizing and/or polymeric material may also comprise additional therapeutic molecules, or molecules for facilitating wound healing (or otherwise to deliver localized treatments). The therapeutic molecules may be particulates that are added as filler within a matrix of polymeric material. The therapeutic molecules may be provided as a surface as a coating on bundles, fibers, and/or filaments. The therapeutic molecules may be embedded within the matrix of polymeric material to a predetermined depth from the surface, in a predetermined amount, and/or according to a predetermined perimetric surface geometry (e.g., characterized by an aspect ratio, such as the aspect ratio for fillers provided herein).

Pore sizes may be dynamic and the choice of material and/or structure will be such as to achieve a plurality of different pore size ranges, degrees of pore interconnectedness, and/or dispersity of pore sizes over one or more periods of time. For example, there may be a first preferred range and/or dispersity of pore size during a first period corresponding with the time of implantation through a first selected date. For example, there may be a second preferred range and/or dispersity of pore size during a second period corresponding with the time of implantation through a second selected date. For example, there may be a third preferred range and/or dispersity of pore size during a third period corresponding with the time of implantation through a third selected date. There may be additional similar periods (e.g., fourth, fifth, or sixth additional periods and so on).

The surface texture, porosity, and passages may be dimensioned to restrict, at least to some extent, washout of ionic species and/or other degradation byproducts from within the envelope to the surrounding environment. In doing so, a buildup of ionic species and/or other degradation byproducts may occur thus regulating pH and promoting degradation. This may be particularly advantageous when polymeric material is employed which does not degrade under normal environmental conditions. The timing of the appearance of pores/passages, and the size, shape, location, interconnectivity, and volume of the porosity/passages can be used to regulate the pH in the envelope.

In medical applications according to the present teachings, the size of the pores is tailored to control the rate at which bone and/or tissue ingrowth occurs. Generally, cellular bodies that promote bone and/or tissue growth (e.g., osteocytes) are between about 10 μm and 60 μm in their largest dimension. Thus, pores that are too small for cellular bodies to enter the composite implant may prevent cellular bodies to penetrate the composite implant. It is also possible to tailor the pore size to prevent infiltration of cellular bodies for at least a limited period of time to prevent bone and/or tissue ingrowth. The pores may be tailored to not exceed 10 μm for at least a limited period of time, more preferably 5 μm, or even more preferably 1 μm. Generally, a pore size of between about 200 μm and 400 μm may be advantageous for improved bone and/or tissue growth as well as neovascularization. The degradation profile may include at least one stage in which the pore size is between about 10 μm and 60 μm in their largest dimension. The degradation profile may include at least one stage in which the pore size is between about 10 μm and 60 μm in their largest dimension for about 1 to 2 weeks after implantation. The degradation profile may include at least one stage in which the pore size is between about 200 μm and 400 μm. The degradation profile may include at least one stage in which the pore size is between about 200 μm and 400 μm from about 2 weeks to about 6 weeks after implantation.

Composite articles (e.g., implants, such as orthopedic implants) and/or constituent elements thereof may include a surface roughness. Surface roughness may function to provide paths of ingress and/or egress of aqueous solution, provide paths of ingress for bone and/or tissue ingrowth, or both. Generally, the surface roughness increases as degradation progresses. Surface roughness may increase at a generally linear rate, progressive rate, or exponential rate. Surface roughness may be formed on the outer region of the composite article, perimetric surfaces of one or more cores, reinforcement elements, fiber composites, fiber bundles, fibers, matrix material, or any combination thereof.

Such composite articles and/or constituent elements thereof may have an initial surface roughness. The initial surface roughness may be deliberately formed during fabrication. Initial surface roughness may refer to the surface roughness prior to any degradation. A composite article and/or constituent elements thereof may have an initial surface roughness of about 1 nm or more, 10 nm or more, 100 nm or more, or even 1 μm or more. The composite article and/or constituent elements thereof may have an initial surface roughness of about 100 μm or less, 50 μm or less, or even 10 μm or less. During one or more stages of degradation, the composite article and/or constituent elements thereof may have a surface roughness of about 1 nm or more, 10 nm or more, 100 nm or more, 1 μm or more, or even 10 μm or more. During one or more stages of degradation, the composite article and/or constituent elements thereof may have a surface roughness of about 1 mm or less, 500 μm or less, 100 μm or less, or even 50 μm or less.

Composite articles and/or constituent elements thereof may have a surface roughness that is generally uniform or non-uniform. The composite article and/or constituent elements thereof may have a surface roughness of one or more regions that is different than a surface roughness of one or more other regions.

The composite material may include one or more washout channels (passages). The washout channels may function to facilitate degradation. The washout channels may do so by exposing components of the composite material to aqueous environments that promote degradation. The washout channels may allow degraded materials to flow out of the composite material and into a surrounding environment. The washout channels may be formed into a composite material during its construction and/or may be formed by degradation of one or more components of a composite material. Washout channels formed by degradation of components of a composite material may extend axially, transversely, at an angle with respect to axial, or any combination thereof. The washout channels may include interstitial passages between filaments. The interstitial passages may arise from degradation of polymeric material. The rate of washout channel propagation may be controlled by selection of polymeric material, selection of filament material, interstitial space between filaments, coatings and/or barriers around composite articles, reinforcement elements, filaments, or any combination thereof. The washout channels may prevent buildup of acidic conditions by allowing acidic solutions to disperse and/or flow out of the composite article. The washout channels may prevent buildup of ions by allowing ionic solutions to disperse and/or flow out of the composite article. The washout channels may provide for bone and/or tissue ingress and/or growth. The washout channels may provide for bone and/or tissue growth withing the first 6 weeks, 4 weeks, or even 2 weeks after the composite article is introduced into a patient.

In medical applications according to the present disclosure, normal blood pH may be about 7.3-7.5. The fibers, matrix material, or both may release degradation byproducts that cause a pH shift by ±0.5 units or more, ±1 unit or more, ±2 units or more, or even ±3 units or more.

Degradation may be modulated for differently dimensioned implants (e.g., volume), the location of the implants, or both. Large volume on-bone implants (e.g., plates) may remain in the body and for longer periods of time (e.g., 6 months or more, 12 months or more, 18 months or more, or even 24 months or more) and degrade slower as compared to small volume implants. Degradation of large volume implants may be modulated to prevent buildup of concentration of degradation products, which may result in physiologically negative effects. Small volume in-bone implants (e.g., pins) may remain in the body for shorter periods of time (e.g., no more than 6 months, no more than 4 months, or even no more than 2 months) and degrade slower as compared to large volume implants. Degradation of small volume implants may be modulated to allow for bone growth. The total volume of glass and/or polymer in small volume implants may not pose a risk of adverse physiological reactions even if the degradation is rapid (e.g., fully degrading in 1 month or more, 2 months or more, or even 3 months or more).

Various composites, including implants (e.g., orthopedic implants), according to the teachings herein may be prepared from a common group of subcomponents. The subcomponents include a preform material including one or more columns of fibers. The preform material has a large concentration of axially aligned fibers. The concentration of fibers in the preform material may be about 30 volume percent or more, 40 volume percent or more, about 50 volume percent or more, about 55 volume percent or more, about 60 volume percent or more, about 65 volume percent or more, about 70 volume percent or more, or about 75 volume percent or more, based on the total volume of the preform. The concentration of fibers in the preform material may be about 85 volume percent or less, or about 80 volume percent or less. The axial fibers may be arranged in one or more bundles of fibers. Preferably the preform includes two or more spaced apart bundles of fibers. Space between individual fibers and/or between bundles of fibers in the preform material are partially, substantially, or entirely filled with a polymeric matrix material. The polymeric matrix material preferably surrounds the bundles of fibers, so that the bundles of fibers are embedded in the matrix material. The preform material typically has a cross-section (transverse to the axial/length direction) with a low aspect ratio. For example, the preform material may have an aspect ratio (width to thickness) of about 4 or less, about 1.7 or less, about 1.5 or less, about 1.3 or less, or about 1.2 or less. The preform material may have an aspect ratio (width to thickness) of about 1.00 or more, or about 1.05 or more. By way of example, the cross-section of the preform material may be circular, square, triangular, elliptical, rectangular, or oval.

Another subcomponent that may be employed in a composite according to the teachings herein is a bias material. The bias material is a tape like material having a cross-section with an aspect ratio of width to thickness of greater than 1.7. The aspect ratio of the bias material may be about 1.01 or more, about 1.9 or more, about 2.0 or more, about 2.5 or more, about 3.0 or more, about 3.5 or more, or about 4.0 or more. The aspect ratio of the bias material may be about 100 or less, about 40 or less, or about 15 or less.

The bias material preferably includes a polymeric matrix material, which may be the same or different from the polymeric matrix material of the preform material. The concentration of fibers in the bias material may be about 30 volume percent or more, about 40 volume percent or more, about 50 volume percent or more, about 55 volume percent or more, about 60 volume percent or more, about 65 volume percent or more, about 70 volume percent or more, or about 75 volume percent or more, based on the total volume of the bias material. The concentration of fibers in the bias material may be about 85 volume percent or less, or about 80 volume percent or less. It will be appreciated that the preform and the bias material may be generally continuous materials and may be prepared in a continuous manner from a fibrous material and a matrix material.

A bias material may have a polygonal profile, such as a triangular or rectangular profile (when viewed in a cross-section transverse to the longitudinal axis). A bias material may have a curved profile, such as a round, oval, or elliptical profile (when viewed in a cross-section transverse to the longitudinal axis). The bias structure may include many discreet bias materials (such as in a woven sheet which is wound around axial fibers). The bias structure (e.g., a weaving, braiding, interlocking, etc.) may include bias material that repeatedly wraps around the axial material, such as in a helical winding. There may be space between adjacent portions of bias material or adjacent portions of bias material may be in contact.

The preform material and the bias material may be combined to form other subcomponents, such as a closed sheathed preform or an open sheathed preform. These sheathed preforms may be prepared by wrapping, braiding, weaving, and or interlocking one or more of the preforms with the bias material (i.e., tape-like material). A sheathed preform may have a core including the preform material and a cover including a bias material. A closed sheathed preform may employ a sufficient amount of bias material so that the surfaces of the preform are minimally, substantially or entirely covered by the bias material. An open sheathed preform may be used. It may be prepared by using an open weave or open braiding, so that apertures are created in the cover. For example, the apertures may be created by skipped carriers in a weaving, braiding, sheathing process. A sheath may include a bias material with bias fibers angled relative to axial fibers of a preform. A sheathed preform may be processed to remove voids between the preform and the sheath or between adjacent or overlapping bias material. Voids may be reduced or eliminated by melting the polymeric matrix material(s) and applying a force to the materials. Voids may be reduced or eliminated by adding a polymeric material or a polymerizable material into the voids. It will be appreciated that a sheathed preform may be formed as a continuous material. A ratio of the concentration of bias fibers to the concentration of axial fibers in a sheathed preform may be about 5:95 or more, about 20:80 or more, about 30:70 or more, about 35:65 or more, about 40:60 or more, about 45:55 or more, or about 50:50 or more, where the amount is a measure of the weight or volume of the fibers. A ratio of the concentration of bias fibers to axial fibers in the sheathed preform may be about 80:20 or less, about 70:30 or less, about 65:35 or less, about 60:40 or less, or about 55:45 or less, where the concentration is a measure of the weight or volume of the fibers.

A sheathed preform may be combined with another subcomponent materials to form a structure that is larger or that is different. A sheathed preform may be combined with one or more additional sheathed preforms, one or more preform materials, one or more bias materials, one or more matrix materials, or any combination thereof.

By way of example, a closed sheathed preform may be produced having a generally circular cross-section. For preparing a first implant, seven of the closed sheaths may be combined with one in the center and the other six surrounding the central sheathed preform. The seven sheathed preforms may be wrapped, braided, woven, or interlocked, by additional bias material. It will be appreciated that additional axial material may also be including in the wrapping, braiding, weaving, or interlocking, so that a targeted axial to bias ratio is maintained. An outer sheath is thus formed around the entire assembly of seven closed sheathed preforms. The combined materials may then be processed by heating the matrix material(s) and pulling the structure through a circular die (e.g., an extrusion die) for forming a circular pin. It will be appreciated that dies having different shapes may be employed for preparing pins having different cross-sections. For example, the die may have an a circular profile, an elongated profile, an oval profile, a polygonal profile, a trapezoid profile, a multi-lobed profile, or any combination thereof. The resulting pin is a solid pin with no openings, and can optionally be cut to a desired length and/or coated with a desired coating material.

As another example, instead of coating the assembly of multiple (e.g., seven, ten, fourteen, twenty-three, thirty seven, or some other arrangement of stacked nested bundles) closed sheathed preforms, an overmolding may be applied to form helical threadings of a screw.

As another example, instead one end of the assembly of seven closed sheathed preforms may be arranged over a mandrel for forming a head for a screw, which can be further be defined by an overmolding.

As another example, a coating may be machined to form barbs or threadings.

As another example, the assembly may be prepared with six of the closed sheathed preforms by eliminating the central component. The sheathed preforms are then wrapped braided, woven or interlocked with the bias material while on a mandrel for defining a cannulation along the implant. Optionally, the wrapping, weaving, braiding, or interlocking may be performed with skipped carriers so that the resulting assembly has an open architecture with openings on the sides of the implant. These openings may extend into the central cannulation.

As such, it will be appreciated that a large number of composite implant structures may be prepared, each having a predetermined and/or generally high concentration of fibers, each having a predetermined ratio of axial to bias fibers, or both.

Furthermore, an assembly including one or more sheathed preforms may be combined with one or more additional materials and/or one or more additional assemblies to form a larger structure.

Similarly, multiple preforms and/or multiple sheathed preforms may be arranged side-by-side in a generally planar arrangement for forming a plate-shaped implant. The plate-shaped implant may have a generally closed architecture, or a generally open architecture based on the way the bias material is applied over the sheathed preforms.

As another example, a sheathed preform and/or an assembly of sheathed preform may be employed in an intermedullary implant. These components, preferably after coating with one or more layers of additional material may be inserted into an intermedullary canal, and then filling space between the components with a polymer or polymerizable or cross-linkable material.

As described above, these common subcomponents may be combined using various methods to prepare a plate, a pin, a rod, an anchor, a nail, or other structure, any of which may optionally have an open architecture and/or a central cannulation. It will be appreciated that additional materials may be used in addition to the common subcomponents. For example, the composite implant may include one or more layers of an additional material. A matrix material or a different material may be employed as an interface material for modulating or otherwise controlling degradation.

The materials according to the teachings herein may be employed in a composite device, such as a composite screw, a composite anchor, a composite nail, or a composite pin, having an elongated structure with a generally uniform cross-section along its length. Preferably the composite device is a composite orthopedic implant. The composite device (e.g., screw, anchor, nail, or pin) may be characterized by a length (L) and a cross-sectional area (A) transverse to the length direction. The composite device may be characterized by a diameter (d), or an equivalent diameter (dₑ), where the equivalent diameter is related to the cross-sectional area (A): A=(¼) πdₑ². Preferably, the diameter or equivalent diameter of the composite device is about 0.5 mm or more, about 1 mm or more, about 2 mm or more, about 3 mm or more, about 4 mm or more, or about 5 mm or more. The diameter or equivalent diameter of the composite device preferably is about 20 mm or less, about 15 mm or less, or about 10 mm or less. For example, the diameter may be from about 0.5 mm to about 3 mm, from about 3 mm to about 6 mm, from about 6 mm to about 10 mm, or from about 10 mm to about 20 mm. The length of composite device may be about 4 mm or more, about 6 mm or more, about 10 mm or more, about 20 mm or more, or about 30 mm or more. The length of the composite device may be about 100 mm or less, about 60 mm or less, about 50 mm or less, or about 40 mm or less. For example, the length of the composite device may be about 4 mm to about 10 mm, from about 10 mm to about 30 mm, from about 30 mm to about 50 mm, or from about 50 mm to about 100 mm.

One end of the composite screw or anchor may have a taper. One end of the composite screw or anchor may be configured for receiving a driving tool. The end of the screw or anchor configured for receiving a driving tool may have a head. The composite screw or anchor typically is characterized by a shaft having a generally circular cross-section. At least a portion of the shaft is threaded. The threaded portion of the shaft preferably starts near at an end of the composite screw opposite from the end configured for receiving a driving tool. The threaded portion may extend only a fraction of the length of the shaft or may substantially or entirely the length of the shaft. For example, the threaded portion may extend about 5% or more of the length of the shaft, about 10% or more of the length of the shaft, about 20% or more of the length of the shaft, about 30% or more of the length of the shaft. The threaded portion may extend about 100% or less, about 80% or less, about 60% or less, about 50% or less, or about 40% or less of the length of the shaft. The threaded portion may include one or more helical threadings. The helical threading may be continuous or discontinuous. The number of helical turns of the threaded portion preferably is about 3 or more, about 4 or more, about 5 or more, about 6 or more, or about 7 or more. A tapered end of the screw or anchor may extend a portion of a turn (e.g., about % turns or more, or about ½ turns or more) or may extend one or more turns (e.g., about 1 turn or more, about 2 turns or more, or about 3 turns or more. The helical turns may be characterized by a crest and a root. A ratio of the crest diameter to the root diameter may be about 4.0 or less, about 3.0 or less, or about 2.5 or less, about 2.2 or less, or about 2.0 or less. A ratio of the crest diameter to the root diameter may be about 1.1 or more, about 1.2 or more, about 1.3 or more, about 1.4 or more, or about 1.5 or more. Unless otherwise stated, the diameter of the screw refers to the diameter measured at the crest. When drilling a gliding hole, the diameter of the hole should be at least the screw diameter (i.e., the threaded diameter measured at the crest). When drilling a hole for internal threading, the diameter of the hole should be less than the threaded diameter. For example, a hole for internal threading may be about the root diameter of the screw (preferably +/−20%, more preferably +/−10%) and/or between the root diameter and the threaded diameter. The helical threads may be characterized by the pitch (i.e., number of turns of the helix per cm length). Preferably, the pitch of the composite screw or anchor is about 0.30 mm or more, about 0.35 mm or more, about 0.40 mm or more, or about 0.43 mm or more. The pitch preferably is about 2.0 mm or less, about 1.80 mm or less, about 1.60 mm or less, or about 1.4 mm or less. A ratio of the screw pitch to the screw diameter preferably is about 0.08 or more, about 0.10 or more, about 0.12 or more, or about 0.14 or more. A ratio of the screw (or anchor) pitch to the screw (or anchor) diameter preferably is about 0.30 or less, about 0.25 or less, or about 0.20 or less. A cross-section of the screw or anchor (along the length of the screw and through the axis of the screw) shows the helical structure. Preferably in the crest region and/or in the root region, the radius of curvature is generally high so that local stresses to materials of the composite is reduced. Applicant has determined that these local stresses may be amplified in a composite material, as compared to a generally monolithic structure. Similarly, the radius of curvature at the tip of the screw or anchor and or at the transition between the shaft and the head of the screw are preferably generally high. For example, the minimum radius of curvature of the crest and/or the root and/or the screw tip and/or the transition between the shaft and the head may be about 0.02 mm or more, about 0.03 mm or more, about 0.04 mm or more, about 0.05 mm or more, about 0.06 mm or more, about 0.07 mm or more, about 0.08 mm or more, about 0.09 mm or more, about 0.10 mm or more, about 0.12 mm or more, about 0.14 or more, about 0.16 mm or more, about 0.18 mm or more, or about 0.20 mm or more. A ratio of the radius of curvature (e.g., of the root, the crest, the tip, the transition between the shaft and the head, or any combination thereof) to the diameter of the threaded screw may be about 2 percent or more, about 3 percent or more, about 4 percent or more, about 6 percent or more, about 8 percent or more, or about 10 percent or more. The composite screw may be a lag screw, a compression screw, an interference screw, a cortical screw, a cancellous screw, a malleolar screw, a cortex screw, or a different implant screw.

The materials according to the teachings herein may be employed in a composite plate, such as a composite orthopedic implant plate. The composite plate (i.e., the plate) may be generally flat or may have a curvature, typically along a width direction. The plate is typically elongated so that the length of the plate is greater than the width and thickness of the plate. A ratio of the length to width may be about 2 or more, about 5 or more, about 7 or more, or about 10 or more. The ratio of the length to the width of the plate may be about 35 or less, about 30 or less, about 25 or less, about 20 or less, or about 15 or less. The ratio of width to the thickness may be about 2 or more, about 3 or more, about 4 or more, about 5 or more or about 6 or more. The ratio of the width to the thickness of the plate may be about 40 or less, about 30 or less, about 20 or less, or about 10 or less. The width of the plate may be uniform along the length of the plate or may vary. If the width varies along the length of the plate, for purposes of calculating the above ratios, the largest value of the width may be used. Generally flat plates have a radius of curvature of about more than 100 mm. Curved plates may have a radius of curvature of about 100 mm or less, about 50 mm or less, about 40 mm or less, about 30 mm or less, about 20 mm or less, about 10 mm or less, or about 8 mm or less. Curved plates typically have a radius of curvature of about 1 mm or more, about 2 mm or more, or about 3 mm or more. The arc of the curved plate may be about 5° or more, about 10° or more, about 15° or more, about 20° or more, or about 25° or more. The arc of the curved plate may be about 220° or less, about 180° or less, about 150° or less, about 120° or less, about 90° or less or about 70° or less. For example, the arc of the curved plate may be from about 5° to about 25°, from about 25° to about 50°, from about 50° to about 70°, from about 70° to about 120°, from about 120° to about 180°, or from about 180 to about 220°. The plate may include holes for attaching the plate to a bone. The number of holes in the plate may be about 2 or more, about 4 or more, or about six or more. Preferably, the number of holes in the plate is about 30 or less, about 20 or less, or about 10 or less. Some or all of the holes may be spaced apart along the length of the plate. The plate may have a length of about 10 mm or more. The plate may have a length of about 500 mm or less. The plate may include holes that are not aligned along a single axis. The length of the plate may be related to the length of the bone to which it is being attached and/or to the distance between bones to which it is being attached. For example, a length of the plate may be about 10 mm to about 25 mm, about 25 mm to about 50 mm, about 50 mm to about 100 mm, or about 100 mm to about 500 mm. Examples of plates include straight plates, L-plates, T-plates, multiple fragment plates, compression plates, tubular plates, cloverleaf plates, oblique L-plates, oblique T-plates, spoon plates, buttress plates, t-buttress plates, and L-buttress plates. The plate may be straight or may be bent.

The composite implant (e.g., screw, pin, or plate) may be prepared using a materials or composites according to the teachings herein. The composite implant (e.g., the composite orthopedic implant) preferably includes axial fibers and bias fibers. Preferably, the axial fibers are aligned with or generally aligned (e.g., within 10° or within 5°) with the length direction of the implant. The composite implant may include a bias material which braids, wraps, weaves, or interlocks bundles of the axial fibers. The bias material may be applied to create an open architecture including apertures for the holes. The axial fibers and bias material may be covered with a polymeric material. For example, a polymeric material may be overmolded over one or more, or even all of the surfaces of the plate. A ratio of the amount of bias fibers to the amount of axial fibers in the implant may be about 20:80 or more, about 30:70 or more, about 35:65 or more, about 40:60 or more, about 45:55 or more, or about 50:50 or more, where the amount is a measure of the weight or volume of the fibers. A ratio of the amount of bias fibers to the amount of axial fibers in the implant may be about 80:20 or less, about 70:30 or less, about 65:35 or less, about 60:40 or less, or about 55:45 or less, where the amount is a measure of the weight or volume of the fibers. The bias fibers and the axial fibers may include fibers that are the same or fibers that are different. Preferably the bias fibers and the axial fibers include glass fibers.

The composite implant (e.g., screw, pin, nail, anchor or plate) may be characterized by a core region including axially aligned fibers. Preferably some or all of the space between the fibers in the core region is filled with a polymeric matrix. The composite implant may have a cover region surrounding the core region. The cover region preferably includes bias fibers which are angled relative to the axially aligned fibers of the core region. Preferably some or all of the space in between the fibers in the cover region is filled with a polymeric matrix, which may be the same or different from the polymeric matrix of the core region. A volume ratio of the core region to the cover region may be about 20:80 or more, about 25:75 or more, about 30:70 or more, about 35:65 or more, about 40:60 or more, about 45:55 or more, or about 50:50 or more. A volume ratio of the core region to the cover region may be about 80:20 or less, about 75:25 or less, about 70:30 or less, about 65:35 or less, about 60:40 or less, or about 45:55 or less. The composite implant may include a coating over the cover region. The coating may be applied by any means. For example the coating may be an overmolded coating, an extruded coating, an coating, a printed coating (e.g, using a 3D printing method), a sprayed coating, or a dipped coating. The coating may have a predetermined surface texture. The coating may have a predetermined surface profile. The coating preferably is free of continuous fibers. The coating preferably is free of axial fibers. The coating preferably is free of bias fibers. The coating preferably is formed of free flowing material that is melt extrudable, melt moldable, or a polymerizable liquid. The material of the coating may include a filler material. Preferably, the material of the coating is degradable and/or resorbable. For composite screws, or other composites having threadings or barbs, the threadings and/or barbs are preferably formed in the coating. Threadings or barbs may be formed when the coating is applied. Threadings or barbs may be formed by removing some of the coating. Preferably the threadings or barbs are free of axial fibers. Preferably, the threadings or barbs are free of bias fibers.

One or both ends of a composite implant device may have a taper. A tapered end may have a cross-sectional area (transverse to the length of the composite implant) that is reduced from the maximum cross-sectional area of the composite implant. The reduction in the diameter at a tapered end may be about 10% or more, about 20% or more, about 30% or more, about 40% or more, about 50% or more, or about 60% or more.

Specific examples of implant structures that can be prepared from a preform material and a bias material are listed in the table below.

| | Composite Pin | | Composite pin | |
|---|---|---|---|---|
| | 2.0 mm diameter | | 2.5 mm diameter | |
| Cannulation | None | | None | |
| Diameter | 0 | | 0 | |
| Cross-sectional area | 0 | | 0 | |
| Axial core | | | | |
| Diameter | 1.41 | mm | 1.77 | mm |
| Cross-sectional area | 1.57 | mm² | 2.453 | mm² |
| Fiber material | Twisted Glass Fibers | | Twisted Glass Fibers | |
| Fiber concentration | 55 volume percent | | 55 volume percent | |
| Core aspect ratio | 1:1 | | 1:1 | |
| Bias Material (tape) | | | | |
| Thickness | 0.148 | mm | 0.183 | mm |
| Fiber material | Twisted Glass Fibers | | Twisted Glass Fibers | |
| Areal weight (g/M²) | | | | |
| Fiber bundle thickness | 0.148 | mm | 0.183 | mm |
| Aspect ratio | 4:1 | | 4:1 | |
| Cover layer | | | | |
| (bias cover) | | | | |
| Thickness | 0.295 | mm | 0.366 | mm |
| Volume | 1.57 | mm² | 2.453 | mm² |
| Fiber concentration | 55 volume percent | | 55 volume percent | |
| Ratio of axial fibers | 1:1 | | 1:1 | |
| to bias fibers | | | | |
| Total Thickness | 2.0 | mm | 2.5 | mm |

One aspect of the teachings herein relates to intermedullary implants, materials for preparing an intermedullary implants, devices for installing an intermedullary implant, and related methods.

An intermedullary implant may be prepared in situ using various components which are selected for one or more of the following benefits: (1) high strength and/or stiffness of the implant; (2) ease of manufacturing the implant; (3) reduction of parts needed in an operating room; and (4) ability to build the implant with a short operating room time.

In order to achieve one or more of these benefits, the intermedullary implant is preferably constructed using reinforcement rods, which includes fibers, and preferably includes glass fibers.

Preferably one or more of the components of the intermedullary implant is delivered to the implant site (i.e., an intermedullary canal) via one or more catheters.

The reinforcement rods may be used for preparing a preform material and a bias material as described herein. The reinforcement rods should be sufficiently flexible so that they can be inserted through an opening in a bone without fracturing the rods. Preferably, the reinforcement rods do not break when bent 90° or 180° around a mandrel having a diameter of about 120 mm. More preferably, the reinforcement rods do not break when bent 90° or 180° around a mandrel having a diameter of about 60 mm. Most preferably, the reinforcement rods do not break when bent 90° or 180° around a mandrel having a diameter of about 40 mm. The reinforcement rods may be shaped so that a spacing is created between adjacent reinforcement rods to allow for flow of a hardenable liquid around the reinforcement rods. The reinforcement rods may be configured to enable flow of a hardenable liquid in an axial direction of the reinforcement rods. The reinforcement rods may be configured to enable flow of a hardenable liquid in a direction angled relative to the axial direction of the reinforcement rods. Some or all of the reinforcement rods may have a spacing feature for assisting in spacing one rod from adjacent rods. Some or all of the reinforcement rods may have a feature for assisting flow of the hardenable liquid in a non-axial direction. For example, a reinforcement rod may have one or more radial groove or a spiral shaped recess. A radial groove or a spiral shaped recess may extend one or more turns around the circumference of the reinforcement rod, or may be a fraction of a turn. Preferably the length of the groove or recess is about 0.05 turns or more, about 0.10 turns or more, about 0.15 turns or more, about 0.30 turns or more, about 0.5 turns or more, about 0.75 turns or more, about 1 turn or more, or about 2 turns or more. The depth, length, and number of grooves and/or recesses should be sufficiently low so that the reinforcement rods can easily slide past one another when arranging together in a parallel manner, such as during insertion into a containment bag. Some or all of the reinforcement rods may have one or both end that is narrowed to allow for easier insertion and or packing into a containment bag. For example, the reinforcement rod may have a pointed or curved tip that can gently push against other reinforcement rods for slightly displacing the other rods and creating a sufficient space for the rod being inserted. Preferably both ends of the reinforcement rods are narrowed, so that a narrow end of a rod being inserted can wedge against narrow ends of rods that have already been inserted.

Hardenable liquid hardens by a chemical reaction between the two parts. The hardenable liquid may include one or more monomers, one or more prepolymer, or both.

The molecular weight of the prepolymer and/or degree of polymerization of the prepolymer should be sufficiently low so that the prepolymer can quickly flow between the reinforcement rods (optionally with the assistance of a vacuum). The hardenable liquid preferably fills the spaces between the reinforcement rods in a time of about 10 minutes or less, about 8 minutes or less, about 6 minutes or less, about 4 minutes or less, about 2 minutes or less, or about 1 minute or less.

A Liquid delivery device may be employed for transporting and/or mixing the hardenable liquid. Preferably the liquid delivery system transports and mixes the hardenable liquid. The liquid delivery device may attach to a plug and/or a containment bag. The liquid delivery device preferably is capable of mixing two or more components just prior to the hardenable liquid entering the containment bag and/or contacting the reinforcement rods. The liquid delivery device preferably has multiple flow channels for separately flowing portions of the hardenable liquid. Two flow channels may be flow channels of a single tube which are separated by a wall along a length of the tube. Two flow channels may be flow channels of separate tubes. Before the hardenable liquid enters the containment bag and/or contacts the reinforcement rod, the different parts are combined and mixed together. The mixing can be at an end of a tube having a mixing component or may be in a separate mixing component which is connected to the multiple flow channels. The mixing component preferably includes or consists essentially of a static mixer. The two flow channels may meet at or near the end of the flow channels or may flow into a mixing unit. The mixing component preferably includes mixing elements which cause the different components to come together. The mixing component (e.g., the static mixer) is preferably flexible so that it can be inserted into an intermedullary canal. For example, the static mixer may be connected to a containment bag or to a plug in the intermedullary canal. As another example, a short connector (tube or otherwise) may be used to connect the static mixer to a containment bag or to a plug in the intermedullary canal, while the static mixer is in the intermedullary canal. In order to achieve this flexibility, the static mixer may include mixing elements that are formed having thin walls, formed of a flexible material, or both. The static mixer may include clockwise mixing elements and counterclockwise mixing elements. The static mixer may include pins, orthogonal walls, or other features that interrupt or split the flow of the combined liquids. The static mixer may include mixing elements which result in a turbulent flow. The static mixer should include a sufficient number of mixing elements to mix the different components of the liquid so that a hardening reaction can commence and the resulting material has a generally uniform distribution of the various components. Preferably, variation in each component (or the minor component) is about 20 volume % or less, about 10% or less, about 4% or less, about 2% or less, or 1 volume % or less, relative to the target concentration. By way of example, if two components are added at a 3:1 ratio, then the minor component has a target composition of 25 volume percent. In this example, a local concentration of 20 or 30 volume percent of the minor component would correspond with a variation of 20 percent, and a local concentration of 24 volume percent or 26 volume percent of the minor component would correspond with a variation of 4%. The static mixer may be a rotary static mixer, which rotates where the flow of the liquid causes the mixing elements to rotate. The static mixer may be a stationary static mixer, having no mixing elements that move during mixing. The static mixer may include a housing. The mixing elements may be in a housing. The mixing elements may be inserted into and/or connected to a delivery tube or catheter. The housing may be configured to hold the mixing elements. The housing may be configured to connect with a plug or a containment bag. The housing may be configured to move through a catheter for positioning at a delivery site (for example, a delivery site including reinforcement rods). It will be appreciated that in order to provide sufficient mixing, the mixing component may extend outside of the bone. Preferably, the flexible static mixer (e.g., the static mixing elements and/or the housing of the static mixer) does not break when bent 90° or 180° around a mandrel having a diameter of about 120 mm. More preferably, the flexible static mixer does not break when bent 90° or 180° around a mandrel having a diameter of about 60 mm. Most preferably, the flexible static mixer does not break when bent 90° or 180° around a mandrel having a diameter of about 40 mm. The flexible static mixer (e.g., the static mixing elements and/or the housing of the static mixer) preferably is formed of one or more materials having a durometer of about 50 shore D or less, more preferably about 87 Shore A or less, even more preferably about 75 Shore A or less, and most preferably about 64 Shore A or less. Preferably, the flexible static is formed of one or more materials having a durometer of about 10 Shore A or more, about 15 Shore A or more, about 20 Shore A or more, about 30 Shore A or more, or about 40 Shore A or more. One or more of the components of the flexible static mixer may be biodegradable and/or biocompatible.

The hardenable liquid may be a two part curable material. One or both parts may have a viscosity (at 23° C.) of about 40,000 cps or less, about 20,000 cps or less, about 8,000 cps or less, about 5,000 cps or less, about 3,000 cps or less, or about 1,000 cps or less. One or both parts may have a viscosity (at 23° C.) of about 1 cps or more, about 5 cps or more or about 10 cps or more. The viscosity of the two parts should be the same or similar, for example, the viscosity ratio preferably is about 10 or less, about 6 or less, about 4 or less, about 2 or less, or about 1.5 or less.

Any volume ratio of the liquid parts may be employed and will depend on the specific materials in each part, such as the reactive groups, the functionality, the molecular weights, and the concentration of non-reactive materials. By way of example, the mix ratio may be about 100:1, about 50:1, about 20:1, about 10:1, about 5:1, about 4:1, about 3:1, about 2:1, or about 1:1. Mix ratio between any of these values may also be employed. By way of example, the mix ratio may be from about 2:1 to about 10:1, or the mix ratio may be from about 1:1 to about 100:1. High mix ratio materials may be more sensitive to the exact ratio. As such, the mix ratio of the two parts preferably is from about 1:1 to about 10:1, more preferably from about 5:1 to about 1:1, even more preferably from about 3:1 to about 1:1, and most preferably from about 2:1 to about 1:1.

Reaction rate of the hardenable liquid. Typically, when using a two part adhesive material for forming a bone implant, the adhesive material is used alone and the reaction rate is generally slow so that the surgeon has sufficient time to mix the adhesive material, fill a treatment location with the adhesive material and make any necessary adjustments. As such, the pot life of the adhesive material is typically about 10 minutes or more. In contrast, by using a static mixer positioned near or adjacent to a treatment site, and by using a large concentration of reinforcement rods containing a non-liquid matrix, it is possible to use faster curing adhesive material. Although slower curing materials may be used, the pot life (or working life) of the hardenable liquid may be less than 10 minutes, about 8 minutes or less, about 5 minutes or less, about 3 minutes or less, about 2 minutes or less, or about 1 minute or less. The pot life or working life may be measured according to ASTM D1144-99 (2021).

Among the many applications of the present teachings is providing the composite orthopedic implant as a screw. The screw may be located into bone. The screw may be located into cortical bone. The screw may be located into and/or through trabecular bone. The screw may be located into and/or through a medullary cavity. The screw may be located into a pre-formed cavity in bone.

The screw may interface with tissue. The screw may create an interference fit between bone and tissue. These types of orthopedic implant screws are typically referred to as interference screws. The tissue may locate between the screw and bone. The screw may fixate and/or tension tissue. By way of example, the screw may fixate a ligament to bone.

The screw may include one or more apertures. The apertures may function to allow bodily fluids to flow throughout the screw and/or the bone cavity occupied by the screw. The bodily fluids may comprise nutrients and/or biological bodies for the regeneration of bone and/or tissue. Increasing the flow of bodily fluids within the bone cavity and/or screw may shorten healing time, ensure proper healing, or both.

Heretofore, screws provided with apertures have been constructed from metal. These metal screws exhibit favorable mechanical properties (e.g., torsional strength, flexural strength, and/or compressive strength) and a ductile failure mode. However, metal screws are typically removed from patients during a second surgical procedure, resulting in further trauma to the patient and increasing the costs of the overall procedure.

Heretofore, screws provided with apertures have been constructed from non-degradable materials. The non-degradable materials may include polymers. The polymers may include polyether ether ketone (PEEK). However, these polymer screws are limited in mechanical properties (e.g., torsional strength, flexural strength, and/or compressive strength) as the ratio of open surface area (i.e., surface area of the screw occupied by apertures) to closed surface area (i.e., surface area of the screw not occupied by apertures) increases. The failure mode of these types of polymer screws is brittle. Moreover, it may be desirable to fabricate a screw from biodegradable and/or bioresorbable materials to avoid the need to remove the screw from the patient.

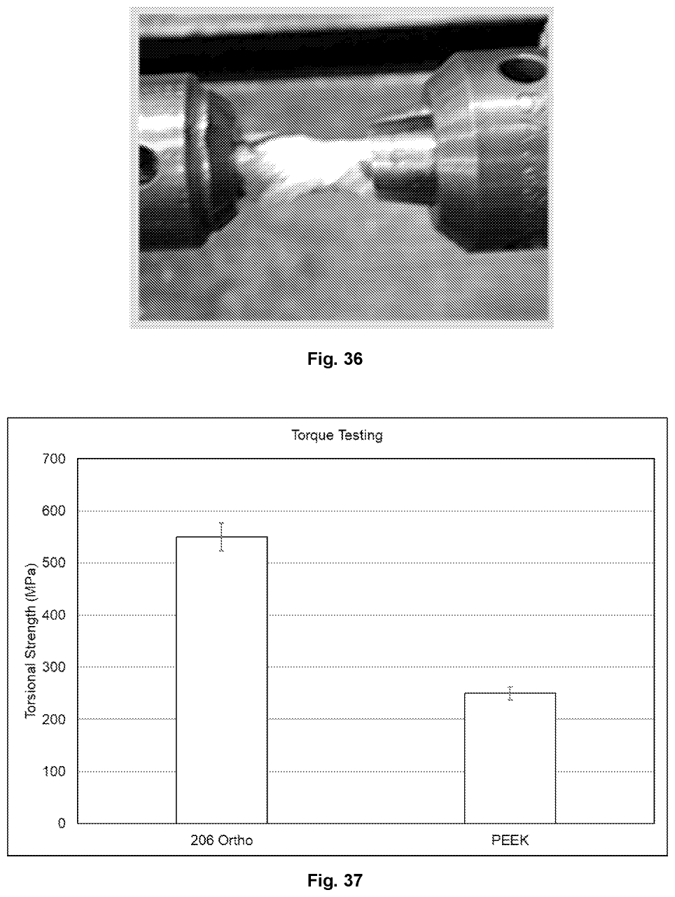

FIG. 37 torsional testing is comparing 206 Ortho composite to PEEK, 7.5 mm diameter. See the notes in OneNote. Prior art teaches the limited mechanical properties bio composites restrict the potential implant designs. Specifically, biologic implant designs with macro pores for tissue ingrowth. The testing shows that open architecture implants made bioresorbable composite exhibit plastic deformation at lower strain than implants made with PEEK. FIG. 36—shows non-brittle failure of 206 Ortho's composite, 7.5 mm diameter, which overcomes safety issue of brittle failure with bio-resorbable and non-resorbable implants. See notes on Carbon fiber reinforced implants with catastrophic failure.

The diameter of the screw may direct the ratio of open to closed surface area that provides suitable mechanical properties. The ratio of open to closed surface area that provides suitable mechanical properties may increase as the diameter of the screw is increased. The screw of the present disclosure, by the material selection and unique and unconventional arrangement of the screw's constituent elements, may surpass the amount of open area of conventional screws while providing suitable mechanical properties. By way of example, screws fabricated from PEEK have the following limits on open:closed surface area, relative to their diameter, while still providing suitable mechanical properties.

| Diameter | | Open: Closed Surface Area | Open:Closed Surface Area |
|---|---|---|---|
| <5 | mm | ~1:20 | >1 to 5 |
| 5-6 | mm | ~17:100 | >1 to 4.5 |
| 7-9 | mm | ~1:5 | >1 to 3.5 |
| 10-12 | mm | ~1:4 | >1 to 2.5 |

The failure torque, plastically deformed, for a 7.5 mm diameter implant with an Open to close surface area of 1 to 3.5 was greater than 16 in*lb.

Advantageously, the present application finds that a screw fabricated from biodegradable and/or bioresorbable polymer may provide suitable mechanical properties and ductile failure mode by fabricating the screw in a unique and unconventional manner.

The screw may have a length of about 10 mm or more, 13 mm or more, 16 mm or more, or even 19 mm or more. The screw may have a length of about 33 mm or less, 30 mm or less, 27 mm or less, or even 24 or less. By way of example, the screw may have a length of about 25 mm. The screw may have a major diameter of about 4 mm or more, 5 mm or more, or even 6 mm or more. The screw may have a major diameter of about 10 mm or less, 9 mm or less, or even 8 mm or less. By way of example, the screw may have a diameter of about 7 mm.

The screw may have two opposing ends. The ends may include bevels, radii, or both. The bevels and radii may function to mitigate or substantially prevent stress risers in tissue, bone, the screw, or any combination thereof. The bevels and radii may extend around a circumference of the ends or at least a portion thereof.

The ends may be referred to herein as a lead end and a distal end. The lead end may be oriented away from the bone. The lead end may interface with a driver. The distal end may be oriented toward the bone. The distal end may enter the bone first as the screw is implanted into the bone.

The distal end (tip) may be pointed, flat, truncated, or rounded. Threading may extend at least partially around the distal end. The major diameter of threading may gradually increase as it extends from the tip to the head.

The distal end (tip) may have a taper. The distal end may taper along a length of the screw. The taper may have a length of about 5 mm or less, 4 mm or less, 3 mm or less, or even 2 mm or less. Threads may extend at least partially along the distal end. The threads may extend for about 3 revolutions or less, 2 revolutions or less, or even 1 revolution or less.

The screw may include a head at one end. The head may be located at one end (lead end) of the screw. The screw may include a transition between the head and a shaft of the screw. The transition may include bevels, radii, or both. The bevels and radii may function to mitigate or substantially prevent stress risers in tissue, bone, the screw, or any combination thereof.

The screw may include a drive socket. The drive socket may cooperate with a driver (e.g., a screwdriver). The drive socket may be located in the head. The drive socket may extend along the longitudinal axis of the screw. The drive socket may extend the length of the head along the longitudinal axis of the screw or at least a portion thereof. The drive socket may extend the length of the shaft of the screw or at least a portion thereof. A cannulation, as described herein, may be configured to function as a drive socket.

The screw may include threads. The threads may extend along the length of the shaft or at least a portion thereof. The threads may helically wind around the shaft.

The threading may be defined by a thread angle. The thread angle may be measured between opposing surfaces of adjacent threads, as viewed along a transverse axis of the screw.

The threading may be defined by a pitch. The pitch may be the distance between crests of adjacent threads, as viewed along a transverse axis of the screw.

The threading may be defined by a crest. The crest may be the most radially distanced end of the threads, as viewed along a transverse axis of the screw.

The threading may be defined by a root. The root may oppose the crest.

The threading may be defined by a major diameter and minor diameter. The major diameter may be the transverse cross-sectional diameter of the root. The minor diameter may be the transverse cross-sectional diameter of the crest.

The screw may include a transition between the threads and a shaft of the screw. The transition may include bevels, radii, or both. The bevels and radii may function to mitigate or substantially prevent stress risers in tissue, bone, the screw, or any combination thereof.

The threads may extend from a minor diameter of the screw, defined by the shaft, to a major diameter of the screw, defined by the ends of the threads. The ends of the threads may include bevels, radii, or both. The bevels and radii may function to mitigate or substantially prevent stress risers in tissue, bone, the screw, or any combination thereof.

The threads may comprise a lead face, a distal face, or both. The lead face may be oriented toward a head of the screw and/or lead end of the screw. The distal face may oppose the lead face. The distal face may be oriented toward bone and/or tissue of a patient as the screw is being inserted into a patient. The distal face may be oriented toward the distal end of the screw.

The distal face may include a slope. The slope of the lead face may be adapted to prevent damage to bone and/or tissue, aid with insertion of the screw into bone, or both. The slope of the lead face may be.

The lead face may include a slope. The slope of the distal face may prevent pullout of the screw, tissue affixed between the screw and bone, or both. The slope of the distal face may be steeper than the slope of the lead face.

The threads may be defined by a height. The height may be the dimension of the threads from the shaft to the end of the threads. In other words, the height may be the difference between the major diameter and the minor diameter of the screw. The height may be about 3 mm or less, 2 mm or less, or even 1 mm or less.

The threads may be defined by a root width. The root width may be defined by the root of the threads between the lead face and the distal face. The root width may be about 3 mm or less, 2 mm or less, or even 1 mm or less.

The threads may helically wind around a shaft of the screw. The threads may be oriented at an angle relative to a longitudinal axis of the screw (the longitudinal axis extending through the length of the shaft and/or head). The angle may be about 30° or more, 35° or more, or even 40° or more. The angle may be about 60° or less, 55° or less, or even 50° or less. By way of example, the angle may be about 45. Advantageously, an angle between about 30 and 60 may provide for suitable transfer of torsional forces throughout the elements (e.g., reinforcement elements) of the screw.

The screw may comprise a cannulation. The cannulation may function to provide for ingress of fluid, to facilitate an inside-out degradation, as a driver socket, or any combination thereof.

The cannulation may be a hollow shaft within a screw. The cannulation may extend at least partially between and/or through distal ends of a screw. The cannulation may extend longitudinally through the screw, through the center of the screw, between two distal ends of the screw, or any combination thereof. The cannulation may have a cross-sectional shape. The shape may be circular, ovoid, triangular, quadrilateral, pentagonal, hexagonal, octagonal, the like, or any combination thereof.

The cannulation may be formed by locating a plurality of precursor elements around a removable mandrel.

The cannulation may function as or define a driver socket. The driver socket may aid introduction of the screw into bone or other suitable substrate using a driver tool. The cannulation may extend at least partially through a screw head. The cannulation may taper from the head to the tip. The taper may provide a positive seating between the cannulation and driver tool.

One or more apertures may be present on the shaft of the screw. The apertures may be present between roots of adjacent threads.

The apertures may extend the length between roots of adjacent threads or at least a portion thereof. The length may be about 1 mm or more, 2 mm or more, or even 3 mm or more. The length may be about 30 mm or less, 20 mm or less, or even 10 mm or less.

The apertures may extend circumferentially around the shaft of the screw or at least a portion thereof. The apertures may extend circumferentially by about 1 mm or more, 2 mm or more, or even 3 mm or more. The apertures may extend circumferentially by about 15 mm or less, 12 mm or less, or even 10 mm or less.

The apertures may extend a depth into the screw. The apertures may interface with a cannulation and/or drive socket.

The screw may comprise a barrier. The barrier may be provided on the outermost region of the screw. The barrier may be located between a core portion of the screw and threads of a screw. The barrier may form a part or all of the threads of the screw.

The barrier may comprise a polymer. The polymer may include PLDLA, although any polymer disclosed herein may be employed.

The polymer may include a filler. The filler may be generally homogenously dispersed throughout the polymer. The filler may include glass filaments. The glass filaments may be bioactive. The glass filaments may have a density of about 2 g/cm³ to about 3 g/cm³, more preferably about 2.4 g/cm³ to 2.8 g/cm³, or even more preferably about 2.6 g/cm³ to 2.7 g/cm³. The glass filaments may have an aspect ratio of about 20:1 or less, 15:1 or less, 10:1 or less, or even 5:1 or less. The aspect ratio may be defined by the ratio of the filament length to the filament width. The glass filaments may be chopped. The glass filaments may be chopped to a length of about 1 mm or more, 5 mm or more, or even 10 mm or more. The glass filaments may be chopped to a length of about 25 mm or less, 20 mm or less, or even 15 mm or less.

The barrier may be defined by a thickness. The thickness may be about 0.001 mm or more, 0.01 mm or more, 0.1 mm or more, or even 0.5 mm or more. The thickness may be about 3 mm or less, 2 mm or less, or even 1 mm or less.

The barrier may have a surface roughness (porosity). The surface roughness (porosity) may include micron size pores, sub-micron size pores, or both. The micron size pores may be about 1 micron or more, 3 microns or more, or even 5 microns or more in their largest dimension. The micron size pores may be about 15 microns or less, 12 microns or less, or even 9 microns or less in their largest dimension. The sub-micron pores may be about 0.001 microns or more or even 0.01 microns or more in their largest dimension. The sub-micron pores may be about 0.99 microns or less, or even 0.1 microns or less.

Composites of the present teaching (e.g., implants, such as orthopedic implants), or any of their constituent reinforcement elements, may have multiple regions along their length. Differing regions may adjoin (directly or indirectly) each other. Regions may differ in material type. Regions may differ in material property. Regions may differ in structure. There may be an inner region (i.e., located toward a central part of the composite). There may be one or more regions that at least partially (or possibly entirely) surrounds the inner region (e.g., an outer region or surrounding region).

The structure, material or both, of adjoining regions (e.g., an inner region that adjoins an outer or surrounding region) may result in a difference in mechanical properties between the different regions. For example, the flexural modulus as between such regions may differ. The regions may differ in regard to flexural modulus by a factor of 1.3, 1.5, 2, 2.5, 4, 6 times or more.

For an orthopedic implant it is possible that the selection and arrangement of regions may be such that an outer region that will initially adjoin bone or tissue will have a first flexural modulus (e.g., between about 6 and 14 GPa) that approximates that of the bone or tissue, or is lower. Such outer region may have a plurality of degradable or resorbable filaments (e.g., chopped fibers) or filler dispersed in a polymeric matrix of the types described herein.

The implant may include another region adjoining the outer region that has a second flexural modulus. That region may include one or more distinct regions that differ relative to each other. Such regions may be concentrically located, axially aligned, parallel aligned, or any combination thereof along the length of the implant or any of its constituent elements. There may be at least one region that has a flexural modulus that differs from a flexural modulus of another region by a factor of 1.3, 1.5, 2, 2.5, 4, 6 times or more. One possible approach is to include an outer core region that at least partially surrounds an inner core region.

To illustrate, without limitation, the outer core region may include at least one layer including polymeric matrix material as described throughout these teachings (in the amounts as described) in which there are embedded fiber bundles. The fiber bundles may include a plurality of twisted filaments. One or more fiber bundles may be nested and secured together. They may be at least partially wrapped with a bias element. A Plurality of bundles may be wrapped with a bias element. The bias element may include a plurality of fiber bundles (some or all having a crosssectional profile shape a circular, triangular, polygonal, rectangular, or other arrangement). The wrapping of the bias may be at an angle relative to a longitudinal axis of the wrapped body that is at an angle >10 degrees and ≤90 degrees.

A resulting flexural modulus of the outer core may be at least 10 GPa, 15 GPa or 20 GPa. It may be below 50 GPa, 40 GPa or 30 GPa.

A similar approach to construction may be employed for the creating nested bundles of fibers for the inner core. Winding angles of bias elements for the inner core may differ from those of the outer core (e.g., it is possible that the angle may be less than 10 degrees.

A resulting flexural modulus of the inner core may be higher than that of the outer core and may be at least 15 GPa, 20 GPa or 25 GPa. It may be below 60 GPa, 50 GPa or 40 GPa.

It is possible that the composite will result in a structure by which the outer region dampens the load transfer between the tissue and inner core, while the outer core provides strength, stiffness and damage protection, and the inner core provides strength, stiffness and responsiveness.

It will be appreciated from the teachings herein that one or more enhancements may be employed in the design of composites to enhance performance. Radii and bevels may be designed and included at transitions between portions of a composite that differ substantially in size, geometry, density or other feature that would provide a stress concentrator. Threads for screws may be angled relative to a longitudinal axis by an angle from 30 to 60 degrees (e.g., 45 degrees). Leading edges of threads may be faced in a direction to reduce force needed for insertion into tissue and to prevent undesired contact with tissue. A distal face of threads may be provided with a sufficiently steep slope to aid in resisting pullout. Threads may have a height of less than 3 mm, a base width of less than 3 mm or both. Threads may have a distal taper at distal end of less than 5 mm. The distal taper may extend for less than 3 revolution, 2 revolutions or one revolution.

The composite may have an outer region having the same or different flexural modulus as a core region(s). Preferably a ratio of the flexural modulus of the outer region to the flexural modulus of the core region (e.g., inner core region including axial fibers or an outer core region including bias fibers) is about 1.0 or less, about 0.90 or less, about 0.80 or less, about 0.70 or less, about 0.60 or less, or about 0.50 or less. The inner core region preferably has a flexural modulus that is greater than a flexural modulus of the outer core region. More preferably, a ratio of the flexural modulus of the inner core to a flexural modulus of the outer core is about 1.10 or more, about 1.20 or more, about 1.30 or more, about 1.40 or more, about 1.50 or more, or about 1.60 or more.

Twisted fiber bundles as used herein preferably refers to a fiber including a plurality of filaments, where the filaments are collectively twisted. Two or more fibers in a fiber bundle may be twisted together. Preferably fibers in a fiber bundle are not twisted together. A twisted fiber bundle may have a circular cross-section, a square cross-section, a polygonal cross-section, or an elongated cross-section (e.g., rectangular, oval or elliptical).

### DETAILED DESCRIPTION OF THE DRAWINGS

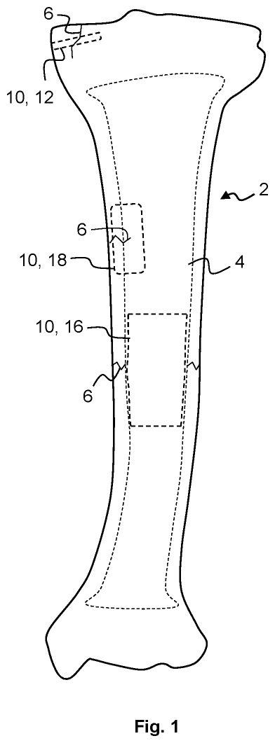

FIG. 1 is a plan view of a bone 2. The bone 2 includes an intramedullary canal 4. Composite orthopedic implants 10 are disposed on or within the bone 2. The composite orthopedic implants 10 are employed at the sites of fractures 6. The composite orthopedic implants 10 illustrated include a pin 12, plate 18, and splint 16. The pin 12 extends through cortical bone. The plate 18 is disposed on a perimetric surface of the bone 2. The splint 16 is disposed within the intramedullary canal 4 of the bone.

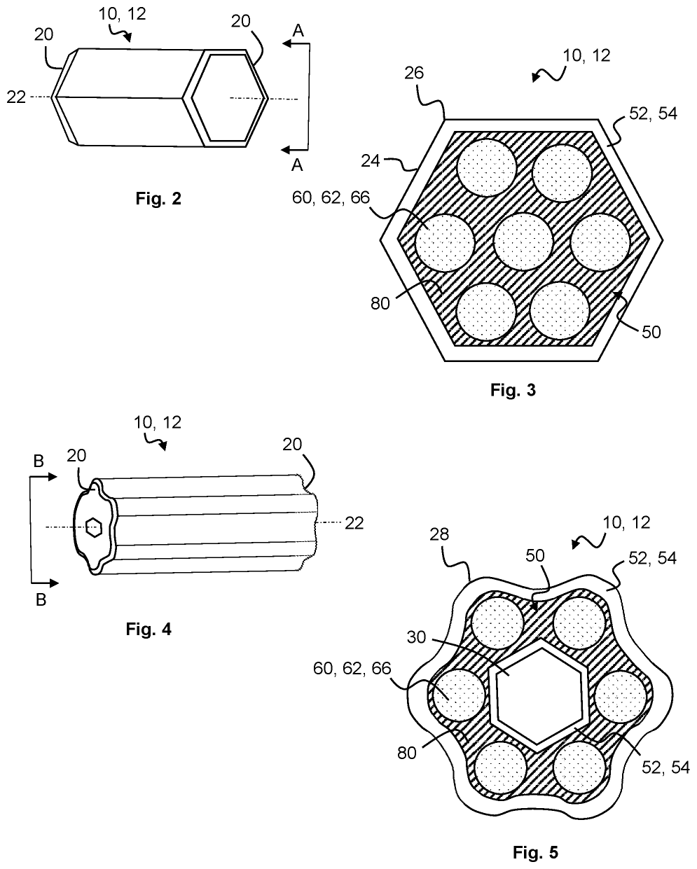

FIG. 2 is a perspective view of a composite orthopedic implant 10 in the form of a pin 12. The pin 12 is an elongate, generally straight structure including two ends 20 in opposing relationship to each other. The pin 12 defines a longitudinal axis 22.

FIG. 3 is a sectional view, along line A-A of FIG. 2, of a composite orthopedic implant 10 in the form of a pin 12. The pin 12 comprises seven reinforcement elements 60 in the form of rods 66. The reinforcement elements 60 are axial reinforcement elements 62 as they are arranged parallel to the longitudinal axis 22 of the pin 12, as shown in FIG. 2. The axial reinforcement elements 62 are disposed in a hexagonal arrangement within the pin 12. The axial reinforcement elements 62 are fixated within matrix material 80. The axial reinforcements 62 and matrix material 80 form a core 50 of the pin 12. The pin 12 comprises an outer region 52, which is a coating 54 circumscribing the core 50. The cross-sectional shape of the pin 12 is hexagonal. The pin 12 defines six corners 26 and six facets 24.

FIG. 4 is a perspective view of a composite orthopedic implant 10 in the form of a pin 12. The pin 12 is an elongate, generally straight structure including two ends 20 in opposing relationship to each other. The pin 12 defines a longitudinal axis 22.

FIG. 5 is a sectional view, along line B-B of FIG. 4, of a composite orthopedic implant in the form of a pin. The pin 12 comprises six reinforcement elements 60 in the form of rods 66. The reinforcement elements 60 are axial reinforcement elements 62 as they are arranged parallel to the longitudinal axis 22 of the pin 12, as shown in FIG. 4. The axial reinforcement elements 62 are disposed in a hexagonal arrangement within the pin 12. The axial reinforcement elements 62 are fixated within matrix material 80. The axial reinforcements 62 and matrix material 80 form a core 50 of the pin 12. The pin 12 comprises an outer region 52, which is a coating 54 circumscribing the core 50. The cross-sectional shape of the pin 12 is lobed. The pin 12 defines six lobes 28. The pin 12 comprises a cannulation 30. The cannulation 30 extends longitudinally through the center of the pin 12 and opens at both of the opposing ends 20, as shown in FIG. 4. The pin 12 comprises an outer region 52, which is a coating 54 circumscribing the cannulation 30.

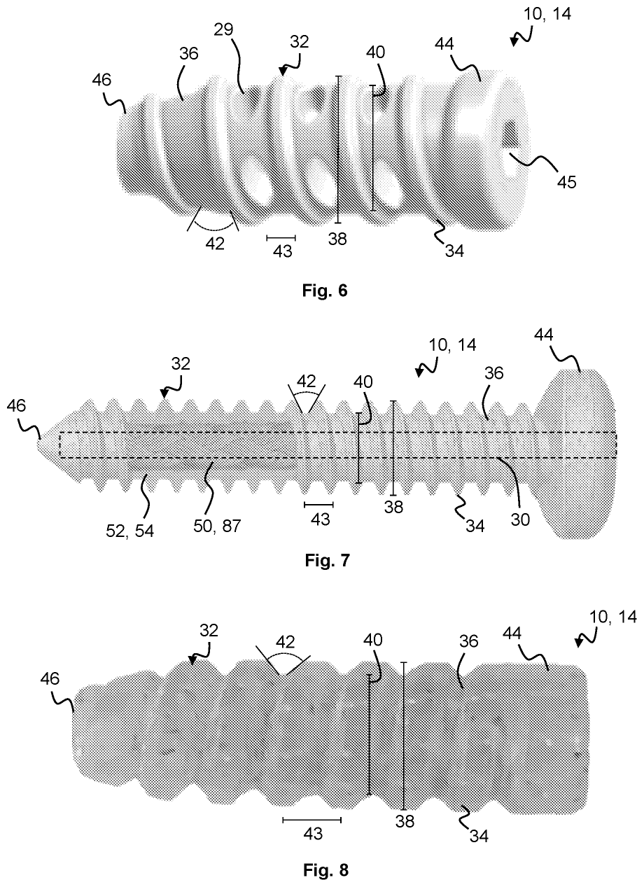

FIGS. 6-8 are perspective views of a composite orthopedic implant 10 in the form of a screw 14. The screw may have spaced apart apertures 29, such as illustrated in FIG. 6. The screw 14 includes a head 44 and a tip 46 in opposing relationship. As shown in FIG. 7, a cannulation 30 extends longitudinally through the screw 14 between the head 44 and tip 46. It is contemplated by the present teachings that the screw 14 illustrated in FIG. 7 may be free of a cannulation 30. The cannulation 30 is a shaped (e.g., hexagonal) cross-section configured to cooperate with a driver (e.g., screwdriver). It is contemplated by the present teachings that the cannulation 30 may have any suitable cross-sectional shape.

As shown in FIG. 7, the screw comprises a core 50 and an outer region 52 disposed around the core 50. The core 50 comprises a textile 87 fabricated from a plurality of fibers. The textile 87, as illustrated, is in the form of a biaxial braid. It is contemplated by the present teachings that the core 50 may be in the form of a uniaxial wrap, triaxial braid, or even quadaxial braid. As illustrated, the biaxial braid comprises a first plurality of fibers oriented about +20° from the longitudinal axis of the screw 14 and a second plurality of fibers oriented about −20° from the longitudinal axis of the screw 14. The outer region 52 is fabricated from a coating 54 (e.g., polymeric material). The plurality of fibers of the core 50 may be individually coated, uncoated, or both (e.g., a first quantity of coated fibers intermingled with a second quantity of uncoated fibers).

The screw 14 includes threading 32, the threading 32 wrapping helically around the screw 14. The threading 32 is defined by crests 34 and roots 36. The diameter between crests 34 is referred to as the major diameter 38. The diameter between roots 36 is referred to as the minor diameter 40. The distance between adjacent crests 34 is referred to as the pitch 43. The threading 32 is also defined by a thread angle 42. The threading 32 may be fabricated from material addition or material removal, according to processes discussed herein.

In one aspect of the present teachings, the threading 32 may be reinforced. Reinforcement may be achieved by disposing one or more fibers, fiber bundles, or reinforcement elements within the threading. As a non-limiting example, a fiber bundle may be helically wound around the screw 14. The core 50 and helically wound fiber bundle may be overmolded with a coating 54. As another non-limiting example, a fiber bundle may be helically wound around the screw 14 and one or more tapes may be wound helically around the screw 14 over the helically wound fiber bundle. As more tape is wound over the fiber bundle, the major diameter 38 of the threading may be increased. The presence of the fiber bundle under the tape may form an impression of threading in the tape.

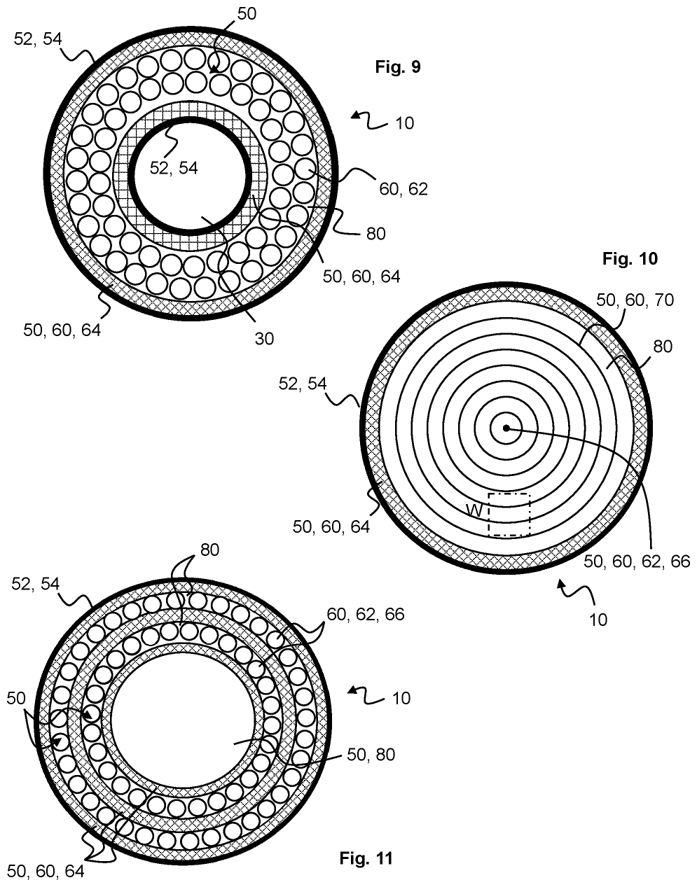

FIG. 9 is a sectional view of a composite orthopedic implant 10 along its transverse axis. The composite orthopedic implant 10 comprises three cores 50. The outermost core 50 is fabricated from reinforcement elements 60 (bias reinforcement elements 64) that are oriented at a bias to the longitudinal axis of the composite orthopedic implant 10. The next adjacent core 50 is fabricated from a plurality of reinforcement elements 60 (axial reinforcement elements 62) that are oriented parallel to the longitudinal axis of the composite orthopedic implant 10. The plurality of axial reinforcement elements 62 are fixated within matrix material 80. The innermost core 50 is fabricated from reinforcement elements 60 (bias reinforcement elements 64) that are oriented at a bias to the longitudinal axis of the composite orthopedic implant 10. The bias reinforcement elements 64 of the outermost core 50 and the bias reinforcement elements 64 or the innermost core 50 are oriented at different angles with respect to each other and the longitudinal axis of the composite orthopedic implant 10.

The composite orthopedic implant 10 comprises an outer region 52, which is a coating 54 circumscribing the outermost core 50. The composite orthopedic implant comprises a cannulation 30 and another outer region 52, which is a coating circumscribing the innermost core 50 and the cannulation 30.

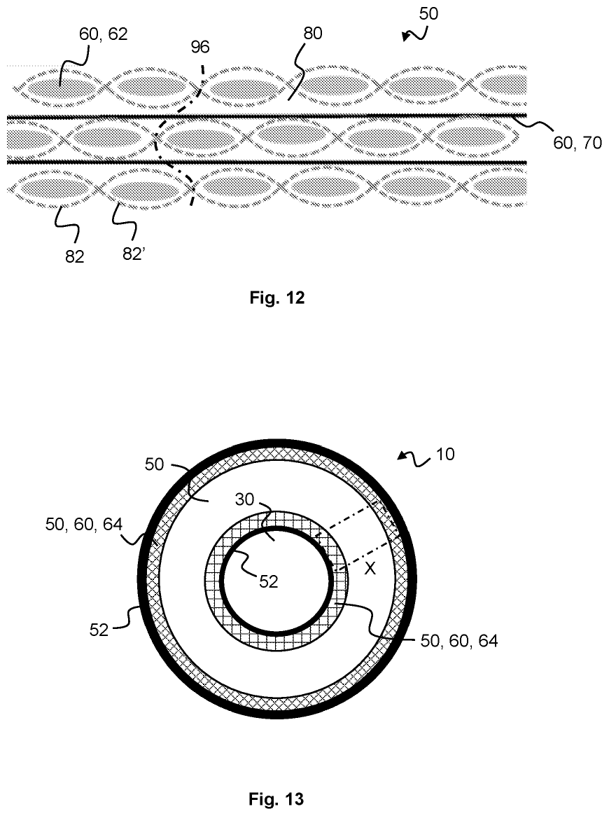

FIG. 10 is a sectional view of a composite orthopedic implant 10 along its transverse axis. The composite orthopedic implant 10 comprises two cores 50. The outermost core 50 is fabricated from reinforcement elements 60 (bias reinforcement elements 64) that are oriented at a bias to the longitudinal axis of the composite orthopedic implant 10. The next adjacent core 50 is fabricated from a series of reinforcement elements 60 in the form of tubes 70 concentrically arranged within one another. Layers of matrix material 80 and axial reinforcement elements 62, as shown in FIG. 12, are disposed between each of the tubes 70. The innermost core 50 is fabricated from a single reinforcement element 60 (axial reinforcement element 62) in the form of a rod 66. The composite orthopedic implant 10 comprises an outer region 52, which is a coating 54 circumscribing the outermost core 50.

FIG. 11 is a sectional view of a composite orthopedic implant 10 along its transverse axis. The composite orthopedic implant 10 comprises six cores 50. The outermost core 50 is fabricated from reinforcement elements 60 (bias reinforcement elements 64) that are oriented at a bias to the longitudinal axis of the composite orthopedic implant 10. The next adjacent core 50 is fabricated from a plurality of reinforcement elements 60 (axial reinforcement elements 62), fixated in matrix material 80, that are oriented parallel to the longitudinal axis of the composite orthopedic implant 10. The next adjacent core 50 is fabricated from reinforcement elements 60 (bias reinforcement elements 64) that are oriented at a bias to the longitudinal axis of the composite orthopedic implant 10. The next adjacent core 50 is fabricated from a plurality of reinforcement elements 60 (axial reinforcement elements 62), fixated in matrix material 80, that are oriented parallel to the longitudinal axis of the composite orthopedic implant 10. The next adjacent core 50 is fabricated from reinforcement elements 60 (bias reinforcement elements 64) that are oriented at a bias to the longitudinal axis of the composite orthopedic implant 10. The innermost core 50 is fabricated from a unitary column of matrix material 80. The composite orthopedic implant 10 comprises an outer region 52, which is a coating 54 circumscribing the outermost core 50.

FIG. 12 is a sectional view of a composite orthopedic implant 10 in the segment W of FIG. 10. The cores 50 comprise a plurality of reinforcement elements 60 (axial reinforcement elements 62) that are oriented in parallel to the longitudinal axis of the orthopedic implant 10. The axial reinforcement elements 62 are arranged side-by-side in a single layer in an ordered arrangement. Fibers 82, 82′ are braided in between the axial reinforcement elements 62 with one set of fibers 82 and another set of fibers 82′ alternate sides of the axial reinforcement elements 62 of which they wrap around, thereby interlocking the axial reinforcement elements 62. Reinforcement elements in the form of tubes 70 are disposed between the axial reinforcement elements 62. The axial reinforcement elements 62, fibers 82, 82′, and tubes 70 are fixated within matrix material 80.

A fracture path 96 extends from one end of the core to the other end. Due to the interlocking fibers 82, 82′, the fracture path 96 is influenced to extend in a tortuous manner from one end to the other end.

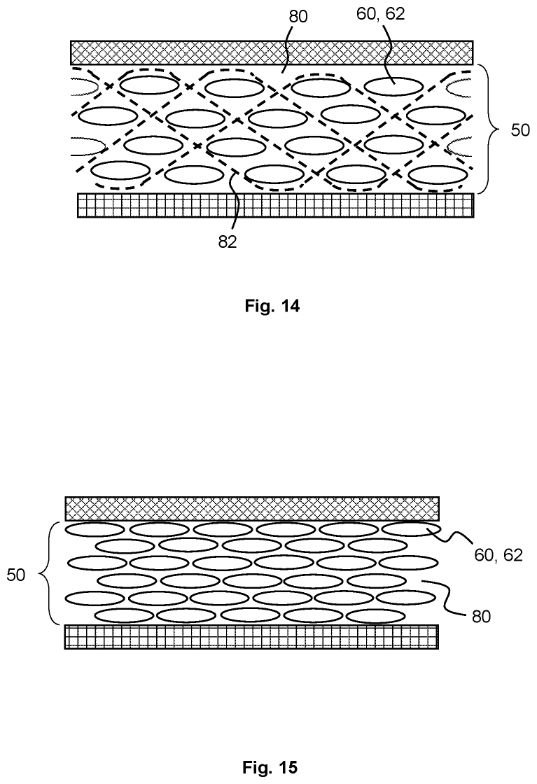

FIG. 13 is a sectional view of a composite orthopedic implant 10 along its transverse axis. The composite orthopedic implant 10 comprises three cores 50. The outermost core 50 is fabricated from reinforcement elements 60 (bias reinforcement elements 64) that are oriented at a bias to the longitudinal axis of the composite orthopedic implant 10. The next adjacent core 50 is fabricated from reinforcement elements 60 and matrix material 80, as shown in FIGS. 14-15. The innermost core 50 is fabricated from reinforcement elements 60 (bias reinforcement elements 64) that are oriented at a bias to the longitudinal axis of the composite orthopedic implant 10. The composite orthopedic implant 10 comprises an outer region 52, which is a coating 54 circumscribing the outermost core 50. The composite orthopedic implant 10 comprises a cannulation 30 and another outer region 52, which is a coating circumscribing the innermost core 50 and the cannulation 30.

FIG. 14 is a sectional view of a composite orthopedic implant 10 in the segment X of FIG. 13, according to one aspect of the present teachings. The core 50 comprises a plurality of reinforcement elements 60 (axial reinforcement elements 62) that are oriented in parallel to the longitudinal axis of the orthopedic implant 10. The axial reinforcement elements 62 are arranged in a staggered and ordered arrangement. Fibers 82, 82′ are extending diagonally through rows of side-by-side axial reinforcement elements 62, thereby interlocking the axial reinforcement elements 62. The axial reinforcement elements 62 and fibers 82, 82′ are fixated within matrix material 80.

FIG. 15 is a sectional view of a composite orthopedic implant 10 in the segment X of FIG. 13, according to another aspect of the present teachings. The core 50 comprises a plurality of reinforcement elements 60 (axial reinforcement elements 62) that are oriented in parallel to the longitudinal axis of the orthopedic implant 10. The axial reinforcement elements 62 are arranged in a staggered and ordered arrangement. The axial reinforcement elements 62 are fixated within matrix material 80.

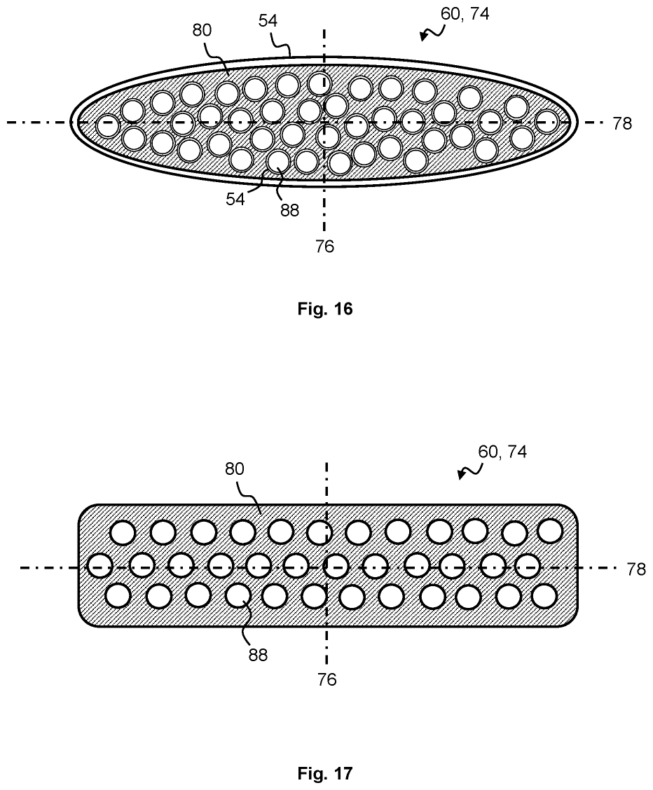

FIG. 16 is a sectional view of a reinforcement element 60 in the form of tape 74. The tape 74 is fabricated from a plurality of fiber bundles 88 in an un-ordered arrangement that are coated with a coating 54. The plurality of fiber bundles 88 are fixated in matrix material 80. The perimeter of the tape 74 is coated with a coating 54. The tape 74 has an elliptical cross-section. The tape 74 is defined by a major axis 78 and a minor axis 76.

FIG. 17 is a sectional view of a reinforcement element 60 in the form of tape 74. The tape 74 is fabricated from a plurality of fiber bundles 88 in an ordered arrangement. The plurality of fiber bundles 88 are fixated in matrix material 80. The tape 74 has a rectangular cross-section. The tape 74 is defined by a major axis 78 and a minor axis 76.

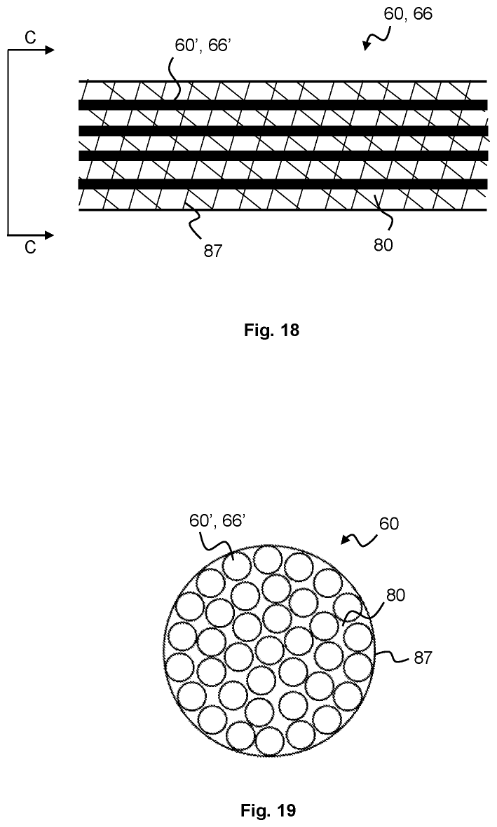

FIG. 18 is a plan view of a reinforcement element 60 in the form of a rod 66. The reinforcement element 60 comprises a plurality of other reinforcement elements 60′ in the form of rods 66′, which are nested in the center of the reinforcement element 60 and bound by a textile 87. The reinforcement element 60 is fixated and impregnated with matrix material 80.

FIG. 19 is a sectional view of a reinforcement element 60 in the form of a rod 66, along line C-C of FIG. 18. The reinforcement element 60 comprises a plurality of other reinforcement elements 60′ in the form of rods 66′, which are nested in the center of the reinforcement element 60 and bound by a textile 87. The reinforcement element 60 is fixated and impregnated with matrix material 80.

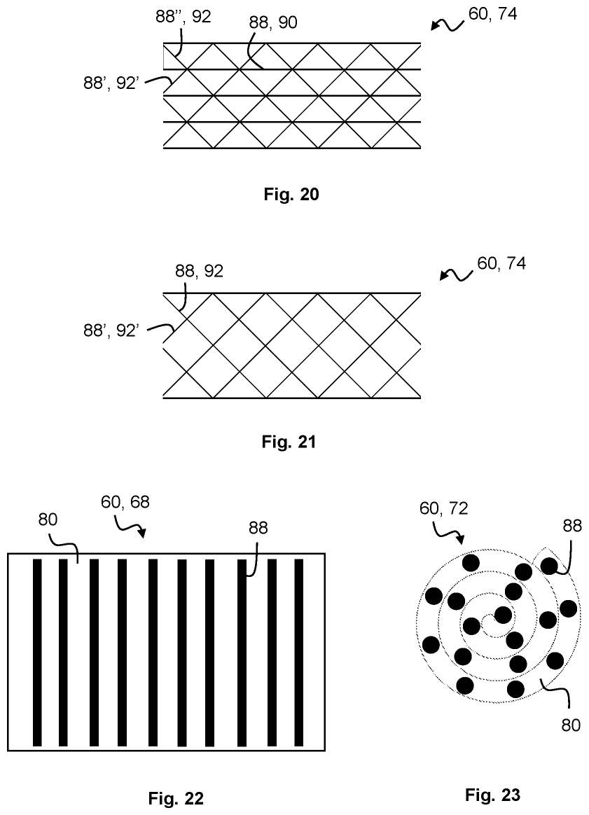

FIG. 20 is a plan view of a reinforcement element 60 in the form of a textile 87. The textile 87 is comprised of a first set of fiber bundles 88 (bias fiber bundles 92) oriented at +45° with respect to the longitudinal axis of the textile 87 and a second set of fiber bundles 88′ (bias fiber bundles 92′) oriented at −45° with respect to the longitudinal axis of the textile 87. The textile 87 comprises a fourth set of fiber bundles 88″ (axial fiber bundles 90). The fiber bundles 88, 88′ are braided together to form the textile 87. It is contemplated by the present disclosure that the textile 87 may be impregnated with matrix material to fabricate a reinforcement element. The reinforcement element may be in the form of tape or a sheet. According to another aspect of the present disclosure, two or more textiles may be laminated together and/or impregnated with matrix material to form a reinforcement element.

FIG. 21 is a plan view of a reinforcement element 60 in the form of a textile 74. The textile 87 is comprised of a first set of fiber bundles 88 (bias fiber bundles 92) oriented at +45° with respect to the longitudinal axis of the textile 87 and a second set of fiber bundles 88′ (bias fiber bundles 92′) oriented at −45° with respect to the longitudinal axis of the textile 87. The fiber bundles 88, 88′ are braided together to form the textile 87. It is contemplated by the present disclosure that the textile 87 may be impregnated with matrix material to fabricate a reinforcement element. The reinforcement element may be in the form of tape or a sheet. According to another aspect of the present disclosure, two or more textiles may be laminated together and/or impregnated with matrix material to form a reinforcement element.

FIG. 22 is a plan view of a reinforcement element 60 in the form of a sheet 68. The sheet 68 comprises a plurality of fiber bundles 88 aligned in parallel to one another. The plurality of fiber bundles 88 are fixated in matrix material 80.

FIG. 23 is a sectional view of a reinforcement element 60 in the form of a roll 72 (rolled sheet). The sheet 68 of FIG. 22 is rolled onto itself. The plurality of fiber bundles 88, which are fixated in matrix material 80, are disposed axially within the roll 72.

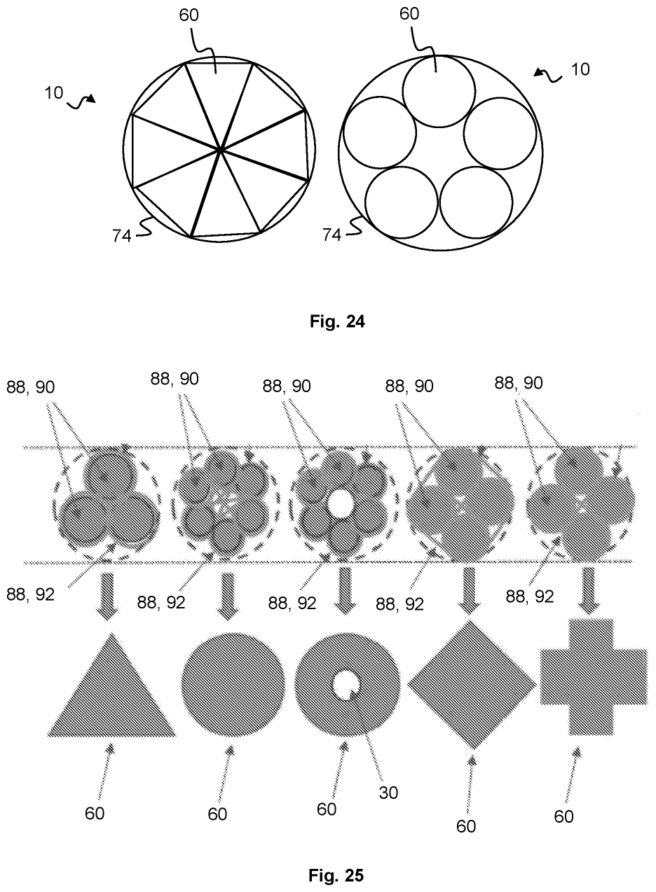

FIG. 24 illustrates sectional views of composites 10. The composite 10 shown to the left comprises eight reinforcement elements 60 with a triangular cross-section. The composite 10 shown to the right comprises 5 reinforcement elements 60 with circular cross-sections. The reinforcement elements 60 shown to the left nest in a tighter arrangement with less interstitial space between the reinforcement elements 60. The reinforcement elements 60 shown to the right nests in a looser arrangement with more interstitial space between the reinforcement elements 60.

FIG. 25 illustrates various arrangements of fiber bundles 88 in reinforcement elements 60. Axial fiber bundles 88 are nested to form various cross-sectional shapes of the reinforcement element 60. From left to right, the axial fiber bundles 88 are arranged to form a triangle, circle, ring, square, and cross. Each of the axial fiber bundles 88 of the reinforcement elements 60 are bound and interlocked by bias fiber bundles 92. In the left-most two and right-most two reinforcement elements 60, the bias fiber bundles 92 cross through the center of the reinforcement element 60. In the center reinforcement element 60 the bias fiber bundles 92 do not pass through the center of the reinforcement element 60 resulting in a cannulation 30.

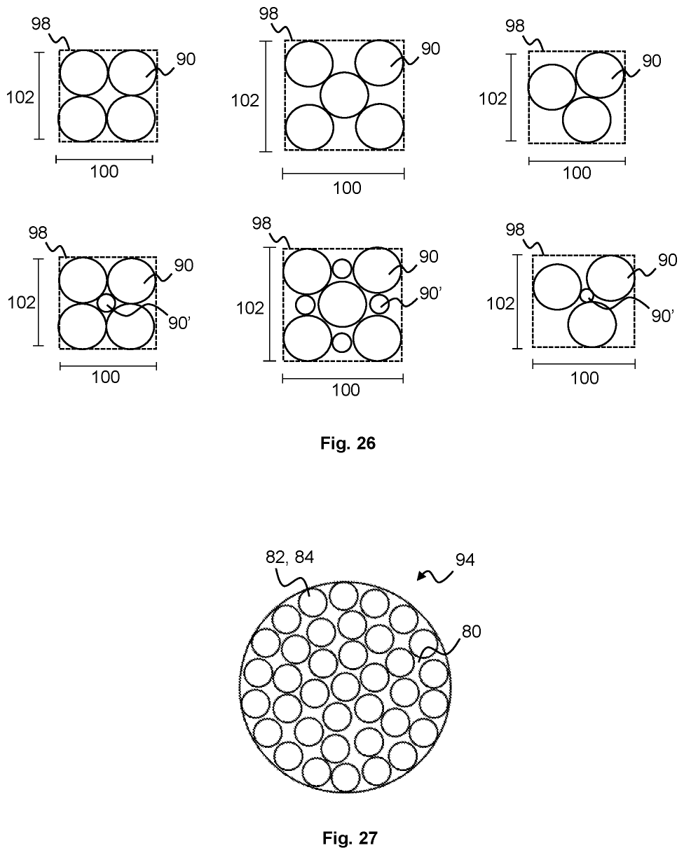

FIG. 26 illustrates various arrangements of axial fiber bundles 90. The axial fiber bundles 90 are defined by a cell size 98. The cell size 98 includes a cell length 100 and cell width 102. The cell size 98 is influenced by the arrangement of adjacent axial fiber bundles 90. The volume of interstitial spaces between axial fiber bundles 90 is influenced by the arrangement of adjacent axial fiber bundles 90. In the bottom row of axial fiber bundle 90 arrangements, the cells include axial fiber bundles 90′ with a smaller diameter than the other axial fiber bundles 90. By the inclusion of the smaller diameter axial fiber bundles 90′ the volume of interstitial spaces therebetween may be reduced without substantially affecting the cell sizes 98.

FIG. 27 is a sectional view of a fiber composite 94. The fiber composite 94 comprises a plurality of fibers 82 (axial fibers 84) nested in an unordered arrangement. The plurality of fibers 82 are fixated by matrix material 80.

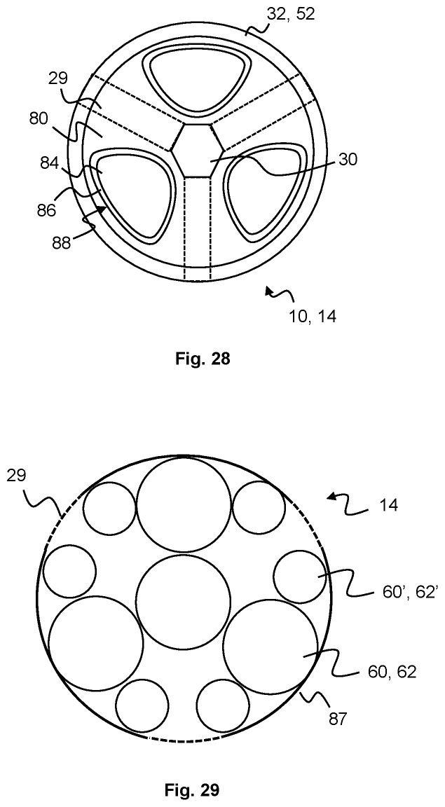

FIG. 28 is a sectional view of a screw 14 (e.g., FIG. 6). The screw 14 comprises three axial fiber bundles 90 each having ovoid cross-sections. The axial fiber bundles 90 include axial fibers 84 and bias fibers 86. The axial fiber bundles 90 are bound and interlocked by a braid of bias fiber bundles (not shown, see e.g., FIG. 25). The interstitial space between the axial fiber bundles 90, which is occupied by the bias fiber bundles is also occupied by matrix material 80. The screw 14 includes a cannulation 30 extending along the central longitudinal axis of the screw 14. The screw 14 includes an outer region 52, which defines threading 32. Apertures 29 are defined in the interstitial spaces between adjacent axial fiber bundles 90. The apertures 29 extend between the cannulation 30 and the perimeter of the outer region 52.

FIG. 29 is a sectional view of a screw 14. The screw 14 comprises four reinforcement elements 60 (axial reinforcement elements 62) each having circular cross-sections and six reinforcement elements 60′ (axial reinforcement elements 62) each having circular cross-sections and smaller diameters than the four reinforcement elements 60. Apertures 29 are defined by the interstitial spaces between adjacent reinforcement elements. The apertures 29 extend partially along the radius of the screw 14.

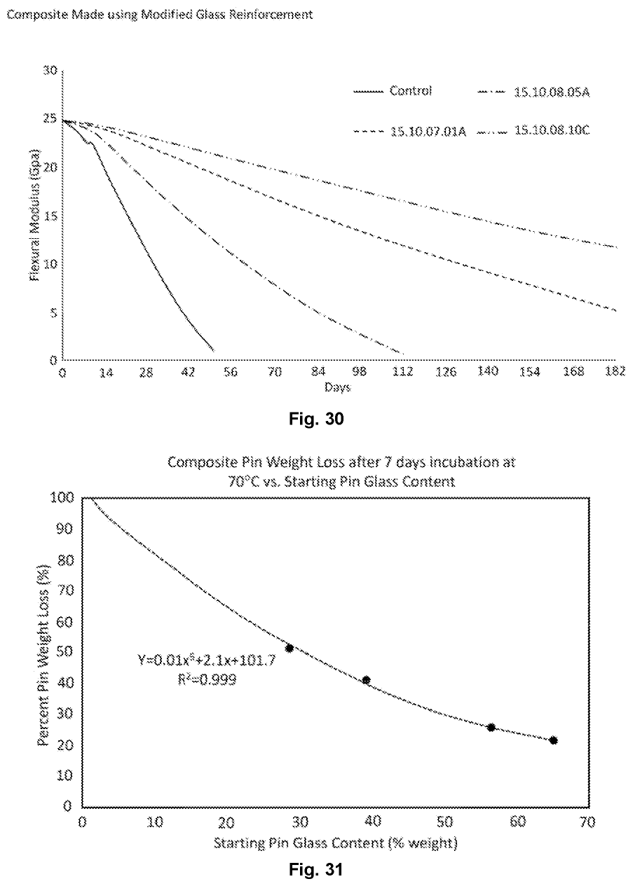

FIG. 30 through FIG. 33 illustrate charts corresponding to Examples 2-5.

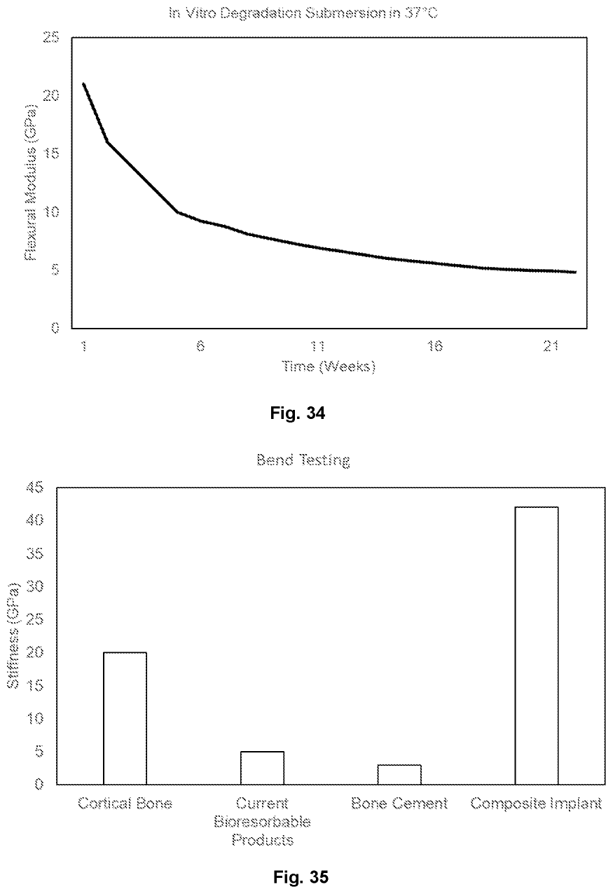

FIG. 34 illustrates a chart. The chart illustrates that the flexural stiffness of the composite implant reduces over time as the composite implant degrades when stored at 37° C. The flexural stiffness of the composite implant reduces in a generally parabolic manner. As discussed herein, the degradation profile of the composite implant may be modulated so the composite implant maintains a particular flexural modulus for a sufficient amount of time to allow the bone to heal.

FIG. 35 illustrates a chart. The chart illustrates that the composite implant according to the present disclosure performs better than current commercially available products. The composite implant of the present disclosure has approximately twice the stiffness as cortical bone. As discussed herein, the mechanical properties (stiffness) may be modulated so the stiffness is less than or possibly greater than what is illustrated.

FIG. 36 and FIG. 37 correspond to Example 10.

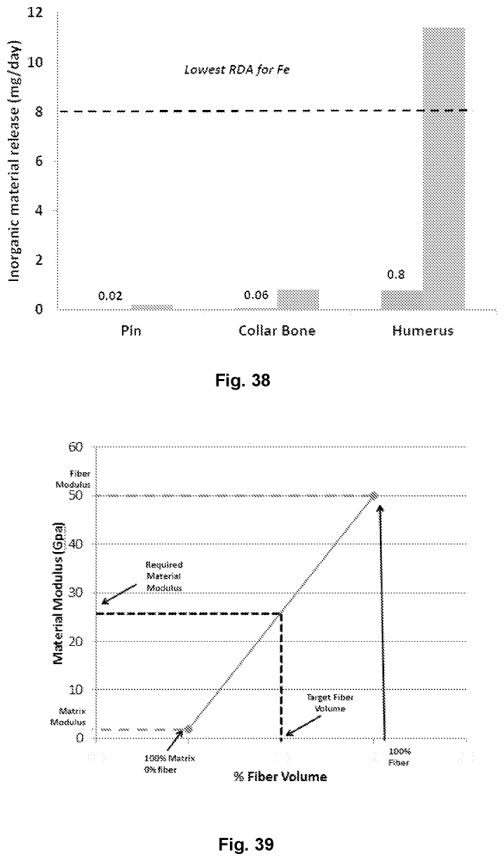

FIG. 38 illustrates a chart corresponding to Example 11.

FIG. 39 illustrates a chart. The chart illustrates the dependence of the composite article's modulus on both the volume matrix material and the volume of fiber material used to fabricate the composite article. A composite article comprising entirely matrix material has a comparatively low modulus while a composite article comprising entirely fiber material has a comparatively high modulus. By balancing the matrix volume with fiber volume, the mechanical properties according to the present disclosure may be realized. While it is contemplated that the composite article of the present disclosure comprises at least some volume of matrix material, it may be advantageous to increase the fiber volume. This may be achieved, at least in part, by selecting for cross-sectional shapes and cross-sectional thicknesses of fiber bundles, fiber composites, and/or reinforcement elements to improve packing and decrease interstitial spaces between these structures. Additionally, a combination of different cross-sectional shapes and cross-sectional thicknesses may improve packing and decrease interstitial spaces between these structures.

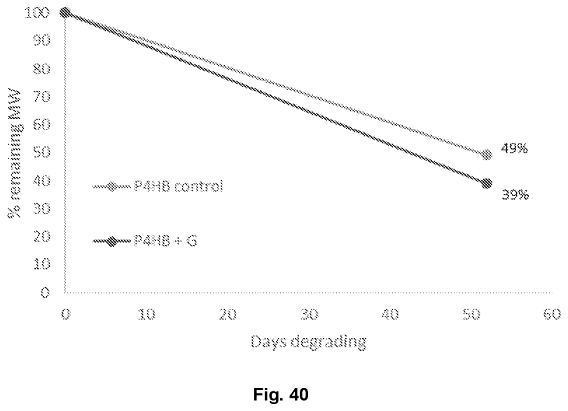

FIG. 40 illustrates a chart corresponding to Example 12.

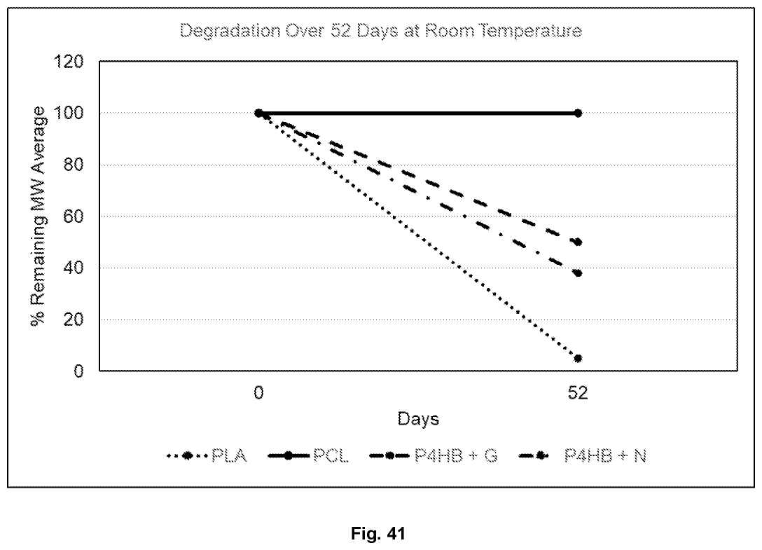

FIGS. 41 through 46 illustrates charts corresponding to Examples 15 through 20.

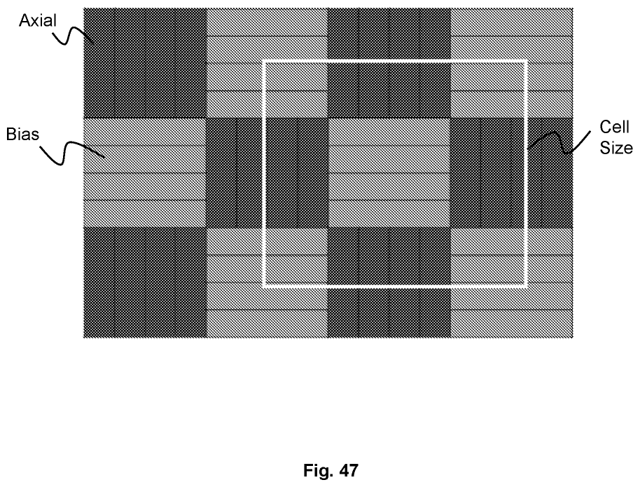

FIG. 47 illustrates a braid. The braid is constructed by a braiding machine employing 12 carriers with 4 filament bundles per carrier and 200 filaments per bundle. The bundles had a 0.7 twist per inch applied.

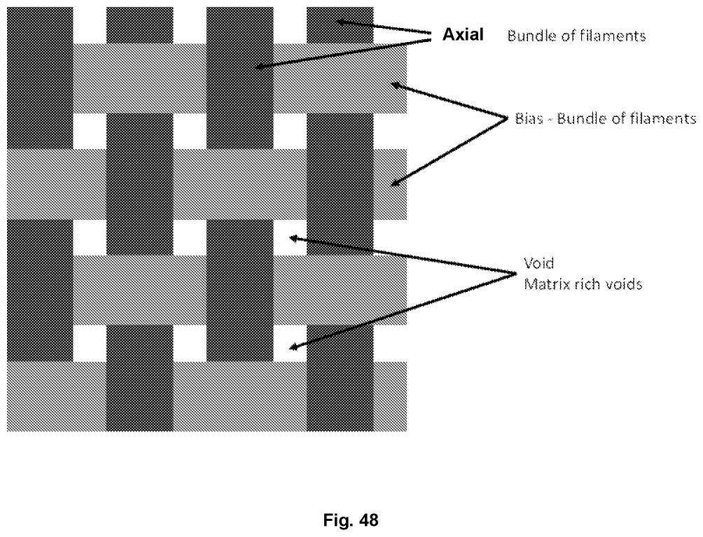

FIG. 48 illustrates a braid. The braid is constructed by a braiding machine employing 12 carriers with 1 filament bundle per carrier and 800 filaments per bundle. The bundles had a 0.7 twist per inch applied.

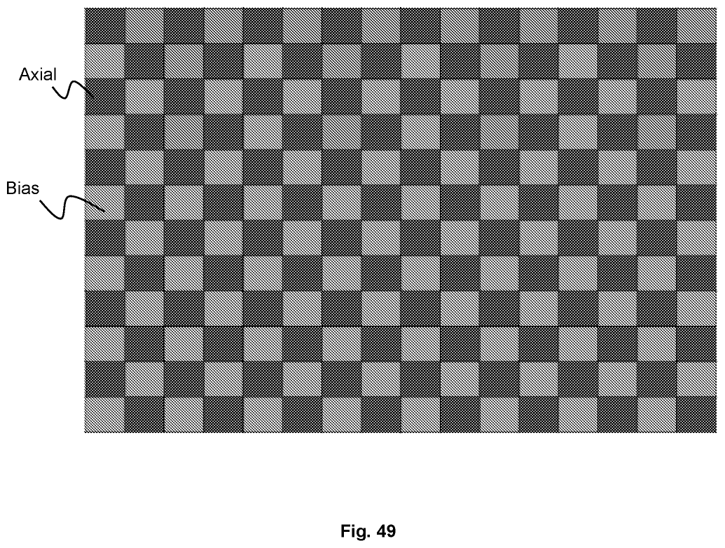

FIG. 49 illustrates a braid. The braid is constructed by a braiding machine employing 48 carriers with 1 filament bundle per carrier and 200 filaments per bundle. The bundles had a 0.7 twist per inch applied. The cell size is ¼.

By way of comparison between FIG. 47, FIG. 48, and FIG. 49, the quantity of bundles per carrier may determine the cell size. The combination of smaller bundles (e.g., 200 filament per bundle vs. 800 filaments per bundle) and more bundles per carrier produce thinner layers, larger cell sizes for a given thickness of layers, higher coverage, smaller matrix rich regions, and less out of plane loading along a filament axis. Twisted bundles, as compared to untwisted bundles, increase the strength of bundles, size of bundles, results in a lower coverage factor, provide larger matrix rich regions, and result in more out of plane loading along a filament axis.

In FIGS. 47, 48, and 49, the bias fibers are illustrated as being perpendicular to the axial fibers. However, it will be appreciated that the bias fibers may have any angle relative to the axial fibers. Typically, the bias fibers have an angle of at least +/−10 degrees relative to the axial fibers, preferably at least +/−15 degrees, and more preferably +/−20 degrees.

When applying a braid, weaving, winding or interlocking structure around axial fiber bundles, the axial fiber bundles preferably are in tension and/or stretched. This has been found to be particularly advantageous when the axial fibers have a twist. The stretching and/or tension of the axial fibers is believed to increase the stiffness of the resulting structure.

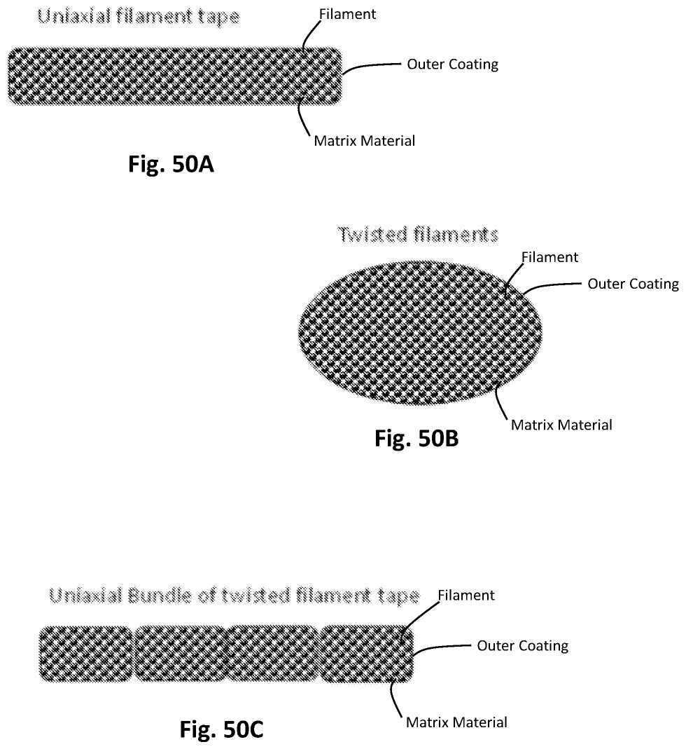

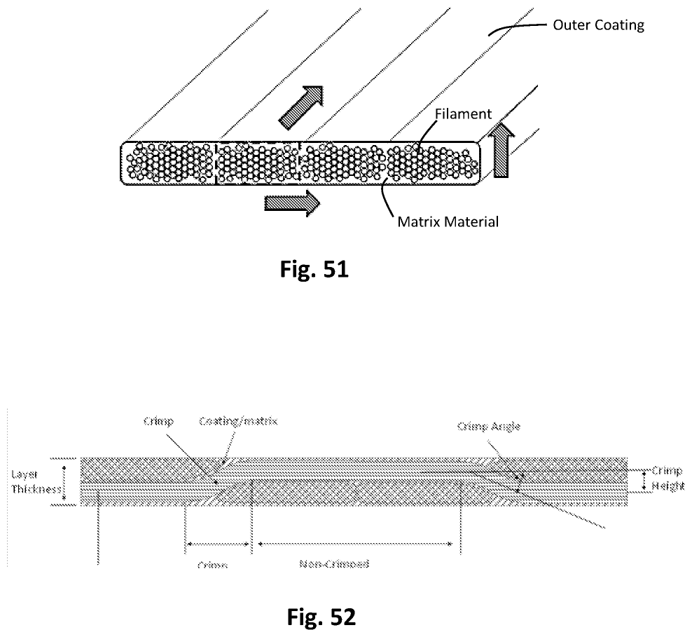

FIG. 50A illustrates a uniaxial braided structure in accordance with the present teachings. FIG. 50B illustrates a twisted filament braided structure in accordance with the present teachings. FIG. 50C illustrates a uniaxial, twisted filament braided structure in accordance with the present teachings.

A bias element preferably has an elongated cross-sectional profile perpendicular to a length or axial direction of the bias element, such as illustrated in FIG. 50A. In FIG. 50A, the bias element is shown as a tape having a plurality of filaments in a matrix. The matrix includes or consists of a polymeric or polymerizable matrix, with a coating. The coating may be the same material or a different material as the matrix. In order to achieve a high width/thickness ratio (i.e., a highly elongated cross-sectional profile), it is preferable that the filaments are generally free of or entirely free of twists. For example, the width to thickness ratio may be about 1.5 or more, about 2.0 or more, about 2.5 or more, about 3.0 or more, or about 4.0 or more, such as illustrated in FIG. 50A. In various aspects according to the teachings herein, it may be advantageous for the filaments to be twisted together (e.g., in a twisted fiber). When the filaments are twisted together in a fiber, or a twisted bundle of fibers, it may be possible to obtain an elongated cross-sectional profile, such as illustrated 50C. However, the twisted filaments may limit the width to thickness ratio to about 2.0 or less, about 1.8 or less, about 1.6 or less, about 1.50 or less, or about 1.4 or less. In order to achieve a high width to thickness ratio of the bias element, it may be necessary to use a plurality of bias tapes that are arranged side by side, such as illustrated in FIG. 50B. Here, the individual bias tapes have a generally low width to thickness ratio, but together, the width to thickness ratio may be much greater, without limit. Thus, a bias element with twisted filaments can be achieved having similar width to thickness ratios as the bias element without twisted filaments. Additionally, it may be possible to join together a plurality of bias tapes, or join together a plurality of fibers, each having twisted filaments, in order to achieve a high width to thickness ratio in a single tape. As such, structures such as FIG. 50B may be achieved where a plurality of the tapes are replaced by a single tape.

By way of example, FIG. 50 illustrates a bias tape having a generally high width to thickness ratio, including a plurality of fibers where each fiber includes twisted filaments.

The bias element may be a tape like structure including multiple fibers arranged side-by-side along the width direction and aligned along an axial direction, such as illustrated in FIG. 50. The thickness of the bias element may be the thickness of a single fiber. The bias element, including fibers having twisted filaments may have a width to thickness ratio of about 1.5 or more, about 2.0 or more, about 2.5 or more, about 3.0 or more, about 4.0 or more, or about 5.0 or more.

A plurality of bias elements may be woven or braided together, such as illustrated in FIG. 52. As illustrated in FIG. 52, a first bias element may be woven or braided with a second bias element arranged in a different direction (e.g., in an orthogonal direction, or a non-orthogonal direction). The first bias element may have a series of periodic crimps as it goes under and then over the second bias elements. The frequency of the crimps may be controlled by the width and/or arrangement of the second bias elements. In FIG. 52, two bias tapes are aligned adjacent to each other and the first bias element extends over both of the tapes. Thus, crimp frequency (or distance between crimps) may be defined by the length of the tapes and the number of tapes between crimps. The height of the crimp is typically about the thickness of the second bias element (e.g., the thickness of the second bias tape). The angle of the crimp may be controlled by the contour of the bias tape. The layer thickness is typically about equal to the combined thickness of the first bias element and the second bias element. The bias layer of FIG. 52 preferably has a coating or matrix that fills the gaps between the bias elements.

### Mechanical Properties and Test Methods

Fracture toughness may refer the resistance of materials to the propagation of flaws under an applied stress. It is generally assumed that the longer the flaw relative to the thickness of the part, the lower the stress is required to cause fracture. High fracture toughness in metals is generally achieved by increasing the ductility at the expense of lower yield strength. Fiber reinforced composites are generally anisotropic with the highest fracture resistance occurring with breakage and pull-out of the fibers and lowest resistance occurring in interlaminar planes.

Brittle failure refers to the breakage of a material due to a sudden fracture. When a brittle failure occurs, the material breaks suddenly instead of deforming or straining under load.

Ductile failure is a type of failure seen in malleable materials characterized by extensive plastic deformation or necking. This usually occurs prior to the actual failure of the material.

Strain at yield is the amount of strain at which a yield point is reached. The yield point is the point on a stress-strain curve that indicates the limit of elastic behavior and the beginning of plastic behavior. Strain is a measure of deformation of a material. The strain may be applied by bending, torsion, or compression. The bending may be tested by 3-point bend testing, 4-point bend testing, gap testing, or any combination thereof. The compression may be tested by standard testing, gap testing, or both.

Strain at failure is the amount of strain at which the failure is reached. The failure may refer to brittle failure and/or ductile failure. Strain is a measure of deformation of a material. The strain may be applied by bending, torsion, or compression. The bending may be tested by 3-point bend testing, 4-point bend testing, gap testing, or any combination thereof. The compression may be tested by standard testing, gap testing, or both.

Elastic modulus is a measure of an object's resistance to being deformed elastically (i.e., non-permanently) when a stress is applied. The elastic modulus may be measured in bending, torsion, or compression. The bending may be tested by 3-point bend testing, 4-point bend testing, gap testing, or any combination thereof. The compression may be tested by standard testing, gap testing, or both.

Unless otherwise specified herein, mechanical properties of a composite material in compression may be measured according to ASTM D3410 and/or D3410M-16, both of which are incorporated herein by reference for all purposes. Unless otherwise specified herein, the mechanical properties may be measured with the axial direction of the axial fibers arranged perpendicular to the direction of compression or parallel to the direction of compression.

Unless otherwise specified herein, mechanical properties of a composite material in torsion may be measured according to ASTM D1043-16, incorporated herein by reference for all purposes. Unless otherwise specified herein, the torsional properties are measured with the axial direction of the axial fibers generally aligned parallel to the direction of the length of the test specimen.

Unless otherwise specified herein, flexural properties of a composite material may be measured according to ASTM D790-17, incorporated herein by reference for all purposes. Unless otherwise specified herein, the flexural properties are measured with the axial direction of the axial fibers parallel to the direction of the length of the test specimen.

Unless otherwise specified herein, tensile properties of a composite material may be measured according to ASTM D638-14, incorporated herein by reference for all purposes. Unless otherwise specified herein, the mechanical properties are measured with the axial direction of the axial fibers parallel to the direction of the length of the test specimen.

Unless otherwise specified herein, the glass transition temperature (Tg) of the reactant can be obtained by measurements or also by calculation using the William Landel Ferry Equation (M. L. Williams, et al., J.Am.Chem.Soc. 77, 3701, 1955), incorporated herein by reference for all purposes. The website http://www.wernerblank.com/equat/ViSCTEMP3.htm, incorporated herein by reference for all purposes, provides a simple method to convert viscosity of an oligomeric polymer to the Tg.

Unless otherwise specified herein, the linear-elastic fracture toughness of a material is determined from the stress intensity factor (K) at which a thin crack in the material begins to grow.

Unless otherwise specified herein, specific gravity is measured according to ASTM D792, incorporated herein by reference for all purposes.

Unless otherwise specified herein, fiber volume is measured according to ASTM D3171-15, incorporated herein by reference for all purposes.

Unless otherwise specified herein, the melt flow rate is measured according to ASTM D1238, incorporated herein by reference for all purposes. Unless otherwise specified, the melt flow rate is measured at 180° C./2.16 kg.

Unless otherwise specified herein, the viscosity is measured according to ASTM D445-19a, incorporated herein by reference for all purposes.

Unless otherwise specified herein, the surface roughness is measured according to Lewandowska et. al., The technique of measurement of intraocular lens surface roughness using Atomic Force Microscopy, Interdisciplinary Journal of Engineering Sciences, Vol. II, No. 1 (2014), incorporated herein by reference for all purposes.

Unless otherwise stated herein, pore size and pore number are measured according to Biggs et al., SEM Measurement of Microporous Film Pore Distributions, Microscopy Today, January 2014, incorporated herein by reference for all purposes.

Unless otherwise stated herein, pore specific surface area, pore volume, and pore size distribution is measured according to Murugesu, Pore Structure Analysis Using Subcritical Gas Adsorption Method, Society of Petroleum Engineers, October 2017, incorporated herein by reference for all purposes.

### Composite Implant Examples

The above teachings may be illustrated by exploration of one family of its many applications. Any of the materials, structures, and techniques described generally above may be employed with the following description. For example, without limitation, reference to polymers contemplate polymers (e.g., biodegradable polymers) described previously, as well as other biocompatible polymers (e.g., polyether ether ketone (PEEK)). Additionally, materials for filaments, fibers, bundles, and/or reinforcement elements, as described herein, contemplate biodegradable or bioabsorbable glasses, as well as other glasses. Previous teachings of controlled degradation techniques are also applicable and useful for the illustrations that follow.

More particularly, the general teachings discussed throughout this application are illustrated by reference to structural elements (e.g., composite implant) that are intended to be subjected in use to torsional loading, axial loading, compressive loading, flexural loading, or any combination thereof. The general teachings discussed throughout this application are also illustrated by reference to structural elements that when subjected in use in their intended environment, will degrade over time (e.g., they will degrade according to a predetermined controllable degradation profile).

Turning now to structure for the structural elements, the present discussion will describe various approaches making use of the materials, techniques, and other aspects described previously. All previously discussed general teachings may be employed for a structural element. The omission in this illustration of any such feature is merely for conciseness purposes and is not intended as any exclusion of that feature from use with a structural element.

In general, one such structural element of the present teachings may include an elongated portion. A structural element may have a portion that is subjected to a driving load by a driving device. The structural element and driving device may include a male-female relationship. The structural element may include a male portion (e.g., bolt head) and the driving device may include a female portion (e.g., socket). The driving device may include a male portion (e.g., screwdriver) and the structural element may include a female portion (e.g., Philips-type indentation).

The structural element may have a generally constant width, thickness, and/or diameter portion that extends over a majority of a length of the element. The structural element may have an enlarged portion (in a transverse cross-section taken orthogonally to a longitudinal axis of the element) as compared with remaining portions of the element (e.g., a shank of the element)) at one or more locations along a length of the element. The structural element may have an enlarged portion at an end of the element. The structural element may have an enlarged portion configured as a driving portion of the element. The structural element may have a portion which may not be an enlarged portion relative to remaining portions of the element, but which is nevertheless configured for being driven by a driving device.

The structural element may include a head portion that configured as a driving portion of the element that is configured to receive a driving device (e.g., a hand-driven tool, a motor driven tool or both). The head portion may be connected to a neck portion. The neck portion may lie between the head portion and a shank portion. The head portion may be part of or the entirety of an enlarged portion of the driving element. The head portion may not be enlarged relative to the neck portion, the shank portion or both. The head portion may taper or otherwise reduce in cross-sectional dimension in a continuous or stepped manner to the neck portion. The neck portion may taper or otherwise reduce in cross-sectional dimension in a continuous or stepped manner to the shank portion.

The structural element may be a fastener, as described herein. It may be a rod, a nail, a pin (see e.g., FIGS. 2-5), or a screw (see e.g., FIGS. 6-8), as described herein.

The structural element may be a device adapted for implantation into a body of a live being. For example, the structural element may be an orthopedic implant, as described herein. The orthopedic implant may be configured for any of the orthopedic implant applications described herein.

The structural element may be a device configured for a construction application. For example, the structural element may be a device configured for use in providing fixation for a predetermined period (e.g., to afford curing of an adhesive, concrete, hardening of a joint, etc.). The structural element may be a device configured for use for a predetermined period, and thereafter to erode according to a predetermined degradation profile, as described herein.

Among the various features of the structural element of this description, including any rods, nails, pins, or screws, are the following features, which may be employed alone, or in any combination as part of the structural element.

The head portion, any neck portion, and the shank portion may share one or more common elements (e.g., filaments, fibers, reinforcement elements, and/or fiber bundles, as described herein). The reinforcement elements may have a radius of curvature in their transverse cross-section that is generally equal to (within ±10%, more preferably ±5%, or even more preferably ±1%) the radius of curvature of the head portion, any neck portion, shank portion, or any combination thereof.

The head portion, any neck portion, and the shank portion may share at least one common polymeric matrix material, as described herein.

The head portion, any neck portion, and the shank portion may share a single common polymeric matrix, as described herein.

The head portion, any neck portion, and the shank portion may share a common polymeric matrix material, as described herein, having reinforcement particulates (e.g., filler, as described herein) dispersed therein.

The head portion may include a recess for receiving a driving device. The recess may be defined by a plurality of walls that adjoin one or a plurality of fiber bundles embedded within a polymeric matrix.

The head portion, the neck portion, and/or the shank portion may include a plurality of elements (e.g., fiber bundles, as described herein) that are assembled in an arrangement that defines the structure and dimensions of the head portion, the neck portion, and/or the shank portion.

The head portion, the neck portion, and/or the shank portion may be fabricated by material removal, material addition, or both. The head portion may be fabricated by wrapping tape, as described herein, repeatedly and circumferentially around the structural element to a desired transverse cross-sectional dimension. The tape may be wrapped and translate along a longitudinal axis of the structural element. A taper may be formed by wrapping tape around a portion of the structural element and increasing the number of overlapping wraps as the tape translates along the longitudinal axis of the structural element.

The head portion, the neck portion, and/or the shank portion may include an outer wall. The outer wall may be tapered at one or more locations along its length.

The head portion, the shank portion and optionally any neck portion may include a helical thread.

The head portion, the shank portion and optionally any neck portion may include a helical thread that has a crest with a radius of curvature. For example, the radius of curvature may be about 0.01 mm or more, 0.05 mm or more, or even 0.1 mm or more, 1.5 mm or less, 1 mm or less, or even 0.5 mm or less.

The head portion, the shank portion and optionally any neck portion may include constant cross section diameter portions in regions between the crests of the helical thread.

The filaments, fibers and/or fiber bundles of the elements may be twisted along a longitudinal axis. The filaments, fibers and/or fiber bundles of the elements may have a generally helical orientation. The filaments, fibers and/or fiber bundles of the elements may be free of any uniaxial orientation.

2 or more, 3 or more, 4 or more, or even 6 or more fiber bundles may be bound by a sheath, as described herein. The sheath may comprise a textile, as described herein. The textile may or may not be impregnated with matrix material. The textile may be braided. The braid may be defined by a unit cell. The unit cell may be defined by a length and width of the most basic repeating unit pattern within the braid. The unit cell may comprise 1×1, 2×2, or 3×3 bias bundles. The unit cell may have a length and/or width of about 0.2 mm or more, 0.6 mm or more, or even 0.8 mm or more. The unit cell may have a length and/or width of about 1.8 mm or less, 1.6 mm or less, or even 1.2 mm or less.

The shank may have a distal tip. The distal tip may be solid. The distal tip may be hollow. The distal tip may include a cavity within a solid portion.

The helical thread may be formed of a polymer material, as described herein. The helical thread may include one or more filaments, fibers, fiber bundles, and/or reinforcement elements. The helical thread may include one of more reinforcement particulates (e.g., filler) as described previously.

The fasteners may be made by forming a core. The core may include one or more filaments, fibers, fiber bundles, and/or reinforcement elements, as described herein. The core may include one or more filaments, fibers, fiber bundles, and/or reinforcement elements embedded within a degradable polymeric matrix, as described herein. At least one outer wall may be applied to the core. The at least one outer wall may include a degradable polymer, as described herein. One or more sheaths may be applied to or formed over the core (or any component thereof), the at least one outer wall, or both). A thread may be applied to or formed in the core. A thread may be applied to or formed in a portion of the at least one outer wall. A thread may be applied or formed in the one or more sheath.

The core, the at least one outer wall, any sheath or each may be formed during by extruding filaments, fibers, fiber bundles, and/or reinforcement elements with a polymer. The core, the at least one outer wall, any sheath or each may be formed during by pultruding filaments, fibers, and/or fiber bundles with a polymer, as described herein. The core, the at least one outer wall, any sheath or each may be formed by molding (e.g., injection and/or compression molding). The core, the at least one outer wall, any sheath or each may be formed by additive manufacturing.

A thread may be formed by adding material (e.g., by injecting a hardenable resin, wrapping, by additive manufacturing) to a core, a sheath, an outer wall, or any combination. A thread may be formed by removing material (e.g., by machining) from a core, a sheath, an outer wall, or any combination.

A thread may be formed by helically wrapping at least one fiber, fiber bundle or each around the core. A coating or other layer of degradable polymer (which may include reinforcement particulates as described previously) may be applied over the thread.

As seen from the above, the teachings are suitable for each of pins, rods, nails, and screws, as described herein. In accordance with previously described features herein, these structural elements may be solid along all or a portion of their length. They may have a longitudinal channel (e.g., cannulation as described herein) over all or a portion of their length. They may have plural spaced apart radial openings (e.g., apertures as described herein) along at least a portion of their length. The radial openings may be rounded (e.g., circular or oval). The radial openings may extend partially into the surfaces of these elements. The radial openings may form through-passages into the elements. For example, the through openings may span from an exterior surface of an element through to a longitudinal channel within the opening.

The structural elements may have a surface texture as described previously. The structural elements may be formed to have a dynamic porous structure that evolves over time to correspond with different events, as previously was described. For example, for an orthopedic implant, the porosity may be evolved according to a controlled degradation profile that corresponds with and accommodates different stages of bone a tissue growth without occasioning an adverse foreign body response (i.e., a rejection of the implant by the body).

One aspect of the present illustration is that a head portion of a structural element (e.g., a screw) has a structure configured to transmit torque when driven into a structure (e.g., a bone), without chipping, or otherwise fracturing. The head may include a plurality of fibers and/or fiber bundles, as described herein, at least partial surrounding a female opening in the head for receiving a driver device. The fiber bundles may have a cross section that it circular, oval, triangular and/or generally trapezoidal. It is also possible that there may be one or a plurality of fibers and/or fiber bundles for defining a male projection that would extend into a socket of a driving device.

A neck portion may be formed between a head portion and a shank portion. The neck portion may have a longitudinal tapered geometry. The neck portion may include on or more annular fibers or fiber bundles.

It is possible that a structural element may have any of the exterior surface structures, and/or may be dimensioned according to any of the dimensions (including open and closed surface areas) that are described in U.S. Pat. No. 9,808,298, hereby incorporated by reference in its entirety (see, e.g., FIGS. 1-11). It is possible, for example, to employ the present teachings to prepare an interference screw having an open-architecture configuration, having a diameter from about 5 to 12 mm, a length of about 20 to 35 mm or both.

It is possible to employ the present teachings to prepare, and the teaching contemplate, an interference screw having an open-architecture configuration having an initial (e.g., for an orthopedic implant, at time of implant) open area to closed area ratio (as defined in U.S. Pat. No. 9,808,298, hereby incorporated by reference in its entirety) of about 0.1:20; 0.2:17; 0.5:17; 1:12; 1:10; 1:8; 1:5; 1:4; 1:3; 1:2; or 1:1. A degradation profile may cause the above ratios to vary overtime. Thus, it may have an initial ratio as set forth above but will degrade to have a ratio deviating from the above by any of 10%, 20%, 30%, 40%, 50% or more over a period of any of at least one week, two weeks, four weeks, six weeks, ten weeks, 12 weeks, 18 weeks, or 24 weeks. The open area to closed area ratios and degradation effects presently described are not limited to this illustration only but are also contemplated as a general teaching applicable to all embodiments described in the present teachings.

It is possible to employ the present teachings to prepare, and the teachings contemplate, an interference screw, as described herein, having an open-architecture configuration (e.g., having one or more apertures) having an initial (e.g., for an orthopedic implant, at time of implant) failure torque (as defined in U.S. Pat. No. 9,808,298, hereby incorporated by reference in its entirety) of at least about 1.1 Newton meters (N-m), 1.4 N-m, 1.7 N-m, 2 N-m or more. The failure torques presently described are not limited to this illustration only but are also contemplated as a general teaching applicable to all embodiments described in the present teachings.

The composite article may be a composite implant in the form of a pin. The pin may comprise nested axial fiber bundles. The pin may comprise 3, 4, 5 or even 6 nested axial fiber bundles. A pin fabricated from 3 axial fiber bundles may have a triangular cross-sectional shape. A pin fabricated from 4 axial fiber bundles may have a square cross-sectional shape. A pin fabricated from 6 axial fiber bundles may have a hexagonal cross-sectional shape. It is contemplated by the present disclosure that any variety of cross-sectional shapes may be achieved by the deliberate arrangement of a number (e.g., 3, 4, 5 or even 6) axial fiber bundles. The axial fiber bundles may be embedded in matrix material. The pin may comprise nested axial fiber bundles that are bound by bias fiber bundles. The nested axial fiber bundles may be bound by bias fiber bundles that are braided therearound. The braided bias fiber bundles may include one or more, two or more, or even three or more concentric layers of binding. The pin may comprise nested axial fiber bundles that are bound and interlocked (see e.g., FIG. 25) by bias fiber bundles. The pin may include apertures fabricated therein according to the present teachings as discussed hereinbefore. The pin may include an outer region (e.g., coating). The outer region may be applied to the core of 3, 4, 5 or even 6 axial fiber bundles bound and/or interlocked by bias fiber bundles). The axial fiber bundles may include a sizing (e.g., aminopropyl silane). The pin may have an axial:bias bundle ratio of from about 1:2 to 2:1 (e.g., 1:1). The pin may comprise about 40-50% (e.g., 47%) axial fiber volume and 50-60% (e.g., 53%) bias fiber volume. The bias fiber bundles that bind and/or interlock the axial fiber bundles may be oriented at an angle of about ±45° (±3°) to the axial fibers. The bias fiber bundles may be in the form of a braid. The braid may comprise 1×1, 2×2, or even 3×3 bias fiber bundles. The bias fiber bundles may be in the form of a tape. The tape may include 2 or more, 4 or more, or even 6 or more bundles laid side-by-side. The tape may include 12 or less, 10 or less, or even 8 or less bundles laid side-by-side. The tape may have a width of about 2 mm or more, 3 mm or more, or even 4 mm or more. The tape may have a width of about 7 mm or less, 6 mm or less, or even 5 mm or less. The tape may have a thickness of between about 0.1 mm and 0.6 mm (e.g., 75 μm to 600 μm). The tape may be wrapped around the core at approximately +45° and +45° to the inner core. The pin may have an areal weight of about 1.3 g/ft. The pin may comprise a fiber volume of about 45-65% (e.g., 50%). The remaining volume may be occupied by matrix material. The pin may have a cross-section that is circular, hexagonal, or lobed. The axial fiber bundles may comprise about 20,000 fibers. The axial fiber bundles may be fabricated from 5 ends, each comprising about 400 fibers. The fibers may have a diameter of about 10 μm. The distance between fibers may be less than about 5 μm. The distance may be occupied by matrix material. The axial fiber bundles may include a twist rate of about 0.7 twists per inch. The axial fiber bundles may have an average diameter of about 0.25 mm. The axial fiber bundles may have a fiber volume of about 50-70% (e.g., 60%). The distance between the several axial fiber bundles may be less than about 50 μm. The distance may be occupied by matrix material. The bias fiber bundles (binding) may comprise about 800 fibers. The bias fiber bundles may be fabricated from 2 ends with about 400 fibers per end. The fibers may have a diameter of about 10 μm. The distance may be occupied by matrix material. The bias bundles may include a twist rate of about 0.7 twists per inch. The bias bundles may have an average diameter of about 0.25 mm. The bundles may have a fiber volume of about 50-70% (e.g., 55%). The distance between the several bundles of axial fibers may be less than about 30 μm. The distance may be occupied by matrix material. The bias fiber bundles (binding) may comprise about 800 fibers. The cell size of the braided fiber bundle may be about 0.5-1.5 mm (e.g., 1 mm). The pin may be embedded with matrix material. The matrix material may be applied to individual axial fibers, bias fibers, axial fiber bundles, and bias fiber bundles. The matrix material may be applied to the bias bundles and axial bundles core as an outer region. The pin may be heated to intersperse and/or intermingle the matrix material coatings of all sub-components of the pin. The pin may be utilized to fabricate other composite implants (e.g., nail, screw, etc.). For example, the pin may have filaments, fibers, fiber bundles, matrix material (e.g., outer region) and/or reinforcement elements applied thereto to form a head and/or threading.

The present teachings provide for a method for making a composite article (e.g., a degradable composite article. The method may comprise one or more of the following steps. Some of the steps may be duplicated, removed or eliminated, rearranged relative to other steps, combined into one or more steps, separated into two or more steps, or a combination thereof.

The method may comprise selecting from an inventory of a predetermined number of sets of fiber bundles. The sets may include a plurality of sets (e.g., two or more sets, 5 or more sets, 7 or more sets, 25 or less sets, 20 or less sets, 15 or less sets, 12 or less sets) of fiber bundles. Each set of fiber bundles may differ from each other (e.g., in size, shape, individual constituents, composition, property or any combination thereof).

The composite article may be defined by an area (surface area) to mass ratio. The area to mass ratio may be about 10:5 or more, 10:7 or more, or even 10:9 or more. The area to mass ratio may be about 10:17 or less, 10:15 or less, or even 10:13 or less.

The fiber bundles may be axial fiber bundles and/or bias fiber bundles. The fiber bundles may be selected to provide a ratio of axial fiber bundles to bias fiber bundles. The ratio may be about 1:0.5 to about 0.5:1 (e.g., 1:1).

The fiber bundles may be defined by a fiber volume. The fiber volume may be between about 30% and 80%, more preferably between about 40% and 70%, more preferably between about 50% and 60%.

The method may comprise arranging the plurality of sets relative to each other so that when assembled with a polymer matrix (e.g., a degradable polymeric matrix) the resulting composite may exhibit mechanical properties that exceed at least one (e.g., two or more) of the respective mechanical properties, as specified elsewhere herein, for each of the respective materials of the composite (e.g., tensile strength, torsional strength, and/or compressive strength) and any failure of the resulting composite will exhibit a strain at yield of at least 5% or more, more preferably 10% or more, more preferably 15% or more, or even more preferably 20% or more.

The method may comprise assembling the fiber bundles with the polymer matrix to fabricate the composite.

The present teachings provide for a method for designing a composite article (e.g., a degradable composite article. The method may comprise one or more of the following steps. Some of the steps may be duplicated, removed or eliminated, rearranged relative to other steps, combined into one or more steps, separated into two or more steps, or a combination thereof.

The method may comprise maintaining an inventory of a plurality of sets (e.g., two or more sets, 5 or more sets, 7 or more sets, 25 or less sets, 20 or less sets, 15 or less sets, 12 or less sets) of fiber bundles. Each set of fiber bundles may differ from each other (e.g., in size, shape, individual constituents, composition, property or any combination thereof).

The method may comprise identifying a structure with or within which the composite (e.g., degradable composite) is intended to be placed in service.

The method may comprise identifying the conditions (e.g., surrounding environmental, temperature, and loading) to which the composite (e.g., degradable composite) will be subjected when placed in service.

The method may comprise selecting one or a plurality of elongated fiber bundles from the inventory of fiber bundle sets.

The method may comprise ascertaining an arrangement of a plurality of fiber bundles for placement within a composite based upon the conditions to which the composite will be subjected when placed in service for achieving mechanical properties, as specified elsewhere herein (e.g., tensile strength, torsional strength, and/or compressive strength) and for assuring any failure of the composite is in a ductile mode.

In medical applications according to the present teachings, the mechanical properties may not diminish more than 20%, more preferably 10%, more preferably 5% during a period of about 4 weeks after implantation into a living being. The mechanical properties may not diminish during a period of ingrowth of bone and/or tissue.

Ascertaining a polymeric matrix volume and distribution for the composite (e.g., degradable composite) based upon the conditions to which the composite will be subjected when placed in service for achieving mechanical properties, as specified elsewhere herein (e.g., tensile strength, torsional strength, and/or compressive strength) and for assuring any failure of the composite is in a ductile mode.

The method may comprise assembling the fiber bundles with the polymer matrix to form the composite.

The inventory of fiber bundles may include fiber bundles that include filaments each having diameters ranging from about 3 to about 25 microns (e.g., about 5 to about 20 microns, or about 10 to about 15 microns).

The inventory of fiber bundles may include filaments, fibers and/or fiber bundles that may be twisted along a longitudinal axis. The twist may be about 0.2 twists/inch or more, 0.4 twists/inch or more, or even 0.6 twists/inch or more. The twist may be about 1.2 twists/inch or less, 1.0 twists/inch or less, or even 0.8 twists per inch or less.

The inventory of fiber bundles may include filaments, fibers, and/or fiber bundles that employ a bias element along a longitudinal axis. The bias element may be oriented at an angle to the longitudinal axis. The angle may be about ±0° or more 5° or more 15° or more 25° or more, 35° or more or even 45° or more. The angle may be ±90° or less 75° or less, 65° or less, or even 55° or less.

The step of identifying a structure with or within which the composite (e.g., degradable composite) is intended to be placed in service includes a structure selected from within a bone, on a bone, a structural joint, tissue, the like, or any combination thereof.

The conditions (e.g., surrounding environmental, temperature, and loading) to which the composite (e.g., degradable composite) will be subjected when placed in service may include one or more of a relatively constant temperature of about 37° C., in the presence of a bodily fluid (e.g., blood), is to be subjected to repeated compressive loads in service, is to be subjected to repeated tensile loads in service, is to be subjected to repeated torsional loads in service, must avoid brittle failure, must degrade according to a predetermined degradation profile to allow growth of bone and tissue.

The step of selecting one or a plurality of elongated fiber bundles from the inventory of fiber bundle sets may include selecting one or more fiber bundles that (i) include a polymeric outer covering (e.g., a biodegradable polymeric sheath), (ii) include at least one bias element, (iii) include 2, 3, 4, 5, 6, 7, 8, 9, 10, or more fiber bundles some or all of which may be at least partially surrounded by a sheath, by at least one bias element or both, or any combination of the foregoing (i)-(iii).

The step of ascertaining an arrangement of a plurality of fiber bundles for placement within a composite may include a step of predictive modeling.

The step of ascertaining an arrangement of a plurality of fiber bundles for placement within a composite (e.g., a composite implant) may include a step of predictive modeling using as inputs for loading conditions (e.g., dimensions or structures obtained from imaging) specific data obtained from an individual or structure into or on which the composite is to be located when in service.

The step of assembling the fiber bundles with the polymer matrix to form the composite may include forming an assembly of a plurality of bundles. The assembly may include (i) bundles that are positioned in nested relationship relative to each other, (ii) bundles that are positioned in parallel relative to each other along their longitudinal axes, (iii) the resulting assembly exhibits plural transverse cross sections along its length, each plurality of 3, 4, 5, 6 or more bundles radially spaced around a longitudinal axis of the assembly; (iv) bundles that include polymer (e.g., degradable polymer) circumferentially disposed about each bundle, around the entirety of the assembly or both; (v) a hollow longitudinal channel runs along a portion of the length of the assembly; (vi) a plurality of throughholes penetrate from an exterior surface of the assembly and optionally defines a flow passage with a longitudinal channel of the assembly; (vii) at transverse cross-sections taken along its length, the assembly includes a plurality of concentric spaced apart polymeric regions (which optionally may be interconnected to each other by polymer distributed along the length); (viii) in the absence of polymer, the fiber bundles exhibit a lattice structure including an interconnected structure of a plurality of generally annular rings, generally helically coiled segments, generally longitudinally oriented struts, and/or generally radially oriented struts.

The fiber bundles may include bioabsorable glass fibers that include one or more elements selected from silicon, boron, phosphorus, magnesium, calcium, sodium, or any combination thereof. The fiber and/or the fiber bundles may be selected so that they degrade sufficiently slow that they do not exceed a degradation amount overall, when used as an orthopedic implant of about 10 mg/day, more preferably 1 mg/day, more preferably 0.1 mg/day.

The composite may have a texture and/or surface porosity, just prior to deployment for its intended use in the range of about 10 μm and 60 μm.

The composite may be configured so it has a texture and/or surface porosity, just after a period of 4 weeks in the range of about 200 μm and 400 μm.

The composite may be configured so it has a degradation profile so that after a period of at 4 weeks from deployment, a network of bone and tissue has infiltrated and become at least partially entangled with at least a portion of the remaining composite.

The step of assembling may include forming a plurality of subassemblies and then assembling the subassemblies together to form the composite.

Each of the subassemblies may be elongated and include a longitudinal axis and the subassemblies are oriented generally parallel to each other in a nesting relationship to define a nonplanar interface between each adjoining subassembly (e.g., each adjoining bundle). The nesting can be in a manner that bundles of fibers are arranged in adjoining relationship to each other. Bundles may be arranged so adjoining bundles contact each other. Bundles may be arranged in stacks, such that each adjoining stack has a plurality of adjoining bundles (e.g., bundles in contact with each other). Each bundle may have a central longitudinal axis so that each respective stack locates its respective central longitudinal axis between the central longitudinal axes of bundles beneath or above it). In this manner, nesting of bundles occurs as between bundles above or below them. It is also possible that each bundle may have a central longitudinal axis so that each respective stack locates its respective central longitudinal axis between the central longitudinal axes of bundles beneath or above it; such arrangement may be such that nesting would not be realized as between stacks above and below. It should be realized that adjoining bundles may have the same or different sizes, or materials relative to each other. It is envisioned that a build-up of bundles may be realized that includes a plurality of stacks. The numbers of bundles in each stack may be the same or different from the number of bundles in each adjoining stack. It is possible for, example, to have an assembly of seven bundles in three stacks (i.e., in stacks of 2-3-2 bundles). One possible approach envisions creating assemblies in which each successive adjoining stack includes N+1 or N−1 bundles, where N is the number of bundles in a first stack. Though each successive stack is described to have N+1 or N−1, another number (whether or not an integer) other than one (1) may be used. To illustrate, for an assembly including 30 bundles, a centrally positioned stack may include 6 bundles, above and below it are stacks for 5, 4, and 3 for an arrangement of 3-4-5-6-5-4-3 (30 bundles). Bundles may be omitted within the interior for creating a channel. For example, an arrangement may have 3-4-4-4-4-4-3, in which the stacks of 4 in the interior central stack (the stack of 6 in the preceding illustration) have 2 bundles at each end of the stack and 2 vacancies in the middle of that stack, and the stack that would have had five, omits its central bundle.

A step of assembling may include assembling together a sub-assembly of one or more filaments, fibers and/or bundles. There may be a step of pultruding one or more filaments, fibers and/or bundles with a biodegradable polymer through a die. There may be a step of extruding one or more filaments, fibers and/or bundles with a matrix of biodegradable polymer through a die. There may be a step of wrapping one or more filaments, fibers and/or bundles with at least one bias element. There may be a step of applying a sheath (e.g., one including a biodegradable polymer) over one or more filaments, fibers and/or bundles. There may be a step of injection molding a biodegradable polymer over one or more filaments, fibers and/or bundles overmolding a polymer on one or more filaments, fibers and/or bundles. There may be a step of cutting one or more filaments, fibers and/or bundles (which optionally may be embedded in a biodegradable polymer) to a defined length. Any combination of the foregoing steps may be employed.

A step of assembling may include applying a coated region on the composite, and/or a coating on at least one or more fiber bundles within the composite of a biodegradable polymer that includes a plurality of nanoparticles (e.g., degradable nanoparticles) distributed in a manner to induce controlled pore formation, to retard the propagation of a crack within the biodegradable polymer, or both.

A composite may include a composite article.

A composite may include an implant. It may be an orthopedic implant. The orthopedic implant may be configured for placement on a bone outer surface, within a medullary channel of a bone, or within a bore formed within a bone.

An orthopedic implant may be configured as a pin, a rod, a nail, a screw, or a plate. An orthopedic implant may be configured to include one or more apertures as taught herein. An orthopedic implant may be configured to include a threaded and/or barbed exterior surface.

During a period of at least one, two, three, four, six or twelve months, articles or implants of the teachings herein may be configured for a controlled deformation and failure, that makes advantageous use of energy dispersion characteristics of polymeric matrix, the ability to retard crack propagation by the presence of fillers (e.g., nanofibers in polymeric matrix), selectively employ regions for creating stress concentrations to foster a particular failure mode (e.g., delamination in lieu of brittle fracture).

The teachings herein may be employed with any of the other general teachings herein. By way of example, without limitation, teachings herein for dimensions, porosity, material selection (e.g., teachings about bioerodable glasses, such as bioglass, teachings about biodegradable polymers such as PLLA, polyurethane, P4HB, PCL, PLA) may be combined in part or in their entirety.

### ILLUSTRATIVE EXAMPLES

The following examples are included to help illustrate some of the technical foundation upon which various combinations depicted are predicated. The Examples should not be deemed as limiting. Moreover, the skilled person should realize that features present in the specific material combinations in the Examples can be more generally applied according to the teachings herein. To the extent any of the examples describe a polymer or other material that is not degradable and resorbable, the teachings contemplate that a degradable and resorbable material as taught in the Summary and Detailed Description could be substituted for the material in the Example and similar or better results are expected. Thus, the Example should be regarded as illustrating the use of the material described along with the contemplated substitution of degradable and resorbable materials taught herein. In addition, to the extent that the Examples specify a dimension, concentration or some other numerical parameter in them, the teachings herein envision that use of values within +/−20%, or 10% as part of the teachings.

Example 1. Thermoplastic P4HB and PLA beads were mixed with phosphate-based soluble glass (additive) and incubated in phosphate-based buffer solution at 50° C. for 52 days in vials, 50/50 by weight. The buffer solution was changed periodically as pH shifted. Control P4HB and PLA beads were also prepared, without phosphate-based soluble glass, and incubated in an identical manner. The beads were dried thoroughly after 52 days and analyzed via GPC. For P4HB, the higher molecular weight portion (Mz) decreased significantly regardless of additive. For P4HB, the lower molecular weight portion (Mn) increased slightly more in control than in samples with additive. For P4HB with glass type 1, degradation rate was impacted for both the high molecular weight portion (Mz) as well as the lower molecular weight portion (Mn) in samples with additive as compared to the control. For PLA, there was a large decrease in molecular weight regardless of additive. For PLA, degradation rate slowed slightly with glass type 1. For PLA, degradation rate was sped up with glass type 2. This example demonstrates that a composite of thermoplastic and soluble glass composite degrades. Additionally, P4HB (known to degrade primarily by enzymatic degradation) is demonstrated to have increased hydrolytic degradation due to the addition of soluble glass. Accordingly, the teachings herein contemplate the general concept that soluble glass may be employed in composites herein.

Example 2. 2 mm diameter, 80 mm length pins were constructed from polyurethane (PU) and soluble phosphate glass fibers with a sizing applied. The glass fibers were axially aligned, and polyurethane (PU) was introduced to fixate the glass fibers. After construction of the pins, they were coated with a barrier material known to retard the ingress of water to a rate of 0.4 g*mm/m²*day. The pins were tested for stiffness at intervals of time. The test results are illustrated in FIG. 30. The pins maintained a Young's modulus of greater than 12 GPa over a 25-day period. Thus, it was shown that coating the pins in a barrier severely slows loss of stiffness.

Example 3. 2 mm diameter, 80 mm length pins were constructed from polyurethane (PU) and soluble phosphate glass fibers. The pins were weighed. The pins were stored in a buffer solution maintained at about 70° C. (to accelerate degradation) for 7 days. After degradation, the pins were weighed. It was shown that the pins degraded in a non-linear fashion, thus demonstrating control of degradation rate. Test results are illustrated in FIG. 31.

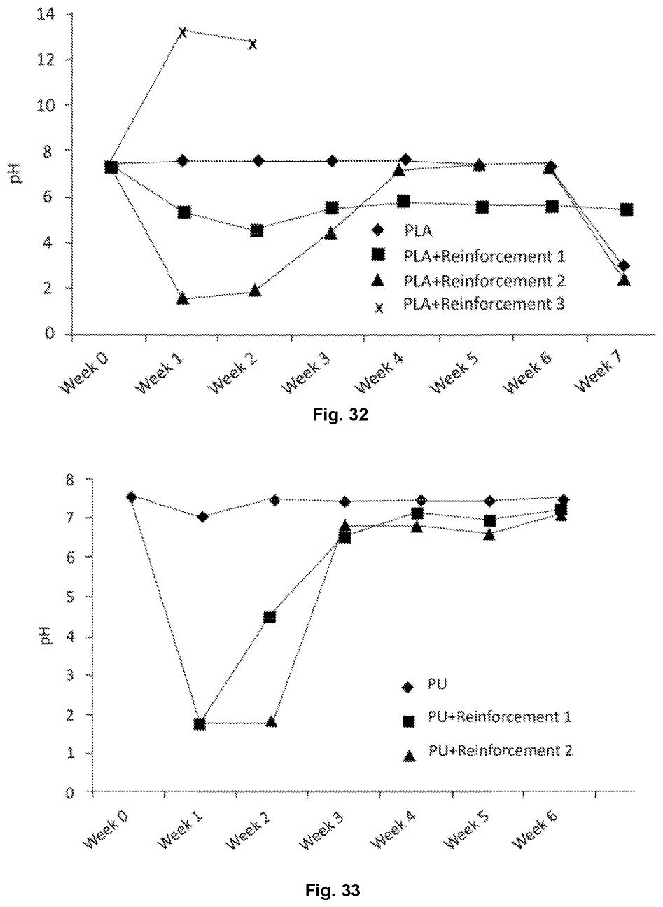

Example 4. Thermoplastic PLA polymer was co-mingled with different types of phosphate based soluble glass fibers and submerged in a 7.4 pH buffer solution with periodic refreshes of the solution. It was shown that each sample modulates the local environment differently. Test results are illustrated in FIG. 32.

Example 5. Thermoplastic Polyurethane polymer is co-mingled with two different types of phosphate based soluble glass fibers and submerged in a 7.4 pH buffer solution with periodic refreshes of the solution. It was shown that each sample modulates the local environment differently. Test results are illustrated in FIG. 33.

Example 6. 2 mm diameter round pins (5 cm) long were fabricated from polyurethane (PU) and soluble phosphate glass fibers. One group of pins was coated with a non-degradable barrier material known to retard water ingress to 0.4 g*mm/m²*day. The thickness of the coating was about 13 μm. A control group remained uncoated. The groups of pins were submerged in distilled water at room temperature (20° C.). The loss of stiffness was measured periodically. The rate of stiffness loss was reduced from 16% per day to 1.10% per day with the coating.

Example 7. Hirvikorpi (T. Hirvikorpi, et al., “Enhanced water vapor barrier properties for biopolymer films by polyelectrolyte multilayer and atomic layer deposited Al2O3double-coating,” Appl. Surf Sci., vol. 257, no. 22, pp. 9451-9454, September 2011) and Shogren (R. Shogren, “Water vapor permeability of biodegradable polymers,” J. Environ. Polym. Degrad., vol. 5, no. 2, pp. 91-95, 1997) describe barrier substances, their fluid aqueous fluid ingress rate, and the required layer thickness to achieve said rate. These substances were studied in the literature at temperatures between 20° C. and 25° C. For a composite article configured for use within a living being, temperatures would be about 37° C., resulting in more rapid diffusion of aqueous fluid (Brownian motion). The figures reported in the literature can be adapted to reflect 37° C. by employing the Arrhenius equation. Layer thickness was also adjusted.

A fully degradable pin with dimension from above can be reduced to practice using a number of available barrier substances described in literature as listed in the table below (all at temperatures between 2° and 25° C.):

| | Literature | Literature | Calc. | |
|---|---|---|---|---|
| | WVP (g | Req | WVP | Calc. Req |
| | mm/m² | Thickness | (g mm/m² | Thickness |
| Material | *day) | (μm) | *day) | (μm) |
| 20 μm thick commercial | 1.1 | 44 | 3.0 | 96 |
| Poly (Lactic Acid) Film | | | | |
| Al₂O₃ Coated PLA Film | 0.7 | 21 | — | — |
| Poly Hydroxybuterate- | 0.3 | 11 | 0.8 | 25 |
| co-valerate (6% valerate) | | | | |
| Poly (Lactic Acid) | 2.1 | 66 | 4.9 | 159 |
| 66% Crystallinity | | | | |
| Poly ε-capralactone | 4.4 | 143 | 10.6 | 343 |

Example 8. The thicknesses of the coatings in Examples 90-93 are high for some of the more readily available and acceptable materials for human implantation. In order to reduce barrier thickness, insoluble solid materials may be suspended in the polymer to reduce permeability rate. It has been reported that amounts as small as 5 wt % of clay added to polymer can halve the permeability (J.-W. Rhim, H.-M. Park, and C.-S. Ha, “Bio-nanocomposites for food packaging applications,” Prog. Polym. Sci., vol. 38, no. 10-11, pp. 1629-1652, October 2013). In the same publication, the effect on permeability rises exponentially for up to 20 wt % of additive.

PCL was compounded with 10 wt % biocompatible insoluble Mg(OH)₂— which has a plate-like morphology. Mg(OH)₂ was estimated to be half as effective as clays, therefore the effect is to reduce the permeability of the material from 10.6 to 5.3 g*mm/m²*day. With the addition of Mg(OH)₂ the required barrier thickness was reduced from 343 to 171p m.

Example 9. Barrier layers were prepared as multiple co-extruded layers of polymers. The calculated permeability of the resulting films of examples 1-4 is 31 g*mm/m²*day. To enact a thinner design with biologic benefits layers of a balloon can be created by co-extrusion of a tube and subsequent expansion using balloon forming methods. Other methods such as dip coating can also be used. The resulting permeability was calculated using a parallel network equation (K. Cooksey, “Interaction of food and packaging contents,” Intell. Act. Packag. Fruits Veg., pp. 201-237, 2007). Thus, it was shown that by layering three dissimilar materials with different water transfer rates, a barrier with similar water permeability as those of examples 1 through 4 (33 g/m2*day vs. 31 g/m2*day) with a relatively thin material (100 μm). In addition, the material has an outer coating infused with Hydroxyapatite which is advantageous if implanted near bone, and the compliance and adhesive capability of the middle layer of PCL gives some resilience to the overall structure.

| | | WVP | | |
|---|---|---|---|---|
| | | (g mm/m² | Thickness | WVTR |
| Layer | Material | *day) | (μm) | (g / m²*day) |
| Out | Poly (Lactic Acid) 66% Crystallinity / | 4.9 | 25.4 | 193 |
| | Hydroxyapatite Suspension | | | |
| Middle | Poly ε-capralactone (PCL) / Mg(OH)2 | 5.3* | 25.4 | 209 |
| | 10 wt % suspension | | | |
| Inside | Poly (Lactic Acid) 66% Crystallinity / | 2.5* | 50.8 | 48 |
| | Mg(OH)₂ 10 wt % suspension | | | |

| | | |
|---|---|---|
| *Estimated halved WVP due to insoluble components at 10 wt % | 100 | 33 |

Example 10. Reinforcement elements (7.5 mm diameter rods) were constructed from 6 axial bundles of fibers nested together and bound with bias fibers of 45° (bias fiber volume of 53% and axial fiber volume of 47%. A rod of glass fibers according to the present disclosure and a rod of conventional, non-degradable PEEK fibers were constructed in this manner and subjected to torsional testing. The samples were tested according to ASTM F-1264-03. The test was proceeded until sample failure. The test apparatus is illustrated in FIG. 32 with the bundle of glass fibers according to the present disclosure. Necking is exhibited in the bundle according to the present disclosure illustrated in FIG. 32, which indicates a ductile failure mode. The sample according to the present disclosure failed by delamination between bundles. FIG. 33 illustrates comparative results as between the glass fibers according to the present disclosure (“206 Ortho”) and PEEK fibers. The torsional performance of the glass fibers according to the present disclosure far exceeds PEEK.

Example 11. Samples were stored in an aqueous solution maintained at 37° C. for about 21 days. Flexural modulus of the samples was measured intermittently throughout the time period. The results are illustrated in FIG. 34.

Example 12. One sample comprised P4HB was combined with PHA and a control sample comprised P4HB. The samples were incubated at 50° C. for about 2 months in an enzyme free phosphate buffer solution. The molecular weight of the samples was then measured using GPC analysis to determine if the material was degrading. There was a noticeable acceleration of the decrease in molecular weight of 10% over the incubation time period. Results are illustrated in FIG. 40. This example shows that the typical primary enzymatic degradation mechanism of P4HB may be manipulated by the addition of additives.

Example 13. 2 mm diameter braids (reinforcement elements) were constructed according to the table below. The braids were fabricated utilizing a typical braiding machine. Bias fibers were loaded onto carriers with two bobbins of bias fibers per carrier. The bias angle, with respect to the longitudinal axis of the axial fibers, was set at an increasing angle for pairs of samples. Axial fibers travel through the center of the braiding machine as the bias fibers braid around the axial fibers. The unit cell size of the bias fibers was measured. The axial fibers comprise 3 or 6 bundles each comprising a number of ends of fibers. As the bias fiber volume increased, the axial fiber volume decreased. As the 2 mm diameter of the braid remained constant, the amount one type of fiber had to change relative to the other. It can be seen, because the bias fiber/carrier remained constant, bias fiber volume and cell size may be modulated by the angle of bias fibers. The axial fiber volume may be modulated by the number of bundles and ends/bundle (and consequently, the number of ends total). As the number of ends increase, so does the axial fiber volume. The number of bundles modulate the cross-sectional profile (T=triangular; C=circular; H=hexagonal) of the resulting braided structure. This is due to the packing arrangement of axial fiber bundles. The areal weight (i.e., weight per unit area (width×length)) of each resulting braided structure remained the same due to the constraint of 2 mm diameter for each braid and the tradeoff between bias fiber volume and axial fiber volume.

| | Bias Fibers | | | | Axial Fibers | | | | Resulting Braid | |
|---|---|---|---|---|---|---|---|---|---|---|
| | | Fibers / | Fiber Vol. | Cell Size | No. | Ends / | No. | Fiber Vol. | Areal Wt. | Cross-Section |
| | Angle | Carrier | (%) | (mm) | Bundles | Bundle | Ends | (%) | (g/ft) | Profile |
| S. 1 | 45° | 2 | 53 | 1.0668 | 3 | 10 | 30 | 47 | 1.3 | T |
| S. 2 | 45° | 2 | 53 | 1.0668 | 6 | 5 | 30 | 47 | 1.3 | C/H |
| S. 3 | 30° | 2 | 47 | 1.8542 | 3 | 12 | 36 | 53 | 1.3 | T |
| S. 4 | 30° | 2 | 47 | 1.8542 | 6 | 6 | 36 | 53 | 1.3 | C/H |
| S. 5 | 55° | 2 | 67 | 0.7366 | 3 | 6 | 18 | 33 | 1.3 | T |
| S. 6 | 55° | 2 | 67 | 0.7366 | 6 | 3 | 18 | 33 | 1.3 | C/H |

Example 14. Fiber bundles were constructed. The resulting fiber bundles, if employed as axial bundles, had circular cross-sections with a diameter that depended on both fiber diameter and the number of fibers in each bundle. The resulting fiber bundles, if employed as bias fiber bundles, had elliptical cross-sections with a major diameter that depended on both fiber diameter and the number of fibers in each bundle. Each of the bundles had a twist applied. The bundles were tested for their tensile strength. Fiber bundle builds and tensile results are illustrated in the following table. As the fiber diameter increased (controlling for number of fibers), the tensile strength increased. For instance, the 9 μm×204 sample had a tensile strength of 15.6 while the 13 μm×204 sample had a tensile strength of 42.7. Controlling for fiber diameter, the tensile strength increased as the number of fibers increased. The bias layer thickness changes depending on the shape of the bias bundles in the bias layer. Increasing the bias angle generally induces originally round cross-section bias bundles to become more ovoid and thereby decreasing the bias layer thickness. Coatings may or may not be applied to these constructions. The cross-sectional thickness of the coatings may be about 10 μm or less. In the case that thermoset matrix material is employed, the bundles may be wet-out with matrix material after assembly of the bundles. Pre-wetting-out with thermoplastic matrix material may prevent cohesion of the matrix material by heating later in the composite fabrication process. In the case that thermoplastic matrix material is employed, the fibers may be pre-wet-out before assembly of the bundles or wet-out after assembly of the bundles. From the yield and bundle diameter, one may calculate the fiber volume of the bundles.

| | Fiber | | Bundle | | | | Bias Layer | Tensile |
|---|---|---|---|---|---|---|---|---|
| | Diameter | No. | Diameter | | Yield | Yield TEX | Thickness | Strength |
| | (μm) | Fibers | (mm) | Twist | (yards/lbs) | (g/1000M) | (μm) | (GPa) |
| S. 1 | 9 | 68 | 0.122 | 0.7 | 45,000 | 11 | 100-130 | |
| S. 2 | 9 | 102 | 0.164 | 0.7 | 30,000 | 22 | 130-170 | |
| S. 3 | 9 | 204 | 0.203 | 0.7 | 15,000 | 33 | | 15.6 |
| S. 4 | 9 | 408 | 0.269 | 0.7 | 7,500 | 66 | | 31.6 |
| S. 5 | 9 | 612 | 0.356 | 0.7 | 5,000 | 99 | | 40.1 |
| S. 6 | 9 | 816 | 0.396 | 0.7 | 3,700 | 134 | | 86.1 |
| S. 7 | 9 | 1632 | 0.568 | 0.7 | 1,900 | 257 | | |
| S. 8 | 13 | 204 | 0.269 | 0.7 | 7,500 | 66 | | 42.7 |
| S. 9 | 13 | 408 | 0.396 | 0.7 | 3,700 | 134 | | |
| S. 10 | 13 | 816 | 0.526 | 0.7 | 1,800 | 275 | | 155.8 |

Example 14.1. Each of the bundles illustrated in the table of Example 14 may be assembled to fabricate a reinforcement elements according to TABLE 1 and TABLE 2. TABLE 1 illustrates constructions of nested axial fiber bundles bound by bias fiber bundles. TABLE 2 illustrates constructions of nested axial fiber bundles interlocked by bias fiber bundles. By selecting axial and/or bias fiber bundles having a different number of fibers and/or fiber diameters, reinforcement elements may be constructed with various dimensions, shapes, and mechanical properties.

TABLE 1

| | S. 1 | S. 2 | S. 3 | S. 4 | S. 5 | S. 6 | S. 7 |
|---|---|---|---|---|---|---|---|
| Nested and Bound RE's | | | | | | | |
| Shape | C, H, S, E. | C, H, S, E. | C, H, S, E. | C, H, S, E. | C, H, S, E. | C, H, S, E. | C, H, S, E. |
| | O, T, P | O, T, P | O, T, P | O, T, P | O, T, P | O, T, P | O, T, P |
| Axial:Bias Ratio | 1:1 | 1:1 | 1:1 | 1:1 | 1:1 | 1:1 | 1:1 |
| Wt. / foot (grams/foot) | 1.3 | 1.3 | 1.3 | 1.3 | 1.3 | 1.3 | 1.3 |
| Fbr Vol. (%) (+/−5%) | 52 | 50 | 50 | 50 | 50 | 50 | 50 |
| Fbr Diam. (μm) | 9 | 9 | 9 | 9 | 9 | 9 | 9 |
| Tot. Fbr | 21600 | 21600 | 21600 | 21600 | 21600 | 24000 | 16800 |
| Inner Core RE's | | | | | | | |
| Tot. Axial Nested | 30 | 60 | 120 | 12 | 6 | 36 | 18 |
| Bundles | | | | | | | |
| Fibers / Axial Bundle | 400 | 200 | 100 | 1000 | 2000 | 400 | 400 |
| Total Axial Fibers | 12000 | 12000 | 12000 | 12000 | 12000 | 14400 | 7200 |
| Shape | C, H, S, E. | C, H, S, E. | C, H, S, E. | C, H, S, E. | C, H, S, E. | C, H, S, E. | C, H, S, E. |
| | O, T, P | O, T, P | O, T, P | O, T, P | O, T, P | O, T, P | O, T, P |
| Outer Core | | | | | | | |
| Bias Angle (±3°) | 45 | 45 | 45 | 45 | 45 | 30 | 55 |
| Tot. Bias bundles | 24 | 48 | 96 | 24 | 24 | 24 | 24 |
| Fbr / Bias RE | 400 | 200 | 100 | 400 | 400 | 400 | 400 |
| Fbr / Bias RE Bndl | 800 | 800 | 800 | 800 | 800 | 800 | 800 |
| Tot. Bias RE Fbr | 9600 | 9600 | 9600 | 9600 | 9600 | 9600 | 9600 |
| Shape | E | E | E | E | E | E | E |

*C = Circle; O = ovoid; E = elliptical; T = triangular; P-polygon; H = hexagonal

TABLE 2

| | 3 axials | | | | 4 axials | 6 axials | | | | | | | 8 axials |
|---|---|---|---|---|---|---|---|---|---|---|---|---|---|
| RE | S. 1 | S. 2 | S. 3 | S. 4 | S. 5 | S. 6 | S. 7 | S. 8 | S. 9 | S. 10 | S. 11 | S. 12 | S. 13 |
| Nested and | | | | | | | | | | | | | |
| Interlocked RE | | | | | | | | | | | | | |
| Shape | T | T | T | T | S, CR | H | H | H | H | H | H | H | O |
| Wt./foot | 1.3 | 1.3 | 1.3 | 1.3 | 1.3 | 1.3 | 1.3 | 1.3 | 1.3 | 1.3 | 1.3 | 1.3 | 1.3 |
| (grams/foot) | | | | | | | | | | | | | |
| Fbr. Vol. (%) | 52 | 50 | 55 | 50 | 50 | 52 | 50 | 50 | 50 | 50 | 50 | 50 | 50 |
| Fbr. Diam. (μm) | 9 | 9 | | 9 | 9 | 9 | 9 | 9 | 9 | 9 | 9 | 9 | 9 |
| Total Fbr. | 21600 | 24000 | | 16800 | 21600 | 21600 | 21600 | 21600 | 21600 | 21600 | 24000 | 16800 | 21600 |
| Axial RE's | | | | | | | | | | | | | |
| Nested RE bndl | 10 | 12 | 6 | 20 | 15 | 5 | 10 | 20 | 2 | 1 | 6 | 3 | 15 |
| per Axial bndl | | | | | | | | | | | | | |
| Qty. Axial Bndl | 3 | 3 | 3 | 3 | 4 | 6 | 6 | 6 | 6 | 6 | 6 | 6 | 8 |
| Total Nested | 30 | 36 | 18 | 60 | 60 | 30 | 60 | 120 | 12 | 6 | 36 | 18 | 120 |
| RE Bndl | | | | | | | | | | | | | |
| Fbr / RE Bndl | 400 | 400 | 400 | 200 | 200 | 400 | 200 | 100 | 1000 | 2000 | 400 | 400 | 100 |
| Fbr / per Axial | 4000 | 4800 | 2400 | 4000 | 3000 | 2000 | 2000 | 2000 | 2000 | 2000 | 2400 | 1200 | 1500 |
| RE Bndl | | | | | | | | | | | | | |
| Total Axial Fbr | 12000 | 14400 | 7200 | 12000 | 12000 | 12000 | 12000 | 12000 | 12000 | 12000 | 14400 | 7200 | 12000 |
| Vol. Axial Fbr | 50 | | | | | | | | | | | | |
| (%) | | | | | | | | | | | | | |
| Shape | C, E | C, E | C, E | C, E | C, E | C, E | C, E | C, E | C, E | C, E | C, E | C, E | |
| Bias RE's | | | | | | | | | | | | | |
| Bias Angle (±3°) | 45 | 30 | 55 | 45 | 45 | 45 | 45 | 45 | 45 | 45 | 30 | 55 | 45 |
| Total Bias RE | 24 | 24 | 24 | 24 | 48 | 24 | 48 | 96 | 24 | 24 | 24 | 24 | 48 |
| bundles | | | | | | | | | | | | | |
| Fbr / Bias RE | 400 | 400 | 400 | 200 | 200 | 400 | 200 | 100 | 400 | 400 | 400 | 400 | 200 |
| Tot. Bias RE | 9600 | 9600 | 9600 | 4800 | 9600 | 9600 | 9600 | 9600 | 9600 | 9600 | 9600 | 9600 | 9600 |
| Fbr | | | | | | | | | | | | | |
| Shape | E, S, | E, S, | E, S, | E, S, | E, S, | E, S, | E, S, | E, S, | E, S, | E, S, | E, S, | E, S, | E, S, |
| | O, | O, | O, | O, | O, | O, | O, | O, | O, | O, | O, | O, | O, |

Example 15. Samples of PLA, PCL, P4HB, P4HB with G glass added, and P4HB with N glass added were degraded in a phosphate-buffered saline (PBS) solution initially at 7.4 pH over a period of about 52 days at room temperature (i.e., about 20° C. to 25° C.). The average molecular weight of the samples was measured at day 0 and day 50. It is shown that the different polymers degrade at different rates and additives affect the degradation. Results are illustrated in FIG. 41. The results show that degradation rate of a composite article fabricated from one or more different polymers may be modulated by material selection.

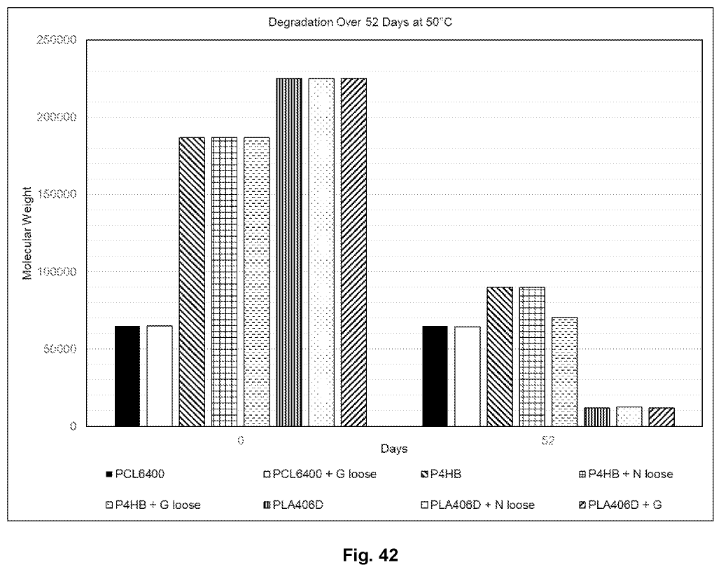

Example 16. Samples of PLA, PCL, P4HB, P4HB with G glass added, and P4HB with N glass added were degraded in a phosphate-buffered saline (PBS) solution initially at 7.4 pH over a period of about 52 days at 50° C. The average molecular weight of the samples was measured at day 0 and day 50. It is shown that the different polymers degrade at different rates and additives affect the degradation. Results are illustrated in FIG. 42. The results show that degradation rate of a composite article fabricated from one or more different polymers may be modulated by material selection. Additionally, the degradation rate of a composite article fabricated from one or more different polymers may be modulated by material selection in view of the temperatures the composite article is contemplated to be exposed to (see Example 15).

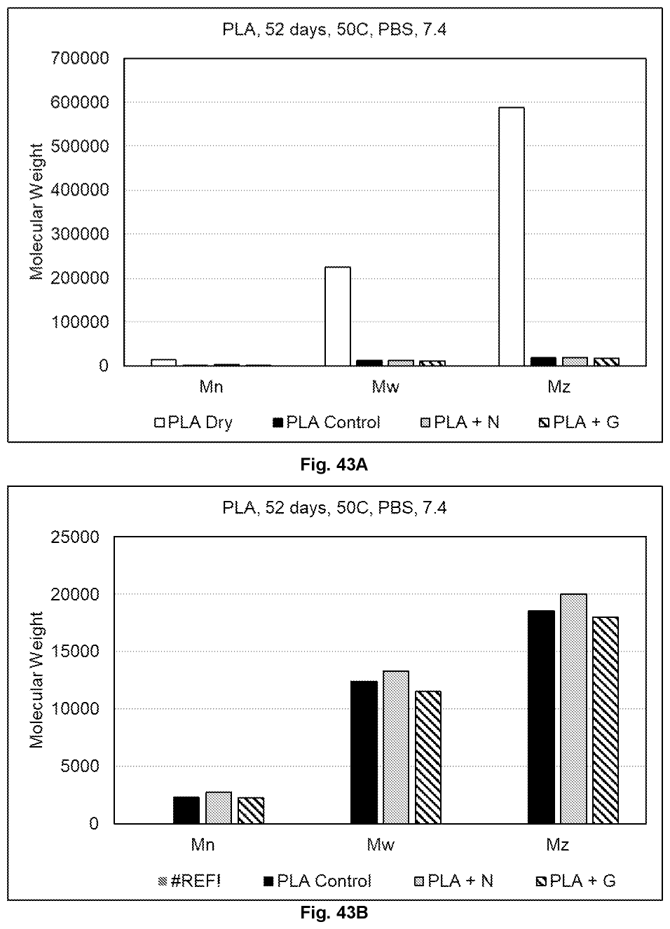

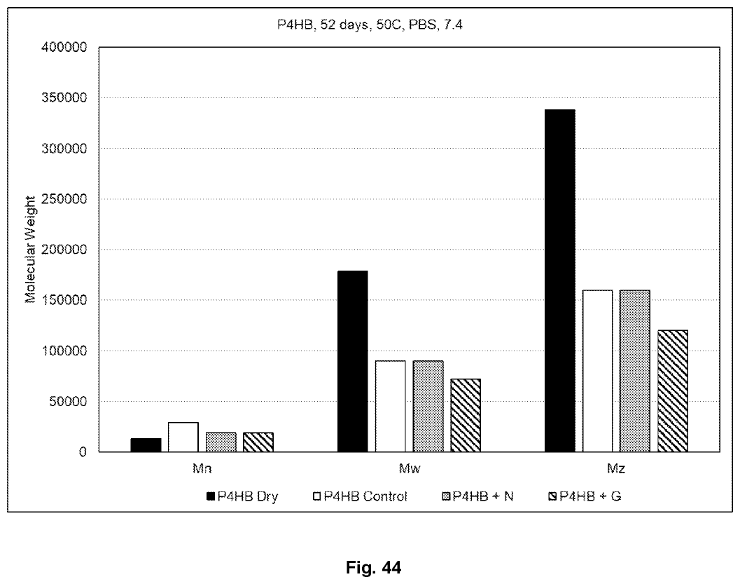

Example 17. Samples of PLA were degraded over a period of about 52 days at 50° C. in a phosphate-buffered saline (PBS) solution initially at 7.4 pH. The average molecular weight (Mw), lowest molecular weight (Mn), and highest molecular weight (Mz) was measured at 52 days. Results are illustrated in FIG. 43. In addition, pH of the solution was measured and the following observations were made. The PLA control maintained a pH of about 7.5 for the first 6 weeks, before decreasing to pH 3 during week 7. The PLA+N sample had a pH of about 5 to 6 during week 7. The PLA+G sample varied between a pH of about 1.5 to 7.5, with the first 2 weeks staying under pH 2, during weeks 4 to 6 remaining at about 7.5, and during week 7 decreasing to pH 2.6. The PLA+N sample provides a slower decrease in both amorphous and crystalline regions of the PLA. The PLA+G decreases both amorphous and crystalline regions of the PLA faster than control.

Example 18. Samples of P4HB were degraded over a period of about 52 days at 50° C. in a phosphate-buffered saline (PBS) solution initially at 7.4 pH. The average molecular weight (Mw), lowest molecular weight (Mn), and highest molecular weight (Mz) was measured at 52 days. Results are illustrated in FIG. 44. In addition, pH of the solution was measured, and the following observations were made. The P4HB control maintained a pH of between about 6.5 and 7.5 for the duration of the study (52 days). The P4HB+N sample held a pH between about 4 to 6 for the duration of the study. The P4HB+G had a pH of under 2 for the first 2 weeks, a pH of under 4 for week 3, and then a pH of about 7 for weeks 4 through 7. The P4HB+N sample had a greater (Mz) than control and a lesser (Mn) than control, meaning the amorphous regions were decreased more rapidly and the crystalline regions were decreased more slowly than the control. The P4HB+G sample had a decrease in the amorphous regions slightly faster than the P4HB+N sample and much faster than control and decreased the crystalline regions much faster than both the control and the P4HB+N.

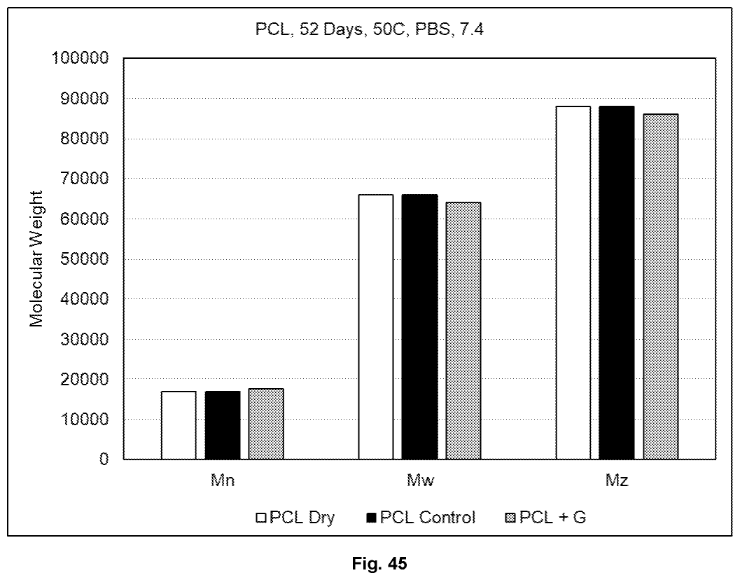

Example 19. PCL was degraded over a period of about 52 days at 50 degC. in a PBS solution initially at 7.4 pH. The average molecular weight (Mw), lowest molecular weight (Mn), and highest molecular weight (Mz) was measured at 52 days. Results are illustrated in FIG. 45. In addition, pH of the solution was measured. PCL control had an initial pH decrease to around 6.5-6.8 for the first 2 weeks of degradation, and then maintained at around 7.3 for weeks 3-7. PCL+G showed a pH under 2 for weeks 1-2, a pH of 4.5 for week 3, and a pH between 7-7.3 for weeks 4-7. G resulted in a slightly slower decrease in amorphous regions than control, and a slightly faster decrease in crystalline regions. The presence of acid decreased the amount of higher MW portion of sample (resulting in increase amorphous and decrease crystalline regions in degraded samples).

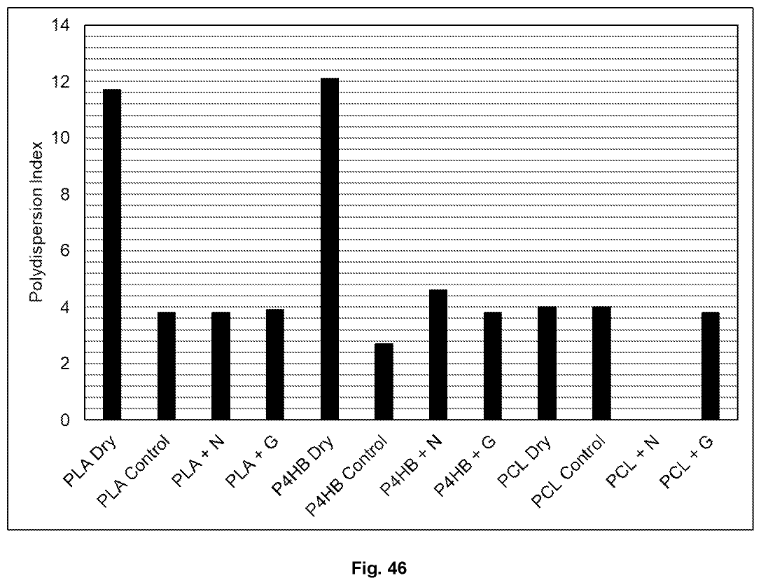

Example 20. Polydispersity index was measured on the samples of Example 17 through Example 19. The results are illustrated in FIG. 46.

The following table summarizes the pH results of Example 17 through Example 19.

| | pH | | | | | | |
|---|---|---|---|---|---|---|---|
| | Wk. 1 | Wk. 2 | Wk. 3 | Wk. 4 | Wk. 5 | Wk. 6 | Wk. 7 |
| PLA Control | ~7.5 | ~7.5 | ~7.5 | ~7.5 | ~7.5 | ~7.5 | ~3 |
| PLA + N | | | | | | | ~5-6 |
| PLA + G | <2 | <2 | | ~7.5 | ~7.5 | ~7.5 | 2.6 |
| P4HB Control | ~6.5-7.5 | ~6.5-7.5 | ~6.5-7.5 | ~6.5-7.5 | ~6.5-7.5 | ~6.5-7.5 | ~6.5-7.5 |
| P4HB + N | ~4-6 | ~4-6 | ~4-6 | ~4-6 | ~4-6 | ~4-6 | ~4-6 |
| P4HB + G | <2 | <2 | <3 | ~7 | ~7 | ~7 | ~7 |
| PCL Control | ~6.5-6.8 | ~6.5-6.8 | ~7.3 | ~7.3 | ~7.3 | ~7.3 | ~7.3 |
| PCL + G | <2 | <2 | ~4.5 | ~7.0-7.3 | ~7.0-7.3 | ~7.0-7.3 | ~7.0-7.3 |

Example 21. The following table illustrates different grades of silicate glass that may be employed as glass fibers and/or glass fillers.

| | SiO2 | Na2O | CaO | P2O5 | P2O3 | ZnO | B2O3 | MgO | Fe2O3 | K2O |
|---|---|---|---|---|---|---|---|---|---|---|
| 45S5 | 45% | 24.5% | 24.5% | 6% | | | | | | |
| S53P4 | 53% | 23% | 20% | 4% | | | | | | |
| 13-93 | 53% | 6% | 20% | 4% | | | | 5% | | 12% |
| 58S | 58% | | 33% | 9% | | | | | | |
| 70S30C | 70% | | 30% | | | | | | | |

Example 22. The following table illustrates different grades of phosphate glass that may be employed as glass fibers and/or glass fillers. The content (e.g., Na2O, etc.) of the glasses is expressed as percentages. Reference to G or N in the previous examples correspond to the same below.

| | | | | | | | | | | | | | | |
|---|---|---|---|---|---|---|---|---|---|---|---|---|---|---|
| | | Mass loss | | | | | | | | | | | | |
| | | Rate | | | | | | | | | | | | |
| ID | Sp ° C. | Mg/cm²/hr | Rel | Na₂O | CaO | P₂O₅ | ZnO | B₂O₃ | MgO | Fe₂O₃ | K₂O | MnO | NaF | Ce₂O₃ |

G Glass

| | | | | | | | | | | | | | | |
|---|---|---|---|---|---|---|---|---|---|---|---|---|---|---|
| 0 | | | | 6 | 26 | 58 | | | 10 | | | | | |
| 1 | 480 | 0.0006 | 1* | 6 | 7 | 60 | | 5 | 9 | 5 | 8 | | | |
| 2 | 557 | 0.0034 | 5.6 | 4.5 | 37 | 48 | | 5.5 | | 1 | | 1.5 | 0.5 | 2 |
| 3 | 560 | 0.0019 | 3.2 | 4 | 38 | 48 | | 5 | | 5 | | | | |
| 4 | 401 | 0.0058 | 9.7 | 20 | 13 | 49 | 8 | 5 | 4.25 | 0.75 | | | | |
| 5 | 402 | 0.0008 | 1.3 | 10 | | 60 | | 5 | 9 | 5 | 11 | | | |
| 6 | 600 | 0.0024 | 4 | | 27.5 | 47 | | 5.5 | 20 | | | | | |
| 7 | 520 | 0.002 | 3.3 | 5 | 15 | 55 | | | 25 | | | | | |

N Glass

| | | | | | | | |
|---|---|---|---|---|---|---|---|
| a | 0.0127 | 21 | 20 | 16 | 40 | 24 | 0 |
| b | 0.0063 | 10 | 18 | 16 | 40 | 24 | 2 |
| c | 0.0027 | 4 | 16 | 16 | 40 | 24 | 4 |
| d | 0.0015 | 3 | 13.5 | 16 | 42.5 | 24 | 4 |
| e | 0.0007 | 1.2 | 11 | 16 | 45 | 24 | 4 |
| f | 0.0008 | 1.3 | 6 | 16 | 50 | 24 | 4 |

PCT/US2020/060612 is incorporated by reference herein in its entirety for all purposes, particularly Examples 1-39.

To the extent not already covered above, any braided structure may be fabricated from filaments, fibers, fiber bundles, fiber composites, and/or reinforcement elements acting as the axial and/or bias elements. Any bundled structure may be fabricated from filaments or fibers. Any binding structures and/or interlocking structures (i.e., textiles, tape, sheets) may be fabricated from filaments, fibers, fiber bundles, fiber composites, and/or reinforcement elements. That is, while some teachings herein reference a braid being fabricated from fiber bundles, it is understood that the braid may also be fabricated from filaments, fibers, fiber bundles, fiber composites, and/or reinforcement elements. Similarly, while some teachings herein reference a bundled structure being fabricated from filaments, it is understood that the bundled structure may also be fabricated from fibers. Similarly, while some teachings herein reference binding and/or interlocking structures as being fabricated from fiber bundles, it is understood that the binding and/or interlocking structures may be fabricated from filaments, fibers, fiber bundles, fiber composites, and/or reinforcement elements.

To the extent not already covered or evident from the above, many variations are possible and are contemplated as part of the general teachings applicable to all aspects described herein. The composite articles herein (including methods of making and using them), including but not limited to orthopedic implants such as fasteners, plates, or rods, may include or be free of a nested arrangement of fiber bundles. The composite articles herein (including methods of making and using them), including but not limited to orthopedic implants such as fasteners, plates, or rods, may include or be free of a layered arrangement of fiber bundles. The composite articles herein (including methods of making and using them), including but not limited to orthopedic implants such as fasteners, plates, or rods, may include or be free of a distal tip that is hollow. The composite articles herein (including methods of making and using them), including but not limited to orthopedic implants such as fasteners, plates, or rods, may include or be free of a distal tip having at least a portion of a helical thread. The composite articles herein (including methods of making and using them), including but not limited to orthopedic implants such as fasteners, plates, or rods, may include or be free of glass that is free of an ion selected from sodium, calcium, magnesium, boron, aluminum or phosphorus. The composite articles herein (including methods of making and using them), including but not limited to orthopedic implants such as fasteners, plates, or rods, may include or be free of glass that has silica present in a mol percent below 68% or above 70% of that overall glass composition. The composite articles herein (including methods of making and using them), including but not limited to orthopedic implants such as fasteners, plates, or rods, may include or be free of filaments, fibers or fiber bundles having a length of at least a portion (e.g., a majority by weight percent) of the total filaments, fibers, or fiber bundles is at least about 50% to 75% of a longitudinal length of the composite (e.g., implant). The composite articles herein (including methods of making and using them), including but not limited to orthopedic implants such as fasteners, plates or rods, may include or be free of filaments, fibers, or fiber bundles having a length of at least a portion (e.g., less than about 70% by weight percent) of the total filaments, fibers, or fiber bundles is at least about 110%, 125% or 150% (but less than about 250%) of a longitudinal length of the composite (e.g., implant). The composite articles herein (including methods of making and using them), including but not limited to orthopedic implants such as fasteners, plates, or rods, may include or be free of a biocompatible implantable polymer (e.g., polyether ether ketone or others). The composite articles herein (including methods of making and using them), including but not limited to orthopedic implants such as fasteners, plates or rods, may include or be free of a bioglass. The composite articles herein (including methods of making and using them), including but not limited to orthopedic implants such as fasteners, plates, or rods, may include or be free of a taper along only a portion of a length of the article. The composite articles herein (including methods of making and using them), including but not limited to orthopedic implants such as fasteners, plates, or rods, may include or be free of uniaxially aligned filament, fibers and/or fiber bundles. The composite articles herein (including methods of making and using them), including but not limited to orthopedic implants such as fasteners, plates, or rods, may include or be free of a hemispherical head portion. The composite articles herein (including methods of making and using them), including but not limited to orthopedic implants such as fasteners, plates, or rods, may include or be free of an arcuate or tapered longitudinal profile between adjoining crests of a helical thread. The composite articles herein (including methods of making and using them), including but not limited to orthopedic implants such as fasteners, plates, or rods, may include or be free of a fiber arrangement in a head portion (for an article that is a fastener) that is radially symmetrical about a longitudinal axis, that has mirror symmetry across a longitudinal plane or both.

It will be appreciated from the above that the present disclosure contemplates among its many general teachings the following Composite Aspects.

Composite Aspect 1. An elongated composite for an implant comprising: i) one or more composite elements includes a core region including a plurality of fibrous bundles (which are preferably braided, woven, bound, interlaced and/or interlocked by bias fiber elements) wherein the core region of the composite elements includes a polymerizable material or polymeric material which fills gaps between the fibrous bundles, (preferably wherein the each of the one or more composite elements has a second region including a polymeric covering formed of a polymerizable material or a polymeric material); and ii)
a covering over the one or more composite elements, preferably wherein the covering provides one or more functional features to the composite; wherein the elongated composite and each of the one or more composite elements have a longitudinal axis; the fibrous bundles include a plurality of filaments that are twisted about the longitudinal axis, and includes a matrix material (preferably a polymerizable material or a polymeric material, which may be the same or different as the material that fills the gaps between the fibrous bundles); wherein the elongated composite is preferably characterized by one or any combination of (e.g., all of) the following: (i) the elongated composite is configured for placement within a body of a live being as an orthopedic implant; or (ii) the elongated composite exhibits a degradation profile that results in sufficient bone and tissue growth within the volume of the implant before degradation so that the elongated composite is load bearing; or (iii) the composite element is characterized by a flexural modulus of about 20 GPa or more.

Composite Aspect 2. The elongated composite of the above Composite Aspect 1, wherein the fibrous bundle has a tensile modulus of about 10 GPa or more (preferably about 25 GPa or more, and more preferably about 40 GPa or more), as measured according to ASTM D638-14, using a fiber shaped specimen; or the fibrous bundle has a flexural modulus of about 5 GPa or more (preferably about 10 GPa or more, about 15 GPa or more, about 20 GPa or more, about 25 GPa or more, about 30 GPa or more, about 35 GPa or more, or about 40 GPa or more), as measured according to ASTM D 790-17; or the fibrous bundle has a bending radius of about 10 cm or less (preferably about 5 cm or less, and more preferably about 2 cm or less), as measured according to ASTM E290-14 (preferably where the coated fiber is bent until the legs contact).

Composite Aspect 3. The elongated composite of any of the above Composite Aspects, wherein the core region is formed of layers of sheet, wherein each layer includes one or more (preferably 3 or more) of the fibrous bundles.

Composite Aspect 4. The elongated composite of any of the above Composite Aspects, wherein the bias elements including bias fibers angled relative to the longitudinal axis, preferably wherein the bias elements include a plurality of aligned fibrous bundles and a polymeric matrix, preferably wherein the bias elements have an elongated cross-section (e.g., elliptical, or rectangular, or oval) perpendicular to a length direction of the bias element, preferably wherein a ratio of the width to thickness is about 2 or more, about 3 or more, or about 4 or more); preferably wherein the bias elements are wrapped, braided, woven, interwoven or wound around the fibrous bundles of the core.

Composite Aspect 5. The elongated composite of any of the above Composite Aspects, wherein the elongated composite includes a plurality of the composite elements, preferably wherein each of the composite elements includes a polymeric coating around the core; preferably wherein the plurality of composite elements are held together and/or integrated (e.g., by wrapped, braided, woven, interwoven, or wound bias elements).

Composite Aspect 6. The elongated composite of any of the above Composite Aspects, wherein the elongated composite includes bias elements that are braided, woven, or interwoven, wherein the bias elements are arranged to provide an open structure.

Composite Aspect 7. The elongated composite of any of the above Composite Aspects, wherein the elongated composite has a cross-sectional profile that is polygonal (preferably triangular, rectangular, square, pentagonal, hexagonal), circular, or curved (preferably elliptical or oval); optionally wherein the elongated composite is partially or completely cannular.

Composite Aspect 8. The elongated composite of any of the above Composite Aspects, wherein the fibrous bundles includes coated fiber, wherein the coated fibers include a plurality of aligned inorganic filaments, wherein the filaments have a polymeric or polymerizable coating over at least a portion of their surface.

Composite Aspect 9. The elongated composite of any of the above Composite Aspects, wherein i) the coated fiber has a tensile modulus of about 10 GPa or more (preferably about 25 GPa or more, and more preferably about 40 GPa or more), as measured according to ASTM D638-14, using a fiber shaped specimen; or ii) the coated fiber has a flexural modulus of about 5 GPa or more (preferably about 10 GPa or more, about 15 GPa or more, about 20 GPa or more, about 25 GPa or more, about 30 GPa or more, about 35 GPa or more, or about 40 GPa or more), as measured according to ASTM D 790-17; or iii) the coated fiber has a bending radius of about 10 cm or less, (preferably about 5 cm or less, and more preferably about 2 cm or less) as measured according to ASTM E290-14 (semi-guided bend test around a mandrel, preferably where the coated fiber is bent about 180°).

Composite Aspect 10. The elongated composite of any of the above Composite Aspects, wherein the inorganic filaments are formed of a material having a specific gravity of about 2.80 or less, preferably about 2.65 or less, and most preferably about 2.50 or less.

Composite Aspect 11. The elongated composite of any of the above Composite Aspects, wherein the coated fibers have a substantially uniform profile along its length.

Composite Aspect 12. The elongated composite of any of the above Composite Aspects, wherein the elongated composite is a pin, a rod, a nail, a screw, or a plate, any one of which optionally contains a plurality of barbs on an outer surface.

Composite Aspect 13. The elongated composite of any of the above Composite Aspects, wherein the elongated composite exhibits a modulus of elasticity, measured about 4 weeks implantation, of about 8,000 MPa to about 40,000 MPa, preferably between about 9,000 MPa and about 30,000 MPa, and more preferably between about 9,500 MPa and about 25,000 MPa.

Composite Aspect 14. The elongated composite of any of the above Composite Aspects, wherein the elongated composite includes an outer region having barbs or threads.

Composite Aspect 15. The elongated composite of any of the above Composite Aspects, wherein the elongated composite is an orthopedic pin (e.g., for bone or soft tissue fixation) having spaced apart barbs, optionally wherein the orthopedic pin is cannulated, optionally wherein the orthopedic pin has apertures on its sides.

Composite Aspect 16. The elongated composite of any of the above Composite Aspects, wherein the elongated composite is an orthopedic screw (e.g., for bone or soft tissue fixation) having a threaded shaft, optionally wherein the orthopedic screw is cannulated, optionally wherein the orthopedic screw has throughhole apertures on its sides that intersect with a channel along a longitudinal axis of the screw.

Composite Aspect 17. The elongated composite of any of the above Composite Aspects, wherein the fibers are degradable, preferably wherein the fibers comprise glass or magnesium hydroxide; preferably, wherein the composite includes a composite orthopedic implant for implantation into a living being and wherein the polymeric matrix material, the degradable fibers, or any combination thereof are configured to be degradable in vivo after implantation into the living being according to a predetermined degradation profile that corresponds with a bone and tissue ingrowth profile so that from the time of implantation until the wound site is healed, the composite orthopedic implant maintains a sufficient compressive load and, in the event of failure, fails in a ductile failure mode.

Composite Aspect 18. The elongated composite of any of the above Composite Aspects, wherein the elongated composite is a screw, pin, anchor, nail, or plate having apertures; preferably wherein the apertures are defined by openings between bias fiber bundles; and more preferably wherein the apertures are defined by openings between bias fiber bundles.

Composite Aspect 19. The elongated composite of any of the above Composite Aspects, wherein the polymeric or polymerizable material of the core layer is degradable and bioabsorbable material; and the plurality of fiber bundles including degradable fibers; preferably wherein the degradable polymeric or polymerizable material, the degradable fibers, or both are configured to be degradable according to a predetermined degradation profile, so the composite maintains a sufficient compressive load and, in the event of failure, fails in a ductile failure mode from a time of implantation until at least about 24 weeks after initiation of degradation; and more preferably, preferably wherein the composite also occupies an envelope defined by a perimetric surface geometry and/or a volume of the composite.

Composite Aspect 20. The elongated composite of any of the above Composite Aspects, wherein an aspect ratio of the elongated composite is about 1:50 or more, preferably about 1:40 or more, and more preferably about 1:30 or more, and/or the aspect ratio is about 1:5 or less, preferably about 1:10 or less, and more preferably about 1:20 or less.

Composite Aspect 21. The elongated composite of any of the above Composite Aspects, and also including (i) any of the Coated Fiber Aspects 1 through 86 described herein, (ii) any of the Degradation Aspects 1 through 54 described herein, or both (i) and (ii).

The present teachings contemplate among its general teachings applicable to all combinations of features, a composite, an implant (e.g., an orthopedic implant of any of the types described herein, kits containing, and methods of making and using unique coated fibers that can be characterized by the following illustrative Coated Fiber Coated Fiber Aspects, any one or more of which can be combined with one or more of the above Composite Aspects and the below Degradation Aspects.

Coated Fiber Aspect 1. A coated fiber adapted for use in a composite with a polymeric matrix comprising a plurality of aligned inorganic filaments, the coated fiber having a substantially uniform profile along its length, wherein the filaments have a polymeric or polymerizable coating over at least a portion of their surface, wherein the structure of the fiber and the materials of the filament and the coating are selected for providing one or more of the following characteristics: (i) the coated fiber has a tensile modulus of about 10 GPa or more (preferably about 25 GPa or more, and more preferably about 40 GPa or more), as measured according to ASTM D638-14, using a fiber shaped specimen; or (ii) the coated fiber has a flexural modulus of about 5 GPa or more (preferably about 10 GPa or more, about 15 GPa or more, about 20 GPa or more, about 25 GPa or more, about 30 GPa or more, about 35 GPa or more, or about 40 GPa or more), as measured according to ASTM D 790-17; or (iii) the coated fiber has a bending radius of about 10 cm or less, (preferably about 5 cm or less, and more preferably about 2 cm or less) as measured according to ASTM E290-14 (semi-guided bend test around a mandrel, preferably where the coated fiber is bent about 180°); wherein the inorganic filaments are preferably formed of a material having a specific gravity of about 2.80 or less, preferably about 2.65 or less, and most preferably about 2.50 or less.

Coated Fiber Aspect 2. The coated fiber of Coated Fiber Aspect 1, wherein the filaments are twisted, preferably at a twist rate of about 0.01 turns per cm or more (preferably about 0.02 turns per cm or more, more preferably about 0.05 turns per cm or more, and most preferably about 0.10 turns per cm or more. (Preferably the twist rate is about 10 turns per cm or less, more preferably about 6 turns per cm or less, and most preferably about 3 turns per cm or less.)

Coated Fiber Aspect 3. The coated fiber of Coated Fiber Aspect 1 or 2 wherein the filaments have a diameter of about 1 μm or more (preferably about 2 μm or more, more preferably about 3 μm or more, even more preferably about 4 μm or more, and most preferably about 5 μm or more). (The diameter of the filaments may be about 50 μm or less, about 40 μm or less, about 30 μm or less, or about 20 μm or less).

Coated Fiber Aspect 4. The coated fibers of any of Coated Fiber Aspects 1 to 3, wherein the coated fiber has a cross-section perpendicular to the length that is generally circular, generally oval shaped, generally elliptical, or generally polygonal (preferred polygonal shapes include rectangular, triangular, and hexagonal).

Coated Fiber Aspect 5. The coated fiber of any of Coated Fiber Aspects 1 to 4, wherein the concentration of the inorganic filaments in the coated fiber is about 55 volume percent or more (preferably about 60 volume percent or more, more preferably about 65 volume percent or more, even more preferably about 68 volume percent or more, and most preferably about 70 volume percent or more).

Coated Fiber Aspect 6. The coated fiber of any of Coated Fiber Aspects 1 to 5, wherein the inorganic filaments are formed of a glass.

Coated Fiber Aspect 7. The coated fiber of Coated Fiber Aspect 6, wherein the glass includes a first alkaline or alkaline earth metal, wherein a concentration of the first alkaline earth or alkaline earth metal in the glass at a region near the surface of the filament is different from a concentration in the glass near the center of the filament. (preferably wherein the first alkaline or alkaline earth metal is Na, Mg, K, Ca, Rb, Sr, Cs, or Ba).

Coated Fiber Aspect 8. The coated fiber of Coated Fiber Aspect 7, wherein a difference in the composition of the glass provides a surface region that is more resistant to dissolution in water compared to a core region.

Coated Fiber Aspect 9. The coated fiber of any of Coated Fiber Aspects 1 to 8, wherein the glass and/or the polymeric material are biodegradable or bioresorbable.

Coated Fiber Aspect 10. The coated fiber of any of Coated Fiber Aspects 1 to 9, wherein the coated fibers are impregnated with the polymeric material.

Coated Fiber Aspect 11. The coated fibers of any of Coated Fiber Aspects 1 to 10, wherein the amount of the polymeric material is about 5 volume percent or more (preferably about 10 volume percent or more, even more preferably about 15 volume percent or more, even more preferably about 18 volume percent or more, and most preferably about 20 volume percent or more), based on the total volume of the coated fibers.

Coated Fiber Aspect 12. The coated fiber of any of Coated Fiber Aspects 1 to 11, wherein the polymeric material includes a thermoset polymer or thermoplastic polymer.

Coated Fiber Aspect 13. The coated fiber of any of Coated Fiber Aspects 1 to 12, wherein the coated fiber has a cross-section perpendicular to the length direction characterized by an Coated Fiber Aspect ratio of about 1.1 or more, about 1.2 or more, about 1.5 or more, about 2.0 or more, or about 3.0 or more.

Coated Fiber Aspect 14. The coated fiber of any of Coated Fiber Aspects 1 to 13, wherein two or more of the filaments contact one another.

Coated Fiber Aspect 15. The coated fiber of any of Coated Fiber Aspects 1 to 14, wherein the number of filaments in the coated fiber is sufficiently low so that the aligned filaments can be infiltrated with a polymeric material or a polymerizable resin while maintaining a filament volume fraction of about 60 volume percent or more (e.g., a filament volume fraction of about 65 volume percent or more, or about 70 volume percent or more). (Preferably, the number of filaments may be about 1000 or less (about 500 or less, more preferably about 200 or less, even more preferably about 100 or less, and most preferably about 50 or less).

Coated Fiber Aspect 16. The coated fiber of any of Coated Fiber Aspects 1 to 15, wherein the filaments are treated to reduce a surface energy of the filament.

Coated Fiber Aspect 17. The coated fiber of any of Coated Fiber Aspects 1 to 16, wherein the filaments are formed of E-glass or S-glass.

Coated Fiber Aspect 18. The coated fiber of any of Coated Fiber Aspects 1 to 17, wherein the filaments include a sizing or other treatment for improving adhesion to the polymeric material.

Coated Fiber Aspect 19. The coated fiber of any of Coated Fiber Aspects 1 to 18, wherein the filaments are formed of glass filaments having a surface modified by ion swapping.

Coated Fiber Aspect 20. The coated fiber of any of Coated Fiber Aspects 1 to 19, wherein the coated fiber includes about 25 volume percent or less of organic fibers, preferably about 20 volume percent or less, and more preferably about 15 volume percent or less. (The amount of the organic fiber may be about 0 percent or more, about 1 volume percent or more, about 3 volume percent or more, or about 5 volume percent or more).

Coated Fiber Aspect 21. A fibrous bundle comprising two or more of the coated fibers of any of Coated Fiber Aspects 1 through 20 and a polymeric polymeric material between the coated fibers, wherein the concentration of filaments in the fibrous bundle is about 55 volume percent or more (about 60 volume percent or more, about 65 volume percent or more, or about 70 volume percent or more), wherein the coated fibers are aligned in an axial direction, and the fibrous bundle has a substantially uniform profile along its length in the axial direction.

Coated Fiber Aspect 22. A fibrous bundle having any of the Coated Fiber Aspects 1 through 21 or otherwise comprising two or more fibers in a polymeric material, wherein each of the fibers includes a plurality of inorganic filaments, the fibers are aligned in a generally axial direction, the fibrous bundle having a substantially uniform profile along its length, wherein the polymeric polymeric material provides a polymeric coating over at least a portion of a surface of the inorganic filaments or are impregnates the filaments, wherein the structure of the fibrous bundle, the fibers and the materials of the filament and the polymeric polymeric material are selected for providing one or more of the following characteristics: the fibrous bundle has a tensile modulus of about 10 GPa or more (preferably about 25 GPa or more, and more preferably about 40 GPa or more), as measured according to ASTM D638-14, using a fiber shaped specimen; or the fibrous bundle has a flexural modulus of about 5 GPa or more (preferably about 10 GPa or more, about 15 GPa or more, about 20 GPa or more, about 25 GPa or more, about 30 GPa or more, about 35 GPa or more, or about 40 GPa or more), as measured according to ASTM D 790-17; or the fibrous bundle has a bending radius of about 10 cm or less (preferably about 5 cm or less, and more preferably about 2 cm or less), as measured according to ASTM E290-14 (preferably where the coated fiber is bent until the legs contact); wherein the concentration of filaments in the fibrous bundle is about 55 volume percent or more (about 60 volume percent or more, about 65 volume percent or more, or about 70 volume percent or more).

Coated Fiber Aspect 23. The fibrous bundle of Coated Fiber Aspect 22, wherein the fibrous bundle includes about 50 volume percent or more of the filaments, based on a total volume of the fibrous bundle (preferably about 55 volume percent or more, more preferably about 60 volume percent or more, even more preferably about 65 volume percent or more, and most preferably about 70 volume percent or more).

Coated Fiber Aspect 24. The fibrous bundle of any of Coated Fiber Aspects 21 to 23, wherein the fibrous bundle includes a first fiber having filaments twisted in a first directions and an adjacent second fiber including filaments twisted in a second direction reverse of the first direction; preferably providing a channel for receiving a liquid, such as via a wicking process.

Coated Fiber Aspect 25. The fibrous bundle of any of Coated Fiber Aspects 1 through 24, wherein the filaments are impregnated with a thermo-formable polymeric material.

Coated Fiber Aspect 26. The fibrous bundle of any of Coated Fiber Aspects 1 through 25, wherein the fibrous bundle is pulled through a heated die to: (i) shape the fibrous bundle; or (ii) increase the concentration of filaments in the fibrous bundle; or (iii) reduce or eliminate voids in the fibrous bundle; or any combination of (i) through (iii).

Coated Fiber Aspect 27. The fibrous bundle of any of Coated Fiber Aspects 1 through 26, wherein the fibrous bundle includes a polymeric material between two fibers, wherein the polymeric material is a same or different polymeric material as a polymeric material in the filaments.

Coated Fiber Aspect 28. The fibrous bundle of any of Coated Fiber Aspects 1 through 27, wherein the filaments extend a length of the fibrous bundle.

Coated Fiber Aspect 29. A fibrous composite comprising a plurality of fibrous bundles of any of Coated Fiber Aspects 21 through 28, wherein the fibrous bundles are attached or connected by a plurality of bias fiber elements that wrap, weave together, braid, interlace, or interlock the fibrous bundles.

Coated Fiber Aspect 30. The fibrous composite of Coated Fiber Aspect 29, wherein each of the bias fiber elements includes one or more axially aligned fibers.

Coated Fiber Aspect 31. The fibrous composite of Coated Fiber Aspect 30, wherein the bias fiber element includes two or more axially aligned fibers, wherein the bias fiber element has a length in an axial direction and a width and thickness in directions orthogonal to the axial direction, wherein a ratio of a thickness of the bias fiber element to a thickness of the fiber is about 2.8 or less, preferably about 2.2 or less, more preferably about 1.8 or less, even more preferably about 1.5 or less, and most preferably about 1.2 or less.

Coated Fiber Aspect 32. The fibrous composite of Coated Fiber Aspect 31, wherein an Coated Fiber Aspect ratio of the width to the thickness of the bias fiber element is about 2 or more or about 3 or more. (Preferably the Coated Fiber Aspect ratio is about 10 or less, about 6 or less, or about 3 or less).

Coated Fiber Aspect 33. The fibrous composite of any of Coated Fiber Aspects 29 to 32, wherein the bias fiber elements are aligned at one or more angles having an absolute value of 100 or more relative to the axial direction of the fibrous bundles.

Coated Fiber Aspect 34. The fibrous composite of Coated Fiber Aspect 30, wherein a first bias fiber element is angled in a first direction and a second bias fiber element is angled in a reverse direction relative to the axial direction of the fibrous bundles.

Coated Fiber Aspect 35. The fibrous composite of Coated Fiber Aspect 34, wherein the first bias fiber element is angled at 25° to 65° and the second bias fiber element is angle at −25° to −65°.

Coated Fiber Aspect 36. The fibrous composite of any of Coated Fiber Aspects 29 to 35, wherein the fibrous bundles are woven together by the bias fiber elements, wherein one or more carriers for weaving the bias fiber elements are skipped so that there are openings in the woven structure.

Coated Fiber Aspect 37. The fibrous composite of any of Coated Fiber Aspects 29 to 36, wherein the fibrous composite has a sufficiently open structure so that a two or more of the fibrous bundles can cooperatively move relative to each other without fracturing the filaments and, or the fibers.

Coated Fiber Aspect 38. The fibrous composite of any of Coated Fiber Aspects 29 to 37, wherein the number of fibrous bundles is 3 or more.

Coated Fiber Aspect 39. The fibrous composite of any of Coated Fiber Aspects 29 to 38, wherein the number of fibrous bundles is about 10 or less.

Coated Fiber Aspect 40. The fibrous composite of any of Coated Fiber Aspects 29 to 39, wherein the number of fibrous bundles is 3 to 7.

Coated Fiber Aspect 41. The fibrous composite of any of Coated Fiber Aspects 29 to 40, wherein the fibrous composite includes wicking channels.

Coated Fiber Aspect 42. A composite element comprising a core region include the fibrous composite of any of Coated Fiber Aspects 29 to 41 at least partially covered by a second region including a polymeric covering formed of a polymeric material.

Coated Fiber Aspect 43. A composite element comprising a core region including a plurality of fibrous bundles which are bound and/or interlocked by bias fiber elements wherein the core region includes a polymerizable material or polymeric material, wherein the composite element has a second region including a polymeric covering formed of a polymerizable material or a polymeric material.

Coated Fiber Aspect 44. The composite element of Coated Fiber Aspect 42 or 43, wherein the polymeric material of the second region is a filled polymeric material.

Coated Fiber Aspect 45. The composite element of Coated Fiber Aspect 44, wherein the filled polymeric material includes particles of a filler that are biodegradable or bioresorbable.

Coated Fiber Aspect 46. The composite element of any of Coated Fiber Aspects 44 or 45, wherein the filled polymeric material is a thermoset material or a thermoplastic material.

Coated Fiber Aspect 47. The composite element of any of Coated Fiber Aspects 42 to 46, wherein: (i) the filled polymeric material has a melt flow rate of about viscosity of about 1 g/10 min or more (preferably about 5 g/10 min or more, about 10 g/10 min or more, about 20 g/10 min or more, about 50 g/10 min or more, about 100 g/10 min or more, or about 150 g/10 min or more) as measured according to ASTM D1238-20 at 200° C./2.16 kg; or (ii) the polymeric material of the core regions (e.g., the coated fiber) has a viscosity of about the filled polymeric material has a melt flow rate of about viscosity of about 1 g/10 min or more (preferably about 5 g/10 min or more, about 10 g/10 min or more, about 20 g/10 min or more, about 50 g/10 min or more, about 100 g/10 min or more, or about 150 g/10 min or more) as measured according to ASTM D1238-20 at 200° C./2.16 kg; or both (i) and (ii).

Coated Fiber Aspect 48. The composite element of any of Coated Fiber Aspects 42 to 47, wherein the composite element can has a bending radius of about 20 cm or less (preferably about 10 cm or less, more preferably about 3 cm or less) at a bending angle of about 45°.

Coated Fiber Aspect 49. The composite element of any of Coated Fiber Aspects 42 to 48, wherein the composite element includes a plurality of the fibrous composites, wherein the fibrous composites are spaced apart in the core region and embedded in a polymeric matrix.

Coated Fiber Aspect 50. The composite element of Coated Fiber Aspect 49, wherein two or more of the fibrous composites are attached, connected, or positioned by a plurality of bias fiber elements that wrap, weave together, braid, interlace, or interlock the fibrous composites.

Coated Fiber Aspect 51. The composite element of any of Coated Fiber Aspects 42 to 50, wherein the composite element has a substantially uniform profile along its length and the filaments of the fibrous bundles extend a length of the composite element.

Coated Fiber Aspect 52. The composite element of Coated Fiber Aspect 51, wherein the profile is generally circular, elliptical, or polygonal, preferably wherein the profile is generally hexagonal or triagonal.

Coated Fiber Aspect 53. The composite element of Coated Fiber Aspect 51 or 52, wherein the core regions is covered by the polymeric material of the second region by a pultrusion process, where the coated core is passed through a die to form the desired profile.

Coated Fiber Aspect 54. The composite element of any of Coated Fiber Aspects 42 to 53, wherein the composite element is characterized by one or any combination of the following: a flexural modulus of about 20 GPa or more; or a compressive strength of about 20 GPa or more; or a compressive strain at failure of about 5% or more (preferably about 10% or more, and more preferably about 15% or more; or a tensile strength of about 20 GPa or more; or a bending radius of about 20 cm or less (preferably about 10 cm or less, 5 cm or less, or 2 cm or less).

Coated Fiber Aspect 55. The composite element of any of Coated Fiber Aspects 42 to 54, wherein the biodegradability or bioresorbability of the second region is different than that first region.

Coated Fiber Aspect 56. The composite element of any of Coated Fiber Aspects 42 to 55, wherein the composite element includes a compatibilizer or sizing material for adhering to an injectable thermosetting resin.

Coated Fiber Aspect 57. The composite element of any of Coated Fiber Aspects 42 to 56, wherein the composite element or the fibrous bundles has a non-circular cross-section so that the maximum packing can be increased, preferably the maximum packing density is about 0.910 or more, about 0.920 or more, about 0.930 or more, about 0.940 or more, about 0.960 or more, about 0.970 or more, about 0.980 or more, about 0.990 or more, or about 1.000; optionally wherein the cross-section is multi-lobed and/or has a rotational symmetry of 2 or more, or 3 or more.

Coated Fiber Aspect 58. The composite element of any of Coated Fiber Aspects 42 to 57, wherein the composite element is an implant.

Coated Fiber Aspect 59. An implant comprising: the composite element of any of Coated Fiber Aspects 42 to 57; and a covering.

Coated Fiber Aspect 60. The implant of Coated Fiber Aspect 59, wherein the implant is a screw or pin.

Coated Fiber Aspect 61. The implant of Coated Fiber Aspect 60, wherein the implant is a screw having a recessed end shaped for receiving a driving tool (recess may be in a head or in a shaft of a headless screw).

Coated Fiber Aspect 62. The implant of any of Coated Fiber Aspects 59 to 61, wherein the covering includes a threaded outer surface.

Coated Fiber Aspect 63. The implant of any of Coated Fiber Aspects 59 to 62, wherein the covering includes openings.

Coated Fiber Aspect 64. The implant of any of Coated Fiber Aspects 59 to 63, wherein the covering has a rough or porous surface.

Coated Fiber Aspect 65. The implant of any of Coated Fiber Aspects 59 to 64, wherein the covering is a polymeric material, preferably including one or more fillers, more preferably including one or more biodegradable or bioresorbable fillers.

Coated Fiber Aspect 66. The implant of any of Coated Fiber Aspects 59 to 65, wherein the implant is capable of being torqued by a driver without failure.

Coated Fiber Aspect 67. The implant of any of Coated Fiber Aspects 59 to 66, wherein the biodegradability or the bioresorbability of the covering is different than the composite element.

Coated Fiber Aspect 68. The use of the implant of any of Coated Fiber Aspects 59 to 67.

Coated Fiber Aspect 69. A polymerizable resin comprising one or more monomers and or one or more prepolymers, and one or more agents for activating or accelerating a cross-linking or polymerization reaction to form a polymer; wherein the polymerizable resin is a liquid at a temperature of about 23° C. (preferably having a viscosity of about 200,00 cps or less, about 20,000 cps or less, about 2,000 cps or less or about 200 cps or less, as measured according to ASTM D445-19a); the polymer does not flow at a temperature of about 40° C.; the polymerizable resin has a setting time (i.e., time until the material no longer flows) of about 2.0 minutes to about 120 minutes, at a temperature of about 35° C.; a temperature of the polymerizable resin does not exceed 45° C. during the setting; and the resulting polymer is biodegradable or bioresorbable.

Coated Fiber Aspect 70. The polymerizable resin of Coated Fiber Aspect 69, wherein the polymerizable resin includes a filler dispersed in a matrix of the monomers or prepolymers, wherein the filler is biodegradable or bioresorbable.

Coated Fiber Aspect 71. A kit for an implantable splint comprising: a container for inserting into a medullary canal; a plurality of the composite elements of any of Coated Fiber Aspects 42 to 57 for packing into the container; and a polymerizable resin for inserting into the container, preferably after the composite elements are introduced; preferably wherein the container is a containment bag.

Coated Fiber Aspect 72. The kit of Coated Fiber Aspect 71, wherein the polymerizable resin polymerizes and/or cross-links at a temperature of about 30° C. to about 50° C.

Coated Fiber Aspect 73. The kit of Coated Fiber Aspect 71 or 72, wherein the container is a containment bag or other container that constrains the flow of the polymerizable resin and prevents or reduces blood or other bodily fluids from entering the container while the polymerizable resin is polymerizing and/or cross-linking.

Coated Fiber Aspect 74. The kit of any of Coated Fiber Aspects 71 to 73, wherein the container (e.g., the containment bag) is bioresorbable and/or biodegradable.

Coated Fiber Aspect 75. The kit of any of Coated Fiber Aspects 71 to 74, wherein the container (e.g., the containment bag) has a surface that promotes osteointegration.

Coated Fiber Aspect 76. The kit of any of Coated Fiber Aspects 71 to 75, wherein the container (e.g., the containment bag) is formed of a polymeric material.

Coated Fiber Aspect 77. The kit of any of Coated Fiber Aspects 71 to 76, wherein the container (e.g., the containment bag) is formed from fibers, preferably fibers that are wove, braided or knitted.

Coated Fiber Aspect 78. The kit of any of Coated Fiber Aspects 71 to 77, wherein the kit includes a tube for inserting into the container (e.g., the containment bag) or the container (e.g., the containment bag) is a double walled bag, so that a hole can be created through a portion or an entire the length of the implant, preferably to connect the medullary canal above and below the implant.

Coated Fiber Aspect 79. The kit of any of Coated Fiber Aspects 71 to 78, wherein the polymerizable resin includes a biodegradable or bioresorbable filler.

Coated Fiber Aspect 80. The kit of any of Coated Fiber Aspects 71 to 79, wherein the kit includes about 6 or more of the composite elements having about the same length.

Coated Fiber Aspect 81. The kit of any of Coated Fiber Aspects 71 to 80, wherein the composite elements has a bending radius of about 10 cm or less for inserting into a catheter or other flexed tube for delivering one or more of the composite elements into a medullary canal.

Coated Fiber Aspect 82. The kit of any of Coated Fiber Aspects 71 to 81, wherein the polymerizable resin is the polymerizable resin of Coated Fiber Aspect 69 to 70.

Coated Fiber Aspect 83. The kit of any of Coated Fiber Aspects 71 to 82, wherein the kit includes a catheter for delivering one or more of the components into a medullar canal or other space.

Coated Fiber Aspect 84. The kit of Coated Fiber Aspect 83, wherein upon immersion in the salt water for an additional 7 weeks, the cylinders have a structure of interconnected pores.

Coated Fiber Aspect 85. The use of the kit of any of Coated Fiber Aspects 71 to 84 for forming an implant in a medullary canal.

Coated Fiber Aspect 86. A scaled (i.e., larger) composite element comprising two or more composite element of any of Coated Fiber Aspects 42 to 57, preferably wherein the composite elements are interlaced or interlocked or bounded by bias fiber elements, such as in a braiding.

By way of summary, without limitation, various of the following Aspects are contemplated within the present teachings. Also contemplated within the present teachings is a combination of any of the following Degradation Aspects with each and every one of the application claims as filed in this disclosure and as stated in the “Cross-Reference to Related Applications” section, as well as each and every one of the Coated Fiber Aspects and/or Composite Aspects previously discussed.

Degradation Aspect 1: a composite comprising: a degradable and bioabsorbable polymeric matrix material, and a plurality of fiber bundles dispersed in the polymeric matrix material, the plurality of fiber bundles including a plurality of degradable fibers; wherein the polymeric matrix material, the degradable fibers, or both are configured to be degradable according to a predetermined degradation profile, so the composite maintains a sufficient compressive load and, in the event of failure, fails in a ductile failure mode from a time of implantation until at least about 24 weeks after initiation of degradation; and wherein the composite occupies an envelope defined by a perimetric surface geometry and/or a volume of the composite; wherein the composite optionally contains any of the Coated Fiber Aspects 1 through 86 previously identified.

Degradation Aspect 2: The composite according to Degradation Aspect 1, wherein the polymeric matrix material includes poly(lactic acid) (e.g., PLA, PDLA, PLLA; most preferably PDLA 70/30), poly(lactic-co-glycolic acid) (e.g., PLGA 94/6), polyurethane, poly(glycolic acid), polyhydroxyalkanoates, citric acid based polymers, or any combination thereof.

Degradation Aspect 3. The composite according to Degradation Aspect 1 or Degradation Aspect 2, wherein the polymeric matrix material optionally includes a particulate filler, in addition to the plurality of degradable fibers dispersed within the polymeric matrix material.

Degradation Aspect 4. The composite according to Degradation Aspect 3, wherein the plurality of degradable fibers and the optional particulate filler comprise glass, magnesium hydroxide, or both.

Degradation Aspect 5. The composite according to Degradation Aspect 3 or Degradation Aspect 4, wherein the plurality of degradable fibers and/or the optional particulate filler, during degradation, release ionic species into an aqueous environment within the envelope.

Degradation Aspect 6. The composite according to Degradation Aspect 5, wherein an identity of the ionic species and/or a concentration of the ionic species in the aqueous environment modulates a pH of the aqueous environment.

Degradation Aspect 7. The composite according to any of Degradation Aspects 3 through 6, wherein the polymeric matrix material, the plurality of fiber bundles, the plurality of degradable fibers, the optional particulate filler, or any combination thereof is coated and/or filled with one or more compatibilizers that function to promote osseointegration.

Degradation Aspect 8. The composite according to any of Degradation Aspects 3 through 7, wherein the polymeric matrix material, the plurality of fiber bundles, the plurality of degradable fibers, the optional particulate filler, or any combination thereof comprise two or more distinct regions of material composition, each of the two or more distinct regions being configured to degrade at different rates.

Degradation Aspect 9. The composite according to Degradation Aspect 8, wherein the two or more distinct regions degrade in a generally sequential manner (i.e., one after another), in a generally staggered manner (i.e., overlapping), or both.

Degradation Aspect 10. The composite according to Degradation Aspect 8 or Degradation Aspect 9, wherein the two or more distinct regions vary in chemical composition over time during degradation.

Degradation Aspect 11. The composite according to any of the preceding Degradation Aspects, wherein at least one stage of the degradation profile includes: a first stage during which the composite includes a surface texture and/or a surface porosity allowing biological materials between about 0.1 nm and 1,000 nm in their largest dimension (e.g., proteins) to enter into the envelope, a first stage or second stage during which the composite includes a surface texture and/or a surface porosity allowing biological materials between about 1 μm and 30 μm in their largest dimension (e.g., macrophages) to enter into the envelope, an additional stage (e.g., third stage) during which the composite includes a surface texture and/or a surface porosity allowing biological materials between about 30 μm and 600 μm in their largest dimension (e.g., tissue and/or bone) to enter into the envelope.

Degradation Aspect 12. The composite according to any of the preceding Degradation Aspects, wherein the degradation profile includes at least one stage in which there is a surface texture and/or a surface porosity in the envelope, the surface texture and/or the surface porosity permitting washout of ionic species and/or other degradation byproducts (e.g., hydrolyzed polymeric matrix material) from within the envelope and into a surrounding environment of the envelope, the surrounding environment being no more than about 1 mm from the envelope, more preferably no more than 2 mm from the envelope, more preferably no more than 3 mm from the envelope, or even more preferably no more than about 4 mm from the envelope.

Degradation Aspect 13. The composite according to Degradation Aspect 12, wherein the surface texture and/or the surface porosity provides for washout of the ionic species and/or other degradation byproducts from within the envelope to the surrounding environment in a sufficient amount so that the pH within the envelope remains within a range of about 5.5 to 10 (e.g., about 5.5 to 7.5), for a period of at least about 24 weeks after initiation of degradation.

Degradation Aspect 14. The composite according to any of the preceding Degradation Aspects, wherein the degradation profile includes at least one stage in which a plurality of passages are formed in the composite, the plurality of passages providing washout from the composite and into a surrounding environment, for eliminating ionic species and/or other degradation byproducts from within the envelope.

Degradation Aspect 15. The composite according to Degradation Aspect 14, wherein the plurality of passages provides for washout of the ionic species and/or the other degradation byproducts from within the envelope to the surrounding environment in a sufficient amount so that the pH within the envelope remains within a range of about 5.5 to 10, for a period of at least about 24 weeks after initiation of degradation.

Degradation Aspect 16. The composite according to Degradation Aspect 14 or Degradation Aspect 15, wherein the plurality of passages include a plurality of axial passages (co-axial with the longitudinal axis of the composite), a plurality of transverse passages (co-axial with the transverse axis of the composite), a plurality of radial passages (radially oriented at an angle to the axial/transverse passages), or any combination thereof.

Degradation Aspect 17. The composite according to any of the preceding Degradation Aspects, wherein the predetermined degradation profile includes one or more stages characterized by a degradation onset, one or more degradation rates, or both.

Degradation Aspect 18. The composite according to any of the preceding Degradation Aspects, wherein a modulus of elasticity of the composite at least about 4 weeks after initiation of degradation is between about 8,000 MPa and 40,000 MPa, more preferably between about 9,000 MPa and 30,000 MPa, and more preferably between about 9,500 MPa and 25,000 MPa.

Degradation Aspect 19. The composite according to any of the preceding Degradation Aspects, wherein a poly dispersion index of the polymeric matrix material at least about 4 weeks after initiation of degradation is about 50% of the poly dispersion index of the polymeric matrix material prior to initiation of degradation.

Degradation Aspect 20. The composite according to any of the preceding Degradation Aspects, wherein at about 4 weeks from initiation of degradation, a weight of the composite is no more than about 10%, more preferably 5%, or even more preferably about 2% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

Degradation Aspect 21. The composite according to any of the preceding Degradation Aspects, wherein at about 12 weeks from initiation of degradation, a weight of the composite is no more than about 20% less, more preferably about 15% less, or even more preferably about 12% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

Degradation Aspect 22. The composite according to any of the preceding Degradation Aspects (and optionally including any of the Coated Fiber Aspects 1 through 86), wherein at about 24 weeks from initiation of degradation, a weight of the composite is about 30% less, more preferably about 25% less, or even more preferably about 22% less than the weight of the composite prior to degradation, as measured by storing the composite in a fixed volume of buffered saline solution maintained at 37° C. exposed to circulation/agitation (e.g., stir bar or pumped fluid) and an initial pH of about 7.5, and drying the composite prior to weighing.

Degradation Aspect 23. The composite according to any of the preceding Degradation Aspects (and optionally including any of the Coated Fiber Aspects 1 through 86), wherein at about 4 weeks from initiation of degradation, a compressive modulus of the composite is about 10 GPa or more, more preferably 15 GPa or more, or more preferably 20 GPa or more, as measured by storing the composite in a fixed volume of buffered saline solution maintained at 37° C. exposed to circulation/agitation (e.g., stir bar or pumped fluid) and an initial pH of about 7.5, and drying the composite prior to compressive testing.

Degradation Aspect 24. The composite according to any of the preceding Degradation Aspects (and optionally including any of the Coated Fiber Aspects 1 through 86), wherein at about 4 weeks from initiation of degradation, a strain at failure of the composite is about 10% or more, more preferably 15% or more, or more preferably 20% or more in bending, torsion, and/or compression, as measured by storing the composite orthopedic implant in a fixed volume of buffered saline solution maintained at 37° C. exposed to circulation/agitation (e.g., stir bar or pumped fluid) and an initial pH of 7.5, as measured by storing the composite in a fixed volume of buffered saline solution maintained at 37° C. exposed to circulation/agitation (e.g., stir bar or pumped fluid) and an initial pH of about 7.5, and drying the composite prior to compressive testing.

Degradation Aspect 25. The composite according to Degradation Aspect 1 (and optionally including any of the Coated Fiber Aspects 1 through 86), wherein the composite includes a composite orthopedic implant for implantation into a living being; and wherein the polymeric matrix material, the degradable fibers, or any combination thereof are configured to be degradable in vivo after implantation into the living being according to a predetermined degradation profile that corresponds with a bone and tissue ingrowth profile so that from the time of implantation until the wound site is healed, the composite orthopedic implant maintains a sufficient compressive load and, in the event of failure, fails in a ductile failure mode.

Degradation Aspect 26. The composite according to Degradation Aspect 25, wherein the composite orthopedic implant is characterized by a volume of between about 50 mm³ and 4 cm³ (i.e., “small volume implant”) or a volume of between about 4 cm³ and 25 cm³ (i.e., “medium volume implant”), or a volume of between about 25 cm³ and 300 cm³ (i.e., “large volume implant”).

Degradation Aspect 27. The composite according to Degradation Aspect 25 or Aspect 26, wherein the composite orthopedic implant is configured to resist torque, bending, or both.

Degradation Aspect 28. The composite according to any of Degradation Aspects 25 through 27, wherein the composite orthopedic implant is configured to affix bone, tissue, or both.

Degradation Aspect 29. The composite according to any of Degradation Aspects 25 through 28, wherein the composite orthopedic implant is in the form of a pin, a screw, an anchor, a nail, an assembly introduced into a containment bag in vivo (e.g., splint), a plate, or any combination thereof.

Degradation Aspect 30. The composite according to Degradation Aspect 29, wherein the composite implant is a screw, pin, anchor, nail, or plate having apertures.

Degradation Aspect 31. The composite implant according to Degradation Aspect 30, wherein the apertures are defined by openings between bias fiber bundles.

Degradation Aspect 32. The composite implant according to Degradation Aspect 31, wherein the bias fiber bundles are interlocked.

Degradation Aspect 33. The composite implant according to Degradation Aspect 30, wherein the apertures are defined by openings between axial fiber bundles.

Degradation Aspect 34. The composite implant according to Degradation Aspect 33, wherein the axial fiber bundles are interlocked by bias fiber bundles.

Degradation Aspect 35. The composite implant according to Degradation Aspect 30, wherein the apertures are defined by openings between bias fiber bundles and axial fiber bundles.

Degradation Aspect 36. The composite implant according to any of Degradation Aspects 31 through 35, wherein the axial fiber bundles comprise more than one fiber.

Degradation Aspect 37. The composite implant according to Degradation Aspect 36, wherein the axial bundle comprises between 2 and 40 fibers.

Degradation Aspect 36. The composite implant according to Degradation Aspect 34 or Degradation Aspect 35, wherein the fibers in the axial fiber bundle are bound together by bias fiber bundles.

Degradation Aspect 37. A method of forming a reinforcement element rod comprising the steps of: feeding one or more bundles of axial fibers through a braider having multiple carriers; and forming a braid around the bundles using bias fibers; wherein one or more carriers of the braider are not employed in the braiding so that a partially open braid structure is formed (optionally including any of the Coated Fiber Aspects 1 through 86).

Degradation Aspect 38. The method of Aspect 37, wherein about 5% or more (optionally about 10% or more, or about 15% or more) of the carriers are not employed in the braiding.

Degradation Aspect 39. A kit (optionally including any of the Coated Fiber Aspects 1 through 86), for an implant, a method for preparing an implant, or a hardenable material for an implant, wherein a hardenable material having with a short working lift or pot life, preferably about 10 minutes or less, about 8 minutes or less, about 5 minutes or less, about 3 minutes or less, about 2 minutes or less, or about 1 minutes or less, as measured according to ASTM D1144-99 (2021). By using a low concentration of thermoset relative to the concentration of the reinforcement rods, it is possible to dissipate heat during the cross-linking and extend the gel time. By using a catheter with a static mixer, it is possible to delay the start of the cross-linking reaction until the hardenable material is being inserted into the implant site.

Degradation Aspect 39. A kit for an implant comprising: a plurality of composite reinforcement rods (of a similar or different size, geometry and/or material) for inserting into a bone opening; and a two-part thermosetting material for filling a space between the composite reinforcement rods; wherein the thermosetting material is characterized by a gel time of about 60 seconds or less (optionally, about 40 seconds or less, about 30 seconds or less, or about 20 seconds or less), as measured according to ASTM D3056.

Degradation Aspect 40. The kit of Degradation Aspect 39, wherein the kit includes a catheter having dual channels, each for delivery of a different part of the two parts of the thermosetting material into the bone opening.

Degradation Aspect 41. The kit of Degradation Aspect 39 or 40, wherein the catheter has a mixing element (e.g., a static mixer) at or near a distal end for mixing the two parts.

Degradation Aspect 42. The kit of any of Degradation Aspects 39 to 41, wherein the reinforcement rods includes about 50 volume percent or less of a polymeric matrix and multiple bundles of axially aligned glass fibers which are attached, woven, or braided together with bias fibers, wherein an amount of fibers in the reinforcement rods is about 50 volume percent or more.

Degradation Aspect 43. An implant formed with the kit of any of Degradation Aspects 39 to 42, wherein the volume of the thermosetting material is about 20 volume percent or less based on a total volume of the thermosetting material and the composite reinforcement rods.

Degradation Aspect 44. A reinforment rod, or a kit including a reinforcement rod, or a method, wherein the reinforcement rod has a high stiffness and/or high strength for preparing a load bearing implant, wherein the reinforcement rod is capable of being inserted into a intermedullary canal of a bone, through a catheter inserted in a side opening of the bone, without breaking the reinforcement rod during the necessary bending.

Degradation Aspect 45. An implant comprising: reinforcement rods; and a thermosetting or thermoplastic polymeric material between the reinforcement rods and attaching the reinforcement rods; wherein the reinforcement rods includes about 50 volume percent or less of a polymeric matrix and multiple bundles of axially aligned glass fibers which are attached, woven, or braided together with bias fibers, wherein an amount of fibers in the reinforcement rods is about 50 volume percent or more; wherein the reinforcement rods are capable of being bent at a radius of about 10 mm, about 5 mm, or about 3 mm without breaking.

Degradation Aspect 46. The implant of Degradation Aspect 45, wherein each of the reinforcement rods has a plurality of fibrous bundles according to any of the above cited Coated Fiber Aspects 2 through 86.

Degradation Aspect 47. An implant (optionally, according to Degradation Aspect 46 or any of Degradation Aspects 1 through 45) having different regions that degrade at different rates, and or have peak degradation rates that occur at different times, preferably a ratio of the time from implantation to peak degradation rate of a first region to the time from implantation to peak degradation rate of a second region is about 1.5 or more, about 2.0 or more, about 2.5 or more, about 3.0 or more, about 4.0 or more, about 6.0 or more, or about 10.0 or more.

Degradation Aspect 48. An implant (optionally, according to Degradation Aspect 46 or 47) comprising: a first region consisting substantially of biodegradable and or biocompatible materials, including a first porogen; and a second region consisting substantially of biodegradable and or biocompatible materials, including a second porogen; wherein the first region and second region degrade at different rates such that upon submerging the implant in flowing water having a pH of about 7 and a temperature of about 35° C., when 30 volume percent of the first region has been removed, at least about 80 volume percent (or at least about 90 volume percent, or at least 95 volume percent) of the second region remains.

Degradation Aspect 49. The implant of Degradation Aspect 48, wherein the first porogen creates pores having a sufficient diameter to promote bone in-growth into the implant.

Degradation Aspect 50. The implant of Degradation Aspect 48 or 49, wherein the second region provides a scaffolding.

Degradation Aspect 51. A composite material (optionally, according to any of Degradation Aspects 1 through 50) including an axial region bound by a bias material, wherein the bias material is applied in such a way (e.g., using skipped carriers) that the bias material does not cover the axial material, and has an open structure.

Degradation Aspect 52. A method of forming a reinforcement rod comprising the stops of: feeding one or more bundles of axial fibers through a braider having multiple carriers; forming a braid around the bundles using bias fibers; wherein one or more carriers of the braider are not employed in the braiding so that a partially open braid structure is formed (optionally according to any of Degradation Aspects 1 through 50).

Degradation Aspect 53. The method of Degradation Aspect 52, wherein about 5% or more (optionally about 10% or more, or about 15% or more) of the carriers are not employed in the braiding.

Degradation Aspect 54. The method of Degradation Aspect 52 or 53, wherein the bias fibers are applied using a bias material that is generally a tape having an aspect ratio of about 2:1 or more, about 3:1 or more, or about 4:1 or more.

Unless otherwise stated herein, cross-section may refer to a cross-section along a transverse plane through the composite article, the transverse plane being orthogonal to the longitudinal axis of the composite article.

It should be understood that many additional changes in the details, materials, steps and arrangements of parts, which have been herein described and illustrated in order to explain the nature of the present invention, may be made by those skilled in the art while still remaining within the principles and scope of the invention.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the comparative teaching of amounts expressed as weight/volume percent for two or more ingredients also encompasses relative weight proportions of the two or more ingredients to each other, even if not expressly stated. For example, if a teaching recites 2% A, and 5% B, then the teaching also encompasses a weight ratio of A:B of 2:5. Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference in their entirety for all purposes.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of (namely, the presence of any additional elements, ingredients, components or steps, does not materially affect the properties and/or benefits derived from the teachings; or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

Unless otherwise stated, or unambiguously clear from the surrounding context, teachings that refer to a cover, or to a coating, also encompass a sheath.

### REFERENCE NUMERALS

2 Bone
4 Intramedullary canal
6 Fracture
10 Composite (e.g., composite orthopedic implant)
12 Pin
14 Screw
16 Splint
17 Containment bag
18 Plate
20 End
22 Longitudinal axis
24 Facet
26 Corner
28 Lobe
29 Aperture
30 Cannulation
32 Thread
34 Crest
36 Root
38 Major diameter
40 Minor diameter
42 Thread angle
43 Pitch
44 Head
45 Drive socket
46 Tip
50 Core
52 Outer region
54 Coating
60 Reinforcement element
62 Axial reinforcement element
64 Bias reinforcement element
66 Rod
68 Sheet
70 Tube
72 Roll
74 Tape
76 Minor axis
78 Major axis
80 Matrix material
82 Fiber
84 Axial fiber
86 Bias fiber
87 Textile
88 Fiber bundle
90 Axial fiber bundle
92 Bias fiber bundle
94 Fiber composite
96 Fracture path
98 Cell
100 Cell length
102 Cell width

1.-20. (canceled)

21. An elongated composite for an implant comprising:
i) a core region including a plurality of fiber bundles and a polymerizable matrix material or polymeric matrix material which fills gaps between the fiber bundles; and
ii) an outer region surrounding the core region;
wherein each of the fiber bundles comprises a plurality of continuous fibers and matrix material filling interstitial spaces between fibers; and
wherein each of the fibers includes a plurality of filaments, each filament coated with matrix material, and the outer region includes a coating free of continuous fibers.

22. The elongated composite of claim 21, wherein the elongated composite is configured for placement within a body of a live being as an orthopedic implant.

23. The elongated composite of claim 21, wherein the elongated composite exhibits a degradation profile that results in sufficient bone and tissue growth within the volume of the implant before degradation so that the elongated composite is load bearing.

24. The elongated composite of claim 21, wherein the core region has a flexural modulus of about 20 GPa or more, as measured according to ASTM D 790-17.

25. The elongated composite of claim 21, wherein the fiber bundle has a tensile modulus of about 10 GPa or more, as measured according to ASTM D638-14, using a fiber shaped specimen; or
the fiber bundle has a flexural modulus of about 5 GPa or more, as measured according to ASTM D 790-17; or
the fiber bundle has a bending radius of about 10 cm or less, as measured according to ASTM E290-14.

26. The elongated composite of claim 21, wherein the core region is formed of layers of sheet, wherein each layer includes one or more of the fiber bundles.

27. The elongated composite of claim 21, wherein the fiber bundles include coated fibers, wherein the coated fibers include a plurality of aligned inorganic filaments, wherein the filaments have a polymeric or polymerizable coating over at least a portion of their surface.

28. The elongated composite of claim 27, wherein
i) the coated fiber has a tensile modulus of about 10 GPa or more, as measured according to ASTM D638-14, using a fiber shaped specimen; or
ii) the coated fiber has a flexural modulus of about 5 GPa or more, as measured according to ASTM D 790-17; or
iii) the coated fiber has a bending radius of about 10 cm or less, as measured according to ASTM E290-14.

29. The elongated composite of claim 27, wherein the inorganic filaments are formed of a material having a specific gravity of about 2.80 or less.

30. The elongated composite of claim 21, wherein each of the fibers are braided or twisted, and/or each of the fiber bundles are braided or twisted.

31. The elongated composite of claim 21, wherein the elongated composite is a pin, a rod, a screw, an anchor, a nail or a plate.

32. The elongated composite of claim 21, wherein the elongated composite exhibits a modulus of elasticity, measured about 4 weeks after implantation, of 8,000 MPa to 40,000 MPa.

33. The elongated composite of claim 21, wherein the elongated composite includes threads formed in the outer region.

34. The elongated composite of claim 21, wherein the fibers are degradable, and wherein the fibers comprise glass or magnesium hydroxide.

35. The elongated composite of claim 21, wherein the polymeric or polymerizable material of the core region is a degradable and bioabsorbable material; and the plurality of fiber bundles include degradable fibers.

36. The elongated composite of claim 21, wherein an aspect ratio of the elongated composite is about 1:50 or more, and/or the aspect ratio is about 1:5 or less.

37. The elongated composite of claim 21, wherein the outer region comprises filler dispersed in a polymeric matrix.

38. The elongated composite of claim 37, wherein the filler is in the form of chopped fibers, nanofibers, nanotubes, flakes, and/or particulates.
